(19) United States
(12) Patent Application Publication
(10) Pub. No.: US 2025/0002455 A1
(43) Pub. Date: Jan. 2, 2025

(54) NEW N-PHENYLPYRROLAMIDE INHIBITORS OF DNA GYRASE AND TOPOISOMERASE IV WITH ANTIBACTERIAL ACTIVITY

(71) Applicant: UNIVERZA V LJUBLJANI, Ljubljana (SI)

(72) Inventors: Nace Zidar, Ljublijana (SI); Tihomir Tomasic, Ljublijana (SI); Janez Ilas, Ljublijana (SI); Martina Durcik, Ljublijana (SI); Anamarija Zega, Ljublijana (SI); Lucija Peterlin Masic, Ljublijana (SI); Danijel Kikelj, Ljublijana (SI)

(21) Appl. No.: 18/266,695

(22) PCT Filed: Dec. 16, 2021
(86) PCT No.: PCT/EP2021/086185

§ 371 Date: Jun. 12, 2023

(30) Foreign Application Priority Data
Dec. 17, 2020 (EP) 20215091.8
Dec. 23, 2020 (LU) LU102333

(51) Int. Cl. C07D 207/34; A61K 31/4025; A61K 31/41; A61K 31/4245; A61K 31/4439; A61K 31/454; A61K 31/496; A61K 31/506; A61K 31/5377; A61P 31/04; C07D 401/12; C07D 401/14; C07D 403/12; C07D 413/12; C07D 413/14

(52) U.S. Cl. CPC C07D 207/34; A61K 31/4025; A61K 31/41; A61K 31/4245; A61K 31/4439; A61K 31/454; A61K 31/496; A61K 31/506; A61K 31/5377; A61P 31/04; C07D 401/12; C07D 401/14; C07D 403/12; C07D 413/12; C07D 413/14

(57) ABSTRACT

The present invention relates to compounds having a structure of general formula (I), processes for their preparation, pharmaceutical compositions containing them as the active ingredient, to their use as medicaments and to their use in the manufacture of medicaments for use in the treatment of bacterial infections in humans and warm-blooded animals.

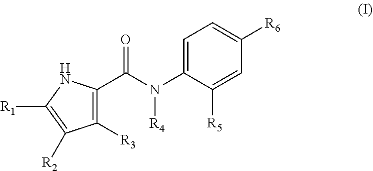

### TECHNICAL FIELD

The present invention relates to compounds that demonstrate antibacterial activity, processes for their preparation, pharmaceutical compositions containing them as the active ingredient, to their use as medicaments and to their use in the manufacture of medicaments for use in the treatment of bacterial infections in humans and warm-blooded animals.

### BACKGROUND ART

Antibiotic-resistant infections kill an estimated 700.000 people each year and the negative trend is becoming increasingly alarming (Tagliabue, A. et al. Front Immunol, 2018, 9, 1068; Brown, E D et al., Nature, 2016, 529, 336). In the last 50 years, only a small number of new chemical classes of antibacterials have reached clinical practice, while at the same time, the number of multi-drug resistant (MDR) bacteria is rising (Klahn, P. et al. Curr Top Microbiol Immunol, 2016, 398, 365). Recently, the World Health Organization (WHO) has issued a list of drug-resistant bacteria that pose the greatest threat to human health (Tacconelli, E., Magrini, N. et al. WHO, 2017; Willyard, C. Nature, 2017, 543, 15). Examples of such difficult-to-treat bacteria include pathogens of the “ESKAPE” group (Enterococcus faecium, Staphylococcus aureus, Klebsiella pneumoniae, Acinetobacter baumannii, Pseudomonas aeruginosa, and Enterobacter species) (Jasarevic, T., Chaib, T. WHO News release, 2020) and mycobacteria. The field of present invention is the discovery of new N-phenylpyrrolamide inhibitors of DNA gyrase and topoisomerase IV with activity against different bacterial strains.

Bacterial type II topoisomerases, DNA gyrase and topoisomerase IV, are enzymes that catalyse changes in DNA topology during DNA replication, transcription and recombination and are essential for bacterial growth (Bisacchi, G. S. et al. ACS Infect Dis, 2015, 1, 4). They are absent in higher eukaryotes, which makes them excellent targets for antibacterial drug discovery. DNA gyrase is composed of two GyrA and two GyrB subunits, while topoisomerase IV is composed of two ParC and two ParE subunits that are similar in structure to GyrA and GyrB, respectively. The main function of GyrA/ParC is cleavage and reunion of the DNA molecule, whereas GyrB/ParE binds ATP, providing through its hydrolysis, the energy for the ligation process (Durcik, M. et al. Expert Opin Ther Pat, 2019, 129).

Drugs targeting DNA gyrase or topoisomerase IV exert their antibacterial activity through two main mechanisms. The first is stabilisation of the covalent enzyme-DNA complex through binding to the GyrA/ParC active site (e.g. fluoroquinolones). The second mechanism, which is so far less exploited, involves inhibition of the ATP binding site on the GyrB/ParE subunit (e.g. novobiocin). Novobiocin is the only ATP-competitive DNA gyrase inhibitor that has ever progressed to the clinic, but it was later withdrawn due to its toxicity and low effectiveness. There are currently no ATPase inhibitors of DNA gyrase or topoisomerase IV in the clinical use. In recent decades, several scaffolds have been identified as ATPase GyrB/ParE inhibitors, such as pyridylureas (Basarab, G. S. et al., J Med Chem, 2013, 56, 8712), pyrimidinoindoles (Tari, L. W. et al. Plos One, 2013, 8, e84409), benzimidazole ureas (Grillot, A. L. et al. J Med Chem, 2014, 57, 8792), pyrrolamides (Basarab, G. S. et al. J Med Chem 2014, 57, 6060), and pyrazolopyridones (Cross, J. B. et al. ACS Med Chem Lett, 2016, 7, 374). Many of these compounds have problems of insufficient activity, low water solubility, cytotoxicity, or production of reactive metabolites. Therefore, there is a need for the development of novel effective GyrB/ParE inhibitors that do not possess the disadvantages of the known inhibitors.

In the recent decades, there has been extensive research directed at discovering new ATP-competitive GyrB/ParE inhibitors (Durcik, M. et al. Expert Opin Ther Pat, 2019, 129; Bisacchi, G. S. et al. ACS Infect Dis, 2015, 1, 4). For example, coumarin-containing compounds are described in patent application WO 99/35155, 5,6-bicyclic heteroaromatic compounds are described in patent application WO 02/060879, pyrazole compounds are described in patent application WO 01/52845 (U.S. Pat. No. 6,608,087) as well as in Daiichi Sankyo patents WO 2017/056012 A1 and WO2009084614A1. AstraZeneca has also published many applications describing antibacterial compounds, e.g. WO2005/026149, WO2006/087544, WO2006/087548, WO2006/087543, WO/2006/092599, WO2006/092608, WO 2007071965, WO2008/020227, WO2008/020229, WO2008/020222, WO2008/152418, WO2009/147431, WO2010/067125 and WO2010/067123.

The present invention relates to the discovery a new class of compounds with N-phenylpyrrolamide general structure that inhibit ATPase domains of DNA gyrase and topoisomerase IV and possess activity against different bacterial strains.

### SUMMARY OF THE INVENTION

We have discovered a new class of compounds that inhibit ATPase domains of DNA gyrase and/or topoisomerase IV. The compounds of the present invention are effective against bacteria.

The present invention relates to compounds of the formula I:

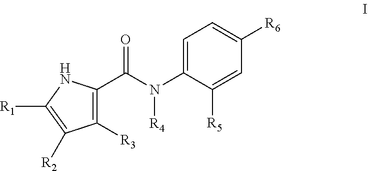

wherein R¹, R², R³, R⁴, R⁵ and R⁶ are as defined herein, and their pharmaceutically acceptable salts, racemates, diastereomers, enantiomers, esters, carbamates, sulphates, phosphates and prodrugs thereof.

Particularly, the present invention can be summarized by the following items:

1. The compound of formula I:

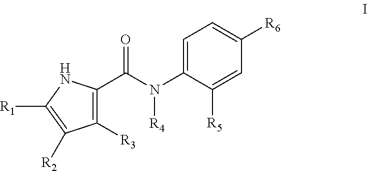

wherein:
R¹, R² and R³ are each independently selected from H, halogen, —CN, —CF₃, optionally substituted C₁₋₆ alkyl, optionally substituted C₁₋₆ alkoxy, and optionally substituted C₃₋₆ cycloalkyl;
R⁴ is (CH₂)₀₋₆-A
   wherein A is H, carboxyl, NR^{6′}R^{6″} or is selected from optionally substituted monocyclic C₃₋₇ cycloalkyl, optionally substituted monocyclic C₃₋₇ cycloalkenyl, optionally substituted saturated or unsaturated monocyclic 3-7 membered heterocycle, optionally substituted saturated or unsaturated fused bicyclic 8-10 membered-heterocycle, optionally substituted C₆₋₁₀ aryl and optionally substituted 5-10 membered heteroaryl;
R⁵ is H, hydroxy, halogen, carboxyl, C₁₋₆ alkyl, C₂₋₆ alkenyl, C₂₋₆ alkynyl, (CH₂)ₘO—C₁₋₆ alkyl, (CH₂)ₘS—C₁₋₆ alkyl, (CH₂)ₘS(═O)—C₁₋₆ alkyl, (CH₂)ₘO(CH₂)ₘ—C₃₋₇ cycloalkyl, (CH₂)ₘ—C₃₋₇ cycloalkyl, (CH₂)ₘO(CH₂)ₘ-6-10-membered aryl, (CH₂)ₘ-6-10-membered aryl, (CH₂)ₘO(CH₂)ₘ-5-10-membered heterocycle, (CH₂)ₘ-5-10-membered heterocycle, halogeno-C₁₋₆ alkyl, cyano or (CH₂)ₘNR^{5′}R^{5″}, wherein each m is an integer independently selected from 0, 1, 2 and 3, and wherein any alkyl, alkenyl, alkynyl, cycloalkyl, heterocycle or aryl may be optionally substituted;
R^{5′} and R^{5″} are each independently selected from H or C₁₋₆ alkyl, C₃₋₆ cycloalkyl and 4-6-membered heterocyclyl, wherein any alkyl, cycloalkyl or heterocyclyl may be optionally substituted, or R^{5′} and R^{5″} join together to form an optionally substituted 4-6-membered heterocyclyl and where each m is an integer independently selected from 0, 1, 2 and 3; preferably R^{5′} is H and R^{5″} is optionally substituted 4-6-membered heterocyclyl; more preferably R^{5′} is H and R^{5″} is optionally substituted piperidine;
R⁶ is (hydroxyimino)methyl, —CO—C₁₋₆ alkyl, —CO—C₂₋₆ alkenyl, —CO—C₂₋₆ alkynyl, carboxyl, —COO—C₁₋₆ alkyl, —CONR^{6′}R^{6″}, —OCONR^{6′}R^{6″}, —OCO—C₁₋₆ alkyl, —COO—C₁₋₆ alkyl (optionally substituted with —COO—C₁₋₆ alkyl), —CONHCH(CO₂R^{6″})R^{6′}, —(CH₂)₀₋₆OH, —O—C₁₋₆ alkyl, —OR^{6′}, —NH—C₁₋₆ alkyl, —NH—C₁₋₆ acyl, —NR^{6′}R^{6″}, hydroxyl-C₁₋₆ alkyl, 5-10-membered heterocyclyl, 6-10-membered aryl, —C(═NOR^{6″})C₁₋₆ alkyl, —C(═NOR^{6″})NR^{6′}R^{6″}, —S(O)pNR^{6′}R^{6″}, —S(O)p-C₁₋₆ alkyl-CONHR^{6″}, —C(O)NHS(O)p-C₁₋₆ alkyl, —C(O)NHS(O)p-aryl, —CH₂CH(COOR^{6′})OH, —C₁₋₆ alkyl-CH(NR^{6′}R^{6″})COOR^{6′} or —C₁₋₆ alkyl-CH(NR^{6′}R^{6″})CONR^{6′}R^{6″}), wherein any alkyl, alkenyl, alkynyl, heterocyclyl or aryl may be optionally substituted;
R^{6′} and R^{6″} are independently at each occurrence selected from H and optionally substituted C₁₋₆ alkyl or C₁₋₆ acyl;
or R^{6′} and R^{6″} may together with the nitrogen to which they are attached form a 5 or 6-membered heterocyclic ring, optionally substituted with 1 or 2 substituents independently selected from C₁₋₆ alkyl, C₂₋₆ alkenyl, C₂₋₆ alkynyl, hydroxyl, C₁₋₆ alkoxy, halogen, cyano, nitro, carboxyl, hydroxy-C₁₋₆ alkyl-, C₁₋₆ alkoxy-C₁₋₆ alkyl, halogeno-C₁₋₆ alkyl-, difluoromethyl, trifluoromethyl, formyl, —CO—C₁₋₆ alkyl, —COO—C₁₋₆ alkyl, —C(O)NH₂, —C(O)NH—C₁₋₆ alkyl, —C(O)N[C₁₋₆ alkyl]₂, —SO₂NH₂, —SO₂NH—C₁₋₆ alkyl, —SO₂N[C₁₋₆ alkyl]₂ and —S(O)p-C₁₋₆ alkyl;
p is (independently at each occurrence) 0, 1 or 2;
or a pharmaceutically acceptable salt, racemate, diastereomer, enantiomer, ester, carbamate, sulphate, phosphate or prodrug thereof.

2. The compound of item 1, wherein
R¹, R² and R³ are each independently selected from H, halogen, —CN, —CF₃, optionally substituted C₁₋₄ alkyl, optionally substituted C₁₋₄ alkoxy, and optionally substituted C₃₋₆ cycloalkyl;
R⁴ is (CH₂)₀₋₆-A
   wherein A is H, carboxyl, NR^{6′}R^{6″} or is selected from optionally substituted monocyclic C₃₋₇ cycloalkyl, optionally substituted monocyclic C₃₋₇ cycloalkenyl, optionally substituted saturated or unsaturated monocyclic 3-7 membered heterocycle, optionally substituted saturated or unsaturated fused bicyclic 8-10 membered-heterocycle, optionally substituted C₆₋₁₀ aryl and optionally substituted 5-10 membered heteroaryl;
R⁵ is H, hydroxy, halogen, carboxyl, C₁₋₆ alkyl, C₂₋₆ alkenyl, C₂₋₆ alkynyl, (CH₂)ₘO—C₁₋₆ alkyl, (CH₂)ₘS—C₁₋₆ alkyl, (CH₂)ₘS(═O)—C₁₋₆ alkyl, (CH₂)ₘO(CH₂)ₘ—C₃₋₇ cycloalkyl, (CH₂)ₘ—C₃₋₇ cycloalkyl, (CH₂)ₘO(CH₂)ₘ-6-10-membered aryl, (CH₂)ₘ-6-10-membered aryl, (CH₂)ₘO(CH₂)ₘ-5-10-membered heterocycle, (CH₂)ₘ-5-10-membered heterocycle, halogeno-C₁₋₆ alkyl, cyano or (CH₂)ₘNR^{5′}R^{5″}, wherein each m is an integer independently selected from 0, 1, 2 and 3, and wherein any alkyl, alkenyl, alkynyl, cycloalkyl, heterocycle or aryl may be optionally substituted;
R^{5′} and R^{5″} are each independently selected from H or C₁₋₆ alkyl, C₃₋₆ cycloalkyl and 4-6-membered heterocyclyl, wherein any alkyl, cycloalkyl or heterocyclyl may be optionally substituted, or R^{5′} and R^{5″} join together to form an optionally substituted 4-6-membered heterocyclyl and where each m is an integer independently selected from 0, 1, 2 and 3; preferably R^{5′} is H and R^{5″} is optionally substituted 4-6-membered heterocyclyl; more preferably R^{5′} is H and R^{5″} is optionally substituted piperidine;
R⁶ is (hydroxyimino)methyl, —CO—C₁₋₆ alkyl, —CO—C₂₋₆ alkenyl, —CO—C₂₋₆ alkynyl, carboxyl, —COO—C₁₋₆ alkyl, —CONR^{6′}R^{6″}, —OCONR^{6′}R^{6″}, —OCO—C₁₋₆ alkyl, —COO—C₁₋₆ alkyl (optionally substituted with —COO—C₁₋₆ alkyl), —CONHCH(CO₂R^{6″})R^{6′}, —(CH₂)₀₋₆OH, —O—C₁₋₆ alkyl, —OR^{6′}, —NH—C₁₋₆ alkyl, —NH—C₁₋₆ acyl, —NR^{6′}R^{6″}, hydroxyl-C₁₋₆ alkyl, 5-10-membered heterocyclyl, 6-10-membered aryl, —C(═NOR^{6″})C₁₋₆ alkyl, —C(═NOR^{6″})NR^{6′}R^{6″}, —S(O)pNR^{6′}R^{6″}, —S(O)p-C₁₋₆ alkyl-CONHR^{6″}, —C(O)NHS(O)p-C₁₋₆ alkyl, —C(O)NHS(O)p-aryl, —CH₂CH(COOR^{6′})OH, —C₁₋₆ alkyl-CH(NR^{6′}R^{6″})COOR^{6′} or —C₁₋₆ alkyl-CH(NR^{6′}R^{6″})CONR^{6′}R^{6″}), wherein any alkyl, alkenyl, alkynyl, heterocyclyl or aryl may be optionally substituted;
R^{6′} and R^{6″} are independently at each occurrence selected from H and optionally substituted C₁₋₆ alkyl or C₁₋₆ acyl;
or R¹ and R^{6″} may together with the nitrogen to which they are attached form a 5 or 6-membered heterocyclic ring, optionally substituted with 1 or 2 substituents independently selected from C₁₋₆ alkyl, C₂₋₆ alkenyl, C₂₋₆ alkynyl, hydroxyl, C₁₋₆ alkoxy, halogen, cyano, nitro, carboxyl, hydroxy-C₁₋₆ alkyl-, C₁₋₆ alkoxy-C₁₋₆ alkyl, halogeno-C₁₋₆ alkyl-, difluoromethyl, trifluoromethyl, formyl, —CO—C₁₋₆ alkyl, —COO—C₁₋₆ alkyl, —C(O)NH₂, —C(O)NH—C₁₋₆ alkyl, —C(O)N[C₁₋₆ alkyl]₂, —SO₂NH₂, —SO₂NH—C₁₋₆ alkyl, —SO₂N[C₁₋₆ alkyl]₂ and —S(O)p-C₁₋₆ alkyl;
p is (independently at each occurrence) 0, 1 or 2.

3. The compound of item 1 or 2, wherein two of R¹, R² and R³ are not H, and at least one of said two of R¹, R² and R³, which are not H, is halogen.

4. The compound of any one of items 1 to 3, wherein two of R¹, R² and R³ are halogens, preferably selected from bromo and chloro.

5. The compound of any one of items 1 to 4, wherein R¹ is methyl.

6. The compound of any one of items 1 to 5, wherein R² and R³ are chloro.

7. The compound of any one of items 1 to 5, wherein R² and R³ are fluoro.

8. The compound of any one of items 1 to 4, wherein R¹ and R² are bromo and R³ is H.

9. The compound of any one of items 1 to 8, wherein R⁴ is H.

10. The compound of any one of items 1 to 8, wherein R⁴ is (CH₂)₁₋₆-A.

11. The compound of any one of items 1 to 10, wherein R⁵ is optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle, optionally substituted (CH₂)ₘ-5-10-membered heterocycle or optionally substituted (CH₂)NR^{5′}R^{6″}, wherein each m is an integer independently selected from 0, 1, 2 and 3.

12. The compound of any one of items 1 to 11, wherein R⁵ is optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle or optionally substituted (CH₂)ₘ-5-10-membered heterocycle, wherein each m is an integer independently selected from 0, 1, 2 and 3.

13. The compound of any one of items 1 to 11, wherein R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3.

14. The compound of any one of items 1 to 11, wherein R⁵ is optionally substituted (CH₂)ₘ-5-10-membered heterocycle with m being an integer selected from 0, 1, 2 and 3.

15. The compound of any one of items 1 to 11, wherein R⁵ is optionally substituted (CH₂)ₘ-5-6-membered heterocycle with m being an integer selected from 0, 1, 2 and 3.

16. The compound of any one of items 1 to 11, wherein R⁵ is optionally substituted (CH₂)ₘ-6-membered heterocycle with m being an integer selected from 0, 1, 2 and 3.

17. The compound of any one of items 1 to 11, wherein R⁵ is optionally substituted (CH₂)ₘNR^{5′}R^{5″}; preferably wherein R^{5′} is H and R^{5″} is optionally substituted 4-6-membered heterocyclyl; more preferably R^{5′} is H and R^{5″} is optionally substituted piperidine.

18. The compound of any one of items 1 to 11, wherein R⁵ is selected from the group consisting of O(CH₂)ₘ isopropyl, O(CH₂)ₘ-pyrrolidine, O(CH₂)ₘ-piperidine, O(CH₂)ₘ-morpholine, O(CH₂)ₘ-pyridine, O(CH₂)ₘ-pyrimidine, O(CH₂)ₘ-thiophene, O(CH₂)ₘ-phenyl, (CH₂)ₘ-isopropyl, (CH₂)ₘ-pyrrolidine, (CH₂)ₘ-piperidine, (CH₂)ₘ-morpholine, (CH₂)ₘ-pyridine, (CH₂)ₘ-pyrimidine, (CH₂)ₘ-thiophene and (CH₂)ₘ-phenyl, wherein each m is an integer independently selected from 0, 1 or 2, and wherein any alkyl, heterocyclyl or aryl may be optionally substituted.

19. The compound of any one of items 1 to 11, wherein R⁵ is selected from the group consisting of O(CH₂)ₘ isopropyl, O(CH₂)ₘ-pyrrolidine, O(CH₂)ₘ-piperidine, O(CH₂)ₘ-morpholine, O(CH₂)ₘ-pyridine, O(CH₂)ₘ-pyrimidine, O(CH₂)ₘ-thiophene, O(CH₂)ₘ-phenyl, wherein each m is an integer independently selected from 0, 1 or 2, and wherein any alkyl, heterocyclyl or aryl may be optionally substituted.

20. The compound of any one of items 1 to 11, wherein R⁵ is selected from the group consisting of O(CH₂)ₘ pyrrolidine, O(CH₂)ₘ-piperidine, O(CH₂)ₘ-morpholine, O(CH₂)ₘ-pyridine, O(CH₂)ₘ-pyrimidine, O(CH₂)ₘ-thiophene, (CH₂)ₘ-pyrrolidine, (CH₂)ₘ-piperidine, (CH₂)ₘ-morpholine, (CH₂)ₘ-pyridine, (CH₂)ₘ-pyrimidine and (CH₂)ₘ thiophene, wherein each m is an integer independently selected from 0, 1 or 2, and wherein any alkyl, heterocyclyl or aryl may be optionally substituted.

21. The compound of any one of items 11 to 20, wherein each m is 0.

22. The compound of any one of items 11 to 20, wherein each m is 1.

23. The compound of any one of items 11 to 20, wherein each m is 2.

24. The compound of any one of items 1 to 11, wherein the optionally substituted heterocycle in R⁵ is selected from the group consisting of piperidine, piperidin-3-amine, piperidin-4-amine, piperidin-3-ylmethanamine, piperazine, pyrrolidine, pyrrolidin-3-amine, morpholine, isoindoline and 1-phenylpiperazine.

25. The compound of any one of items 1 to 10, wherein R⁵ is H.

26. The compound of any one of items 1 to 10, wherein R⁵ is OR^{5′}.

27. The compound of any one of items 1 to 26, wherein R⁶ is an optionally substituted 5-10-membered heterocyclyl, optionally substituted 6-10-membered aryl, —CONR^{6′}R^{6″}, —C(O)NHS(O)p-C₁₋₆ alkyl or —CONHCH(CO₂R^{6″})R^{6′}.

28. The compound of any one of items 1 to 26, wherein R⁶ is an optionally substituted 5-10-membered heterocyclyl, optionally substituted 6-10-membered aryl, or —CONHCH(CO₂R^{6″})R^{6′}.

29. The compound of any one of items 1 to 26, wherein R⁶ is an optionally substituted 5-10-membered heterocyclyl, —CONR^{6′}R^{6″} or —C(O)NHS(O)p-C₁₋₆ alkyl.

30. The compound of any one of items 1 to 26, wherein R⁶ is an optionally substituted 5-10-membered heterocyclyl or —CONHCH(CO₂R^{6″})R^{6′}.

31. The compound of any one of items 1 to 26, wherein R⁶ is an optionally substituted 5-10-membered heterocyclyl.

32. The compound of any one of items 1 to 26, wherein R⁶ is an optionally substituted 5-membered heterocyclyl.

33. The compound of any one of items 27 to 32, wherein the optionally substituted 5-10-membered heterocyclyl is selected from the group consisting of 1,2,4-oxadiazol-5-one, tetrazole, 1,3,4-oxadiazol-2-one and 1,4-dihydro-tetrazol-5-one.

34. The compound of any one of items 1 to 26, wherein R⁶ is —CONHCH(CO₂R^{6″})R^{6′}.

35. The compound of any one of items 1 to 26, wherein R⁶ is —CONHCH₂(CO₂H).

36. The compound of any one of items 1 to 26, wherein R⁶ is —CONR^{6′}R^{6″}.

37. The compound of any one of items 1 to 26, wherein R⁶ is —C(O)NHS(O)p-C₁₋₆ alkyl.

38. The compound of any one of items 1 to 26, wherein R⁶ is carboxyl or optionally alkylated or acylated nitrogen.

39. The compound of item 1, wherein:
R¹ is methyl;
R² and R³ are each independently selected from chloro, bromo or fluoro; preferably R² and R³ are chloro;
R⁴ is selected from hydrogen, —(CH₂)₁₋₆-A, wherein A is optionally substituted aryl or optionally substituted heterocyclyl;
R⁵ is selected from hydrogen, OR^{5′}, optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2, preferably wherein each m is an integer independently selected from 0 and 1; and
R⁶ is optionally substituted 6-10-membered aryl, optionally substituted 5-10-membered heterocyclyl or —CONHCH(CO₂R^{6″})R^{6′}.

40. The compound of item 1, wherein:
R¹ is methyl;
R² and R³ are each independently selected from chloro, bromo or fluoro; preferably R² and R³ are chloro;
R⁴ is hydrogen;
R⁵ is selected from hydrogen, OR^{5′}, optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2, preferably wherein each m is 0 or 1, more preferably wherein each m is 0; and
R⁶ is optionally substituted 5-10-membered heterocyclyl, optionally substituted 6-10-membered aryl, or —CONHCH(CO₂R^{6″})R^{6′}.

41. The compound of item 1, wherein:
R¹ is methyl;
R² and R³ are each independently selected from chloro, bromo or fluoro; preferably R² and R³ are chloro;
R⁴ is selected from hydrogen, —(CH₂)₁₋₆-A, wherein A is optionally substituted aryl or optionally substituted heterocyclyl;
R⁵ is selected from optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2, preferably wherein each m is an integer independently selected from 0 and 1; and
R⁶ is optionally substituted 6-10-membered aryl, optionally substituted 5-10-membered heterocyclyl or —CONHCH(CO₂R^{6″})R^{6′}.

42. The compound of item 1, wherein:
R¹ is methyl;
R² and R³ are each independently selected from chloro, bromo or fluoro; preferably R² and R³ are chloro;
R⁴ is hydrogen;
R⁵ is selected from optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2, preferably wherein each m is 0 or 1, more preferably wherein each m is 0; and R⁶ is optionally substituted 5-10-membered heterocyclyl, optionally substituted 6-10-membered aryl, or —CONHCH(CO₂R^{6″})R^{6′}.

43. The compound of item 1, wherein:
R¹ is methyl;
R² and R³ are each independently selected from chloro, bromo or fluoro; preferably R² and R³ are chloro;
R⁴ is hydrogen;
R⁵ is hydrogen; and
R⁶ is optionally substituted 5-10-membered heterocyclyl, optionally substituted 6-10-membered aryl, or —CONHCH(CO₂R^{6″})R^{6′}.

44. The compound of item 1, wherein:
R¹ is methyl;
R² and R³ are each independently selected from chloro, bromo or fluoro; preferably R² and R³ are chloro;
R⁴ is hydrogen;
R⁵ is OR^{5′}; and
R⁶ is optionally substituted 5-10-membered heterocyclyl, optionally substituted 6-10-membered aryl, or —CONHCH(CO₂R^{6″})R^{6′}.

45. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2, preferably wherein each m is 0 or 1, more preferably wherein each m is 0; and
R⁶ is optionally substituted 5-10-membered heterocyclyl, optionally substituted 6-10-membered aryl, or —CONHCH(CO₂R^{6″})R^{6′}.

46. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2, preferably wherein each m is 0 or 1, more preferably wherein each m is 0; and
R⁶ is optionally substituted 5-10-membered heterocyclyl or —CONHCH(CO₂R^{6″})R^{6′}.

47. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2, preferably wherein each m is 0 or 1, more preferably wherein each m is 0; and
R⁶ is optionally substituted 5-10-membered heterocyclyl, optionally substituted 6-10-membered aryl, or —CONHCH(CO₂R^{6″})R^{6′}.

48. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2, preferably wherein each m is 0 or 1, more preferably wherein each m is 0; and
R⁶ is optionally substituted 5-10-membered heterocyclyl or —CONHCH(CO₂R^{6″})R^{6′}.

49. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2, preferably wherein each m is 0 or 1, more preferably wherein each m is 0; and
R⁶ is carboxyl or optionally alkylated or acylated nitrogen.

50. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5′}, wherein each m is an integer independently selected from 0, 1 and 2, preferably wherein each m is 0 or 1, more preferably wherein each m is 0; and
R⁶ is optionally substituted 5-10-membered heterocyclyl or —CONHCH(CO₂R^{6″})R^{6′}.

51. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle or optionally substituted (CH₂)ₘ-5-10-membered heterocycle, wherein each m is an integer independently selected from 0, 1 and 2, preferably wherein each m is 0 or 1, more preferably wherein each m is 0; and
R⁶ is optionally substituted 5-10-membered heterocyclyl or —CONHCH(CO₂R^{6″})R^{6′}.

52. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘ-5-10-membered heterocycle, wherein m is an integer selected from 0, 1 and 2, preferably wherein m is 0 or 1, more preferably wherein m is 0; and
R⁶ is optionally substituted 5-10-membered heterocyclyl or —CONHCH(CO₂R^{6″})R^{6′}.

53. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, wherein each m is an integer independently selected from 0, 1 and 2, preferably wherein each m is 0 or 1, more preferably wherein each m is 0; and
R⁶ is optionally substituted 5-10-membered heterocyclyl or —CONHCH(CO₂R^{6″})R^{6′}.

54. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘNR^{5′}R^{5′}, wherein each m is an integer independently selected from 0, 1 and 2, preferably wherein each m is 0 or 1, more preferably wherein each m is 0; and
R⁶ is optionally substituted 5-10-membered heterocyclyl or —CONHCH(CO₂R^{6″})R^{6′}.

55. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, wherein each m is an integer independently selected from 0, 1 and 2, preferably wherein each m is 0 or 1, more preferably wherein each m is 0; and
R⁶ is optionally substituted 5-10-membered heterocyclyl.

56. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘNR^{5′}R^{5′}, wherein each m is an integer independently selected from 0, 1 and 2, preferably wherein each m is 0 or 1, more preferably wherein each m is 0; and
R⁶ is optionally substituted 5-10-membered heterocyclyl.

57. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘ-5-10-membered heterocycle, wherein m is an integer selected from 0, 1 and 2, preferably wherein m is 0 or 1, more preferably wherein m is 0; and
R⁶ is optionally substituted 5-10-membered heterocyclyl.

58. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2, preferably wherein each m is 0 or 1, more preferably wherein each m is 0; and
R⁶ is 1,2,4-oxadiazol-5-one, tetrazole, 1,3,4-oxadiazol-2-one or 1,4-dihydro-tetrazol-5-one.

59. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle or optionally substituted (CH₂)ₘ-5-10-membered heterocycle, wherein each m is an integer independently selected from 0, 1 and 2, preferably wherein each m is 0 or 1, more preferably wherein each m is 0; and
R⁶ is 1,2,4-oxadiazol-5-one, tetrazole, 1,3,4-oxadiazol-2-one or 1,4-dihydro-tetrazol-5-one.

60. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘ-5-10-membered heterocycle, wherein m is an integer selected from 0, 1 and 2, preferably wherein m is 0 or 1, more preferably wherein m is 0; and
R⁶ is 1,2,4-oxadiazol-5-one, tetrazole, 1,3,4-oxadiazol-2-one or 1,4-dihydro-tetrazol-5-one.

61. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, wherein each m is an integer independently selected from 0, 1 and 2, preferably wherein each m is 0 or 1, more preferably wherein each m is 0; and
R⁶ is 1,2,4-oxadiazol-5-one, tetrazole, 1,3,4-oxadiazol-2-one or 1,4-dihydro-tetrazol-5-one.

62. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘNR^{5′}R^{5″}, wherein each m is an integer independently selected from 0, 1 and 2, preferably wherein each m is 0 or 1, more preferably wherein each m is 0; and
R⁶ is 1,2,4-oxadiazol-5-one, tetrazole, 1,3,4-oxadiazol-2-one or 1,4-dihydro-tetrazol-5-one.

63. The compound of item 1, wherein:
R¹ is methyl;
R² and R³ are each independently selected from chloro, bromo or fluoro; preferably R² and R³ are chloro;
R⁴ is hydrogen;
R⁵ is selected from the group consisting of O(CH₂)ₘ-isopropyl, O(CH₂)ₘ-pyrrolidine, O(CH₂)ₘ-piperidine, O(CH₂)ₘ-morpholine, O(CH₂)ₘ-pyridine, O(CH₂)ₘ-pyrimidine, O(CH₂)ₘ-thiophene, O(CH₂)ₘ-phenyl, (CH₂)ₘ isopropyl, (CH₂)ₘ-pyrrolidine, (CH₂)ₘ-piperidine, (CH₂)ₘ-morpholine, (CH₂)ₘ-pyridine, (CH₂)ₘ-pyrimidine, (CH₂)ₘ thiophene and (CH₂)ₘ-phenyl, wherein each m is an integer independently selected from 0, 1 or 2, preferably wherein each m is 0 or 1, more preferably wherein each m is 0, and wherein any alkyl, heterocyclyl or aryl may be optionally substituted; and
R⁶ is 1,2,4-oxadiazol-5-one, tetrazole, 1,3,4-oxadiazol-2-one or 1,4-dihydro-tetrazol-5-one.

64. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted heterocyclyl selected from the group consisting of piperidine, piperidin-3-amine, piperidin-4-amine, piperidin-3-ylmethanamine, piperazine, pyrrolidine, pyrrolidin-3-amine, morpholine, isoindoline and 1-phenylpiperazine;
R⁶ is 1,2,4-oxadiazol-5-one, tetrazole, 1,3,4-oxadiazol-2-one or 1,4-dihydro-tetrazol-5-one.

65. The compound of item 1, wherein:
R¹ is methyl;
R² and R³ are chloro;
R⁴ is hydrogen;
R⁵ is optionally substituted heterocyclyl selected from the group consisting of piperidine, piperidin-3-amine, piperidin-4-amine, piperidin-3-ylmethanamine, piperazine, pyrrolidine, pyrrolidin-3-amine, morpholine, isoindoline and 1-phenylpiperazine; and
R⁶ is 1,2,4-oxadiazol-5-one, tetrazole, 1,3,4-oxadiazol-2-one or 1,4-dihydro-tetrazol-5-one.

66. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2, preferably wherein each m is 0 or 1, more preferably wherein each m is 0; and
R⁶ is optionally substituted 5-membered heterocyclyl, preferably selected from the group consisting of 1,2,4-oxadiazol-5-one, tetrazole, 1,3,4-oxadiazol-2-one and 1,4-dihydro-tetrazol-5-one.

67. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘ-5-10-membered heterocycle, wherein m is an integer independently selected from 0, 1 and 2, preferably wherein m is 0 or 1, more preferably wherein m is 0; and
R⁶ is optionally substituted 5-membered heterocyclyl, preferably selected from the group consisting of 1,2,4-oxadiazol-5-one, tetrazole, 1,3,4-oxadiazol-2-one and 1,4-dihydro-tetrazol-5-one.

68. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, wherein m each is an integer independently selected from 0, 1 and 2, preferably wherein each m is 0 or 1, more preferably wherein each m is 0; and R⁶ is optionally substituted 5-membered heterocyclyl, preferably selected from the group consisting of 1,2,4-oxadiazol-5-one, tetrazole, 1,3,4-oxadiazol-2-one and 1,4-dihydro-tetrazol-5-one.

69. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘNR^{5′}R^{5″}, wherein m is an integer independently selected from 0, 1 and 2, preferably wherein m is 0 or 1, more preferably wherein each m is 0; and
R⁶ is optionally substituted 5-membered heterocyclyl, preferably selected from the group consisting of 1,2,4-oxadiazol-5-one, tetrazole, 1,3,4-oxadiazol-2-one and 1,4-dihydro-tetrazol-5-one.

70. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2, preferably wherein each m is 0 or 1, more preferably wherein each m is 0; and
R⁶ is —CONR^{6′}R^{6″}.

71. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, wherein each m is an integer independently selected from 0, 1 and 2, preferably wherein each m is 0 or 1, more preferably wherein each m is 0; and
R⁶ is —CONR^{6′}R^{6″}.

72. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘ-5-10-membered heterocycle, wherein m is an integer independently selected from 0, 1 and 2, preferably wherein m is 0 or 1, more preferably wherein m is 0; and
R⁶ is —CONR^{6′}R^{6″}.

73. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘNR^{5′}R^{5″}, wherein m is an integer independently selected from 0, 1 and 2, preferably wherein m is 0 or 1, more preferably wherein m is 0; and
R⁶ is —CONR^{6′}R^{6″}.

74. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2, preferably wherein each m is 0 or 1, more preferably wherein each m is 0; and
R⁶ is —C(O)NHS(O)p-C₁₋₆ alkyl.

75. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, wherein each m is an integer independently selected from 0, 1 and 2, preferably wherein each m is 0 or 1, more preferably wherein each m is 0; and
R⁶ is —C(O)NHS(O)p-C₁₋₆ alkyl.

76. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘ-5-10-membered heterocycle, wherein m is an integer independently selected from 0, 1 and 2, preferably wherein m is 0 or 1, more preferably wherein m is 0; and
R⁶ is —C(O)NHS(O)p-C₁₋₆ alkyl.

77. The compound of item 1, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘNR^{5′}R^{5″}, wherein m is an integer independently selected from 0, 1 and 2, preferably wherein m is 0 or 1, more preferably wherein m is 0; and
R⁶ is —C(O)NHS(O)p-C₁₋₆ alkyl.

78. The compound of item 1, wherein:
R¹ is methyl;
R² and R³ are each independently selected from chloro, bromo or fluoro; preferably R² and R³ are chloro;
R⁴ is hydrogen;
R⁵ is selected from the group consisting of O(CH₂)ₘ-pyrrolidine, O(CH₂)ₘ-piperidine, O(CH₂)ₘ-morpholine, O(CH₂)ₘ-pyridine, O(CH₂)ₘ-pyrimidine, O(CH₂)ₘ-thiophene, (CH₂)ₘ-pyrrolidine, (CH₂)ₘ-piperidine, (CH₂)ₘ morpholine, (CH₂)ₘ-pyridine, (CH₂)ₘ-pyrimidine, and (CH₂)ₘ-thiophene, wherein each m is an integer independently selected from 0, 1 or 2, preferably wherein each m is 0 or 1, more preferably wherein each m is 0, and wherein any alkyl, heterocyclyl or aryl may be optionally substituted; and
R⁶ is optionally substituted 5-membered heterocyclyl selected from the group consisting of 1,2,4-oxadiazol-5-one, tetrazole, 1,3,4-oxadiazol-2-one and 1,4-dihydro-tetrazol-5-one.

79. The compound of item 1, wherein:
R¹ is methyl;
R² and R³ are each independently selected from chloro, bromo or fluoro; preferably R² and R³ are chloro;
R⁴ is hydrogen;
R⁵ is selected from the group consisting of O(CH₂)ₘ-pyrrolidine, O(CH₂)ₘ-piperidine, O(CH₂)ₘ-morpholine, O(CH₂)ₘ-pyridine, O(CH₂)ₘ-pyrimidine, O(CH₂)ₘ-thiophene, wherein each m is an integer independently selected from 0, 1 or 2, preferably wherein each m is 0 or 1, more preferably wherein each m is 0, and wherein any alkyl, heterocyclyl or aryl may be optionally substituted; and
R⁶ is optionally substituted 5-membered heterocyclyl selected from the group consisting of 1,2,4-oxadiazol-5-one, tetrazole, 1,3,4-oxadiazol-2-one and 1,4-dihydro-tetrazol-5-one.

80. The compound of item 1, wherein:
R¹ is methyl;
R² and R³ are each independently selected from chloro, bromo or fluoro; preferably R² and R³ are chloro;
R⁴ is hydrogen;
R⁵ is selected from the group consisting of (CH₂)ₘ-pyrrolidine, (CH₂)ₘ-piperidine, (CH₂)ₘ-morpholine, (CH₂)ₘ pyridine, (CH₂)ₘ-pyrimidine, and (CH₂)ₘ-thiophene, wherein each m is an integer independently selected from 0, 1 or 2, preferably wherein each m is 0 or 1, more preferably wherein each m is 0, and wherein any alkyl, heterocyclyl or aryl may be optionally substituted; and
R⁶ is optionally substituted 5-membered heterocyclyl selected from the group consisting of 1,2,4-oxadiazol-5-one, tetrazole, 1,3,4-oxadiazol-2-one and 1,4-dihydro-tetrazol-5-one.

81. A compound according to item 1, which is selected from the group consisting of:
1-(2-(3,4-Dichloro-5-methyl-1H-pyrrole-2-carboxamido)-5-(5-oxo-4,5-dihydro-1,2,4-oxadiazol-3-yl)phenyl)piperidin-4-aminium chloride,
1-(2-(3,4-Dichloro-5-methyl-1H-pyrrole-2-carboxamido)-5-(5-oxo-4,5-dihydro-1,2,4-oxadiazol-3-yl)phenyl)piperidin-3-aminium chloride,
(1-(2-(3,4-Dichloro-5-methyl-1H-pyrrole-2-carboxamido)-5-(5-oxo-4,5-dihydro-1,2,4-oxadiazol-3-yl)phenyl)piperidin-3-yl)methanaminium chloride,
4-((2-(3,4-Dichloro-5-methyl-1H-pyrrole-2-carboxamido)-5-(5-oxo-4,5-dihydro-1,2,4-oxadiazol-3-yl)phenyl)amino)piperidin-1-ium chloride,
4-(2-(3,4-Dichloro-5-methyl-1H-pyrrole-2-carboxamido)-5-(5-oxo-4,5-dihydro-1,2,4-oxadiazol-3-yl)phenyl)piperazin-1-ium chloride,
3,4-Dichloro-5-methyl-N-(4-(5-oxo-4,5-dihydro-1,2,4-oxadiazol-3-yl)-2-(pyrrolidin-1-yl)phenyl)-1H-pyrrole-2-carboxamide,
3,4-Dichloro-5-methyl-N-(4-(5-oxo-4,5-dihydro-1,2,4-oxadiazol-3-yl)-2-(4-phenylpiperazin-1-yl)phenyl)-1H-pyrrole-2-carboxamide,
3,4-Dichloro-5-methyl-N-(2-morpholino-4-(5-oxo-4,5-dihydro-1,2,4-oxadiazol-3-yl)phenyl)-1H-pyrrole-2-carboxamide,
(1-(2-(3,4-Dichloro-5-methyl-1H-pyrrole-2-carboxamido)-5-(2H-tetrazol-5-yl)phenyl)piperidin-3-yl)methanaminium chloride,
3,4-Dichloro-5-methyl-N-(2-morpholino-4-(2H-tetrazol-5-yl)phenyl)-1H-pyrrole-2-carboxamide,
N-(2-(Benzyloxy)-4-(5-oxo-4,5-dihydro-1,3,4-oxadiazol-2-yl)phenyl)-3,4-dichloro-5-methyl-1H-pyrrole-2-carboxamide,
4-(5-Carboxy-2-(3,4-dichloro-5-methyl-1H-pyrrole-2-carboxamido)phenyl)piperazin-1-ium chloride,
(1-(5-Carboxy-2-(3,4-dichloro-5-methyl-1H-pyrrole-2-carboxamido)phenyl)piperidin-3-yl)methanaminium chloride,
4-(5-Carboxy-2-(3,4-dichloro-5-methyl-1H-pyrrole-2-carboxamido)phenyl)piperazin-1-ium chloride,
4-((5-Carboxy-2-(3,4-dichloro-5-methyl-1H-pyrrole-2-carboxamido)phenyl)amino)piperidin-1-ium chloride,
4-(3,4-Dichloro-5-methyl-1H-pyrrole-2-carboxamido)-3-(4-phenylpiperazin-1-yl)benzoic acid,
Methyl (S)-3-(3-((tert-butoxycarbonyl)amino)pyrrolidin-1-yl)-4-(3,4-dichloro-5-methyl-1H-pyrrole-2-carboxamido)benzoate,
(S)-1-(2-(3,4-Dichloro-5-methyl-1H-pyrrole-2-carboxamido)-5-(5-oxo-4,5-dihydro-1,3,4-oxadiazol-2-yl)phenyl)piperidin-3-aminium chloride,
4-((2-(3,4-Dichloro-5-methyl-1H-pyrrole-2-carboxamido)-5-(5-oxo-4,5-dihydro-1,3,4-oxadiazol-2-yl)phenyl)amino)piperidin-1-ium chloride,
(S)-1-(2-(3,4-Dichloro-5-methyl-1H-pyrrole-2-carboxamido)-5-(5-oxo-4,5-dihydro-1,3,4-oxadiazol-2-yl)phenyl)pyrrolidin-3-aminium chloride,
3,4-Dichloro-N-(2-isopropoxy-4-(5-methyl-1,3,4-oxadiazol-2-yl)phenyl)-5-methyl-1H-pyrrole-2-carboxamide,
(S)-1-(2-(3,4-Dichloro-5-methyl-1H-pyrrole-2-carboxamido)-5-(5-oxo-4,5-dihydro-1H-tetrazol-1-yl)phenyl)pyrrolidin-3-aminium chloride,
1-(2-(3,4-Dichloro-5-methyl-1H-pyrrole-2-carboxamido)-5-(5-oxo-4,5-dihydro-1H-tetrazol-1-yl)phenyl)piperidin-4-aminium chloride,
3,4-Dichloro-5-methyl-N-(4-(5-oxo-4,5-dihydro-1H-tetrazol-1-yl)-2-(4-phenylpiperazin-1-yl)phenyl)-1H-pyrrole-2-carboxamide,
3,4-Dichloro-N-(2-(isoindolin-2-yl)-4-(5-oxo-4,5-dihydro-1H-tetrazol-1-yl)phenyl)-5-methyl-1H-pyrrole-2-carboxamide,
3-(2-(3,4-Dichloro-5-methyl-1H-pyrrole-2-carboxamido)-5-(5-oxo-4,5-dihydro-1,3,4-oxadiazol-2-yl)phenoxy)pyrrolidin-1-ium chloride,
4-(2-(3,4-Dichloro-5-methyl-1H-pyrrole-2-carboxamido)-5-(5-oxo-4,5-dihydro-1,3,4-oxadiazol-2-yl)phenoxy)piperidin-1-ium chloride,
3-((2-(3,4-Dichloro-5-methyl-1H-pyrrole-2-carboxamido)-5-(5-oxo-4,5-dihydro-1,3,4-oxadiazol-2-yl)phenoxy)methyl)piperidin-1-ium chloride,
4-(2-(2-(3,4-Dichloro-5-methyl-1H-pyrrole-2-carboxamido)-5-(5-oxo-4,5-dihydro-1,3,4-oxadiazol-2-yl)phenoxy)ethyl)piperidin-1-ium chloride,
3,4-Dichloro-5-methyl-N-(4-(5-oxo-4,5-dihydro-1,3,4-oxadiazol-2-yl)-2-(pyridin-4-ylmethoxy)phenyl)-1H-pyrrole-2-carboxamide,
3,4-Dichloro-5-methyl-N-(4-(5-oxo-4,5-dihydro-1,3,4-oxadiazol-2-yl)-2-(pyrimidin-2-ylmethoxy)phenyl)-1H-pyrrole-2-carboxamide,
3,4-Dichloro-N-(2-isopropoxy-4-(5-oxo-4,5-dihydro-1,3,4-oxadiazol-2-yl)phenyl)-5-methyl-1H-pyrrole-2-carboxamide,
3,4-Dichloro-5-methyl-N-(4-(5-oxo-4,5-dihydro-1,3,4-oxadiazol-2-yl)-2-(thiophen-2-ylmethoxy)phenyl)-1H-pyrrole-2-carboxamide,
(R)-1-(5-((Carboxymethyl)carbamoyl)-2-(3,4-dichloro-5-methyl-1H-pyrrole-2-carboxamido)phenyl)pyrrolidin-3-aminium chloride,
(S)-1-(5-((Carboxymethyl)carbamoyl)-2-(3,4-dichloro-5-methyl-1H-pyrrole-2-carboxamido)phenyl)pyrrolidin-3-aminium chloride,
4-(5-((Carboxymethyl)carbamoyl)-2-(3,4-dichloro-5-methyl-1H-pyrrole-2-carboxamido)phenyl)piperazin-1-ium chloride,
(R)-1-(5-((Carboxymethyl)carbamoyl)-2-(4,5-dibromo-1H-pyrrole-2-carboxamido)phenyl)pyrrolidin-3-aminium chloride,
(S)-1-(5-((Carboxymethyl)carbamoyl)-2-(4,5-dibromo-1H-pyrrole-2-carboxamido)phenyl)pyrrolidin-3-aminium chloride,
4-(5-((Carboxymethyl)carbamoyl)-2-(4,5-dibromo-1H-pyrrole-2-carboxamido)phenyl)piperazin-1-ium chloride, and
4-(2-(3,4-Dichloro-5-methyl-1H-pyrrole-2-carboxamido)-5-(((5-oxo-4,5-dihydro-1,3,4-oxadiazol-2-yl)methyl)carbamoyl)phenyl)piperazin-1-ium chloride.
82. A compound according to item 1, which is selected from the group consisting of:
1-(2-(3,4-Dichloro-5-methyl-1H-pyrrole-2-carboxamido)-5-(5-oxo-4,5-dihydro-1,2,4-oxadiazol-3-yl)phenyl)piperidin-4-aminium chloride,
1-(2-(3,4-Dichloro-5-methyl-1H-pyrrole-2-carboxamido)-5-(5-oxo-4,5-dihydro-1,2,4-oxadiazol-3-yl)phenyl)piperidin-3-aminium chloride,
(1-(2-(3,4-Dichloro-5-methyl-1H-pyrrole-2-carboxamido)-5-(5-oxo-4,5-dihydro-1,2,4-oxadiazol-3-yl)phenyl)piperidin-3-yl)methanaminium chloride,
3,4-Dichloro-5-methyl-N-(4-(5-oxo-4,5-dihydro-1,2,4-oxadiazol-3-yl)-2-(pyrrolidin-1-yl)phenyl)-1H-pyrrole-2-carboxamide,
3,4-Dichloro-5-methyl-N-(4-(5-oxo-4,5-dihydro-1,2,4-oxadiazol-3-yl)-2-(4-phenylpiperazin-1-yl)phenyl)-1H-pyrrole-2-carboxamide,
3,4-Dichloro-5-methyl-N-(2-morpholino-4-(5-oxo-4,5-dihydro-1,2,4-oxadiazol-3-yl)phenyl)-1H-pyrrole-2-carboxamide,
(1-(2-(3,4-Dichloro-5-methyl-1H-pyrrole-2-carboxamido)-5-(2H-tetrazol-5-yl)phenyl)piperidin-3-yl)methanaminium chloride,
3,4-Dichloro-5-methyl-N-(2-morpholino-4-(2H-tetrazol-5-yl)phenyl)-1H-pyrrole-2-carboxamide,
(1-(5-Carboxy-2-(3,4-dichloro-5-methyl-1H-pyrrole-2-carboxamido)phenyl)piperidin-3-yl)methanaminium chloride,
(S)-1-(2-(3,4-Dichloro-5-methyl-1H-pyrrole-2-carboxamido)-5-(5-oxo-4,5-dihydro-1,3,4-oxadiazol-2-yl)phenyl)piperidin-3-aminium chloride,
4-((2-(3,4-Dichloro-5-methyl-1H-pyrrole-2-carboxamido)-5-(5-oxo-4,5-dihydro-1,3,4-oxadiazol-2-yl)phenyl)amino)piperidin-1-ium chloride,
(S)-1-(2-(3,4-Dichloro-5-methyl-1H-pyrrole-2-carboxamido)-5-(5-oxo-4,5-dihydro-1,3,4-oxadiazol-2-yl)phenyl)pyrrolidin-3-aminium chloride,
3,4-Dichloro-5-methyl-N-(4-(5-oxo-4,5-dihydro-1H-tetrazol-1-yl)-2-(4-phenylpiperazin-1-yl)phenyl)-1H-pyrrole-2-carboxamide,
3,4-Dichloro-N-(2-(isoindolin-2-yl)-4-(5-oxo-4,5-dihydro-1H-tetrazol-1-yl)phenyl)-5-methyl-1H-pyrrole-2-carboxamide,
4-(2-(3,4-Dichloro-5-methyl-1H-pyrrole-2-carboxamido)-5-(5-oxo-4,5-dihydro-1,3,4-oxadiazol-2-yl)phenoxy)piperidin-1-ium chloride, and
3-((2-(3,4-Dichloro-5-methyl-1H-pyrrole-2-carboxamido)-5-(5-oxo-4,5-dihydro-1,3,4-oxadiazol-2-yl)phenoxy)methyl)piperidin-1-ium chloride.

83. A compound according to item 1, which is selected from the group consisting of:
4-(2-(3,4-Dichloro-5-methyl-1H-pyrrole-2-carboxamido)-5-(5-oxo-4,5-dihydro-1,3,4-oxadiazol-2-yl)phenyl)piperazin-1-ium chloride,
3,4-Dichloro-5-methyl-N-(2-morpholino-4-(5-oxo-4,5-dihydro-1,3,4-oxadiazol-2-yl)phenyl)-1H-pyrrole-2-carboxamide,
3,4-dichloro-5-methyl-N-(2-(2-methylmorpholino)-4-(5-oxo-4,5-dihydro-1,3,4-oxadiazol-2-yl)phenyl)-1H-pyrrole-2-carboxamide,
3,4-Dichloro-N-(2-(2,6-dimethylmorpholino)-4-(hydrazinecarbonyl)phenyl)-5-methyl-1H-pyrrole-2-carboxamide, and
3,4-Dichloro-5-methyl-N-(4-((methylsulfonyl)carbamoyl)-2-morpholinophenyl)-1H-pyrrole-2-carboxamide.

84. The compound of any one of items 1 to 83 for use in medicine.

85. The compound of any one of items 1 to 84 for use in the treatment of a bacterial infection in a warm-blooded animal.

86. The compound for use of item 85, wherein the warm-blooded animal is a human.

87. The compound for use of any one of items 85 or 86, wherein the bacterial infection is selected from the group consisting of community-acquired pneumonia, hospital-acquired pneumonia, skin and skin structure infections, acute exacerbation of chronic bronchitis, conjunctivitis, meningitis, gastrointestinal tract infections, pelvic inflammatory disease, acute sinusitis, acute otitis media, bloodstream infections (bacteraemia), catheter-related sepsis, febrile neutropenia, osteomyelitis, endocarditis, urinary tract infections and infections caused by drug resistant bacteria.

88. The compound for use of any one of items 85 to 87, wherein the bacterial infection is caused by a Gram-negative, a Gram-positive, or a Gram-variable bacterium.

89. The compound for use of any one of items 85 to 87, wherein the bacterial infection is caused by a Gram-negative bacterium.

90. The compound for use of any one of items 85 to 87, wherein the bacterial infection is caused by a Gram-positive bacterium.

91. The compound for use of any one of items 85 to 87, wherein the bacterial infection is caused by a Gram-variable bacterium.

92. The compound for use of any one of items 85 to 87, wherein the bacterial infection is caused by a pathogenic bacterium, an opportunistic bacterial pathogen, or other bacterium.

93. The compound for use of any one of items 85 to 87, wherein the bacterial infection is caused by any of the bacteria selected from the group of Clostridium species, including Clostridium difficile, Clostridium perfringens and Clostridium tetani, Bacillus species, including Bacillus anthracis, Haemophilus species including Haemophilus influenzae, Helicobacter species, including Helicobacter pylori, Neisseria species, including Neisseria gonorrhoeae and including drug-resistant Neisseria gonorrhoeae, the Enterobacteriaceae family, including Carbapenem-resistant Enterobacteriaceae (CRE), extended spectrum p-lactamase producing Enterobacteriaceae (ESBLs), Enterobacter species, including Enterobacter cloacae, Enterobacter aerogenes, Salmonella species, including drug-resistant Non-typhoidal Salmonella and Salmonella Typhi, Shigella, including drug-resistant Shigella, Citrobacter species, Escherichia species, including Escherichia coli, including Enterotoxigenic E. coli (ETEC), Enteropathogenic E. coli (EPEC), uropathogenic E. coli (UPEC), Enteroinvasive E. coli (EIEC), Enterohemorrhagic E. coli (EHEC), Acinetobacter species, including Acinetobacter baumannii, drug- and multidrug-resistant Acinetobacter, Enterococcus species, including Vancomycin-resistant Enterococcus (VRE), Enterococcus faecium, Enterococcus faecalis, Pseudomonas species, including Pseudomonas aeruginosa, including drug- and multidrug-resistant Pseudomonas aeruginosa, Staphylococcus aureus, including methicillin-resistant Staphylococcus aureus (MRSA) and Vancomycin-resistant Staphylococcus aureus (VRSA), Staphylococcus epidermidis and methicillin-resistant Staphylococcus epidermidis (MRSE), Staphylococcus haemolyticus, Staphylococcus saprophyticus, Streptococcus species and their drug-resistant variants, including Streptococcus agalactiae, Streptococcus pyogenes, Streptococcus pneumoniae and including their drug-resistant variants.

94. The compound for use of any one of items 85 to 87, wherein the bacterial infection is caused by any of the ESKAPE pathogens, including Enterococcus faecium, Staphylococcus aureus, Klebsiella pneumoniae, Acinetobacter baumannii, Pseudomonas aeruginosa, Enterobacter species, any mycobacteria or any of their drug-resistant variants.

95. A pharmaceutical composition comprising a compound of any one of items 1 to 83 and a pharmaceutically acceptable excipient or carrier.

96. A method of inhibiting bacterial DNA gyrase and/or bacterial topoisomerase IV in a warm-blooded animal in need of such treatment, comprising administering to the animal an effective amount of compound of any one of items 1 to 83.

97. A method of producing an antibacterial effect in a warm-blooded animal in need of such treatment, comprising administering to the animal an effective amount of compound of any one of items 1 to 83.

98. A method of treating a bacterial infection in a warm-blooded animal in need thereof, comprising administering to the animal an effective amount of compound of any one of items 1 to 83.

99. The method of item 98, wherein the bacterial infection is selected from the group consisting of community-acquired pneumonia, hospital-acquired pneumonia, skin and skin structure infections, acute exacerbation of chronic bronchitis, conjunctivitis, meningitis, gastrointestinal tract infections, pelvic inflammatory disease, acute sinusitis, acute otitis media, bloodstream infections (bacteraemia), catheter-related sepsis, febrile neutropenia, osteomyelitis, endocarditis, urinary tract infections and infections caused by drug resistant bacteria.

100. The method of item 98, wherein the bacterial infection is caused by a Gram-negative, a Gram-positive, or a Gram-variable bacterium.

101. The method of item 98, wherein the bacterial infection is caused by a pathogenic bacterium, an opportunistic bacterial pathogen, or other bacterium.

102. The method of item 98, wherein the bacterial infection is caused by any of the bacteria selected from the group of Clostridium species, including Clostridium difficile, Clostridium perfringens and Clostridium tetani, Bacillus species, including Bacillus anthracis, Haemophilus species including Haemophilus influenzae, Helicobacter species, including Helicobacter pylori, Neisseria species, including Neisseria gonorrhoeae and including drug-resistant Neisseria gonorrhoeae, the Enterobacteriaceae family, including Carbapenem-resistant Enterobacteriaceae (CRE), extended spectrum p-lactamase producing Enterobacteriaceae (ESBLs), Enterobacter species, including Enterobacter cloacae, Enterobacter aerogenes, Salmonella species, including drug-resistant Non-typhoidal Salmonella and Salmonella Typhi, Shigella, including drug-resistant Shigella, Citrobacter species, Escherichia species, including Escherichia coli, including Enterotoxigenic E. coli(ETEC), Enteropathogenic E. coli, Enteroinvasive E. coli (EIEC), Enterohemorrhagic E. coli (EHEC), Acinetobacter species, including Acinetobacter baumannii, drug- and multidrug-resistant Acinetobacter, Enterococcus species, including Vancomycin-resistant Enterococcus (VRE), Enterococcus faecium, Enterococcus faecalis, Pseudomonas species, including Pseudomonas aeruginosa, including drug- and multidrug-resistant Pseudomonas aeruginosa, Staphylococcus aureus, including methicillin-resistant Staphylococcus aureus (MRSA) and Vancomycin-resistant Staphylococcus aureus (VRSA), Staphylococcus epidermidis and methicillin-resistant Staphylococcus epidermidis (MRSE), Staphylococcus haemolyticus, Staphylococcus saprophyticus, Streptococcus species and their drug-resistant variants, including Streptococcus agalactiae, Streptococcus pyogenes, Streptococcus pneumoniae and including their drug-resistant variants.

103. The method of item 98, wherein the bacterial infection is caused by any of the ESKAPE pathogens, including Enterococcus faecium, Staphylococcus aureus, Klebsiella pneumoniae, Acinetobacter baumannii, Pseudomonas aeruginosa, Enterobacter species or any of their drug-resistant variants.

104. The method of any one of items 98 to 103, wherein the warm-blooded animal is a human.

105. The use of a compound of any one of items 1 to 83 for the manufacture of a medicament for use for the production of an antibacterial effect in a warm-blooded animal.

106. The use of a compound of any one of items 1 to 83 for the manufacture of a medicament for use in inhibition of bacterial DNA gyrase and/or topoisomerase IV in a warm-blooded animal.

107. The use of a compound of any one of items 1 to 83 for the manufacture of a medicament for use for the treatment of a bacterial infection in a warm-blooded animal.

108. The use of item 107, wherein the bacterial infection is selected from the group consisting of community-acquired pneumonia, hospital-acquired pneumonia, skin and skin structure infections, acute exacerbation of chronic bronchitis, conjunctivitis, meningitis, gastrointestinal tract infections, pelvic inflammatory disease, acute sinusitis, acute otitis media, bloodstream infections (bacteraemia), catheter-related sepsis, febrile neutropenia, osteomyelitis, endocarditis, urinary tract infections and infections caused by drug resistant bacteria.

109. The use of item 107 or 108, wherein the bacterial infection is caused by a Gram-negative, a Gram-positive, or a Gram-variable bacterium.

110. The use of item 107 or 108, wherein the bacterial infection is caused by a pathogenic bacterium, an opportunistic bacterial pathogen, or other bacterium.

111. The use of item 107 or 108, wherein the bacterial infection is caused by any of the bacteria selected from the group of Clostridium species, including Clostridium difficile, Clostridium perfringens and Clostridium tetani, Bacillus species, including Bacillus anthracis, Haemophilus species including Haemophilus influenzae, Helicobacter species, including Helicobacter pylori, Neisseria species, including Neisseria gonorrhoeae and including drug-resistant Neisseria gonorrhoeae, the Enterobacteriaceae family, including Carbapenem-resistant Enterobacteriaceae (CRE), extended spectrum p-lactamase producing Enterobacteriaceae (ESBLs), Enterobacter species, including Enterobacter cloacae, Enterobacter aerogenes, Salmonella species, including drug-resistant Non-typhoidal Salmonella and Salmonella Typhi, Shigella, including drug-resistant Shigella, Citrobacter species, Escherichia species, including Escherichia coli, including Enterotoxigenic E. coli(ETEC), Enteropathogenic E. coli, Enteroinvasive E. coli (EIEC), Enterohemorrhagic E. coli (EHEC), Acinetobacter species, including Acinetobacter baumannii, drug- and multidrug-resistant Acinetobacter, Enterococcus species, including Vancomycin-resistant Enterococcus (VRE), Enterococcus faecium, Enterococcus faecalis, Pseudomonas species, including Pseudomonas aeruginosa, including drug- and multidrug-resistant Pseudomonas aeruginosa, Staphylococcus aureus, including methicillin-resistant Staphylococcus aureus (MRSA) and Vancomycin-resistant Staphylococcus aureus (VRSA), Staphylococcus epidermidis and methicillin-resistant Staphylococcus epidermidis (MRSE), Staphylococcus haemolyticus, Staphylococcus saprophyticus, Streptococcus species and their drug-resistant variants, including Streptococcus agalactiae, Streptococcus pyogenes, Streptococcus pneumoniae and including their drug-resistant variants. 112. The use of item 107 or 108, wherein the bacterial infection is caused by any of the ESKAPE pathogens, including Enterococcus faecium, Staphylococcus aureus, Klebsiella pneumoniae, Acinetobacter baumannii, Pseudomonas aeruginosa, Enterobacter species or any of their drug-resistant variants.

113. The use of any one of items 105 to 112, wherein the warm-blooded animal is a human.

114. A process for preparing a compound as defined in any one of items 1 to 83 (with the variable groups being as defined in any one of items 1 to 80), which comprises:

### Process Step a) Reacting a Compound of Formula II:

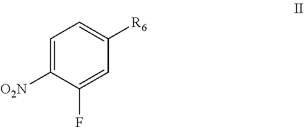

with compound of formula III:

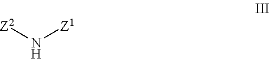

wherein Z¹ and Z² are independently selected from H, —R^{6′}R^{6″}, —CH(CO₂R^{6″})R^{6′}, heterocyclyl, aryl, to a compound of formula IV:

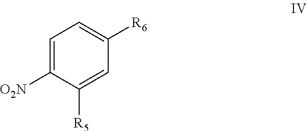

or

### Process Step b) Coupling a Compound of Formula V:

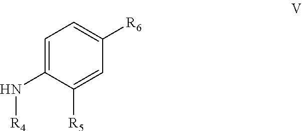

with compound of formula VI:

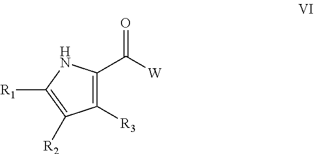

wherein W is selected from hydroxyl, halogen (preferably Cl, Br or I) and 1,1,1-trichloromethyl, to a compound of formula I:

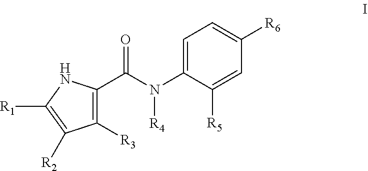

or

### Process Step c) Reacting a Compound of Formula Ia:

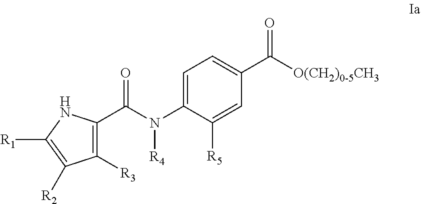

to a compound of formula Ib:

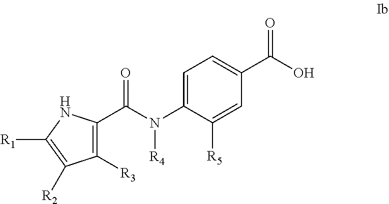

or

### Process Step d) Reacting a Compound of Formula Ic:

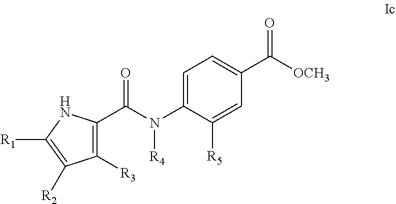

to a compound of formula Id:

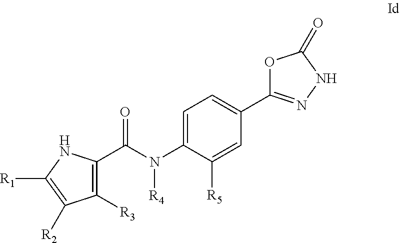

in a two-step procedure, using hydrazine monohydrate (step 1) and 1,1′-carbonyldiimidazole (step 2), as reagents.

### Process Step e) Reacting a Compound of Formula Ie:

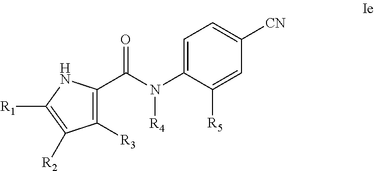

to a compound of formula If:

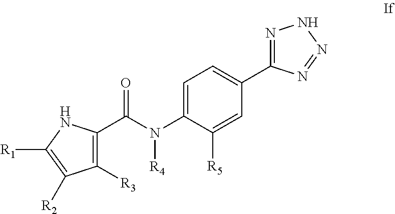

in a procedure using sodium azide as reagents.

### DETAILED DESCRIPTION OF THE INVENTION

### Definitions

In this specification the term alkyl includes both straight and branched chain alkyl groups but references to individual alkyl groups such as propyl are specific for the straight chain version only. An analogous convention applies to other generic terms. Unless otherwise stated the term alkyl advantageously refers to chains with 1-6 carbon atoms, preferably 1-4 carbon atoms. In this specification, the terms alkenyl, alkynyl and cycloalkenyl include all positional and geometrical isomers.

In this specification the term alkoxy means an alkyl group as defined herein before linked to an oxygen atom.

Where optional substituents are chosen from 0, 1, 2 or 3 groups it is to be understood that this definition includes all substituents being chosen from one of the specified groups or the substituents being chosen from two or more of the specified groups. An analogous convention applies to substituents chosen from 0, 1 or 2 groups; 1, 2 or 3 substituents; and 1 or 2 groups.

It is to be understood that where substituents contain two substituents on an alkyl chain, in which both are linked by a heteroatom (for example two alkoxy substituents), then these two substituents are not substituents on the same carbon atom of the alkyl chain. It will be understood that unstable compounds are not contemplated as part of this invention.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

Where “R^{6′} and R^{6″} may together with the nitrogen to which they are attached form a 5 or 6-membered heterocyclyl ring” said “5 or 6-membered heterocyclyl ring” is a saturated, partially saturated or fully unsaturated, monocyclic ring containing one nitrogen atom to which R^{6′} and R^{6″} are attached, and the other atoms are either all carbon atoms or they are carbon atoms and 1, 2 or 3 heteroatoms chosen from nitrogen, sulphur or oxygen, wherein a —CH₂— group can optionally be replaced by a —C(O)—, and a ring nitrogen atom or a ring sulphur atom may be optionally oxidised to form the N- and/or S-oxide(s). Examples and suitable values of “R^{6′} and R^{6″} may together with the nitrogen to which they are attached form a 5 or 6-membered heterocyclyl ring such as piperidino, piperazino and morpholino.

Heterocyclyl is a saturated, partially saturated or unsaturated, optionally substituted monocyclic or bicyclic ring system containing 5 to 10 atoms of which 1, 2, 3 or 4 ring atoms are chosen from nitrogen, sulphur or oxygen, which may, unless otherwise specified, be carbon or nitrogen linked, wherein a —CH₂— group can optionally be replaced by a —C(O)—, a ring sulphur atom may be optionally oxidised to form the S-oxide(s), and a ring nitrogen atom may be optionally oxidised to form the N-oxide. Examples and suitable values of the term heterocyclyl are morpholino, morpholinyl, piperidino, piperidyl, pyridyl, pyridyl-N-oxide, pyranyl, pyrrolyl, imidazolyl, thiazolyl, thienyl, dioxolanyl, thiadiazolyl, piperazinyl, isothiazolidinyl, triazolyl, tetrazolyl, pyrrolidinyl, 2-oxazolidinonyl, 5-isoxazolonyl, thiomorpholino, pyrrolinyl, homopiperazinyl, 3,5-dioxapiperidinyl, 3-oxopyrazolin-5-yl, tetrahydropyranyl, tetrahydrothiopyranyl, 1-oxotetrahydrothiopyranyl, 1,1-dioxotetrahydrothiopyranyl, pyrimidyl, pyrazinyl, pyridazinyl, pyrazolyl, pyrazolinyl, isoxazolyl, 4-oxopydridyl, 2-oxopyrrolidyl, 4-oxothiazolidyl, furyl, thienyl, oxazolyl, oxadiazolyl, 2-[(5-oxo)-[oxa-3,4-diazolyl] and 3-[oxa-2,4-diazolyl].

Suitably a heterocyclyl is morpholino, morpholinyl, piperidino, piperidyl, pyridyl, pyranyl, pyrrolyl, imidazolyl, thiazolyl, thienyl, thiadiazolyl, piperazinyl, isothiazolidinyl, 1,3,4-triazolyl, tetrazolyl, pyrrolidinyl, thiomorpholino, pyrrolinyl, homopiperazinyl, 3,5-dioxapiperidinyl, pyrimidyl, pyrazinyl, pyridazinyl, pyrazolyl, pyrazolinyl, isoxazolyl, 4-oxopydridyl, 2-oxopyrrolidyl, 4-oxothiazolidyl, furyl, thienyl, oxazolyl, 1,3,4-oxadiazolyl, 1,2,4-oxadiazolyl 2-[(5-oxo)-[oxa-3,4-diazolyl] and 3-[oxa-2,4-diazolyl].

Conveniently heterocyclyl is oxazolyl, 1,3,4-oxadiazolyl, 1,2,4-oxadiazolyl, 2-[(5-oxo)-[oxa-3,4-diazolyl], 3-[oxa-2,4-diazolyl], tetrazolyl, thiazolyl, thiadiazolyl, pyridyl, imidazolyl, furyl, thienyl, morpholine, pyrimidyl, pyrazinyl, pyridazinyl, pyrazolyl, pyrazolinyl, and piperazinyl.

Suitable optional substituents for heterocyclyl as a saturated or partially saturated ring system are, unless otherwise defined, 1, 2 or 3 substituents independently selected from halogen, cyano, hydroxy, C₁₋₄ alkyl, C₁₋₄ alkoxy, C₁₋₄ alkylS(O)_{b} (wherein b is 0, 1 or 2), 6-membered monocyclic aryl or heteroaryl, —NH-Boc and -Boc. Further suitable substituents for “heterocyclyl” as a saturated or partially saturated ring system are 1, 2 or 3 substituents independently selected from fluoro, chloro, cyano, hydroxy, amino, carboxyl, methyl, ethyl, methoxy, methylthio, methylsulfinyl, methylsulfonyl, phenyl, —NH-Boc and -Boc.

Suitable optional substituents for heterocyclyl as an unsaturated ring system are, unless otherwise defined, 1, 2 or 3 substituents independently selected from halogen, cyano, nitro, amino, hydroxy, C₁₋₄ alkyl, C₁₋₄ alkoxy, C₁₋₄ alkylS(O)_{b} (wherein b is 0, 1 or 2), N—(C₁a alkyl)amino, NN—(C₁a alkyl)₂amino, 6-membered monocyclic aryl or heteroaryl, —NH-Boc and -Boc. Further suitable optional substituents for “heterocyclyl” as an unsaturated ring system are 1, 2 or 3 substituents independently selected from fluoro, chloro, cyano, nitro, amino, carboxyl, methylamino, dimethylamino, hydroxy, methyl, ethyl, methoxy, methylthio, methylsulfinyl, methylsulfonyl, phenyl, —NH-Boc and -Boc.

For the avoidance of doubt, optional substituents on heterocyclyl groups are generally substituents on carbon atoms of the ring, but may where appropriate be on an N atom, for example N-alkylpyridine.

In this context, the prefixes 3-, 4-, 5-, 6-, 7-, 8-, 9- and 10-membered denote the number of ring atoms, or range of ring atoms, whether carbon atoms or heteroatoms. For example, the term “3-10 membered heterocyclyl”, as used herein, pertains to a heterocyclyl group having 3, 4, 5, 6, 7, 8, 9 or 10 ring atoms or a range comprising any of two of those integers. Examples of heterocyclyl groups include 5-6-membered monocyclic heterocyclyls and 9-10 membered fused bicyclic heterocyclyls. Examples of monocyclic heterocyclyl groups include, but are not limited to, those containing one nitrogen atom such as aziridine (3-membered ring), azetidine (4-membered ring), pyrrolidine (tetrahydropyrrole), pyrroline (e.g., 3-pyrroline, 2,5-dihydropyrrole), 2H-pyrrole or 3H-pyrrole (isopyrrole, isoazole) or pyrrolidinone (5-membered rings), piperidine, dihydropyridine, tetrahydropyridine (6-membered rings), and azepine (7-membered ring); those containing two nitrogen atoms such as imidazoline, pyrazolidine (diazolidine), imidazoline, pyrazoline (dihydropyrazole) (5-membered rings), piperazine (6-membered ring); those containing one oxygen atom such as oxirane (3-membered ring), oxetane (4-membered ring), oxolane (tetrahydrofuran), oxole (dihydrofuran) (5-membered rings), oxane (tetrahydropyran), dihydropyran, pyran (6-membered rings), oxepin (7-membered ring); those containing two oxygen atoms such as dioxolane (5-membered ring), dioxane (6-membered ring), and dioxepane (7-membered ring); those containing three oxygen atoms such as trioxane (6-membered ring); those containing one sulfur atom such as thiirane (3-membered ring), thietane (4-membered ring), thiolane (tetrahydrothiophene) (5-membered ring), thiane (tetrahydrothiopyran) (6-membered ring), thiepane (7-membered ring); those containing one nitrogen and one oxygen atom such as tetrahydrooxazole, dihydrooxazole, tetrahydroisoxazole, dihydroisoxazole (5-membered rings), morpholine, tetrahydrooxazine, dihydrooxazine, oxazine (6-membered rings); those containing one nitrogen and one sulfur atom such as thiazoline, thiazolidine (5-membered rings), thiomorpholine (6-membered ring); those containing two nitrogen and one oxygen atom such as oxadiazine (6-membered ring); those containing one oxygen and one sulfur such as: oxathiole (5-membered ring) and oxathiane (thioxane) (6-membered ring); and those containing one nitrogen, one oxygen and one sulfur atom such as oxathiazine (6-membered ring). Heterocyclyls also encompass aromatic heterocyclyls and non-aromatic heterocyclyls. Such groups may be substituted or unsubstituted.

The term “aromatic heterocyclyl” may be used interchangeably with the term “heteroaromatic substituent/ring” or the term “heteroaryl” or “hetaryl”. The heteroatoms in the aromatic heterocyclyl group may be independently selected from N, S and O. “Heteroaryl” is used herein to denote a heterocyclic group having aromatic character and embraces aromatic monocyclic ring systems and polycyclic (e.g. bicyclic) ring systems containing one or more aromatic rings. The term aromatic heterocyclyl also encompasses pseudoaromatic heterocyclyls. The term “pseudoaromatic” refers to a ring system which is not strictly aromatic, but which is stabilized by means of delocalization of electrons and behaves in a similar manner to aromatic rings. The term aromatic heterocyclyl therefore covers polycyclic ring systems in which all of the fused rings are aromatic as well as ring systems where one or more rings are non-aromatic, provided that at least one ring is aromatic. In polycyclic systems containing both aromatic and non-aromatic rings fused together, the group may be attached to another moiety by the aromatic ring or by a non-aromatic ring.

Examples of heteroaryl groups are monocyclic and bicyclic groups containing from five to ten ring members. The heteroaryl group can be, for example, a five membered or six membered monocyclic ring or a bicyclic structure formed from fused five and six membered rings or two fused six membered rings or two fused five membered rings. Each ring may contain up to four heteroatoms typically selected from nitrogen, sulfur and oxygen. The heteroaryl ring will contain up to 4 heteroatoms, more typically up to 3 heteroatoms, more usually up to 2, for example a single heteroatom. In one case, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five. Aromatic heterocyclyl groups may be 5-membered or 6-membered mono-cyclic aromatic ring systems.

Examples of 5-membered monocyclic heteroaryl groups include but are not limited to furanyl, thienyl, pyrrolyl, oxazolyl, oxadiazolyl (including 1,2,3 and 1,2,4 oxadiazolyls and furazanyl i.e. 1,2,5-oxadiazolyl), thiazolyl, isoxazolyl, isothiazolyl, pyrazolyl, imidazolyl, triazolyl (including 1,2,3, 1,2,4 and 1,3,4 triazolyls), oxatriazolyl, tetrazolyl, thiadiazolyl (including 1,2,3 and 1,3,4 thiadiazolyls) and the like.

Examples of 6-membered monocyclic heteroaryl groups include but are not limited to pyridinyl, pyrimidinyl, pyridazinyl, pyrazinyl, triazinyl, pyranyl, oxazinyl, dioxinyl, thiazinyl, thiadiazinyl and the like. Examples of 6-membered heteroaryl groups containing nitrogen include pyridyl (1 nitrogen), pyrazinyl, pyrimidinyl and pyridazinyl (2 nitrogens). It will be understood that, such as in the case of pyridyl when substituted with an oxo (═O) substituent the group may be interchangeably referred to as a pyridinone group.

Aromatic heterocyclyl groups may also be bicyclic or polycyclic heteroaromatic ring systems such as fused ring systems (including purinyl, pteridinyl, napthyridinyl, 1H-thieno[2,3-c]pyrazolyl, thieno[2,3-b]furyl and the like) or linked ring systems (such as oligothiophene, polypyrrole and the like). Fused ring systems may also include aromatic 5-membered or 6-membered heterocyclyls fused to carbocyclic aromatic rings such as phenyl, naphthyl, indenyl, azulenyl, fluorenyl, anthracenyl and the like, such as 5- or 6-membered aromatic heterocyclyls fused to a phenyl ring including 5-membered aromatic heterocyclyls containing nitrogen fused to a phenyl ring, 5-membered aromatic heterocyclyls containing 1 or 2 nitrogens fused to a phenyl ring and such as 5- or 6-membered aromatic heteroaryls fused to a 6-membered aromatic or non-aromatic heterocyclyls.

A bicyclic heteroaryl group may be, for example, a group selected from: a) a benzene ring fused to a 5- or 6-membered ring containing 1, 2 or 3 ring heteroatoms; b) a pyridine ring fused to a 5- or 6-membered ring containing 1, 2 or 3 ring heteroatoms; c) a pyrimidine ring fused to a 5- or 6-membered ring containing 1 or 2 ring heteroatoms; d) a pyrrole ring fused to a 5- or 6-membered ring containing 1, 2 or 3 ring heteroatoms; e) a pyrazole ring fused to a 5- or 6-membered ring containing 1 or 2 ring heteroatoms; f) an imidazole ring fused to a 5- or 6-membered ring containing 1 or 2 ring heteroatoms; g) an oxazole ring fused to a 5- or 6-membered ring containing 1 or 2 ring heteroatoms; h) an isoxazole ring fused to a 5- or 6-membered ring containing 1 or 2 ring heteroatoms; i) a thiazole ring fused to a 5- or 6-membered ring containing 1 or 2 ring heteroatoms; j) an isothiazole ring fused to a 5- or 6-membered ring containing 1 or 2 ring heteroatoms; k) a thiophene ring fused to a 5- or 6-membered ring containing 1, 2 or 3 ring heteroatoms; 1) a furan ring fused to a 5- or 6-membered ring containing 1, 2 or 3 ring heteroatoms; m) a cyclohexyl ring fused to a 5- or 6-membered ring containing 1, 2 or 3 ring heteroatoms; and n) a cyclopentyl ring fused to a 5- or 6-membered ring containing 1, 2 or 3 ring heteroatoms. Particular examples of bicyclic heteroaryl groups containing a five membered ring fused to another five membered ring i.e. 8-membered fused bicyclic rings include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring, i.e. 9-membered fused bicyclic rings include but are not limited to benzofuran, benzothiophene, benzimidazole, benzoxazole, isobenzoxazole, benzisoxazole, benzothiazole, benzoisothiazole, isobenzofuran, indole, isoindole, indolizine, indoline, isoindoline, purine (e.g. adenine, guanine), indazole, imidazopyridine (e.g. imidazo[1,2-a]pyridine and imidazo[4,5-b]pyridine], pyrazolopyrimidine (e.g. pyrazolo[1,5-a]pyrimidine), benzodioxole and pyrazolopyridine (e.g. pyrazolo[1,5-a]pyridine) groups. A further example of a six membered ring fused to a five membered ring is a pyrrolopyridine group such as a pyrrolo[2,3-b]pyridine group.

Particular examples of bicyclic heteroaryl groups containing two fused six membered rings i.e. 10-membered fused bicyclic rings include but are not limited to quinoline, isoquinoline, chroman, thiochroman, chromene (including those optionally substituted with oxo (═O) group e.g. oxochromene), isochromene, isochroman, benzodioxan, quinolizine, benzoxazine, benzodiazine, pyridopyridine, quinoxaline, quinazoline, cinnoline, phthalazine, naphthyridine and pteridine groups.

Examples of heteroaryl groups containing an aromatic ring and a non-aromatic ring include tetrahydronaphthalene, tetrahydroisoquinoline, tetrahydroquinoline, dihydrobenzothiophene, dihydrobenzofuran, 2,3-dihydro-benzo[1,4]dioxine, benzo[1,3]dioxole, 4,5,6,7-tetrahydrobenzofuran, indoline, isoindoline and indane groups.

Examples of aromatic heterocyclyls fused to carbocyclic aromatic rings may therefore include but are not limited to benzothiophenyl, indolyl, isoindolyl, benzofuranyl, isobenzofuranyl, benzimidazolyl, indazolyl, benzoxazolyl, benzisoxazolyl, isobenzoxazoyl, benzothiazolyl, benzisothiazolyl, quinolinyl, isoquinolinyl, quinoxalinyl, quinazolinyl, cinnolinyl, benzotriazinyl, phthalazinyl, carbolinyl and the like.

The term “non-aromatic heterocyclyl” encompasses optionally substituted saturated and unsaturated rings which contain at least one heteroatom selected from N, S and O.

Non-aromatic heterocyclyls may be 3-7 membered mono-cyclic rings. The term “3-7 membered monocyclic”, as used herein, pertains to a mono-cyclic group having 3, 4, 5, 6 or 7 ring atoms or a range comprising any of two of those integers. Examples of 5-membered non-aromatic heterocyclyl rings include 2H-pyrrolyl, 1-pyrrolinyl, 2-pyrrolinyl, 3-pyrrolinyl, pyrrolidinyl, 1-pyrrolidinyl, 2-pyrrolidinyl, 3-pyrrolidinyl, tetrahydrofuranyl, tetrahydrothiophenyl, pyrazolinyl, 2-pyrazolinyl, 3-pyrazolinyl, pyrazolidinyl, 2-pyrazolidinyl, 3-pyrazolidinyl, imidazolidinyl, 3-dioxalanyl, thiazolidinyl, isoxazolidinyl, 2-imidazolinyl and the like.

Examples of 6-membered non-aromatic heterocyclyls include piperidinyl, piperidinonyl, pyranyl, dihydropyranyl, tetrahydropyranyl, 2H-pyranyl, 4H-pyranyl, thianyl, thianyl oxide, thianyl dioxide, piperazinyl, dioxanyl, 1,4-dioxinyl, 1,4-dithianyl, 1,3,5-triozalanyl, 1,3,5-trithianyl, 1,4-morpholinyl, thiomorpholinyl, 1,4-oxathianyl, triazinyl, 1,4-thiazinyl and the like.

Examples of 7-membered non-aromatic heterocyclyls include azepanyl, oxepanyl, thiepanyl and the like.

Non-aromatic heterocyclyl rings may also be bicyclic heterocyclyl rings such as linked ring systems (for example uridinyl and the like) or fused ring systems. Fused ring systems include non-aromatic 5-membered, 6-membered or 7-membered heterocyclyls fused to carbocyclic aromatic rings such as phenyl, naphthyl, indenyl, azulenyl, fluorenyl, anthracenyl and the like. Examples of non-aromatic 5-membered, 6-membered or 7-membered heterocyclyls fused to carbocyclic aromatic rings include indolinyl, benzodiazepinyl, benzazepinyl, dihydrobenzofuranyl and the like. The term “spiro ring system” means a bicyclic ring system in which the rings are connected via a single shared atom or “spiroatom” more particularly a quaternary carbon (“spiro carbon”) and encompasses spiro bicyclic 7-11-membered carbocyclic rings and spiro bicyclic 7-11-membered heterocyclic rings containing one, two, three or four heteroatoms independently selected from O, N and S.

Examples of heterocyclyl-C₁₋₄ alkyl are morpholinomethyl, morpholinoethyl, morpholinylmethyl, morpholinylethyl, piperidinomethyl, piperidinoethyl, piperidylmethyl, piperidylethyl, imidazolylmethyl, imidazolylethyl, tetrazolylmethyl, tetrazolylethyl, oxazolylmethyl, oxazolylethyl, 1,3,4-oxadiazolylmethyl, 1,2,4-oxadiazolylmethyl, 1,2,4-oxadiazolylethyl, pyridylmethyl, pyridylethyl, furylmethyl, furylethyl, (thienyl)methyl, (thienyl)ethyl, pyrazinylmethyl, pyrazinylethyl, piperazinylmethyl and piperazinylethyl.

Aryl is a partially saturated or unsaturated, mono or bicyclic carbocyclic ring that contains 3-12 atoms, preferably 6-10 atoms; wherein a —CH₂— group can optionally be replaced by a —C(O)—. Particularly aryl is a monocyclic ring containing 5 or 6 atoms or a bicyclic ring containing 9 or 10 atoms. In another aspect aryl is a totally unsaturated ring. Suitable values for aryl include phenyl, naphthyl, indanyl or 1-oxoindanyl.

The term “cycloalkyl” denotes a monovalent saturated monocyclic or bicyclic hydrocarbon group of 3 to 10 ring carbon atoms, particularly a monovalent saturated monocyclic hydrocarbon group of 3 to 8 ring carbon atoms. Bicyclic means consisting of two saturated or partially saturated carbocycles having two carbon atoms in common. Particular cycloalkyl groups are cyclopropyl, cyclobutyl, cyclopentyl, cyclohexyl, cycloheptenyl, bicyclo[2.2.2]heptanyl, bicyclo[2.2.2]octanyl, cyclohexenyl, substituted bycyclo[2.2.2]heptanyl and substituted bicyclo[2.2.2]octenyl.

The term “halogen” is used to denote fluoro, chloro, bromo, or iodo. Particular halogens are chloro and bromo. More particular halogen is chloro.

The term “heterocycloalkyl” denotes a monovalent saturated or partly unsaturated mono- or bicyclic ring system of 4 to 9 ring atoms, comprising 1, 2, or 3 ring heteroatoms selected from N, O and S, the remaining ring atoms being carbon. Bicyclic means consisting of two cycles having two ring atoms in common, i.e. the bridge separating the two rings is either a single bond or a chain of one or two ring atoms. Examples for monocyclic saturated heterocycloalkyl are 4,5-dihydro-oxazolyl, oxetanyl, azetidinyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocycloalkyl are 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocycloalkyl are dihydrofuryl, imidazolinyl, dihydro-oxazolyl, tetrahydro-pyridinyl, or dihydropyranyl. Further particular examples of heterocycloalkyl group are 4,5-dihydro-oxazolyl and pyrrolidinyl.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are the hydrochloride salts, methanesulfonic acid salts and citric acid salts.

“Pharmaceutically acceptable esters” means that compounds of general formula (I) may be derivatised at carboxylic or hydroxy groups to provide derivatives which are capable of conversion back to the parent compounds in vivo. Examples of such compounds include physiologically acceptable and metabolically labile ester derivatives, such as methoxymethyl esters, methylthiomethyl esters and pivaloyloxymethyl esters. Additionally, any physiologically acceptable equivalents of the compounds of general formula (I), similar to the metabolically labile esters, which are capable of producing the parent compounds of general formula (I) in vivo, are within the scope of this invention.

The compounds of formula (I) can contain several asymmetric centers and can be present in the form of pure enantiomers, mixtures of enantiomers such as, for example, racemates, pure diastereioisomers, and mixtures of diastereoisomers.

The term “sulfate” refers to the group OS(O)₂OH and includes groups having the hydrogen replaced with, for example a C₁₋₆ alkyl group (“alkylsulfates”), an aryl (“arylsulfate”), an aralkyl (“aralkylsulfate”) and so on. C₁₋₃ sulfates are preferred, such as for example, OS(O)₂OMe, OS(O)₂OEt and OS(O)₂OPr. The term “sulfonate” refers to the group SO3H and includes groups having the hydrogen replaced with, for example a C₁₋₆ alkyl group (“alkylsulfonate”), an aryl (“arylsulfonate”), an aralkyl (“aralkylsulfonate”) and so on. C₁₋₃ sulfonates are preferred, such as for example, SO₃Me, SO₃Et and SO₃Pr.

The term “phosphate” refers to a group —OP(O)(OH)₂ and includes groups having each hydrogen independently optionally replaced with, for example a C₁₋₆ alkyl group (“alkylphosphate”), an aryl (“arylphosphate”), an aralkyl (“aralkylphosphate”) and so on.

Unless otherwise defined, the term “optionally substituted” or “optional substituent” as used herein refers to a group which may or may not be further substituted with 1, 2, 3, 4 or more groups, preferably 1, 2 or 3, more preferably 1 or 2 groups selected from the group consisting of C₁₋₆ alkyl, C₂₋₆ alkenyl, C₂₋₆ alkynyl, C₃₋₈ cycloalkyl, hydroxyl, oxo, C₁₋₆ alkoxy, aryloxy, C₁₋₆ alkoxyaryl, halogen, C₁₋₆ alkylhalogen (such as CF₃ and CHF₂), C₁₋₆ alkoxyhalogen (such as OCF₃ and OCHF₂), carboxyl, alkoxycarbonyl, cyano, nitro, amino, mono substituted amino, disubstituted amino, acyl, amides, aminoacyl, substituted amides, disubstituted amides, carbamic acid, carbamates, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, phosphates, phosphonates, aryl, aryl-C₁₋₆ alkyl, heterocyclyl, heteroaryl and spiro ring systems wherein each alkyl, alkenyl, alkynyl, cycloalkyl, aryl, heterocyclyl, heteroaryl and spiro ring system and groups containing them may be further optionally substituted. Unless otherwise defined, particularly preferred optional substituents in one case include 1, 2, 3 or 4, preferably 1 or 2 substituents each independently selected from the group consisting of C₁₋₄ alkyl (particularly methyl), halogen (particularly F), halogeno-C₁₋₃ alkyl (particularly CHF₂ and CF₃), OH, C₁₋₄ alkoxyl (particularly OCH₃), COOH, COO—C₁₋₄ alkyl (particularly COOCH₃), NH₂, NH—C₁₋₄ alkyl (particularly NHCH₃), N(C₁₋₄ alkyl)₂ (particularly N(CH₃)₂), NHC(═O)—C₁₋₄ alkyl, NHC(═O)-4-6-membered heterocyclyl, OP(═O)(OR)₂(where each R is independently H or C₁₋₄ alkyl), P(═O)(OR)₂(where each R is independently H or C₁₋₄ alkyl), C₃₋₆ cycloalkyl (particularly cyclopropyl, cyclobutyl, cyclopenyl and cyclohexyl), phenyl, 4-6-membered heterocylyl (particularly oxetanyl, azetidinyl, tetrahydrofuranyl, pyrrolidinyl, tetrahydropyranyl, morpholinyl, thiomorpholinyl, oxothiazinyl, dioxothiazinyl, thianyl (also known as tetrahydrothiopyranyl), oxothianyl, dioxothianyl, piperidinyl, and piperazinyl) and further where C₁₋₄ alkyl either alone or as part of a substituent group includes methyl, ethyl, n-propyl, isopropyl, n-butyl, iso-butyl, sec-butyl and tert-butyl and may be further optionally substituted.

Optional substituents in the case of heterocycles, heteroaryls and spiro bicyclic heterocyclic ring systems containing N may also include but are not limited to alkyl, e.g. N—C₁₋₃ alkyl, more preferably methyl, particularly N-methyl. It will be understood that suitable derivatives of aromatic heterocyclyls containing nitrogen include N-oxides thereof.

Examples of C₁₋₄ alkyl include methyl, ethyl, propyl, butyl, tert-butyl and isopropyl; examples of C₁₋₆ alkyl include C₁₋₄ alkyl, pentyl and hexyl; examples of C₂₋₄ alkenyl include vinyl, propenyl, allyl, but-2-enyl and but-3-enyl; examples of C₂₋₆ alkenyl include C₂₋₄ alkenyl, pent-2-enyl, pent-3-enyl, and hex-5-enyl; examples of C₂₋₄ alkynyl include ethynyl, prop-2-ynyl, but-2-ynyl and but-3-ynyl; examples of C₂₋₆ alkynyl include C₂₋₄ alkynyl, pent-3-ynyl and hex-4-ynyl; examples of C₁₋₆ alkoxy and —O—C₁₋₆ alkyl include methoxy, ethoxy, propoxy, iso-propoxy, butoxy, tert-butoxy, pentoxy and hexoxy; examples of C₁₋₄ alkoxy include methoxy, ethoxy and propoxy; examples of C₁₋₄ alkoxy-C₁₋₄ alkyl include methoxymethyl, ethoxymethyl, methoxyethyl and propoxy methyl; examples of C₃₋₆ cycloalkyl include cyclopropyl, cyclobutyl, cyclopentyl and cyclohexyl; examples of halogen groups include fluoro, chloro and bromo; examples of halogeno-C₁₋₄ alkyl groups include fluoromethyl, fluoroethyl, chloromethyl, chloroethyl and bromomethyl; examples of hydroxyl-C₁₋₄ alkyl and hydroxyl-C₁₋₆-alkyl include hydroxymethyl, 1-hydroxyethyl, 2-hydroxyethyl and 3-hydroxypropyl; examples of —CO—C₁₋₄ alkyl include methylcarbonyl, ethylcarbonyl, propyl carbonyl, isopropylcarbonyl and tert-butylcarbonyl; examples of —CO—C₁₋₆ alkyl include —CO—C₁₋₄ alkyl and pentylcarbonyl; examples of —COO—C₁₋₄ alkyl include methoxycarbonyl, ethoxycarbonyl, propoxycarbonyl, isopropoxy carbonyl and tert-butoxycarbonyl; examples of —COO—C₁₋₆ alkyl include —COO—C₁₋₄ alkyl and pentoxycarbonyl; examples of —OCO—C₁₋₄ alkyl include methylcarbonyloxy, ethylcarbonyloxy, propylcarbonyloxy, isopropylcarbonyloxy and tert-butylcarbonyloxy; examples of —OCO—C₁₋₆ alkyl include —OCO—C₁₋₄ alkyl and pentylcarbonyloxy; examples of —C₁₋₄ alkyl-COO—C₁₋₄ alkyl include methoxycarbonylmethyl, ethoxycarbonylmethyl, methoxycarbonylethyl, propoxycarbonylmethyl, isopropoxycarbonylmethyl and tert-butoxycarbonylmethyl; examples of —C(O)NH—C₁₋₄ alkyl include methylaminocarbonyl, ethylaminocarbonyl, propylaminocarbonyl, isopropylaminocarbonyl and tert-butylaminocarbonyl; examples of —C(O)N[C₁₋₄ alkyl]₂ include N-dimethylaminocarbonyl and N-methyl-N-ethylaminocarbonyl; examples of —S(O)₂NH—C₁₋₄alkyl include N-methylaminosulfonyl and N-ethylaminosulfonyl; examples of —S(O)₂N[C₁₋₄ alkyl]₂ include N,N-dimethylaminosulfonyl, N,N-diethylaminosulfonyl and N-methyl-N-ethylaminosulfonyl; examples of —S(O)p-C₁₋₄ alkyl include methylthio, methylsulfinyl, methylsulfonyl, ethylthio, propylthio, isopropylthio, ethylsulfinyl and ethylsulfonyl.

Another embodiment of the invention provides a compound selected from the group consisting of any one or more of the compounds described in the Examples section, or a pharmaceutically acceptable salt thereof. Moreover, if such compound is represented as a salt, the present invention is intended to include free bases, free acids, or alternative salts of these particular compounds. Additional embodiments comprise compositions and medicaments containing the same (including aforementioned free bases, free acids, or alternative salts, as well as processes for the preparation and use of such compounds, compositions and medicaments. Moreover, it should be noted that each of these compounds and salts thereof, are also intended to be separate embodiments, and in this regard, each species listed in Examples, and salt thereof, should be considered to be an individual embodiment. Moreover, it should be understood that the present invention is intended to include any novel compound or pharmaceutical composition described herein.

Unless otherwise stated, variables in the embodiments below are defined as for formula I.

According to certain embodiments, R¹ is hydrogen.

According to certain embodiments, R¹ is methyl.

According to certain embodiments, R¹ is ethyl.

According to certain embodiments, R¹ is propyl.

According to certain embodiments, R¹ is butyl.

According to certain embodiments, R¹ is isopropyl.

According to certain embodiments, R¹ is cyclopropyl.

According to certain embodiments, R¹ is chloro.

According to certain embodiments, R¹ is bromo.

According to certain embodiments, R¹ is fluoro.

According to certain embodiments, R¹ is iodo.

According to certain embodiments, R² is hydrogen.

According to certain embodiments, R² is chloro.

According to certain embodiments, R² is bromo.

According to certain embodiments, R² is fluoro.

According to certain embodiments, R² is iodo.

According to certain embodiments, R² is cyano.

According to certain embodiments, R² is trifluoromethyl.

According to certain embodiments, R³ is hydrogen.

According to certain embodiments, R³ is chloro.

According to certain embodiments, R³ is bromo.

According to certain embodiments, R³ is fluoro.

According to certain embodiments, R³ is iodo.

According to certain embodiments, R³ is cyano.

According to certain embodiments, R³ is trifluoromethyl.

According to certain embodiments, R⁴ is hydrogen.

According to certain embodiments, R⁴ is represented by formula (CH₂)₀₋₆-A and A is hydrogen and may be optionally substituted.

According to certain embodiments, R⁴ is represented by formula (CH₂)₀₋₆-A and A is monocyclic C₃₇ cycloalkyl and may be optionally substituted.

According to certain embodiments, R⁴ is represented by formula (CH₂)₀₋₆-A and A is monocyclic C₃₇ cycloalkenyl and may be optionally substituted.

According to certain embodiments, R⁴ is represented by formula (CH₂)₀₋₆-A and A is saturated or unsaturated monocyclic 3-7 membered heterocycle and may be optionally substituted.

According to certain embodiments, R⁴ is represented by formula (CH₂)₀₋₆-A and A is saturated or unsaturated fused bicyclic 8-10 membered-heterocycle and may be optionally substituted.

According to certain embodiments, R⁴ is represented by formula (CH₂)₀₋₆-A and A is C₆₋₁₀ aryl and may be optionally substituted.

According to certain embodiments, R⁴ is represented by formula (CH₂)₀₋₆-A and A is 5-10 membered heteroaryl and may be optionally substituted.

According to certain embodiments, R⁵ is selected from a group consisting of H, hydroxy, halogen, carboxyl, optionally substituted C₁₋₆ alkyl, optionally substituted C₂₋₆ alkenyl, optionally substituted C₂₋₆ alkynyl, optionally substituted (CH₂)ₘO—C₁₋₆ alkyl, optionally substituted (CH₂)ₘS—C₁₋₆ alkyl, optionally substituted (CH₂)ₘS(═O)—C₁₋₆ alkyl, optionally substituted (CH₂)ₘO(CH₂)ₘ—C₃₋₇ cycloalkyl, optionally substituted (CH₂)ₘ—C₃₋₇ cycloalkyl, optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle, optionally substituted (CH₂)ₘ-5-10-membered heterocycle, optionally substituted halogeno-C₁₋₆ alkyl, cyano or optionally substituted (CH₂)ₘNR^{5′}R^{5″} and where each m is an integer independently selected from 0, 1, 2 and 3, preferably where each m is an integer independently selected from 0, 1 and 2, more preferably where each m is an integer independently selected from 0 and 1.

According to certain embodiments, R⁵ is selected from a group consisting of optionally substituted (CH₂)ₘO(CH₂)ₘ—C₃₋₇ cycloalkyl, optionally substituted (CH₂)ₘ—C₃₋₇ cycloalkyl, optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle, optionally substituted (CH₂)ₘ-5-10-membered heterocycle and optionally substituted (CH₂)ₘNR^{5′}R^{5″}, where each m is an integer independently selected from 0, 1, 2 and 3, preferably where each m is an integer independently selected from 0, 1 and 2, more preferably where each m is an integer independently selected from 0 and 1.

According to certain embodiments, R⁵ is selected from a group consisting of optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle, optionally substituted (CH₂)ₘ-5-10-membered heterocycle and optionally substituted (CH₂)ₘNR^{5′}R^{5″}, where each m is an integer independently selected from 0, 1, 2 and 3, preferably where each m is an integer independently selected from 0, 1 and 2, more preferably where each m is an integer independently selected from 0 and 1.

According to certain embodiments, R⁵ is selected from a group consisting of optionally substituted (CH₂)ₘO(CH₂)ₘ-5-10-membered heterocycle, optionally substituted (CH₂)ₘ-5-10-membered heterocycle and optionally substituted (CH₂)ₘNR^{5′}R^{5″}, where each m is an integer independently selected from 0, 1, 2 and 3, preferably where each m is an integer independently selected from 0, 1 and 2, more preferably where each m is an integer independently selected from 0 and 1.

According to certain embodiments, R⁵ is selected from a group consisting of optionally substituted (CH₂)ₘO(CH₂)ₘ-5-10-membered heterocycle and optionally substituted (CH₂)ₘ-5-10-membered heterocycle, where each m is an integer independently selected from 0, 1, 2 and 3, preferably where each m is an integer independently selected from 0, 1 and 2, more preferably where each m is an integer independently selected from 0 and 1.

According to certain embodiments, R⁵ is an optionally substituted (CH₂)ₘO(CH₂)ₘ-5-10-membered heterocycle with m being an integer independently selected from 0, 1, 2 and 3, preferably where m is an integer independently selected from 0, 1 and 2, more preferably where m is an integer independently selected from 0 and 1.

According to certain embodiments, R⁵ is an optionally substituted (CH₂)ₘ-5-10-membered heterocycle with m being an integer independently selected from 0, 1, 2 and 3, preferably where m is an integer independently selected from 0, 1 and 2, more preferably where m is an integer independently selected from 0 and 1.

According to certain embodiments, R⁵ is optionally substituted (CH₂)ₘNR^{5′}R^{5″} with m being an integer independently selected from 0, 1, 2 and 3, preferably where m is an integer independently selected from 0, 1 and 2, more preferably where m is an integer independently selected from 0 and 1.

According to certain embodiments, R⁵ is selected from the group consisting of O(CH₂)ₘ-isopropyl, O(CH₂)ₘ pyrrolidine, O(CH₂)ₘ-piperidine, O(CH₂)ₘ-morpholine, O(CH₂)ₘ-pyridine, O(CH₂)ₘ-pyrimidine, O(CH₂)ₘ-thiophene, O(CH₂)ₘ-phenyl, (CH₂)ₘ-isopropyl, (CH₂)ₘ-pyrrolidine, (CH₂)ₘ-piperidine, (CH₂)ₘ-morpholine, (CH₂)ₘ-pyridine, (CH₂)ₘ-pyrimidine, (CH₂)ₘ-thiophene and (CH₂)ₘ-phenyl, wherein each m is an integer independently selected from 0, 1 or 2, and wherein any alkyl, heterocyclyl or aryl may be optionally substituted.

According to certain embodiments, R⁵ is selected from the group consisting of O(CH₂)ₘ-isopropyl, O(CH₂)ₘ pyrrolidine, O(CH₂)ₘ-piperidine, O(CH₂)ₘ-morpholine, O(CH₂)ₘ-pyridine, O(CH₂)ₘ-pyrimidine, O(CH₂)ₘ-thiophene, O(CH₂)ₘ-phenyl, wherein each m is an integer independently selected from 0, 1 or 2, and wherein any alkyl, heterocyclyl or aryl may be optionally substituted.

According to certain embodiments, R⁵ is selected from the group consisting of O(CH₂)ₘ-pyrrolidine, O(CH₂)ₘ piperidine, O(CH₂)ₘ-morpholine, O(CH₂)ₘ-pyridine, O(CH₂)ₘ-pyrimidine, O(CH₂)ₘ-thiophene, (CH₂)ₘ-pyrrolidine, (CH₂)ₘ-piperidine, (CH₂)ₘ-morpholine, (CH₂)ₘ-pyridine, (CH₂)ₘ-pyrimidine and (CH₂)ₘ-thiophene, wherein each m is an integer independently selected from 0, 1 or 2, and wherein any alkyl, heterocyclyl or aryl may be optionally substituted.

According to certain embodiments, R⁵ is selected from the group consisting of O(CH₂)ₘ-pyrrolidine, O(CH₂)ₘ piperidine, O(CH₂)ₘ-morpholine, O(CH₂)ₘ-pyridine, O(CH₂)ₘ-pyrimidine and O(CH₂)ₘ-thiophene, wherein each m is an integer independently selected from 0, 1 or 2, and wherein any alkyl, heterocyclyl or aryl may be optionally substituted.

According to certain embodiments, R⁵ is selected from the group consisting of (CH₂)ₘ-pyrrolidine, (CH₂)ₘ piperidine, (CH₂)ₘ-morpholine, (CH₂)ₘ-pyridine, (CH₂)ₘ-pyrimidine and (CH₂)ₘ-thiophene, wherein each m is an integer independently selected from 0, 1 or 2, and wherein any alkyl, heterocyclyl or aryl may be optionally substituted.

According to certain embodiments, each m in variable R⁵ is 0, 1 or 2.

According to certain embodiments, each m in variable R⁵ is 0 or 1.

According to certain embodiments, each m in variable R⁵ is 0.

According to certain embodiments, each m in variable R⁵ is 1.

According to certain embodiments, each m in variable R⁵ is 2.

According to certain embodiments, the optionally substituted heterocycle in R⁵ is selected from the group consisting of piperidine, piperidin-3-amine, piperidin-4-amine, piperidin-3-ylmethanamine, piperazine, pyrrolidine, pyrrolidin-3-amine, morpholine, isoindoline and 1-phenylpiperazine.

According to certain embodiments, R^{5′} and R^{5″} are each independently selected from H or optionally substituted C₁₋₆ alkyl, optionally substituted C₃₋₆ cycloylkyl and optionally substituted 4-6-membered heterocyclyl or R^{5′} and R^{5″} join together to form an optionally substituted 4-6-membered heterocyclyl and where each m is an integer independently selected from 0, 1, 2 and 3, preferably where each m is an integer independently selected from 0, 1 and 2, more preferably where each m is an integer independently selected from 0 and 1.

According to certain embodiments, R^{5′} and R^{5″} are each independently selected from H and optionally substituted 4-6-membered heterocyclyl.

According to certain embodiments, R^{5′} is H and R^{5″} is optionally substituted 4-6-membered heterocyclyl.

According to certain embodiments, R^{5′} is H and R^{5″} is optionally substituted piperidine.

According to certain embodiments, R⁶ is an optionally substituted 5-10-membered heterocyclyl, optionally substituted 6-10-membered aryl, —CONR^{6′}R^{6″} and —CONHCH(CO₂R^{6″})R^{6′}.

According to certain embodiments, R⁶ is an optionally substituted 5-10-membered heterocyclyl, optionally substituted 6-10-membered aryl, and —CONHCH(CO₂R^{6″})R^{6′}.

According to certain embodiments, R⁶ is an optionally substituted 5-10-membered heterocyclyl or —CONHCH(CO₂R^{6″})R^{6′}.

According to certain embodiments, R⁶ is an optionally substituted 5-10-membered heterocyclyl, —CONR^{6′}R^{6″} and —C(O)NHS(O)p-C₁₋₆ alkyl.

According to certain embodiments, R⁶ is an optionally substituted 5-membered heterocyclyl, —CONR^{6′}R^{6″} and —C(O)NHS(O)p-C₁₋₆ alkyl.

According to certain embodiments, R⁶ is an optionally substituted 5-10-membered heterocyclyl.

According to certain embodiments, R⁶ is an optionally substituted 5-membered heterocyclyl.

According to certain embodiments, the optionally substituted 5-10-membered heterocyclyl in R⁶ is selected from the group consisting of 1,2,4-oxadiazol-5-one, tetrazole, 1,3,4-oxadiazol-2-one and 1,4-dihydro-tetrazol-5-one.

According to certain embodiments, R⁶ is —CONHCH(CO₂R^{6″})R^{6′}.

According to certain embodiments, R⁶ is —CONHCH₂(CO₂H).

According to certain embodiments, R⁶ is (hydroxyimino)methyl.

According to certain embodiments, R⁶ is selected from optionally substituted —CO—C₁₋₆ alkyl.

According to certain embodiments, R⁶ is selected from optionally substituted —CO—C₂₋₆ alkenyl.

According to certain embodiments, R⁶ is selected from optionally substituted —CO—C₂₋₆ alkynyl.

According to certain embodiments, R⁶ is carboxyl.

According to certain embodiments, R⁶ is selected from optionally substituted —COO—C₁₋₆ alkyl.

According to certain embodiments, R⁶ is selected from optionally substituted —COO—C₁₋₆ alkyl (optionally substituted with —COO—C₁₋₆ alkyl).

According to certain embodiments, R⁶ is selected from —CONR^{6′}R^{6″}.

According to certain embodiments, R⁶ is selected from —O(CO)NR^{6′}R^{6″}.

According to certain embodiments, R⁶ is selected from —O—C₁₋₆ alkyl.

According to certain embodiments, R⁶ is selected from —OR^{6′}.

According to certain embodiments, R⁶ is selected from —NH—C₁₋₆ alkyl.

According to certain embodiments, R⁶ is selected from —NH—C₁₋₆ acyl.

According to certain embodiments, R⁶ is selected from —NR^{6′}R^{6″}.

According to certain embodiments, R⁶ is selected from optionally substituted —(CH₂)₁₋₆OH.

According to certain embodiments, R⁶ is selected from optionally substituted —C(═NOR^{6″})—C₁₋₄ alkyl. According to certain embodiments, R⁶ is selected from —C(═NOR^{6″})NR^{6′}R^{6″} According to certain embodiments, R⁶ is selected from —S(O)pNR^{6′}R^{6″}.

According to certain embodiments, R⁶ is selected from optionally substituted —S(O)p-C₁₋₄ alkyl-CONHR^{6″}.

According to certain embodiments, R⁶ is selected from optionally substituted —C(O)NHS(O)p-C₁₋₄ alkyl.

According to certain embodiments, R⁶ is selected from optionally substituted —C(O)NHS(O)p-aryl.

According to certain embodiments, R⁶ is selected from —CH₂CH(COOR^{6′})OH.

According to certain embodiments, R⁶ is selected from optionally substituted —C₁₋₄ alkyl-CH(NR^{6′}R^{6″})COOR⁶.

According to certain embodiments, R⁶ is selected from optionally substituted —C₁₋₄ alkyl-CH(NR^{6′}R^{6″})CONR^{6′}R^{6″}) According to certain embodiments, R^{6′} is hydrogen.

According to certain embodiments, R^{6′} is selected from optionally substituted C₁₋₆ alkyl.

According to certain embodiments, R^{6′} is selected from optionally substituted C₁₋₆ acyl.

According to certain embodiments, R^{6″} is hydrogen.

According to certain embodiments, R^{6″} is selected from optionally substituted C₁₋₆ alkyl.

According to certain embodiments, R^{6′} and R^{6″} are both hydrogen.

According to certain embodiments, R^{6′} and R^{6″} may together with the nitrogen to which they are attached form a 5 or 6-membered heterocyclic ring, optionally substituted with 1 or 2 substituents independently selected from C₁₋₄ alkyl, C₂₋₄ alkenyl, (C₂₋₄ alkynyl, hydroxy, C₁₋₄ alkoxy, halogen, cyano, nitro, carboxyl, hydroxyl-C₁₋₄ alkyl-, C₁₋₄ alkoxy-C₁₋₄ alkyl-, halogeno-C₁₋₄ alkyl-, difluoromethyl, trifluoromethyl, formyl, —CO—C₁₋₄ alkyl, —COO—C₁₋₄ alkyl, —C(O)NH₂, —C(O)NH—C₁₋₄ alkyl, —C(O)N[C₁₋₄ alkyl]₂, —SO₂NH₂, —SO₂NH—C₁₋₄ alkyl, —SO₂[C₁₋₄ alkyl]₂ and —S(O)p-C₁₋₄ alkyl, wherein p is (independently at each occurrence) 0, 1 or 2.

According to certain embodiments, a compound of formula I is provided, wherein:
R¹ is selected from methyl, chloro and bromo;
R² and R³ are each independently selected from chloro, bromo or fluoro;
R⁴ is (CH₂)₀₋₆-A
   wherein A is H, carboxyl, NR^{6′}R^{6″} or is selected from optionally substituted monocyclic C₃₋₇ cycloalkyl, optionally substituted monocyclic C₃₋₇cycloalkenyl, optionally substituted saturated or unsaturated monocyclic 3-7 membered heterocycle, optionally substituted saturated or unsaturated fused bicyclic 8-10 membered-heterocycle, optionally substituted C₆₋₁₀ aryl and optionally substituted 5-10 membered heteroaryl;
R⁵ is selected from a group consisting of H, hydroxy, halogen, carboxyl, optionally substituted C₁₋₆ alkyl, optionally substituted C₂₋₆ alkenyl, optionally substituted C₂₋₆ alkynyl, optionally substituted (CH₂)ₘO—C₁₋₆ alkyl, optionally substituted (CH₂)ₘS—C₁₋₆ alkyl, optionally substituted (CH₂)ₘS(═O)—C₁₋₆ alkyl, optionally substituted (CH₂)ₘO(CH₂)ₘ—C₃₋₇ cycloalkyl, optionally substituted (CH₂)ₘ—C₃₋₇ cycloalkyl, optionally substituted (CH₂)ₘO(CH₂)ₘ-6-10-membered aryl, optionally substituted (CH₂)ₘ6-10-membered aryl, optionally substituted (CH₂)ₘO(CH₂)ₘ-5-10-membered heterocycle, optionally substituted (CH₂)ₘ-5-10-membered heterocycle, optionally substituted halogen C₃₋₇ alkyl, cyano or optionally substituted (CH₂)ₘNR^{5′}R^{5″} and where each m is an integer independently selected from 0, 1, 2 and 3, preferably where each m is an integer independently selected from 0, 1 and 2, more preferably where each m is an integer independently selected from 0 or 1;
R^{5′} and R^{5″} are each independently selected from H or optionally substituted C₁₋₆ alkyl, optionally substituted C₃₋₆ cycloylkyl and optionally substituted 4-6-membered heterocyclyl or R^{5′} and R^{5″} join together to form an optionally substituted 4-6-membered heterocyclyl and where each m is an integer independently selected from 0, 1, 2 and 3;
R⁶ is (hydroxyimino)methyl, —CO—C₁₋₆ alkyl, —CO—C₂₋₆ alkenyl, —CO—C₂₋₆ alkynyl, carboxyl, —COO—C₁₋₆ alkyl, —CONR^{6′}R^{6″}, —OCONR^{6′}R^{6″}, —OCO—C₁₋₆ alkyl, —COO—C₁₋₆ alkyl (optionally substituted with —COO—C₁₋₆ alkyl), —CONHCH(CO₂R^{6″})R^{6′}, —(CH₂)₀₋₆OH, —O—C₁₋₆ alkyl, —OR^{6′}, —NH—C₁₋₆ alkyl, —NH—C₁₋₆ acyl, —NR^{6′}R^{6″}, hydroxyl-C₁₋₆ alkyl, 5-10-membered heterocyclyl, 6-10-membered aryl, —C(═NOR^{6″})C₁₋₆ alkyl, —C(═NOR^{6″})NR^{6′}R^{6″}, —S(O)pNR^{6′}R^{6″}, —S(O)p-C₁₋₆ alkyl-CONHR^{6″}, —C(O)NHS(O)p-C₁₋₆ alkyl, —C(O)NHS(O)p-aryl, —CH₂CH(COOR⁶)OH, —C₁₋₆ alkyl-CH(NR^{6′}R^{6″})COOR^{6′} or —C₁₋₆ alkyl-CH(NR^{6′}R^{6″})CONR^{6′}R^{6″}), wherein any alkyl, alkenyl, alkynyl, heterocyclyl or aryl may be optionally substituted; and
R^{6′} and R^{6″} are independently at each occurrence selected from H and optionally substituted C₁₋₆ alkyl or C₁₋₆ acyl;
or R^{6′} and R^{6″} may together with the nitrogen to which they are attached form a5 or 6-membered heterocyclic ring, optionally substituted with 1 or 2 substituents independently selected from C₁₋₄ alkyl, C₂₋₄ alkenyl, C₂₋₄ alkynyl, hydroxyl, C₁₋₄alkoxy, halogen, cyano, nitro, carboxyl, hydroxy-C₁₋₄alkyl-, C₁₋₄alkoxy-C₁₋₄alkyl-, halogeno-C₁₋₄alkyl-, difluoromethyl, trifluoromethyl, formyl, —CO—C₁₋₄alkyl, —COO—C₁₋₄alkyl, —C(O)NH₂, —C(O)NH—C₁₋₄alkyl, —C(O)N[C₁₋₄ alkyl]₂, —SO₂NH₂, —SO₂NH—C₁₋₄ alkyl, —SO₂[C₁₋₄alkyl] and —S(O)p-C₁₄ alkyl; p is (independently at each occurrence) 0, 1 or 2.

According to certain embodiments, a compound of formula I is provided, wherein:
R¹ is selected from methyl, chloro and bromo;
R² and R³ are each independently selected from chloro or bromo;
R⁴ is (CH₂)₀₋₆-A
   wherein A is H, carboxyl, NR^{6′}R^{6″} or is selected from optionally substituted monocyclic C₃₋₇ cycloalkyl, optionally substituted monocyclic C₃₋₇cycloalkenyl, optionally substituted saturated or unsaturated monocyclic 3-7 membered heterocycle, optionally substituted saturated or unsaturated fused bicyclic 8-10 membered-heterocycle, optionally substituted C₆₋₁₀ aryl and optionally substituted 5-10 membered heteroaryl;
R⁵ is selected from a group consisting of H, hydroxy, halogen, carboxyl, optionally substituted C₁₋₆ alkyl, optionally substituted C₂₋₆ alkenyl, optionally substituted C₂₋₆ alkynyl, optionally substituted (CH₂)ₘO—C₁₋₆ alkyl, optionally substituted (CH₂)ₘS—C₁₋₆ alkyl, optionally substituted (CH₂)ₘS(═O)—C₁₋₆ alkyl, optionally substituted (CH₂)ₘO(CH₂)ₘ—C₃₋₇ cycloalkyl, optionally substituted (CH₂)ₘ—C₃₋₇ cycloalkyl, optionally substituted (CH₂)ₘO(CH₂)ₘ-6-10-membered aryl, optionally substituted (CH₂)ₘ-6-10-membered aryl optionally substituted (CH₂)ₘO(CH₂)ₘ-5-10-membered heterocycleheterocycle, optionally substituted (CH₂)ₘ-5-10-membered heterocycleheterocycle, optionally substituted halogen C₃₋₇ alkyl, cyano or optionally substituted (CH₂)ₘNR^{5′}R^{5″} and where each m is an integer independently selected from 0, 1, 2 and 3, preferably where each m is an integer independently selected from 0, 1 and 2, more preferably where each m is an integer independently selected from 0 or 1;
R^{5′} and R^{Y}“are each independently selected from H or optionally substituted C₁₋₆ alkyl, optionally substituted C₃₋₆ cycloylkyl and optionally substituted 4-6-membered heterocyclyl or R^{5′} and R” join together to form an optionally substituted 4-6-membered heterocyclyl and where each m is an integer independently selected from 0, 1, 2 and 3;
R⁶ is (hydroxyimino)methyl, —CO—C₁₋₆ alkyl, —CO—C₂₋₆ alkenyl, —CO—C₂₋₆ alkynyl, carboxyl, —COO—C₁₋₆ alkyl, —CONR^{6′}R^{6″}, —OCONR^{6′}R^{6″}, —OCO—C₁₋₆ alkyl, —COO—C₁₋₆ alkyl (optionally substituted with —COO—C₁₋₆ alkyl), —CONHCH(CO₂R^{6″})R⁶, —(CH₂)₀₋₆OH, —O—C₁₋₆ alkyl, —OR^{6′}, —NH—C₁₋₆ alkyl, —NH—C₁₋₆ acyl, —NR^{6′}R^{6″}, hydroxyl-C₁₋₆ alkyl, 5-10-membered heterocyclyl, 6-10-membered aryl, —C(═NOR^{6″})C₁₋₆ alkyl, —C(═NOR^{6″})NR^{6′}R^{6″}, —S(O)pNR^{6′}R^{6″}, —S(O)p-C₁₋₆ alkyl-CONHR^{6″}, —C(O)NHS(O)p-C₁₋₆ alkyl, —C(O)NHS(O)p-aryl, —CH₂CH(COOR^{6′})OH, —C₁₋₆ alkyl-CH(NR^{6′}R^{6″})COOR^{6′} or —C₁₋₆ alkyl-CH(NR^{6′}R^{6″})CONR^{6′}R^{6″}), wherein any alkyl, alkenyl, alkynyl, heterocyclyl or aryl may be optionally substituted; and
R^{6′} and R^{6″} are independently at each occurrence selected from H and optionally substituted C₁₋₆ alkyl or C₁₋₆ acyl;
or R^{6′} and R^{6″} may together with the nitrogen to which they are attached form a 5 or 6-membered heterocyclic ring, optionally substituted with 1 or 2 substituents independently selected from C₁₋₄ alkyl, C₂₋₄ alkenyl, C₂₋₄ alkynyl, hydroxyl, C₁₋₄ alkoxy, halogen, cyano, nitro, carboxyl, hydroxy-C₁₋₄ alkyl-, C₁₋₄ alkoxy-C₁₋₄ alkyl-, halogeno-C₁₋₄ alkyl-, difluoromethyl, trifluoromethyl, formyl, —CO—C₁₋₄ alkyl, —COO—C₁₋₄ alkyl, —C(O)NH₂, —C(O)NH—C₁₋₄ alkyl, —C(O)N[C₁₋₄alkyl]₂, —SO₂NH₂, —SO₂NH—C₁₋₄ alkyl, —SO₂[C₁₋₄alkyl] and —S(O)p-C₁₄ alkyl; p is (independently at each occurrence) 0, 1 or 2.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is selected from a group consisting of H, hydroxy, halogen, carboxyl, optionally substituted C₁₋₆ alkyl, optionally substituted C₂₋₆ alkenyl, optionally substituted C₂₋₆ alkynyl, optionally substituted (CH₂)ₘO—C₁₋₆ alkyl, optionally substituted (CH₂)ₘS—C₁₋₆ alkyl, optionally substituted (CH₂)ₘS(═O)—C₁₋₆ alkyl, optionally substituted (CH₂)ₘO(CH₂)ₘ—C₃₋₇ cycloalkyl, optionally substituted (CH₂)ₘ—C₃₇ cycloalkyl, optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle, optionally substituted (CH₂)ₘ-5-10-membered heterocycle, optionally substituted halogen C₃₇ alkyl, cyano or optionally substituted (CH₂)ₘNR^{5′}R^{5″} and where each m is an integer independently selected from 0, 1, 2 and 3, preferably where each m is an integer independently selected from 0, 1 and 2, more preferably where each m is an integer independently selected from 0 or 1;
R^{5′} and R^{5″} are each independently selected from H or optionally substituted C₁₋₆ alkyl, optionally substituted C₃₋₆ cycloylkyl and optionally substituted 4-6-membered heterocyclyl or R^{5′} and R^{5″} join together to form an optionally substituted 4-6-membered heterocyclyl and where each m is an integer independently selected from 0, 1, 2 and 3;
R⁶ is (hydroxyimino)methyl, —CO—C₁₋₆ alkyl, —CO—C₂₋₆ alkenyl, —CO—C₂₋₆ alkynyl, carboxyl, —COO—C₁₋₆ alkyl, —CONR^{6′}R^{6″}, —OCONR^{6′}R^{6″}, —OCO—C₁₋₆ alkyl, —COO—C₁₋₆ alkyl (optionally substituted with —COO—C₁₋₆ alkyl), —CONHCH(CO₂R^{6″})R^{6′}, —(CH₂)₀₋₆OH, —O—C₁₋₆ alkyl, —OR^{6′}, —NH—C₁₋₆ alkyl, —NH—C₁₋₆ acyl, —NR^{6′}R^{6″}, hydroxyl-C₁₋₆ alkyl, 5-10-membered heterocyclyl, 6-10-membered aryl, —C(═NOR^{6″})C₁₋₆ alkyl, —C(═NOR^{6″})NR^{6′}R^{6″}, —S(O)pNR^{6′}R^{6″}, —S(O)p-C₁₋₆ alkyl-CONHR^{6″}, —C(O)NHS(O)p-C₁₋₆ alkyl, —C(O)NHS(O)p-aryl, —CH₂CH(COOR^{6′})OH, —C₁₋₆ alkyl-CH(NR^{6′}R^{6″})COOR^{6′} or —C₁₋₆ alkyl-CH(NR^{6′}R^{6″})CONR^{6′}R^{6″}), wherein any alkyl, alkenyl, alkynyl, heterocyclyl or aryl may be optionally substituted; and
R^{6′} and R^{6″} are independently at each occurrence selected from H and optionally substituted C₁₋₆ alkyl or C₁₋₆ acyl;
or R^{6′} and R^{6″} may together with the nitrogen to which they are attached form a5 or 6-membered heterocyclic ring, optionally substituted with 1 or 2 substituents independently selected from C₁₋₄ alkyl, C₂₋₄ alkenyl, C₂₋₄ alkynyl, hydroxyl, C₁₋₄alkoxy, halogen, cyano, nitro, carboxyl, hydroxy-C₁₋₄alkyl-, C₁₋₄alkoxy-C₁₋₄alkyl-, halogeno-C₁₋₄alkyl-, difluoromethyl, trifluoromethyl, formyl, —CO—C₁₋₄alkyl, —COO—C₁₋₄alkyl, —C(O)NH₂, —C(O)NH—C₁₋₄alkyl, —C(O)N[C₁₋₄ alkyl]₂, —SO₂NH₂, —SO₂NH—C₁₋₄ alkyl, —SO₂[C₁₋₄alkyl] and —S(O)p-C14 alkyl; p is (independently at each occurrence) 0, 1 or 2.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is H;
R⁶ is (hydroxyimino)methyl, —CO—C₁₋₆ alkyl, —CO—C₂₋₆ alkenyl, —CO—C₂₋₆ alkynyl, carboxyl, —COO—C₁₋₆ alkyl, —CONR^{6′}R^{6″}, —OCONR^{6′}R^{6″}, —OCO—C₁₋₆ alkyl, —COO—C₁₋₆ alkyl (optionally substituted with —COO—C₁₋₆ alkyl), —CONHCH(CO₂R^{6″})R⁶, —(CH₂)₀₋₆OH, —O—C₁₋₆ alkyl, hydroxyl-C₁₋₆ alkyl, 5-10-membered heterocyclyl, 6-10-membered aryl, —C(═NOR^{6″})C₁₋₆ alkyl, —C(═NOR^{6″})NR^{6′}R^{6″}, —S(O)pNR^{6′}R^{6″}, —S(O)p-C₁₋₆ alkyl-CONHR^{6″}, —C(O)NHS(O)p-C₁₋₆ alkyl, —C(O)NHS(O)p-aryl, —CH₂CH(COOR⁶)OH, —C₁₋₆ alkyl-CH(NR^{6′}R^{6″})COOR^{6′} or —C₁₋₆ alkyl-CH(NR^{6′}R^{6″})CON R^{6′}R^{6″}), wherein any alkyl, alkenyl, alkynyl, heterocyclyl or aryl may be optionally substituted; and
R^{6′} and R^{6″} are independently at each occurrence selected from H and C₁₋₆ alkyl;
or R^{6′} and R^{6″} may together with the nitrogen to which they are attached form a5 or 6-membered heterocyclic ring, optionally substituted with 1 or 2 substituents independently selected from C₁₋₄ alkyl, C₂₋₄ alkenyl, C₂₋₄ alkynyl, hydroxyl, C₁₋₄alkoxy, halogen, cyano, nitro, carboxyl, hydroxy-C₁₋₄alkyl-, C₁₋₄alkoxy-C₁₋₄alkyl-, halogeno-C₁₋₄alkyl-, difluoromethyl, trifluoromethyl, formyl, —CO—C₁₋₄alkyl, —COO—C₁₋₄alkyl, —C(O)NH₂, —C(O)NH—C₁₋₄alkyl, —C(O)N[C₁₋₄ alkyl]₂, —SO₂NH₂, —SO₂NH—C₁₋₄ alkyl, —SO₂[C₁₋₄alkyl] and —S(O)p-C14 alkyl; p is (independently at each occurrence) 0, 1 or 2.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is OR^{5″};
R^{5′} is independently selected from H or optionally substituted C₁₋₆ alkyl, optionally substituted C₃₋₆ cycloylkyl and optionally substituted 4-6-membered heterocyclyl or R^{5′} and R^{5″} join together to form an optionally substituted 4-6-membered heterocyclyl and where each m is an integer independently selected from 0, 1, 2 and 3;
R⁶ is (hydroxyimino)methyl, —CO—C₁₋₆ alkyl, —CO—C₂₋₆ alkenyl, —CO—C₂₋₆ alkynyl, carboxyl, —COO—C₁₋₆ alkyl, —CONR^{6′}R^{6″}, —OCONR^{6′}R^{6″}, —OCO—C₁₋₆ alkyl, —COO—C₁₋₆ alkyl (optionally substituted with —COO—C₁₋₆ alkyl), —CONHCH(CO₂R^{6″})R^{6′}, —(CH₂)₀₋₆OH, —O—C₁₋₆ alkyl, —OR^{6′}, —NH—C₁₋₆ alkyl, —NH—C₁₋₆ acyl, —NR^{6′}R^{6″}, hydroxyl-C₁₋₆ alkyl, 5-10-membered heterocyclyl, 6-10-membered aryl, —C(═NOR^{6″})C₁₋₆ alkyl, —C(═NOR^{6″})NR^{6′}R^{6″}, —S(O)pNR^{6′}R^{6″}, —S(O)p-C₁₋₆ alkyl-CONHR^{6″}, —C(O)NHS(O)p-C₁₋₆ alkyl, —C(O)NHS(O)p-aryl, —CH₂CH(COOR^{6′})OH, —C₁₋₆ alkyl-CH(NR^{6′}R^{6″})COOR^{6′} or —C₁₋₆ alkyl-CH(NR^{6′}R^{6″})CONR^{6′}R^{6″}), wherein any alkyl, alkenyl, alkynyl, heterocyclyl or aryl may be optionally substituted; and
R^{6′} and R^{6″} are independently at each occurrence selected from H and optionally substituted C₁₋₆ alkyl or C₁₋₆ acyl;
or R^{6′} and R^{6″} may together with the nitrogen to which they are attached form a5 or 6-membered heterocyclic ring, optionally substituted with 1 or 2 substituents independently selected from C₁₋₄ alkyl, C₂₋₄ alkenyl, C₂₋₄ alkynyl, hydroxyl, C₁₋₄alkoxy, halogen, cyano, nitro, carboxyl, hydroxy-C₁₋₄alkyl-, C₁₋₄alkoxy-C₁₋₄alkyl-, halogeno-C₁₋₄alkyl-difluoromethyl, trifluoromethyl, formyl, —CO—C₁₋₄alkyl, —COO—C₁₋₄alkyl, —C(O)NH₂, —C(O)NH—C₁₋₄alkyl, —C(O)N[C₁₋₄ alkyl]₂, —SO₂NH₂, —SO₂NH—C₁₋₄alkyl, —SO₂[C₁₋₄alkyl] and —S(O)p-C14 alkyl; p is (independently at each occurrence) 0, 1 or 2.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is optionally substituted (CH₂)ₘO(CH₂)ₘ-6-10-membered aryl, optionally substituted (CH₂)ₘ-6-10-membered aryl, optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine;
R⁶ is (hydroxyimino)methyl, —CO—C₁₋₆ alkyl, —CO—C₂₋₆ alkenyl, —CO—C₂₋₆ alkynyl, carboxyl, —COO—C₁₋₆ alkyl, —CONR^{6′}R^{6″}, —OCONR^{6′}R^{6″}, —OCO—C₁₋₆ alkyl, —COO—C₁₋₆ alkyl (optionally substituted with —COO—C₁₋₆ alkyl), —CONHCH(CO₂R^{6″})R⁶, —(CH₂)₀₋₆OH, —O—C₁₋₆ alkyl, —OR^{6′}, —NH—C₁₋₆ alkyl, —NH—C₁₋₆ acyl, —NR^{6′}R^{6″}, hydroxyl-C₁₋₆ alkyl, 5-10-membered heterocyclyl, 6-10-membered aryl, —C(═NOR^{6″})C₁₋₆ alkyl, —C(═NOR^{6″})NR^{6′}R^{6″}, —S(O)pNR^{6′}R^{6″}, —S(O)p-C₁₋₆ alkyl-CONHR^{6″}, —C(O)NHS(O)p-C₁₋₆ alkyl, —C(O)NHS(O)p-aryl, —CH₂CH(COOR^{6′})OH, —C₁₋₆ alkyl-CH(NR^{6′}R^{6″})COOR^{6′} or —C₁₋₆ alkyl-CH(NR^{6′}R^{6″})CONR^{6′}R^{6″}), wherein any alkyl, alkenyl, alkynyl, heterocyclyl or aryl may be optionally substituted; and
R^{6′} and R^{6″} are independently at each occurrence selected from H and optionally substituted C₁₋₆ alkyl or C₁₋₆ acyl;
or R^{6′} and R^{6″} may together with the nitrogen to which they are attached form a5 or 6-membered heterocyclic ring, optionally substituted with 1 or 2 substituents independently selected from C₁₋₄alkyl, C₂₋₄ alkenyl, C₂₋₄ alkynyl, hydroxyl, C₁₋₄alkoxy, halogen, cyano, nitro, carboxyl, hydroxy-C₁₋₄alkyl-, C₁₋₄alkoxy-C₁₋₄alkyl-, halogeno-C₁₋₄alkyl-, difluoromethyl, trifluoromethyl, formyl, —CO—C₁₋₄alkyl, —COO—C₁₋₄alkyl, —C(O)NH₂, —C(O)NH—C₁₋₄alkyl, —C(O)N[C₁₋₄ alkyl]₂, —SO₂NH₂, —SO₂NH—C₁₋₄alkyl, —SO₂[C₁₋₄alkyl] and —S(O)p-C14 alkyl; p is (independently at each occurrence) 0, 1 or 2.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine;
R⁶ is —CONHCH(CO₂R^{6″})R^{6′}, optionally substituted 5-10-membered heterocyclyl or optionally substituted 6-10-membered aryl; and
R^{6′} and R^{6″} are independently at each occurrence selected from H and optionally substituted C₁₋₆ alkyl or C₁. 6 acyl;
or R^{6′} and R^{6″} may together with the nitrogen to which they are attached form a5 or 6-membered heterocyclic ring, optionally substituted with 1 or 2 substituents independently selected from C₁₋₄ alkyl, C₂₋₄ alkenyl, C₂₋₄ alkynyl, hydroxyl, C₁₋₄alkoxy, halogen, cyano, nitro, carboxyl, hydroxy-C₁₋₄alkyl-, C₁₋₄alkoxy-C₁₋₄alkyl-, halogeno-C₁₋₄alkyl-, difluoromethyl, trifluoromethyl, formyl, —CO—C₁₋₄alkyl, —COO—C₁₋₄alkyl, —C(O)NH₂, —C(O)NH—C₁₋₄alkyl, —C(O)N[C₁₋₄ alkyl]₂, —SO₂NH₂, —SO₂NH—C₁₋₄alkyl, —SO₂[C₁₋₄alkyl] and —S(O)p-C14 alkyl; p is (independently at each occurrence) 0, 1 or 2.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine;
R⁶ is —CONHCH(CO₂R^{6″})R^{6′} or optionally substituted 5-10-membered heterocyclyl; and
R^{6′} and R^{6″} are independently at each occurrence selected from H and optionally substituted C₁₋₆ alkyl or C₁₋₆ acyl;
or R^{6′} and R^{6″} may together with the nitrogen to which they are attached form a5 or 6-membered heterocyclic ring, optionally substituted with 1 or 2 substituents independently selected from C₁₋₄ alkyl, C₂₋₄ alkenyl, C₂₋₄ alkynyl, hydroxyl, C₁₋₄alkoxy, halogen, cyano, nitro, carboxyl, hydroxy-C₁₋₄alkyl-, C₁₋₄alkoxy-C₁₋₄alkyl-, halogeno-C₁₋₄alkyl-, difluoromethyl, trifluoromethyl, formyl, —CO—C₁₋₄alkyl, —COO—C₁₋₄alkyl, —C(O)NH₂, —C(O)NH—C₁₋₄alkyl, —C(O)N[C₁₋₄ alkyl]₂, —SO₂NH₂, —SO₂NH—C₁₋₄alkyl, —SO₂[C₁₋₄alkyl] and —S(O)p-C14 alkyl; p is (independently at each occurrence) 0, 1 or 2.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine;
R⁶ is carboxyl or optionally alkylated or acylated nitrogen.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine;
R⁶ is carboxyl.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine;
R⁶ is optionally alkylated or acylated nitrogen.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is —CONHCH₂(CO₂H) or optionally substituted 5-10-membered heterocyclyl.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is optionally substituted (CH₂)ₘO(CH₂)ₘ-5-10-membered heterocycle or optionally substituted (CH₂)ₘ-5-10-membered heterocycle, wherein each m is an integer independently selected from 0, 1, 2 and 3, preferably where each m is an integer independently selected from 0, 1 and 2, more preferably where each m is an integer independently selected from 0 and 1; and
R⁶ is —CONHCH₂(CO₂H) or optionally substituted 5-10-membered heterocyclyl.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is —CONHCH₂(CO₂H) or optionally substituted 5-10-membered heterocyclyl selected from 1,2,4-oxadiazol-5-one, tetrazole, 1,3,4-oxadiazol-2-one and 1,4-dihydro-tetrazol-5-one.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is optionally substituted (CH₂)ₘO(CH₂)ₘ-5-10-membered heterocycle or optionally substituted (CH₂)ₘ-5-10-membered heterocycle, wherein each m is an integer independently selected from 0, 1, 2 and 3, preferably where each m is an integer independently selected from 0, 1 and 2, more preferably where each m is an integer independently selected from 0 and 1; and
R⁶ is —CONHCH₂(CO₂H) or optionally substituted 5-10-membered heterocyclyl selected from 1,2,4-oxadiazol-5-one, tetrazole, 1,3,4-oxadiazol-2-one and 1,4-dihydro-tetrazol-5-one.

According to certain embodiments, a compound of formula I is provided, wherein:
R¹ is selected from methyl, chloro and bromo;
R² and R³ are each independently selected from chloro, bromo or fluoro;
R⁴ is H;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is-CONHCH₂(CO₂H).

According to certain embodiments, a compound of formula I is provided, wherein:
R¹ is selected from methyl, chloro and bromo;
R² and R³ are each independently selected from chloro, bromo or fluoro;
R⁴ is H;
R⁵ is optionally substituted (CH₂)ₘO(CH₂)ₘ-5-10-membered heterocycle or optionally substituted (CH₂)ₘ-5-10-membered heterocycle, wherein each m is an integer independently selected from 0, 1, 2 and 3, preferably where each m is an integer independently selected from 0, 1 and 2, more preferably where each m is an integer independently selected from 0 and 1; and
R⁶ is-CONHCH₂(CO₂H).

According to certain embodiments, a compound of formula I is provided, wherein:
R¹ is selected from methyl, chloro and bromo;
R² and R³ are each independently selected from chloro, bromo or fluoro; preferably R² and R³ are chloro;
R⁴ is H;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is optionally substituted 5-10-membered heterocyclyl selected from 1,2,4-oxadiazol-5-one, tetrazole, 1,3,4-oxadiazol-2-one and 1,4-dihydro-tetrazol-5-one.

According to certain embodiments, a compound of formula I is provided, wherein:
R¹ is selected from methyl, chloro and bromo;
R² and R³ are each independently selected from chloro, bromo or fluoro; preferably R² and R³ are chloro;
R⁴ is H;
R⁵ is optionally substituted (CH₂)ₘO(CH₂)ₘ-5-10-membered heterocycle or optionally substituted (CH₂)ₘ-5-10-membered heterocycle, wherein each m is an integer independently selected from 0, 1, 2 and 3, preferably where each m is an integer independently selected from 0, 1 and 2, more preferably where each m is an integer independently selected from 0 and 1; and
R⁶ is optionally substituted 5-10-membered heterocyclyl selected from 1,2,4-oxadiazol-5-one, tetrazole, 1,3,4-oxadiazol-2-one and 1,4-dihydro-tetrazol-5-one.

According to certain embodiments, a compound of formula I is provided, wherein:
R¹ is selected from methyl, chloro and bromo;
R² and R³ are each independently selected from chloro, bromo or fluoro; preferably R² and R³ are chloro;
R⁴ is H;
R⁵ is selected from the group consisting of piperidine, piperidin-3-amine, piperidin-4-amine, piperidin-3-ylmethanamine, piperazine, pyrrolidine, pyrrolidin-3-amine, morpholine, isoindoline and 1-phenylpiperazine; and
R⁶ is —CONHCH₂(CO₂H)′.

According to certain embodiments, a compound of formula I is provided, wherein:
R¹ is selected from methyl, chloro and bromo;
R² and R³ are each independently selected from chloro, bromo or fluoro; preferably R² and R³ are chloro;
R⁴ is H;
R⁵ is selected from the group consisting of piperidine, piperidin-3-amine, piperidin-4-amine, piperidin-3-ylmethanamine, piperazine, pyrrolidine, pyrrolidin-3-amine, morpholine, isoindoline and 1-phenylpiperazine; and
R⁶ is optionally substituted 5-10-membered heterocyclyl selected from 1,2,4-oxadiazol-5-one, tetrazole, 1,3,4-oxadiazol-2-one and 1,4-dihydro-tetrazol-5-one.

According to certain embodiments, a compound of formula I is provided, wherein:
R¹ is methyl;
R² and R³ are each independently selected from chloro, bromo or fluoro; preferably R² and R³ are chloro;
R⁴ is selected from hydrogen, —(CH₂)₁₋₆-A, wherein A is optionally substituted aryl or optionally substituted heterocyclyl;
R⁵ is selected from hydrogen, OR^{5′}, optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is —CONHCH₂(CO₂H), optionally substituted 5-10-membered heterocyclyl or optionally substituted 6-10-membered aryl.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is selected from hydrogen, OR^{5′}, optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is —CONHCH₂(CO₂H), optionally substituted 5-10-membered heterocyclyl or optionally substituted 6-10-membered aryl.

According to certain embodiments, a compound of formula I is provided, wherein:
R¹ is methyl;
R² and R³ are each independently selected from chloro, bromo or fluoro;
R⁴ is hydrogen;
R⁵ is hydrogen;
R⁶ is —CONHCH₂(CO₂H), optionally substituted 5-10-membered heterocyclyl or optionally substituted 6-10-membered aryl.

According to certain embodiments, a compound of formula I is provided, wherein:
R¹ is methyl;
R² and R³ are each independently selected from chloro, bromo or fluoro;
R⁴ is hydrogen;
R⁵ is OR^{5′};
R⁶ is —CONHCH₂(CO₂H), optionally substituted 5-10-membered heterocyclyl or optionally substituted 6-10-membered aryl.

According to certain embodiments, a compound of formula I is provided, wherein:
R¹ is methyl;
R² and R³ are each independently selected from chloro, bromo or fluoro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is —CONHCH₂(CO₂H), optionally substituted 5-10-membered heterocyclyl or optionally substituted 6-10-membered aryl.

According to certain embodiments, a compound of formula I is provided, wherein:
R¹ is methyl;
R² and R³ are each independently selected from chloro, bromo or fluoro;
R⁴ is hydrogen;
R⁵ is optionally substituted (CH₂)ₘO(CH₂)ₘ-5-10-membered heterocycle or optionally substituted (CH₂)ₘ-5-10-membered heterocycle, wherein each m is an integer independently selected from 0, 1, 2 and 3, preferably where each m is an integer independently selected from 0, 1 and 2, more preferably where each m is an integer independently selected from 0 and 1;
R⁶ is —CONHCH₂(CO₂H), optionally substituted 5-10-membered heterocyclyl or optionally substituted 6-10-membered aryl.

According to certain embodiments, a compound of formula I is provided, wherein:
R¹ is methyl;
R² and R³ are each independently selected from chloro, bromo or fluoro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is —CONHCH₂(CO₂H).

According to certain embodiments, a compound of formula I is provided, wherein:
R¹ is methyl;
R² and R³ are each independently selected from chloro, bromo or fluoro;
R⁴ is hydrogen;
R⁵ is optionally substituted (CH₂)ₘO(CH₂)ₘ-5-10-membered heterocycle or optionally substituted (CH₂)ₘ-5-10-membered heterocycle, wherein each m is an integer independently selected from 0, 1, 2 and 3, preferably where each m is an integer independently selected from 0, 1 and 2, more preferably where each m is an integer independently selected from 0 and 1;
R⁶ is —CONHCH₂(CO₂H).

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is optionally substituted 5-10-membered heterocyclyl.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R⁵ is H and R^{5″} is optionally substituted piperidine; and
R⁶ is optionally substituted 5-membered heterocyclyl.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle or optionally substituted (CH₂)ₘ-5-10-membered heterocycle, wherein each m is an integer independently selected from 0, 1, 2 and 3, preferably where each m is an integer independently selected from 0, 1 and 2, more preferably where each m is an integer independently selected from 0 and 1;
R⁶ is optionally substituted 5-10-membered heterocyclyl.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle or optionally substituted (CH₂)ₘ-5-10-membered heterocycle, wherein each m is an integer independently selected from 0, 1, 2 and 3, preferably where each m is an integer independently selected from 0, 1 and 2, more preferably where each m is an integer independently selected from 0 and 1;
R⁶ is optionally substituted 5-membered heterocyclyl.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘ-5-10-membered heterocycle, wherein m is an integer independently selected from 0, 1, 2 and 3, preferably where m is an integer independently selected from 0, 1 and 2, more preferably where m is an integer independently selected from 0 and 1;
R⁶ is —CONHCH₂(CO₂H).

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘ-5-10-membered heterocycle, wherein m is an integer independently selected from 0, 1, 2 and 3, preferably where m is an integer independently selected from 0, 1 and 2, more preferably where m is an integer independently selected from 0 and 1;
R⁶ is optionally substituted 5-10-membered heterocyclyl.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘ-5-10-membered heterocycle, wherein m is an integer independently selected from 0, 1, 2 and 3, preferably where m is an integer independently selected from 0, 1 and 2, more preferably where m is an integer independently selected from 0 and 1;
R⁶ is optionally substituted 5-membered heterocyclyl.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘNR^{5′}R^{5″}, wherein m is an integer independently selected from 0, 1, 2 and 3, preferably where m is an integer independently selected from 0, 1 and 2, more preferably where m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is —CONHCH₂(CO₂H).

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘNR^{5′}R^{5″}, wherein m is an integer independently selected from 0, 1, 2 and 3, preferably where m is an integer independently selected from 0, 1 and 2, more preferably where m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is optionally substituted 5-10-membered heterocyclyl.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘNR^{5′}R^{5″}, wherein m is an integer independently selected from 0, 1, 2 and 3, preferably where m is an integer independently selected from 0, 1 and 2, more preferably where m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is optionally substituted 5-membered heterocyclyl.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3, preferably where each m is an integer independently selected from 0, 1 and 2, more preferably where each m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is 1,2,4-oxadiazol-5-one, tetrazole, 1,3,4-oxadiazol-2-one or 1,4-dihydro-tetrazol-5-one.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle or optionally substituted (CH₂)ₘ-5-10-membered heterocycle, wherein each m is an integer independently selected from 0, 1, 2 and 3, preferably where each m is an integer independently selected from 0, 1 and 2, more preferably where each m is an integer independently selected from 0 and 1;
R⁶ is 1,2,4-oxadiazol-5-one, tetrazole, 1,3,4-oxadiazol-2-one or 1,4-dihydro-tetrazol-5-one.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘO(CH₂)ₘ-5-10-membered heterocycle, wherein m is an integer independently selected from 0, 1, 2 and 3, preferably where m is an integer independently selected from 0, 1 and 2, more preferably where m is an integer independently selected from 0 and 1;
R⁶ is 1,2,4-oxadiazol-5-one, tetrazole, 1,3,4-oxadiazol-2-one or 1,4-dihydro-tetrazol-5-one.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘ-5-10-membered heterocycle, wherein m is an integer independently selected from 0, 1, 2 and 3, preferably where m is an integer independently selected from 0, 1 and 2, more preferably where m is an integer independently selected from 0 and 1;
R⁶ is 1,2,4-oxadiazol-5-one, tetrazole, 1,3,4-oxadiazol-2-one or 1,4-dihydro-tetrazol-5-one.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is optionally substituted (CH₂)ₘNR^{5′}R^{5″}, wherein m is an integer independently selected from 0, 1, 2 and 3, preferably where m is an integer independently selected from 0, 1 and 2, more preferably where m is an integer independently selected from 0 and 1;
R^{5′} is H and R^{5″} is optionally substituted 4-6-membered heterocyclyl; preferably R^{5′} is H and R^{5″} is optionally substituted piperidine; and
R⁶ is 1,2,4-oxadiazol-5-one, tetrazole, 1,3,4-oxadiazol-2-one or 1,4-dihydro-tetrazol-5-one.

According to certain embodiments, a compound of formula I is provided, wherein:
R¹ is methyl;
R² and R³ are each independently selected from chloro, bromo or fluoro; preferably R² and R³ are chloro;
R⁴ is hydrogen;
R⁵ is piperidine, piperidin-3-amine, piperidin-4-amine, piperidin-3-ylmethanamine, piperazine, pyrrolidine, pyrrolidin-3-amine, morpholine, isoindoline or 1-phenylpiperazine;
R⁶ is 1,2,4-oxadiazol-5-one, tetrazole, 1,3,4-oxadiazol-2-one or 1,4-dihydro-tetrazol-5-one.

According to certain embodiments, a compound of formula I is provided which is selected from the group consisting of:
1-(2-(3,4-Dichloro-5-methyl-1H-pyrrole-2-carboxamido)-5-(5-oxo-4,5-dihydro-1,2,4-oxadiazol-3-yl)phenyl)piperidin-4-aminium chloride,
1-(2-(3,4-Dichloro-5-methyl-1H-pyrrole-2-carboxamido)-5-(5-oxo-4,5-dihydro-1,2,4-oxadiazol-3-yl)phenyl)piperidin-3-aminium chloride,
(1-(2-(3,4-Dichloro-5-methyl-1H-pyrrole-2-carboxamido)-5-(5-oxo-4,5-dihydro-1,2,4-oxadiazol-3-yl)phenyl)piperidin-3-yl)methanaminium chloride,
4-((2-(3,4-Dichloro-5-methyl-1H-pyrrole-2-carboxamido)-5-(5-oxo-4,5-dihydro-1,2,4-oxadiazol-3-yl)phenyl)amino)piperidin-1-ium chloride,
4-(2-(3,4-Dichloro-5-methyl-1H-pyrrole-2-carboxamido)-5-(5-oxo-4,5-dihydro-1,2,4-oxadiazol-3-yl)phenyl)piperazin-1-ium chloride,
3,4-Dichloro-5-methyl-N-(4-(5-oxo-4,5-dihydro-1,2,4-oxadiazol-3-yl)-2-(pyrrolidin-1-yl)phenyl)-1H-pyrrole-2-carboxamide,
3,4-Dichloro-5-methyl-N-(4-(5-oxo-4,5-dihydro-1,2,4-oxadiazol-3-yl)-2-(4-phenylpiperazin-1-yl)phenyl)-1H-pyrrole-2-carboxamide,
3,4-Dichloro-5-methyl-N-(2-morpholino-4-(5-oxo-4,5-dihydro-1,2,4-oxadiazol-3-yl)phenyl)-1H-pyrrole-2-carboxamide,
(1-(2-(3,4-Dichloro-5-methyl-1H-pyrrole-2-carboxamido)-5-(2H-tetrazol-5-yl)phenyl)piperidin-3-yl)methanaminium chloride,
3,4-Dichloro-5-methyl-N-(2-morpholino-4-(2H-tetrazol-5-yl)phenyl)-1H-pyrrole-2-carboxamide,
N-(2-(Benzyloxy)-4-(5-oxo-4,5-dihydro-1,3,4-oxadiazol-2-yl)phenyl)-3,4-dichloro-5-methyl-1H-pyrrole-2-carboxamide,
4-(5-Carboxy-2-(3,4-dichloro-5-methyl-1H-pyrrole-2-carboxamido)phenyl)piperazin-1-ium chloride,
(1-(5-Carboxy-2-(3,4-dichloro-5-methyl-1H-pyrrole-2-carboxamido)phenyl)piperidin-3-yl)methanaminium chloride,
4-(5-Carboxy-2-(3,4-dichloro-5-methyl-1H-pyrrole-2-carboxamido)phenyl)piperazin-1-ium chloride,
4-((5-Carboxy-2-(3,4-dichloro-5-methyl-1H-pyrrole-2-carboxamido)phenyl)amino)piperidin-1-ium chloride,
4-(3,4-Dichloro-5-methyl-1H-pyrrole-2-carboxamido)-3-(4-phenylpiperazin-1-yl)benzoic acid,
Methyl (S)-3-(3-((tert-butoxycarbonyl)amino)pyrrolidin-1-yl)-4-(3,4-dichloro-5-methyl-1H-pyrrole-2-carboxamido)benzoate,
(S)-1-(2-(3,4-Dichloro-5-methyl-1H-pyrrole-2-carboxamido)-5-(5-oxo-4,5-dihydro-1,3,4-oxadiazol-2-yl)phenyl)piperidin-3-aminium chloride,
4-((2-(3,4-Dichloro-5-methyl-1H-pyrrole-2-carboxamido)-5-(5-oxo-4,5-dihydro-1,3,4-oxadiazol-2-yl)phenyl)amino)piperidin-1-ium chloride,
(S)-1-(2-(3,4-Dichloro-5-methyl-1H-pyrrole-2-carboxamido)-5-(5-oxo-4,5-dihydro-1,3,4-oxadiazol-2-yl)phenyl)pyrrolidin-3-aminium chloride,
3,4-Dichloro-N-(2-isopropoxy-4-(5-methyl-1,3,4-oxadiazol-2-yl)phenyl)-5-methyl-1H-pyrrole-2-carboxamide,
(S)-1-(2-(3,4-Dichloro-5-methyl-1H-pyrrole-2-carboxamido)-5-(5-oxo-4,5-dihydro-1H-tetrazol-1-yl)phenyl)pyrrolidin-3-aminium chloride,
1-(2-(3,4-Dichloro-5-methyl-1H-pyrrole-2-carboxamido)-5-(5-oxo-4,5-dihydro-1H-tetrazol-1-yl)phenyl)piperidin-4-aminium chloride,
3,4-Dichloro-5-methyl-N-(4-(5-oxo-4,5-dihydro-1H-tetrazol-1-yl)-2-(4-phenylpiperazin-1-yl)phenyl)-1H-pyrrole-2-carboxamide,
3,4-Dichloro-N-(2-(isoindolin-2-yl)-4-(5-oxo-4,5-dihydro-1H-tetrazol-1-yl)phenyl)-5-methyl-1H-pyrrole-2-carboxamide,
3-(2-(3,4-Dichloro-5-methyl-1H-pyrrole-2-carboxamido)-5-(5-oxo-4,5-dihydro-1,3,4-oxadiazol-2-yl)phenoxy)pyrrolidin-1-ium chloride,
4-(2-(3,4-Dichloro-5-methyl-1H-pyrrole-2-carboxamido)-5-(5-oxo-4,5-dihydro-1,3,4-oxadiazol-2-yl)phenoxy)piperidin-1-ium chloride,
3-((2-(3,4-Dichloro-5-methyl-1H-pyrrole-2-carboxamido)-5-(5-oxo-4,5-dihydro-1,3,4-oxadiazol-2-yl)phenoxy)methyl)piperidin-1-ium chloride,
4-(2-(2-(3,4-Dichloro-5-methyl-1H-pyrrole-2-carboxamido)-5-(5-oxo-4,5-dihydro-1,3,4-oxadiazol-2-yl)phenoxy)ethyl)piperidin-1-ium chloride,
3,4-Dichloro-5-methyl-N-(4-(5-oxo-4,5-dihydro-1,3,4-oxadiazol-2-yl)-2-(pyridin-4-ylmethoxy)phenyl)-1H-pyrrole-2-carboxamide,
3,4-Dichloro-5-methyl-N-(4-(5-oxo-4,5-dihydro-1,3,4-oxadiazol-2-yl)-2-(pyrimidin-2-ylmethoxy)phenyl)-1H-pyrrole-2-carboxamide,
3,4-Dichloro-N-(2-isopropoxy-4-(5-oxo-4,5-dihydro-1,3,4-oxadiazol-2-yl)phenyl)-5-methyl-1H-pyrrole-2-carboxamide,
3,4-Dichloro-5-methyl-N-(4-(5-oxo-4,5-dihydro-1,3,4-oxadiazol-2-yl)-2-(thiophen-2-ylmethoxy)phenyl)-1H-pyrrole-2-carboxamide,
(R)-1-(5-((Carboxymethyl)carbamoyl)-2-(3,4-dichloro-5-methyl-1H-pyrrole-2-carboxamido)phenyl)pyrrolidin-3-aminium chloride,
(S)-1-(5-((Carboxymethyl)carbamoyl)-2-(3,4-dichloro-5-methyl-1H-pyrrole-2-carboxamido)phenyl)pyrrolidin-3-aminium chloride,
4-(5-((Carboxymethyl)carbamoyl)-2-(3,4-dichloro-5-methyl-1H-pyrrole-2-carboxamido)phenyl)piperazin-1-ium chloride,
(R)-1-(5-((Carboxymethyl)carbamoyl)-2-(4,5-dibromo-1H-pyrrole-2-carboxamido)phenyl)pyrrolidin-3-aminium chloride,
(S)-1-(5-((Carboxymethyl)carbamoyl)-2-(4,5-dibromo-1H-pyrrole-2-carboxamido)phenyl)pyrrolidin-3-aminium chloride,
4-(5-((Carboxymethyl)carbamoyl)-2-(4,5-dibromo-1H-pyrrole-2-carboxamido)phenyl)piperazin-1-ium chloride,
4-(2-(3,4-Dichloro-5-methyl-1H-pyrrole-2-carboxamido)-5-(((5-oxo-4,5-dihydro-1,3,4-oxadiazol-2-yl)methyl)carbamoyl)phenyl)piperazin-1-ium chloride,
4-(2-(3,4-Dichloro-5-methyl-1H-pyrrole-2-carboxamido)-5-(5-oxo-4,5-dihydro-1,3,4-oxadiazol-2-yl)phenyl)piperazin-1-ium chloride,
3,4-Dichloro-5-methyl-N-(2-morpholino-4-(5-oxo-4,5-dihydro-1,3,4-oxadiazol-2-yl)phenyl)-1H-pyrrole-2-carboxamide,
3,4-dichloro-5-methyl-N-(2-(2-methylmorpholino)-4-(5-oxo-4,5-dihydro-1,3,4-oxadiazol-2-yl)phenyl)-1H-pyrrole-2-carboxamide,
3,4-Dichloro-N-(2-(2,6-dimethylmorpholino)-4-(hydrazinecarbonyl)phenyl)-5-methyl-1H-pyrrole-2-carboxamide, and
3,4-Dichloro-5-methyl-N-(4-((methylsulfonyl)carbamoyl)-2-morpholinophenyl)-1H-pyrrole-2-carboxamide.

According to certain embodiments, a compound of formula I is provided which is selected from the group consisting of:
1-(2-(3,4-Dichloro-5-methyl-1H-pyrrole-2-carboxamido)-5-(5-oxo-4,5-dihydro-1,2,4-oxadiazol-3-yl)phenyl)piperidin-4-aminium chloride,
1-(2-(3,4-Dichloro-5-methyl-1H-pyrrole-2-carboxamido)-5-(5-oxo-4,5-dihydro-1,2,4-oxadiazol-3-yl)phenyl)piperidin-3-aminium chloride,
(1-(2-(3,4-Dichloro-5-methyl-1H-pyrrole-2-carboxamido)-5-(5-oxo-4,5-dihydro-1,2,4-oxadiazol-3-yl)phenyl)piperidin-3-yl)methanaminium chloride,
4-((2-(3,4-Dichloro-5-methyl-1H-pyrrole-2-carboxamido)-5-(5-oxo-4,5-dihydro-1,2,4-oxadiazol-3-yl)phenyl)amino)piperidin-1-ium chloride,
4-(2-(3,4-Dichloro-5-methyl-1H-pyrrole-2-carboxamido)-5-(5-oxo-4,5-dihydro-1,2,4-oxadiazol-3-yl)phenyl)piperazin-1-ium chloride,
3,4-Dichloro-5-methyl-N-(4-(5-oxo-4,5-dihydro-1,2,4-oxadiazol-3-yl)-2-(pyrrolidin-1-yl)phenyl)-1H-pyrrole-2-carboxamide,
3,4-Dichloro-5-methyl-N-(4-(5-oxo-4,5-dihydro-1,2,4-oxadiazol-3-yl)-2-(4-phenylpiperazin-1-yl)phenyl)-1H-pyrrole-2-carboxamide,
3,4-Dichloro-5-methyl-N-(2-morpholino-4-(5-oxo-4,5-dihydro-1,2,4-oxadiazol-3-yl)phenyl)-1H-pyrrole-2-carboxamide,
(1-(2-(3,4-Dichloro-5-methyl-1H-pyrrole-2-carboxamido)-5-(2H-tetrazol-5-yl)phenyl)piperidin-3-yl)methanaminium chloride,
3,4-Dichloro-5-methyl-N-(2-morpholino-4-(2H-tetrazol-5-yl)phenyl)-1H-pyrrole-2-carboxamide,
N-(2-(Benzyloxy)-4-(5-oxo-4,5-dihydro-1,3,4-oxadiazol-2-yl)phenyl)-3,4-dichloro-5-methyl-1H-pyrrole-2-carboxamide,
4-(5-Carboxy-2-(3,4-dichloro-5-methyl-1H-pyrrole-2-carboxamido)phenyl)piperazin-1-ium chloride,
(1-(5-Carboxy-2-(3,4-dichloro-5-methyl-1H-pyrrole-2-carboxamido)phenyl)piperidin-3-yl)methanaminium chloride,
4-(5-Carboxy-2-(3,4-dichloro-5-methyl-1H-pyrrole-2-carboxamido)phenyl)piperazin-1-ium chloride,
4-((5-Carboxy-2-(3,4-dichloro-5-methyl-1H-pyrrole-2-carboxamido)phenyl)amino)piperidin-1-ium chloride,
4-(3,4-Dichloro-5-methyl-1H-pyrrole-2-carboxamido)-3-(4-phenylpiperazin-1-yl)benzoic acid,
Methyl (S)-3-(3-((tert-butoxycarbonyl)amino)pyrrolidin-1-yl)-4-(3,4-dichloro-5-methyl-1H-pyrrole-2-carboxamido)benzoate,
(S)-1-(2-(3,4-Dichloro-5-methyl-1H-pyrrole-2-carboxamido)-5-(5-oxo-4,5-dihydro-1,3,4-oxadiazol-2-yl)phenyl)piperidin-3-aminium chloride,
4-((2-(3,4-Dichloro-5-methyl-1H-pyrrole-2-carboxamido)-5-(5-oxo-4,5-dihydro-1,3,4-oxadiazol-2-yl)phenyl)amino)piperidin-1-ium chloride,
(S)-1-(2-(3,4-Dichloro-5-methyl-1H-pyrrole-2-carboxamido)-5-(5-oxo-4,5-dihydro-1,3,4-oxadiazol-2-yl)phenyl)pyrrolidin-3-aminium chloride,
3,4-Dichloro-N-(2-isopropoxy-4-(5-methyl-1,3,4-oxadiazol-2-yl)phenyl)-5-methyl-1H-pyrrole-2-carboxamide,
(S)-1-(2-(3,4-Dichloro-5-methyl-1H-pyrrole-2-carboxamido)-5-(5-oxo-4,5-dihydro-1H-tetrazol-1-yl)phenyl)pyrrolidin-3-aminium chloride,
1-(2-(3,4-Dichloro-5-methyl-1H-pyrrole-2-carboxamido)-5-(5-oxo-4,5-dihydro-1H-tetrazol-1-yl)phenyl)piperidin-4-aminium chloride,
3,4-Dichloro-5-methyl-N-(4-(5-oxo-4,5-dihydro-1H-tetrazol-1-yl)-2-(4-phenylpiperazin-1-yl)phenyl)-1H-pyrrole-2-carboxamide,
3,4-Dichloro-N-(2-(isoindolin-2-yl)-4-(5-oxo-4,5-dihydro-1H-tetrazol-1-yl)phenyl)-5-methyl-1H-pyrrole-2-carboxamide,
3-(2-(3,4-Dichloro-5-methyl-1H-pyrrole-2-carboxamido)-5-(5-oxo-4,5-dihydro-1,3,4-oxadiazol-2-yl)phenoxy)pyrrolidin-1-ium chloride,
4-(2-(3,4-Dichloro-5-methyl-1H-pyrrole-2-carboxamido)-5-(5-oxo-4,5-dihydro-1,3,4-oxadiazol-2-yl)phenoxy)piperidin-1-ium chloride,
3-((2-(3,4-Dichloro-5-methyl-1H-pyrrole-2-carboxamido)-5-(5-oxo-4,5-dihydro-1,3,4-oxadiazol-2-yl)phenoxy)methyl)piperidin-1-ium chloride,
4-(2-(2-(3,4-Dichloro-5-methyl-1H-pyrrole-2-carboxamido)-5-(5-oxo-4,5-dihydro-1,3,4-oxadiazol-2-yl)phenoxy)ethyl)piperidin-1-ium chloride,
3,4-Dichloro-5-methyl-N-(4-(5-oxo-4,5-dihydro-1,3,4-oxadiazol-2-yl)-2-(pyridin-4-ylmethoxy)phenyl)-1H-pyrrole-2-carboxamide,
3,4-Dichloro-5-methyl-N-(4-(5-oxo-4,5-dihydro-1,3,4-oxadiazol-2-yl)-2-(pyrimidin-2-ylmethoxy)phenyl)-1H-pyrrole-2-carboxamide,
3,4-Dichloro-N-(2-isopropoxy-4-(5-oxo-4,5-dihydro-1,3,4-oxadiazol-2-yl)phenyl)-5-methyl-1H-pyrrole-2-carboxamide,
3,4-Dichloro-5-methyl-N-(4-(5-oxo-4,5-dihydro-1,3,4-oxadiazol-2-yl)-2-(thiophen-2-ylmethoxy)phenyl)-1H-pyrrole-2-carboxamide,
(R)-1-(5-((Carboxymethyl)carbamoyl)-2-(3,4-dichloro-5-methyl-1H-pyrrole-2-carboxamido)phenyl)pyrrolidin-3-aminium chloride,
(S)-1-(5-((Carboxymethyl)carbamoyl)-2-(3,4-dichloro-5-methyl-1H-pyrrole-2-carboxamido)phenyl)pyrrolidin-3-aminium chloride,
4-(5-((Carboxymethyl)carbamoyl)-2-(3,4-dichloro-5-methyl-1H-pyrrole-2-carboxamido)phenyl)piperazin-1-ium chloride,
(R)-1-(5-((Carboxymethyl)carbamoyl)-2-(4,5-dibromo-1H-pyrrole-2-carboxamido)phenyl)pyrrolidin-3-aminium chloride,
(S)-1-(5-((Carboxymethyl)carbamoyl)-2-(4,5-dibromo-1H-pyrrole-2-carboxamido)phenyl)pyrrolidin-3-aminium chloride,
4-(5-((Carboxymethyl)carbamoyl)-2-(4,5-dibromo-1H-pyrrole-2-carboxamido)phenyl)piperazin-1-ium chloride, and
4-(2-(3,4-Dichloro-5-methyl-1H-pyrrole-2-carboxamido)-5-(((5-oxo-4,5-dihydro-1,3,4-oxadiazol-2-yl)methyl)carbamoyl)phenyl)piperazin-1-ium chloride.
According to certain embodiments, a compound of formula I is provided which is selected from the group consisting of:
1-(2-(3,4-Dichloro-5-methyl-1H-pyrrole-2-carboxamido)-5-(5-oxo-4,5-dihydro-1,2,4-oxadiazol-3-yl)phenyl)piperidin-4-aminium chloride,
1-(2-(3,4-Dichloro-5-methyl-1H-pyrrole-2-carboxamido)-5-(5-oxo-4,5-dihydro-1,2,4-oxadiazol-3-yl)phenyl)piperidin-3-aminium chloride,
(1-(2-(3,4-Dichloro-5-methyl-1H-pyrrole-2-carboxamido)-5-(5-oxo-4,5-dihydro-1,2,4-oxadiazol-3-yl)phenyl)piperidin-3-yl)methanaminium chloride,
3,4-Dichloro-5-methyl-N-(4-(5-oxo-4,5-dihydro-1,2,4-oxadiazol-3-yl)-2-(pyrrolidin-1-yl)phenyl)-1H-pyrrole-2-carboxamide,
3,4-Dichloro-5-methyl-N-(4-(5-oxo-4,5-dihydro-1,2,4-oxadiazol-3-yl)-2-(4-phenylpiperazin-1-yl)phenyl)-1H-pyrrole-2-carboxamide,
3,4-Dichloro-5-methyl-N-(2-morpholino-4-(5-oxo-4,5-dihydro-1,2,4-oxadiazol-3-yl)phenyl)-1H-pyrrole-2-carboxamide,
(1-(2-(3,4-Dichloro-5-methyl-1H-pyrrole-2-carboxamido)-5-(2H-tetrazol-5-yl)phenyl)piperidin-3-yl)methanaminium chloride,
3,4-Dichloro-5-methyl-N-(2-morpholino-4-(2H-tetrazol-5-yl)phenyl)-1H-pyrrole-2-carboxamide,
(1-(5-Carboxy-2-(3,4-dichloro-5-methyl-1H-pyrrole-2-carboxamido)phenyl)piperidin-3-yl)methanaminium chloride,
(S)-1-(2-(3,4-Dichloro-5-methyl-1H-pyrrole-2-carboxamido)-5-(5-oxo-4,5-dihydro-1,3,4-oxadiazol-2-yl)phenyl)piperidin-3-aminium chloride,
4-((2-(3,4-Dichloro-5-methyl-1H-pyrrole-2-carboxamido)-5-(5-oxo-4,5-dihydro-1,3,4-oxadiazol-2-yl)phenyl)amino)piperidin-1-ium chloride,
(S)-1-(2-(3,4-Dichloro-5-methyl-1H-pyrrole-2-carboxamido)-5-(5-oxo-4,5-dihydro-1,3,4-oxadiazol-2-yl)phenyl)pyrrolidin-3-aminium chloride,
3,4-Dichloro-5-methyl-N-(4-(5-oxo-4,5-dihydro-1H-tetrazol-1-yl)-2-(4-phenylpiperazin-1-yl)phenyl)-1H-pyrrole-2-carboxamide,
3,4-Dichloro-N-(2-(isoindolin-2-yl)-4-(5-oxo-4,5-dihydro-1H-tetrazol-1-yl)phenyl)-5-methyl-1H-pyrrole-2-carboxamide,
4-(2-(3,4-Dichloro-5-methyl-1H-pyrrole-2-carboxamido)-5-(5-oxo-4,5-dihydro-1,3,4-oxadiazol-2-yl)phenoxy)piperidin-1-ium chloride, and
3-((2-(3,4-Dichloro-5-methyl-1H-pyrrole-2-carboxamido)-5-(5-oxo-4,5-dihydro-1,3,4-oxadiazol-2-yl)phenoxy)methyl)piperidin-1-ium chloride.

According to certain embodiments, a compound of formula I is provided which is selected from the group consisting of:
4-(2-(3,4-Dichloro-5-methyl-1H-pyrrole-2-carboxamido)-5-(5-oxo-4,5-dihydro-1,3,4-oxadiazol-2-yl)phenyl)piperazin-1-ium chloride,
3,4-Dichloro-5-methyl-N-(2-morpholino-4-(5-oxo-4,5-dihydro-1,3,4-oxadiazol-2-yl)phenyl)-1H-pyrrole-2-carboxamide,
3,4-dichloro-5-methyl-N-(2-(2-methylmorpholino)-4-(5-oxo-4,5-dihydro-1,3,4-oxadiazol-2-yl)phenyl)-1H-pyrrole-2-carboxamide,
3,4-Dichloro-N-(2-(2,6-dimethylmorpholino)-4-(hydrazinecarbonyl)phenyl)-5-methyl-1H-pyrrole-2-carboxamide, and
3,4-Dichloro-5-methyl-N-(4-((methylsulfonyl)carbamoyl)-2-morpholinophenyl)-1H-pyrrole-2-carboxamide.

A compound of formula I may form stable acid or basic salts, and in such cases administration of a compound as a salt may be appropriate, and pharmaceutically acceptable salts may be made by conventional methods such as those described below.

Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, dibenzylamine, mopholine, N-ethylmorpholine, N-methylpiperidine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine (i.e., 2-amino-2-hydroxymethyl-propane-1,3-diol), tris-(2-hydroxyethyl)amine, and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, malonic, salicyclic, ascorbic, benzoic, succinic, suberic, fumaric, mandelic, phthalic, a-glycerophosphoric, benzenesulfonic, p-tolylsulfonic, citric, tartaric, methanesulfonic, and the like. Also included are salts of amino acids such as argininate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts. A preferred pharmaceutically-acceptable salt is the sodium salt.

However, to facilitate isolation of the salt during preparation, salts which are less soluble in the chosen solvent may be preferred whether pharmaceutically-acceptable or not.

Within the present invention it is to be understood that a compound of the formula I or a salt thereof may exhibit the phenomenon of tautomerism and that the formulae drawings within this specification can represent only one of the possible tautomeric forms. It is to be understood that the invention encompasses any tautomeric form which inhibits DNA gyrase and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein.

It will be appreciated by those skilled in the art that certain compounds of formula I contain an asymmetrically substituted carbon and/or sulphur atom, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which posses properties useful in the inhibition of DNA gyrase, it being well known in the art how to prepare optically-active forms (for example, by resolution of the racemic form by recrystallization techniques, by synthesis from optically-active starting materials, by stereoselective synthesis, by enzymatic resolution, by biotransformation, or by chromatographic separation using a chiral stationary phase).

It is also to be understood that certain compounds of the formula I and salts thereof can exist in solvated as well as unsolvated forms such as, for example, hydrated forms. It is to be understood that the invention encompasses all such solvated forms which inhibit DNA gyrase.

In addition to salt forms, the invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. A prodrug may improve the physical properties of the parent drug and/or it may also improve overall drug efficacy, for example through the reduction of toxicity and unwanted effects of a drug by controlling its absorption, blood levels, metabolic distribution and cellular uptake.

As stated before, we have discovered a range of compounds that are good inhibitors of DNA gyrase. They have good physical and/or pharmacokinetic properties in general. The following compounds possess preferred pharmaceutical and/or physical and/or pharmacokinetic properties.

### Process

In a further aspect, the present invention provides a process of preparing a compound of formula I or a pharmaceutically-acceptable salt thereof.

If not commercially available, the necessary starting materials for the procedures such as those described below may be made by procedures which are selected from standard organic chemistry techniques, techniques which are analogous to the synthesis of known structurally similar compounds, or techniques, which are analogous to the procedures described in the examples.

It will also be appreciated that in some of the reactions mentioned herein it may be necessary/desirable to protect any sensitive groups in compounds. The instances where protection is necessary or desirable are known to those skilled in the art, as are suitable methods for such protection.

Example of a suitable protecting group for a hydroxy group is, for example, an acyl group, for example an alkanol group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanol or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris (trifluoroacetate). A suitable alternative protecting group for a primary amino group is, for example, a phthalogenyl group which may be removed by treatment with an alkylamine, for example dimethylaminopropylamine or 2-hydroxyethylamine, or with hydrazine.

The protecting groups may be removed at any convenient stage in the synthesis using conventional techniques well known in the chemical art, or they may be removed during a later reaction step or work-up.

The present invention also provides that the compounds of the formula I and pharmaceutically-acceptable salts thereof can be prepared by a process comprising (wherein the variables are as defined above unless otherwise stated):

### Process Step a) Reacting a Compound of Formula II:

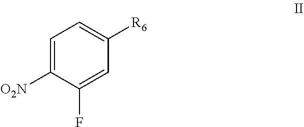

with compound of formula III:

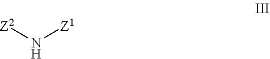

wherein Z¹ and Z² are independently selected from H, —R^{6′}R^{6″}, —CH(CO₂R^{6″})R^{6′}, heterocyclyl, aryl, to a compound of formula IV:

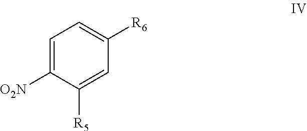

or

### Process Step b) Coupling a Compound of Formula V:

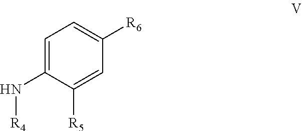

with compound of formula VI:

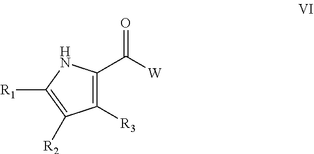

wherein W is selected from hydroxyl, halogen (preferably Cl, Br or I) and 1,1,1-trichloromethyl,
to a compound of formula I:

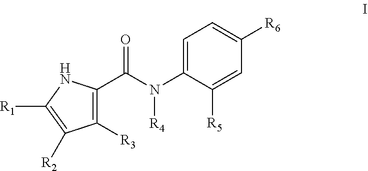

or

### Process Step c) Reacting a Compound of Formula La:

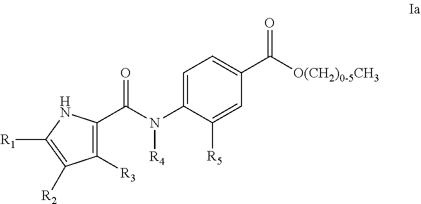

to a compound of formula Ib:

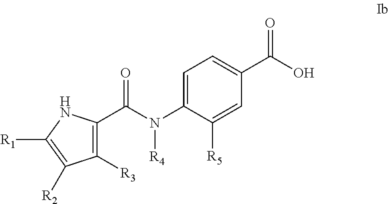

or

### Process Step d) Reacting a Compound of Formula Ic:

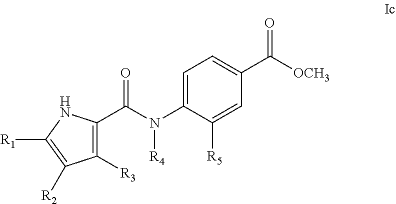

to a compound of formula Id:

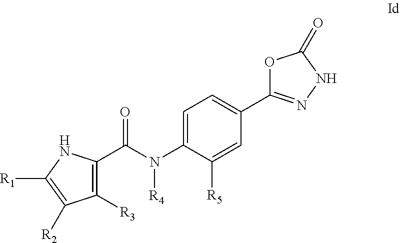

in a two-step procedure, using hydrazine monohydrate (step 1) and 1,1′-carbonyldiimidazole (step 2), as reagents.

### Process Step e) Reacting a Compound of Formula Ie:

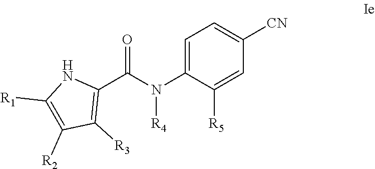

to a compound of formula If:

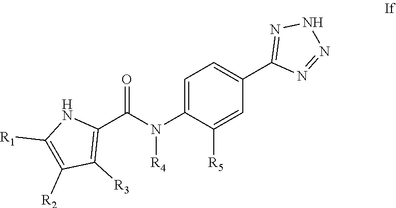

in a procedure using sodium azide as reagents.

### Medical Uses

The invention herein provides a method or ingredient for treatment or the prevention of bacterial infection in a human or animal comprising administering the human or animal a therapeutically effective amount of a compound of formula I of the present invention, and more specifically any one of Examples 1-40 disclosed below. Particularly, the invention provides a compound of formula I of the present invention, and more specifically any one of Examples 1-40 disclosed below, for use in medicine, and more specifically for use in the treatment of a bacterial infection in a warm-blooded animal. The warm-blooded animal may be a human. The present invention further provides a pharmaceutical composition comprising a compound of formula I of the present invention and a pharmaceutically acceptable excipient or carrier.

Specifically, the bacterium is a Gram-negative, a Gram-positive, or a Gram-variable bacterium. According to certain embodiments, the bacterium is a Gram-negative bacterium. According to certain embodiments, the bacterium is a Gram-positive bacterium. According to certain embodiments, the bacterium is a Gram-variable bacterium.

According to certain embodiments, the bacterium is a pathogenic bacterium, an opportunistic bacterial pathogen, or other bacterium.

The bacteria in accordance to the present invention may be one selected from the group consisting of, but not limited to, the Proteobacteria phylum, including the family of Pseudomonodaceae, including the Pseudomonas genus and the unclassified Pseudomonads, the family of Moraxellaceae, including the Acinetobacter genus, the Gammaproteobacteria class, including the Enterobacteriaceae family, including Citrobacter, Edwardsiella, Enterobacillus, Enterobacter, Cronobacter, Erwinia, Hafnia, Escherichia, Klebsiella, Pantoea, Proteus, Salmonella, Serratia, Shigella, Yersinia genus and species, Alphaproteobacteria class, including Magnetococcidae, Rickettsidae, Caulobacteridae with Rhizobiales, the Betaproteobacteria class, including Burkholderiale, Neisseriales, including the Neisseriaceae family, Vibrio, including Vibrio cholera, the Epsilonproteobacteria class, including the Campylobacter and Helicobacter genus, the Spirochaetes phylum, the Firmicutes phylum, including Clostridia and Bacilli, the Fusobacteria phylum, the Tenericutes phylum, including the Mycoplasma, Spiroplasma, and Ureaplasma genera, the Actinobacteria phylum, including Mycobacteriaceae and the Mycobacterium genus, Pasteurellaceae family, including the Haemophilus and Pasteurella genus, Legionellaceae family, including Legionella, the Bacteroidetes phylum, including the family of Bacteroidetes and Porphyromonadaceae, or any of their drug-resistant, synthetic or engineered variants.

According to certain embodiments, the bacterium is a pathogen (disease-causing), e.g. a human, animal or plant pathogen.

According to certain embodiments, the pathogen is selected from the group consisting of Clostridium species, including Clostridium difficile, Clostridium perfringens and Clostridium tetani, Bacillus species, including Bacillus anthracis, Haemophilus species including Haemophilus influenzae, Helicobacter species, including Helicobacter pylori, Bacteroides species, including Bacteroides fragilis, Bordetella pertussis, Borrelia species, including Borrelia burgdorferi, Borrelia garinii, Borrelia afzelii, Brucella species, including Brucella abortus, Brucella canis, Brucella melitensis, Brucella suis, Chlamydia species, Coxiella species, Legionella species, including Legionella pneumophila, Corynebacterium diphtheriae, Ehrlichia species, including Ehrlichia canis, Ehrlichia chaffeensis, Leptospira species, Treponema species, including Treponema pallidum, Neisseria species, including Neisseria gonorrhoeae and including drug-resistant Neisseria gonorrhoeae, Listeria species including Listeria monocytogenes, the Enterobacteriaceae family, including Carbapenem-resistant Enterobacteriaceae (CRE), extended spectrum 1-lactamase producing Enterobacteriaceae (ESBLs), Enterobacter species, including Enterobacter cloacae, Enterobacter aerogenes, Proteus species, Providencia species, Salmonella including drug-resistant Non-typhoidal Salmonella and Salmonella Typhi, Shigella, including drug-resistant Shigella, Citrobacter species, Escherichia species, including drug- and multidrug resistant Escherichia coli, including Enterotoxigenic E. coli (ETEC), Enteropathogenic E. coli (EPEC), uropathogenic E. coli (UPEC), Enteroinvasive E. coli (EIEC), Enterohemorrhagic E. coli (EHEC), Acinetobacter species, including Acinetobacter baumannii, drug- and multidrug-resistant Acinetobacter, Campylobacter, including drug-resistant Campylobacter, Enterococcus species, including Vancomycin-resistant Enterococcus (VRE), Enterococcus faecium, Enterococcus faecalis, Enterococcus gallinarum, Pseudomonas species, including Pseudomonas aeruginosa, including drug- and multidrug-resistant Pseudomonas aeruginosa, Staphylococcus aureus, including methicillin-resistant Staphylococcus aureus (MRSA) and Vancomycin-resistant Staphylococcus aureus (VRSA), Staphylococcus epidermidis and methicillin-resistant Staphylococcus epidermidis (MRSE), Staphylococcus haemolyticus, Staphylococcus saprophyticus, Streptococcus intermedius, Streptococcus species and their drug-resistant variants, including Streptococcus agalactiae, Streptococcus bovis, Streptococcus constellatus, Streptococcus mitis, Streptococcus mutans, Streptococcus pyogenes, Streptococcus oralis, Streptococcus sanguis, Streptococcus pneumoniae and including any of their drug-resistant variants, Stenotrophomonas maltophilia, Pasteurella multocida, Burkholderia species and their drug-resistant variants, including Burkholderia cepacia, Burkholderia mallei, Burkholderia pseudomalilei, Burkholderia thailandensis, Propionibacterium species including Propionibacterium acnes, Yersinia species, including Yersinia pestis, Mycobacterium tuberculosis, including drug-resistant and extremely-drug-resistant Mycobacterium tuberculosis, Mycobacterium leprae, Mycoplasma species including Mycoplasmapneumoniae, Nocardia asteroids, Vibrio species or any of the ESKAPE pathogens, including Enterococcus faecium, Staphylococcus aureus, Klebsiella pneumoniae, Acinetobacter baumannii, Pseudomonas aeruginosa, Enterobacter species or any of their drug-resistant variants.

According to certain embodiments, the drug is an antibacterial agent, which is a chemotherapeutic agent that has the capacity to inhibit the growth of or to kill, one or more microorganism. Antibiotics are well-known to those skilled in the art. Classes of antibiotics include, but are not limited to, aminoglycosides (e.g., amikacin, gentamicin, kanamycin, neomycin, netilmicin, streptomycin, tobramycin, paromomycin and the like), ansamycins (e.g., geldanamycin, herbimycin and the like), carbacephem (e.g., loracarbef), carbapenems (e.g., ertapenem, doripenem, imipenem/cilastatin, meropenem and the like) cephalogensporins (e.g., first generation (e.g., cefadroxil, cefazolin, cefalotin, cefalexin and the like), second generation (e.g., cefaclor, cefamandole, cefoxitin, cefprozil, cefuroxime and the like), third generation (e.g., cefixime, cefdinir, cefditoren, cefoperazone, cefotaxime, cefpodoxime, ceftazidime, ceftibuten, ceftizoxime, ceftriaxone and the like), fourth generation (e.g., cefepime and the like) and fifth generation (e.g., ceftobiprole and the like)), glycopeptides (e.g., teicoplanin, vancomycin and the like), macrolides (e.g., azithromycin, clarithromycin, dirithromycin, erythromycin, roxithromycin, troleandomycin, telithromycin, spectinomycin and the like), monobatams (e.g., aztreonam and the like), penicillins (e.g., amoxicillin, ampicillin, azlocillin, carbenicillin, cloxacillin, dicloxacillin, flucloxacillin, mezlocillin, methicillin, nafcillin, oxacillin, penicillin, piperacillin, ticacillin and the like), polypeptides (e.g., bacitracin, colistin, nisin, polymyxin B, PGLA, TPII and the like) quinolones (e.g., ciprofloxacin, enoxacin, gatifloxacin, delafloxacin, levofloxacin, lomefloxacin, moxifloxacin, norfloxacin, ofloxacin, trovafloxacin and the like), sulfonamides (e.g., mafenide, prontosil, sulfacetamide, sulfamethizole, sulfanilamide, sulfasalazine, sulfisoxazole, trimethoprim, trimethoprim-sulfamethoxazole and the like), tetracyclines (e.g., demeclocycline, doxycycline, minocycline, oxytetracycline, tetracycline and the like) and others (e.g., arsphenamine, chloramphenicol, clindamycin, lincomycin, ethambutol, fosfomycin, fusidic acid, furazolidone, novobiocin, isoniazid, linezolid, metronidazole, mupirocin, nitrofurantoin, platensimycin, pyrazinamide, quinupristin, dalfopristin, rifampin, tinidazol and the like) (See e.g., Robert Berkow (ed.) The Merck Manual of Medical Information—Home Edition. Pocket (September 1999), ISBN 0-671-02727-1). Other antibiotics will be readily apparent to those skilled in the art.

### EXAMPLES

The invention is illustrated but not limited by the following Examples in which unless otherwise stated:
(i) evaporation were carried out by rotary evaporation in vacuo and work-up procedures were carried out after removal of residual solids after filtration;
(ii) operations were generally carried out at ambient temperature, that is typically between 18 and 26° C. and without exclusion of air unless otherwise stated, or unless skilled person would otherwise work under an inert atmosphere;
(iii) flash column chromatography was used to purify compounds and was performed on Merck Silica Gel 60 unless otherwise stated;
(iv) yields are given for illustration only and are not necessarily the maximum attainable;
(v) the structure of the end-products was generally confirmed by NMR and mass spectral techniques; proton NMR spectra are quoted and were determined using a Bruker Avance Ill 400 MHz spectrometer operating at 400 MHz. Chemical shifts are reported in part per million downfield from tetramethylsilane as an internal standard (5 scale) and peak multiplicities are shown thus: s, singlet; d, doublet; dd, doublet of doublets; dt, doublet of triplets; t, triplet; m, multiplet; br, broad;
(vi) mass spectra were obtained using a Q TOF Premier mass spectrometer (Micromass, Waters, Manchester, UK) and Exactive Plus Orbitrap mass spectrometer (Thermo Fisher Scientific, Waltham, Massachusetts, ZDA).
(vii) each intermediate was generally purified to the standard required for the subsequent stage and was characterised in sufficient detail to confirm that the assigned structure was correct; purity was assessed by high pressure liquid chromatography, thin layer chromatography, or NMR and identity was determined by mass spectrometry and NMR spectroscopy as appropriate.

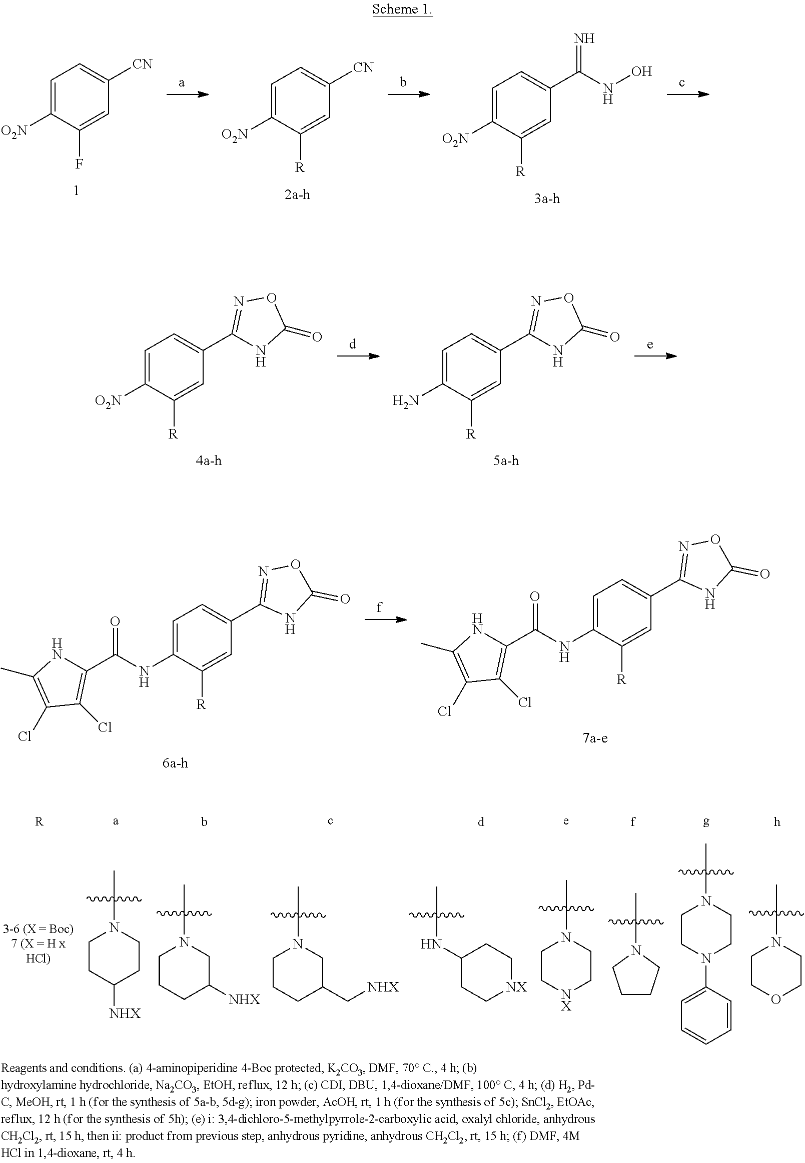

### (Example 1) 1-(2-(3,4-Dichloro-5-methyl-1H-pyrrole-2-carboxamido)-5-(5-oxo-4,5-dihydro-1,2,4-oxadiazol-3-yl)phenyl)piperidin-4-aminium chloride (7a)

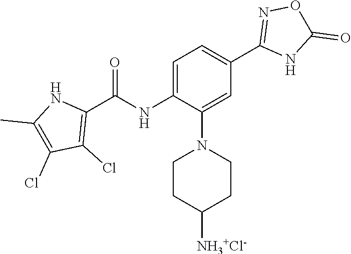

Step 1: Synthesis of tert-butyl (1-(5-cyano-2-nitrophenyl)piperidin-4-yl)carbamate (2a). General procedure A. To a suspension of 3-fluoro-4-nitrobenzonitrile (1, 2.00 g, 12.0 mmol) and potassium carbonate (3.32 g, 24.0 mmol) in DMF (50 ml), 4-aminopiperidine 4-Boc protected (2.40 g, 12.0 mmol) was added. The mixture was heated at 70° C. for 12 h. The solvent was evaporated under reduced pressure. EtOAc (70 mL) and H₂O (50 mL) were added and the compound was extracted to the organic layer. The organic phase was washed with H₂O (40 mL) and brine (2×40 mL), dried over Na₂SO₄, filtered and the solvent removed under reduced pressure to obtain the product (4.16 g) as orange solid. Yield: 98% (4.16 g); orange solid; mp 137-139° C. ¹H NMR (400 MHz, CDCl₃): δ 1.46 (s, 9H, tBu), 1.54-1.64 (m, 2H, CH₂), 2.01-2.13 (m, 2H, CH₂), 2.92-3.01 (m, 2H, CH₂), 3.23-3.30 (m, 2H, CH₂), 3.64 (s, 1H, CH), 4.50 (s, 1H, NH), 7.24 (dd, 1H, J=8.3, 1.6 Hz, ArH), 7.37 (d, 1H, J=1.6 Hz, ArH), 7.79 (d, 1H, J=8.3 Hz, ArH) ppm. MS (ESI) m/z=369.3 ([M+Na]+).

Step 2: Synthesis of tert-butyl (1-(5-(N-hydroxycarbamimidoyl)-2-nitrophenyl)piperidin-4-yl)carbamate (3a). General procedure B. A mixture of 2a (2.00 g, 5.77 mmol), hydroxylamine hydrochloride (1.61 g, 23.1 mmol) and sodium carbonate (1.84 g, 17.4 mmol) was refluxed in absolute ethanol (60 mL) for 12 h. The solvent was evaporated under reduced pressure. EtOAc (60 mL) and H₂O (40 mL) were added to the residue. The organic phase was washed with H₂O (2×40 mL) and brine (2×40 mL), dried over Na₂SO₄, filtered and the solvent removed under reduced pressure. The crude product was purified with flash column chromatography using ethyl acetate/petroleum ether (1:2) as an eluent to afford product (2.19 g) as orange solid. Yield 100% (2.19 g); orange solid; mp 79-81° C. ¹H NMR (400 MHz, DMSO-d₆): δ 1.39 (s, 9H, tBu), 1.45-1.58 (m, 2H, CH₂), 1.80 (d, 2H, J=10.9 Hz, CH₂), 2.86 (t, 2H, J=11.0 Hz, CH₂), 3.19 (d, 2H, J=12.5 Hz, CH₂), 3.36-3.49 (m, 1H, CH), 6.02 (s, 2H, 2×NH), 6.93 (d, 1H, J=7.8 Hz, NH), 7.36 (dd, 1H, J=8.6, 1.6 Hz, ArH), 7.52 (d, 1H, J=1.6 Hz, ArH), 7.80 (d, 1H, J=8.6 Hz, ArH), 9.99 (s, 1H, OH) ppm. MS (ESI) m/z=378.3 ([M−H]⁻).

Step 3: Synthesis of tert-butyl (1-(2-nitro-5-(5-oxo-4,5-dihydro-1,2,4-oxadiazol-3-yl)phenyl)piperidin-4-yl)carbamate (4a). General procedure C. To the solution of 3a (1.90 g, 5.01 mmol) and 1,1′-carbonyldiimidazole (2.84 g, 17.5 mmol) in 1,4-dioxane (40 mL), DBU (0.899 mL, 6.01 mmol) was added and the reaction mixture was stirred at 100° C. for 15 h. The solvent was removed under reduced pressure, and EtOAc (60 mL) and H₂O (40 mL) were added to the residue. The pH of the water phase was adjusted to 3 with HCl 1 M solution, the phases were separated and the organic phase was washed with brine (2×40 mL), dried over Na₂SO₄, filtered and the solvent removed under reduced pressure. The crude product was purified with flash column chromatography using dichloromethane/methanol (30/1) as an eluent to afford the product (2.00 g) as orange solid. Yield: 98% (2.00 g); orange solid; mp 131-133° C. ¹H NMR (400 MHz, DMSO-d₆): δ 1.39 (s, 9H, tBu), 1.45-1.60 (m, 2H, CH₂), 1.81 (d, 2H, J=10.5 Hz, CH₂), 2.90 (t, 2H, J=11.0 Hz, CH₂), 3.21 (d, 2H, J=12.5 Hz, CH₂), 3.42 (s, 1H, CH), 6.94 (d, 1H, J=7.7 Hz, NH), 7.44 (dd, 1H, J=8.5, 1.6 Hz, ArH), 7.64 (d, 1H, J=1.5 Hz, ArH), 7.93 (d, 1H, J=8.5 Hz, ArH) ppm. Signal for NH not seen. MS (ESI) m/z=404.4 ([M−H]⁻).

Step 4: Synthesis of tert-butyl (1-(2-amino-5-(5-oxo-4,5-dihydro-1,2,4-oxadiazol-3-yl)phenyl)piperidin-4-yl)carbamate (5a). General procedure D. 4a (1.67 g, 4.12 mmol) was dissolved in MeOH (150 mL) and the mixture was stirred for 10 min under argon atmosphere. Pd/C (0.400 g) was added and the reaction mixture was stirred under hydrogen atmosphere for 1 h. The catalyst was filtered off and the solvent removed under reduced pressure. The crude product was purified with flash column chromatography using dichloromethane/methanol (15:1) as an eluent to afford the product (0.477 g) as pink solid. Yield: 31% (0.477 g); pink solid; mp 115-117° C. ¹H NMR (400 MHz, DMSO-d₆): δ 1.40 (s, 9H, tBu), 1.58 (q, 2H, J=14.3, 12.9 Hz, CH₂), 1.84 (d, 2H, J=12.3 Hz, CH₂), 2.57 (d, 2H, J=11.6 Hz, CH₂), 3.01 (d, 2H, J=11.8 Hz, CH₂), 3.37-3.41 (m, 1H, CH), 5.45 (s, 2H, NH₂), 6.75 (d, 1H, J=8.3 Hz, ArH), 6.93 (d, 1H, J=7.5 Hz, NH), 7.27 (dd, 1H, J=8.3, 2.0 Hz, ArH), 7.33 (d, 1H, J=2.0 Hz, ArH), 12.54 (s, 1H, NH) ppm. MS (ESI) m/z=374.4 ([M−H]⁻).

Step 5: Synthesis of tert-butyl (1-(2-(3,4-dichloro-5-methyl-1H-pyrrole-2-carboxamido)-5-(5-oxo-4,5-dihydro-1,2,4-oxadiazol-3-yl)phenyl)piperidin-4-yl)carbamate (6a). General procedure E. To a solution of 3,4-dichloro-5-methyl-1H-pyrrole-2-carboxylic acid (0.124 g, 0.640 mmol) in anhydrous dichloromethane (10 mL), oxalyl chloride (0.191 mL, 2.22 mmol) was added dropwise and the solution was stirred at rt for 15 h under argon atmosphere. The solvent was evaporated under reduced pressure. Fresh anhydrous dichloromethane (5 mL), 5a (0.200 g, 3.41 mmol) and pyridine (2 mL) were added and the reaction mixture was stirred under argon atmosphere at rt for 15 h. Solvent was removed under reduced pressure, the residue was dissolved in ethyl acetate (15 mL) and washed with H₂O (10 mL), HCl 1 M solution (15 mL) and brine (2×15 mL). During the extraction the product precipitated and was filtered off. The crude product was triturated with water and the undissolved solid was filtered off. The product was then triturated with diethyl ether and the undissolved solid was filtered off to give the product (0.113 g) as off-white solid. Yield 39% (0.113 g); off-white solid; mp 236-238° C. ¹H NMR (400 MHz, DMSO-d₆): δ 1.41 (s, 9H, tBu), 1.60-1.69 (m, 2H, CH₂), 1.86 (d, 2H, J=12.3 Hz, CH₂), 2.25 (s, 3H, CH₃), 2.76 (td, 2H, J=11.6, 2.5 Hz, CH₂), 2.95 (d, 2H, J=11.4 Hz, CH₂), 3.38-3.49 (m, 1H, CH), 7.12 (d, 1H, J=7.8 Hz, NH), 7.63 (dd, 1H, J=8.6, 2.0 Hz, ArH), 7.80 (d, 1H, J=2.0 Hz, ArH), 8.57 (d, 1H, J=8.7 Hz, ArH), 9.79 (s, 1H, NH), 12.43 (s, 1H, NH), 12.91 (s, 1H, NH) ppm. HRMS for C₂₄H₂₇O₅N₆Cl₂: calculated 549.14255. found 549.14262. HPLC (30-90% ACN in 0.1% TFA in 10 min, UPLC): tᵣ 5.430 min (95.86% at 280 nm).

Step 6: Synthesis of 1-(2-(3,4-dichloro-5-methyl-1H-pyrrole-2-carboxamido)-5-(5-oxo-4,5-dihydro-1,2,4-oxadiazol-3-yl)phenyl)piperidin-4-aminium chloride (7a). General procedure F. Compound 6a (0.070 g, 0.13 mmol) was dissolved in DMF (3 mL) and HCl 4 M solution in dioxane (5 mL) was added. The mixture was stirred for 4 h. After the completion of the reaction, the solvent was removed under reduced pressure. The solid was washed with acetonitrile and with methanol to obtain 7a (0.059 g) as white solid. Yield: 95% (0.059 g); white solid; mp>300° C. ¹H NMR (400 MHz, DMSO-d₆): δ 1.78 (td, 2H, J=12.8, 11.9, 3.8 Hz, CH₂), 2.01-2.12 (m, 2H, CH₂), 2.25 (s, 3H, CH₃), 2.77-2.88 (m, 2H, CH₂), 2.97-3.10 (m, 2H, CH₂), 3.15-3.25 (m, 1H, CH), 7.68 (dd, 1H, J=8.6, 2.0 Hz, ArH), 7.88 (d, 1H, J=2.1 Hz, ArH), 8.21 (d, 3H, J=4.8 Hz, NH₃), 8.59 (d, 1H, J=8.6 Hz, ArH), 9.72 (s, 1H, NH), 12.46 (s, 1H, NH), 13.08 (s, 1H, NH) ppm. ¹³C NMR (100 MHz, DMSO-d₆): δ 11.25, 30.72, 47.74, 51.26, 109.15, 110.68, 118.53, 119.07, 119.80, 120.57, 124.15, 130.54, 137.60, 142.54, 157.14, 157.42, 160.35 ppm. HRMS (ESI−) m/z for C₁₉H₂₁Cl₂N₆O₃ ([M+H]⁺): calculated 451.1047. found 451.1045. HPLC (30-90% ACN in 0.1% TFA in 20 min): tᵣ 3.410 min (97.29% at 254 nm, 97.56% at 280 nm).

### (Example 2) 1-(2-(3,4-Dichloro-5-methyl-1H-pyrrole-2-carboxamido)-5-(5-oxo-4,5-dihydro-1,2,4-oxadiazol-3-yl)phenyl)piperidin-3-aminium chloride (7b)

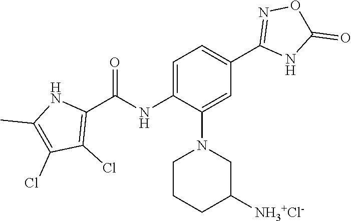

Step 1: Synthesis of tert-butyl (1-(5-cyano-2-nitrophenyl)piperidin-3-yl)carbamate (2b). Synthesised according to General procedure A from 3-fluoro-4-nitrobenzonitrile (2.00 g, 12.0 mmol), potassium carbonate (3.32 g, 24.0 mmol) and 3-aminopiperidine 3-Boc protected (2.40 g, 12.0 mmol). Yield 99% (4,124 g); orange solid; mp 135-136° C. ¹H NMR (400 MHz, DMSO-d₆) δ 1.38 (s, 10H, tBu, CH), 1.53 (dt, 1H, J=13.5, 10.9 Hz, CH), 1.68-1.77 (m, 1H, CH), 1.82 (dd, 1H, J=12.5, 4.2H, CH), 2.63-2.73 (m, 1H, CH), 2.81 (t, 1H, J=11.4 Hz, CH), 3.12 (d, 1H, J=12.8 Hz, CH), 3.21 (dd, 1H, J=12.0, 3.9 Hz, CH), 3.44 (d, 1H, J=4.6 Hz, CH), 6.90 (d, 1H, J=7.5 Hz, NH), 7.47 (dd, 1H, J=8.3, 1.6 Hz, ArH), 7.79 (d, 1H, J=1.6 Hz, ArH), 7.93 (d, 1H, J=8.3 Hz, ArH) ppm. MS (ESI) m/z=369.3 ([M+Na]+).

Step 2: Synthesis of tert-butyl (1-(5-(N-hydroxycarbamimidoyl)-2-nitrophenyl)piperidin-3-yl)carbamate (3b). Synthesised according to General procedure B from 2b (2.50 g, 7.22 mmol), hydroxylamine hydrochloride (2.01 g, 28.9 mmol) and sodium carbonate (2.30 g, 21.7 mmol). Yield 86% (2,364 g); orange solid; mp 142-145° C. ¹H NMR (400 MHz, DMSO-d₆): δ 1.38 (s, 10H, tBu, CH), 1.54-1.63 (m, 1H, CH), 1.68-1.79 (m, 1H, CH), 1.79-1.91 (m, 1H, CH), 2.57-2.65 (m, 1H, CH), 2.70-2.79 (m, 1H, CH), 3.06-3.16 (m, 1H, CH), 3.16-3.25 (m, 1H, CH), 3.41-3.54 (m, 1H, CH), 6.03 (s, 2H, 2×NH), 6.87 (d, 1H, J=7.9 Hz, NH), 7.37 (dd, 1H, J=8.6, 1.7 Hz, ArH), 7.50 (d, 1H, J=1.7 Hz, ArH), 7.80 (d, 1H, J=8.6 Hz, ArH), 9.98 (d, 1H, J=1.0 Hz, OH) ppm. MS (ESI) m/z=378.4 ([M−H]⁻).

Step 3: Synthesis of tert-butyl (1-(2-nitro-5-(5-oxo-4,5-dihydro-1,2,4-oxadiazol-3-yl)phenyl)piperidin-3-yl)carbamate (4b). Synthesised according to General procedure C from 3b (1.77 g, 4.66 mmol) and 1,1′-carbonyldiimidazole (2.65 g, 16.3 mmol). Instead of purification with column chromatography, the crude product was triturated with diethyl ether and filtered off to afford 4b (1.74 g) as orange solid. Yield 92% (1.74 g); orange solid; mp 142-145° C. ¹H NMR (400 MHz, DMSO-d₆): δ 1.38 (s, 1OH, tBu, CH), 1.59 (q, 1H, J=11.9 Hz, CH), 1.72-1.78 (m, 1H, CH), 1.80-1.93 (m, 1H, CH), 2.62 (t, 1H, J=10.5 Hz, CH), 2.73 (t, 1H, J=11.4 Hz, CH), 3.05-3.14 (m, 1H, CH), 3.14-3.21 (m, 1H, CH), 3.44-3.52 (m, 1H, CH), 6.89 (d, 1H, J=7.8 Hz, NH), 7.09 (d, 1H, J=1.0 Hz, NH), 7.46 (dd, 1H, J=8.4, 1.6 Hz, ArH), 7.61 (d, 1H, J=1.6 Hz, ArH), 7.88 (d, 1H, J=8.4 Hz, ArH) ppm. MS (ESI) m/z=404.4 ([M−H]⁻).

Step 4: Synthesis of tert-butyl (1-(2-amino-5-(5-oxo-4,5-dihydro-1,2,4-oxadiazol-3-yl)phenyl)piperidin-3-yl)carbamate (5b). Synthesised according to General procedure D from 4b (1.70 g, 4.19 mmol). The crude product after column chromatography was additionally purified. A part of the product was purified by crystallization from ethyl acetate/hexane (1:1) and washed with ethyl acetate, acetonitrile and diethyl ether to give 4b (0.152 g) as white solid, while the rest was purified with flash column chromatography using ethyl acetate/hexane (1/1) as an eluent to give 4b (0.050 g) as white solid. Yield 13% (0.202 g); white solid; mp 194-195° C. ¹H NMR (400 MHz, DMSO-d₆): δ 1.38 (s, 10H, tBu, CH), 1.58-1.70 (m, 1H, CH), 1.70-1.89 (m, 2H, 2×CH), 2.37-2.44 (m, 1H, CH), 2.55-2.65 (m, 1H, CH), 2.71-2.86 (m, 1H, CH), 2.98 (d, 1H, J=11.0 Hz, CH), 3.56-3.73 (m, 1H, CH), 5.60 (s, 2H, NH₂), 6.73 (d, 1H, J=8.7 Hz, ArH), 7.02 (d, 1H, J=7.9 Hz, NH), 7.26-7.29 (m, 2H, 2×ArH), 12.53 (s, 1H, NH) ppm. MS (ESI) m/z=374.4 ([M−H]⁻).

Step 5: Synthesis of tert-butyl (1-(2-(3,4-dichloro-5-methyl-1H-pyrrole-2-carboxamido)-5-(5-oxo-4,5-dihydro-1,2,4-oxadiazol-3-yl)phenyl)piperidin-3-yl)carbamate (6b). Synthesised according to General procedure E from 5b (0.170 g, 0.45 mmol), 3,4-dichloro-5-methyl-1H-pyrrole-2-carboxylic acid (0,126 g, 0.54 mmol) and oxalyl chloride (0,163 ml, 1.90 mmol). The crude product was additionally triturated with methanol and the undissolved solid was filtered off to give 6b (0.020 g) as white-grey solid. Yield 8% (0.020 g); mp>300° C. ¹H NMR (400 MHz, DMSO-d₆): δ 1.35 (s, 10H, tBu, CH), 1.61-1.76 (m, 1H, CH), 1.77-1.90 (m, 2H, 2×CH), 2.24 (s, 3H, CH₃), 2.53-2.62 (m, 2H, 2×CH), 2.84 (d, 1H, J=11.3 Hz, CH), 2.93-3.03 (m, 1H, CH), 3.60 (q, 1H, J=10.9, 8.4 Hz, CH), 6.95 (d, 1H, J=7.5 Hz, NH), 7.66 (dd, 1H, J=8.6, 2.0 Hz, ArH), 7.73 (d, 1H, J=2.0 Hz, ArH), 8.53 (d, 1H, J=8.6 Hz, ArH), 9.76 (s, 1H, NH), 12.46 (s, 1H, NH), 12.91 (s, 1H, NH) ppm. HRMS for C₂₄H₂₉O₅N₆Cl₂: calculated 551.15710. found 551.15565.

Step 6: Synthesis of 1-(2-(3,4-dichloro-5-methyl-1H-pyrrole-2-carboxamido)-5-(5-oxo-4,5-dihydro-1,2,4-oxadiazol-3-yl)phenyl)piperidin-3-aminium chloride (7b). Synthesised according to General procedure F from 6b (0.015 g, 0.03 mmol). Yield 77% (0.010 g); white solid; mp>300° C. ¹H NMR (400 MHz, DMSO-d₆): δ 1.51 (td, 1H, J=11.9, 3.9 Hz, CH), 1.64-1.77 (m, 1H, CH), 1.85-1.97 (m, 1H, CH), 2.04-2.15 (m, 1H, CH), 2.66-2.79 (m, 2H, 2×CH), 2.91 (d, 1H, J=11.4 Hz, CH), 3.14-3.23 (m, 1H, CH), 3.44 (m, 1H, CH), 7.69 (dd, 1H, J=8.6, 2.0 Hz, ArH), 7.76 (d, 1H, J=2.0 Hz, ArH), 8.12 (d, 3H, J=5.3 Hz, NH₃), 8.48 (d, 1H, J=8.7 Hz, ArH), 9.59 (s, 1H, NH), 12.56 (s, 1H, NH), 13.06 (s, 1H, NH) ppm. HRMS for C₁₉H₂₁O₃N₆Cl₂: calculated 451.10467. found 451.10388. HPLC (10-90% ACN in 0.1% TFA in 10 min, UPLC): tᵣ 4.713 min (95.02% at 254 nm, 95.43% at 280 nm).

### (Example 3) (1-(2-(3,4-Dichloro-5-methyl-1H-pyrrole-2-carboxamido)-5-(5-oxo-4,5-dihydro-1,2,4-oxadiazol-3-yl)phenyl)piperidin-3-yl)methanaminium chloride (7c)

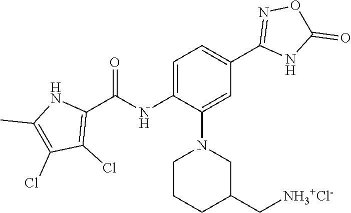

Step 1: Synthesis of tert-butyl ((1-(5-cyano-2-nitrophenyl)piperidin-3-yl)methyl)carbamate (2c). Synthesised according to General procedure A from 3-fluoro-4-nitrobenzonitrile (3.00 g, 12.0 mmol) and 3-(aminomethyl)piperidine 3-Boc protected (3.87 g, 18.1 mmol). Yield 93% (6,045 g); orange solid; mp 106-108° C. ¹H NMR (400 MHz, CDCl₃): δ 1.14-1.26 (m, 1H, CH), 1.45 (s, 9H, tBu), 1.65-1.77 (m, 1H, CH), 1.77-1.89 (m, 2H, 2×CH), 1.92 (m, 1H, CH), 2.66 (dd, 1H, J=12.1, 9.6 Hz, CH), 2.86 (ddd, 1H, J=12.0, 10.6, 2.9 Hz, CH), 3.01-3.14 (m, 2H, 2×CH), 3.14-3.28 (m, 2H, 2×CH), 4.64 (s, 1H, NH), 7.20 (dd, 1H, J=8.3, 1.7 Hz, ArH), 7.37 (d, 1H, J=1.6 Hz, ArH), 7.77 (d, 1H, J=8.4 Hz, ArH) ppm. MS (ESI) m/z=383.3 ([M+Na]+).

Step 2: Synthesis of tert-butyl ((1-(5-(N-hydroxycarbamimidoyl)-2-nitrophenyl)piperidin-3-yl)methyl)carbamate (3c). Synthesised according to General procedure B from 2c (4.00 g, 11.1 mmol), hydroxylamine hydrochloride (3.09 g, 44.4 mmol) and sodium carbonate (3.53 g, 33.3 mmol). The crude product was purified with flash column chromatography using dichloromethane/methanol (gradient from 30:1 to 15:1) as an eluent to afford 3c (3.60 g) as orange solid. Yield 82% (3.60 g); mp 71-74° C. ¹H NMR (400 MHz, DMSO-d₆): δ 1.06 (q, 1H, J=12.2, 11.5 Hz, CH), 1.37 (s, 9H, tBu), 1.48-1.59 (m, 1H, CH), 1.72 (d, 3H, J=10.5 Hz, 3×CH), 2.51 (m, 2H, 2×CH), 2.71-2.84 (m, 2H, 2×CH), 2.86-2.93 (m, 1H, CH), 3.14 (d, 2H, J=11.4 Hz, 2×CH), 6.00 (s, 2H, 2×NH), 6.89 (t, 1H, J=6.0 Hz, NH), 7.34 (dd, 1H, J=8.5, 1.7 Hz, ArH), 7.50 (d, 1H, J=1.8 Hz, ArH), 7.79 (d, 1H, J=8.5 Hz, ArH), 9.97 (s, 1H, OH) ppm. MS (ESI) m/z=394.3 ([M+H]⁺).

Step 3: Synthesis of tert-butyl ((1-(2-nitro-5-(5-oxo-4,5-dihydro-1,2,4-oxadiazol-3-yl)phenyl)piperidin-3-yl)methyl)carbamate (4c). Synthesised according to General procedure C from 3c (3.60 g, 9.15 mmol) and 1,1′-carbonyldiimidazole (5.19 g, 32.0 mmol). Instead of purification with column chromatography, the crude product was triturated with diethyl ether and acetonitrile to obtain 0.800 g of 4c as orange solid. Solvent was removed from the mother liquor and flash column chromatography was performed with the residue using dichloromethane/methanol (gradient from 30:1 to 15:1) as an eluent to afford 2.50 g of 4c as orange solid. Yield 86% (3.30 g); orange solid; mp 87-89° C. ¹H NMR (400 MHz, DMSO-d₆): δ 1.04-1.17 (m, 1H, CH), 1.38 (s, 9H, tBu), 1.59-1.60 (m, 1H, CH), 1.73 (d, 3H, J=10.0 Hz, 3×CH), 2.51-2.59 (m, 1H, CH), 2.76-2.96 (m, 3H, 3×CH), 3.17 (d, 2H, J=11.3 Hz, 2×CH), 6.91 (t, 1H, J=5.9 Hz, NH), 7.43 (dd, 1H, J=8.5, 1.7 Hz, ArH), 7.63 (d, 1H, J=1.7 Hz, ArH), 7.96 (d, 1H, J=8.4 Hz, ArH), 13.21 (s, 1H, NH) ppm. MS (ESI) m/z=418.4 ([M−H]⁻).

Step 4: Synthesis of tert-butyl ((1-(2-amino-5-(5-oxo-4,5-dihydro-1,2,4-oxadiazol-3-yl)phenyl)piperidin-3-yl)methyl)carbamate (5c). General procedure G. 4c (2.50 g, 5.96 mmol) was dissolved in 100 mL of glacial acetic acid, iron powder (3.33 g, 59.6 mmol) was added after which the mixture turned slowly dark green within 3 min. The mixture was stirred for 1 hour at rt upon which a green suspension has formed. Water was added to the mixture to obtain a solution that was then filtered over Celite. EtOAc was added and the phases were separated. The organic layer was washed with brine, dried over Na₂SO₄, filtered and evaporated. Residues of acetic acid were removed with azeptropic distillation with toluene. Crude product was purified with flash column chromatography using ethyl acetate/hexane (1:1) as an eluent to afford 5c (0.314 g) as white solid. Yield: 14% (0.314 g); white solid; mp 184-186° C. ¹H NMR (400 MHz, DMSO-d₆): δ 0.95-1.09 (m, 1H, CH), 1.36 (s, 9H, tBu), 1.61-1.77 (m, 3H, 3×CH), 1.77-1.89 (m, 1H, CH), 2.24 (t, 1H, J=10.6 Hz, CH), 2.39-2.47 (m, 1H, CH), 2.79-3.04 (m, 4H, 4×CH), 5.47 (s, 2H, NH₂), 6.75 (d, 1H, J=8.3 Hz, ArH), 6.90 (t, 1H, J=5.8 Hz, NH), 7.26 (dd, 1H, J=8.3, 2.0 Hz, ArH), 7.32 (d, 1H, J=2.0 Hz, ArH), 12.54 (s, 1H, NH) ppm. MS (ESI) m/z=388.4 ([M−H]⁻).

Step 5: Synthesis of tert-butyl ((1-(2-(3,4-dichloro-5-methyl-1H-pyrrole-2-carboxamido)-5-(5-oxo-4,5-dihydro-1,2,4-oxadiazol-3-yl)phenyl)piperidin-3-yl)methyl)carbamate (6c). Synthesised according to General procedure E from 5c (0.160 g, 0.41 mmol) and 3,4-dichloro-5-methyl-1H-pyrrole-2-carboxylic acid (0.096 g, 0.49 mmol). During the extraction the product precipitated and was filtered off. The crude product was triturated with water and the undissolved solid was filtered off. The product was then triturated with diethyl ether and the undissolved solid was filtered off to give 5c (0.155 g) as off-white solid. Yield 67% (0.155 g); off-white solid; mp 246-248° C. ¹H NMR (400 MHz, DMSO-d₆): δ 1.02 (q, 1H, J=12.2, 11.7 Hz, CH), 1.31 (s, 9H, tBu), 1.59-1.72 (m, 1H, CH), 1.73-1.84 (m, 2H, 2×CH), 1.84-1.95 (m, 1H, CH), 2.24 (s, 3H, CH₃), 2.31-2.38 (m, 1H, CH), 2.64-2.68 (m, 1H, CH), 2.71-2.79 (m, 1H, CH), 2.85-2.99 (m, 3H, 3×CH), 6.90 (t, 1H, J=5.8 Hz, NH), 7.64 (dd, 1H, J=8.6, 2.0 Hz, ArH), 7.78 (d, 1H, J=2.0 Hz, ArH), 8.54 (d, 1H, J=8.6 Hz, ArH), 9.82 (s, 1H, NH), 12.44 (s, 1H, NH), 12.92 (s, 1H, NH) ppm. HRMS for C₂₅H₃₁O₅N₆Cl₂: calculated 565.17275. found 565.17200.

Step 6: Synthesis of (1-(2-(3,4-dichloro-5-methyl-1H-pyrrole-2-carboxamido)-5-(5-oxo-4,5-dihydro-1,2,4-oxadiazol-3-yl)phenyl)piperidin-3-yl)methanaminium chloride (7c). Synthesised according to General procedure F from 6c (0.120 g, 0.212 mmol). Yield 62% (0.066 g); white solid; mp>300° C. ¹H NMR (400 MHz, DMSO-d₆): δ 1.06-1.20 (m, 1H, CH), 1.60-1.76 (m, 1H, CH), 1.86 (dd, 2H, J=33.2, 12.7 Hz, 2×CH), 2.00-2.13 (m, 1H, CH), 2.25 (s, 3H, CH₃), 2.32-2.42 (m, 1H, CH), 2.64-2.68 (m, 1H, CH), 2.72-2.80 (m, 2H, 2×CH), 2.89-2.96 (m, 1H, CH), 3.01-3.13 (m, 1H, CH), 7.67 (dd, 1H, J=8.6, 2.0 Hz, ArH), 7.70-7.94 (m, 4H, NH₃, ArH), 8.55 (d, 1H, J=8.6 Hz, ArH), 9.82 (s, 1H, NH), 12.49 (s, 1H, NH), 13.02 (s, 1H, NH) ppm. ¹³C NMR (100 MHz, DMSO-d₆): 11.25, 25.40, 27.70, 35.36, 42.35, 53.84, 56.28, 109.15, 110.47, 118.52, 119.20, 119.63, 120.51, 124.17, 130.52, 137.69, 143.15, 157.09, 157.40, 160.34 ppm. HRMS for C₂₀H₂₃O₃N₆Cl₂: calculated 465.12032. found 465.11954. HPLC (10-90% ACN in 0.1% TFA in 10 min, UPLC): tᵣ 3.613 min (95.62% at 220 nm, 95.30% at 280 nm).

### (Example 4) 4-((2-(3,4-Dichloro-5-methyl-1H-pyrrole-2-carboxamido)-5-(5-oxo-4,5-dihydro-1,2,4-oxadiazol-3-yl)phenyl)amino)piperidin-1-ium chloride (7d)

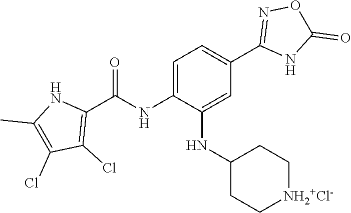

Step 1: Synthesis of tert-butyl 4-((5-cyano-2-nitrophenyl)amino)piperidine-1-carboxylate (2d). Synthesised according to General procedure A from 3-fluoro-4-nitrobenzonitrile (2.00 g, 12.0 mmol) and 4-aminopiperidine N₁-Boc-protected (2.40 g, 12.0 mmol). Yield 94% (3.89 g); orange solid; mp 148-150° C. ¹H NMR (400 MHz, CDCl₃): δ 1.48 (s, 9H, tBu), 1.51-1.60 (m, 2H, CH₂), 2.07 (dd, 2H, J=13.3, 3.9 Hz, CH₂), 3.06 (t, 2H, J=12.5 Hz, CH₂), 3.60-3.69 (m, 1H, CH), 4.06 (br s, 2H, CH₂), 6.88 (dd, 1H, J=8.8, 1.6 Hz, ArH), 7.18 (d, 1H, J=1.6 Hz, ArH), 8.07 (d, 1H, J=7.4 Hz, NH), 8.27 (d, 1H, J=8.7 Hz, ArH) ppm. MS (ESI) m/z=345.4 ([M−H]⁻).

Step 2: Synthesis of tert-butyl 4-((5-(N-hydroxycarbamimidoyl)-2-nitrophenyl)amino)piperidine-1-carboxylate (3d). Synthesised according to General procedure B from 2d (1.00 g, 2.89 mmol), hydroxylamine hydrochloride (0.802 g, 11.5 mmol) and sodium carbonate (0.918 g, 8.66 mmol). The product was used in the next step without purification with column chromatography. Yield 96% (1.05 g); orange solid; mp 179-181° C. ¹H NMR (400 MHz, DMSO-d₆): δ 1.42 (s, 11H, tBu, CH₂), 1.92-2.04 (m, 2H, CH₂), 2.99 (br s, 2H, CH₂), 3.86-4.01 (m, 3H, CH, CH₂), 6.08 (s, 2H, 2×NH), 7.04 (dd, 1H, J=9.1, 1.7 Hz, ArH), 7.29 (d, 1H, J=1.8 Hz, ArH), 8.02-8.06 (m, 2H, ArH, NH), 10.13 (s, 1H, OH) ppm. MS (ESI) m/z=378.4 ([M−H]⁻).

Step 3: Synthesis of tert-butyl 4-((2-nitro-5-(5-oxo-4,5-dihydro-1,2,4-oxadiazol-3-yl)phenyl)amino)piperidine-1-carboxylate (4d). Synthesised according to General procedure C from 3d (1.12 g, 2.96 mmol) and 1,1′-carbonyldiimidazole (1.44 g, 8.88 mmol). Instead of purification with flash column chromatography, the crude product was triturated with acetonitrile and filtered off to afford 4d (0.698 g) as orange solid. Yield 58% (0.698 g); orange solid; mp 204-206° C. ¹H NMR (400 MHz, DMSO-d₆): δ 1.42 (s, 9H, tBu), 1.45-1.55 (m, 2H, CH₂), 1.92-2.04 (m, 2H, CH₂), 3.00 (br s, 2H, CH₂), 3.83-4.00 (m, 3H, CH, CH₂), 7.10 (dd, 1H, J=8.9, 1.7 Hz, ArH), 7.50 (s, 1H, ArH), 8.01 (d, 1H, J=7.9 Hz, NH), 8.19 (d, 1H, J=9.0 Hz, ArH) ppm. Signal for one NH not seen. MS (ESI) m/z=404.4 ([M−H]⁻).

Step 4: Synthesis of tert-butyl 4-((2-amino-5-(5-oxo-4,5-dihydro-1,2,4-oxadiazol-3-yl)phenyl)amino)piperidine-1-carboxylate (5d). Synthesised according to General procedure D from 4d (0.300 g, 0.74 mmol). After purification with flash column chromatography, the crude product was washed with acetonitrile and subsequently with methanol to give 5d (0.096 g) as light pink solid. Yield 35% (0.096 g); light pink solid; mp 168-171° C. ¹H NMR (400 MHz, DMSO-d₆): δ 1.21-1.32 (m, 2H, CH₂), 1.41 (s, 9H, tBu), 1.94 (dd, 2H, J=13.2, 3.6 Hz, CH₂), 2.92 (br s, 2H, CH₂), 3.39-3.50 (m, 1H, CH), 3.93 (d, 2H, J=13.2 Hz, CH₂), 4.54 (d, 1H, J=7.6 Hz, NH), 5.36 (s, 2H, NH₂), 6.60 (d, 1H, J=8.1 Hz, ArH), 6.83 (d, 1H, J=1.9 Hz, ArH), 6.92 (dd, 1H, J=8.1, 1.9 Hz, ArH), 12.51 (s, 1H, NH) ppm. MS (ESI) m/z=374.4 ([M−H]⁻).

Step 5: Synthesis of tert-butyl 4-((2-(3,4-dichloro-5-methyl-1H-pyrrole-2-carboxamido)-5-(5-oxo-4,5-dihydro-1,2,4-oxadiazol-3-yl)phenyl)amino)piperidine-1-carboxylate (6d). Synthesised according to General procedure E from 3,4-dichloro-5-methyl-1H-pyrrole-2-carboxylic acid (0.109 g, 0.56 mmol) and 5d (0.175 g, 0.47 mmol). During the extraction the product precipitated and was filtered off. The crude product was washed with acetonitrile and then with methanol to give 6d (0.150 g) as grey solid. Yield 58% (0.150 g); grey solid; mp 231-233° C. ¹H NMR (400 MHz, DMSO-d₆): δ 1.34 (t, 2H, J=12.2 Hz, CH₂), 1.41 (s, 9H, tBu), 1.93 (d, 2H, J=12.7 Hz, CH₂), 2.24 (s, 3H, CH₃), 2.95 (s, 2H, CH₂), 3.52 (s, 1H, CH), 3.90 (d, 2H, J=13.2 Hz, CH₂), 5.28 (d, 1H, J=7.0 Hz, NH), 7.17 (d, 2H, J=8.2 Hz, 2×ArH), 7.73 (d, 1H, J=8.0 Hz, ArH), 8.82 (s, 1H, NH), 12.28 (s, 1H, NH), 12.92 (s, 1H, NH) ppm. HRMS for C₂₄H₂₉O₅N₆Cl₂: calculated 551.15710. found 551.15493. HPLC (30-90% ACN in 0.1% TFA in 10 min, UPLC): tᵣ 5.087 min (97.19% at 254 nm, 98.23% at 280 nm).

Step 6: Synthesis of 4-((2-(3,4-dichloro-5-methyl-1H-pyrrole-2-carboxamido)-5-(5-oxo-4,5-dihydro-1,2,4-oxadiazol-3-yl)phenyl)amino)piperidin-1-ium chloride (7d). Synthesised according to General procedure F from 6d (0.064 g, 0.12 mmol). Yield 81% (0.052 g); grey solid; mp>300° C. ¹H NMR (400 MHz, DMSO-d₆): δ 1.69 (q, 2H, J=10.8 Hz, CH₂), 2.10 (d, 2H, J=15.5 Hz, CH₂), 2.25 (s, 3H, CH₃), 3.05 (t, 2H, J=11.1 Hz, CH₂), 3.33 (d, 2H, J=12.5 Hz, CH₂), 3.66 (m, 1H, CH), 7.21 (dd, 1H, J=8.3, 1.9 Hz, ArH), 7.24 (d, 1H, J=1.9 Hz, ArH), 7.78 (d, 1H, J=8.3 Hz, ArH), 8.69 (s, 1H, NH from NH₂), 8.80 (s, 1H, NH from NH₂), 9.08 (s, 1H, NH), 12.55 (s, 1H, NH), 13.03 (s, 1H, NH) ppm. ¹³C NMR (100 MHz, DMSO-d₆): δ 10.17, 27.41, 41.31, 46.00, 108.07, 109.73, 111.34, 114.35, 118.52, 119.32, 123.91, 127.61, 127.79, 139.05, 156.63, 156.89, 159.37 ppm. HRMS for C₁₉H₂₁O₃N₆Cl₂: calculated 451.10467. found 451.10390. HPLC (10-90% ACN in 0.1% TFA in 10 min, UPLC): tᵣ 5.280 min (98.53% at 254 nm, 98.40% at 280 nm).

### (Example 5) 4-(2-(3,4-Dichloro-5-methyl-1H-pyrrole-2-carboxamido)-5-(5-oxo-4,5-dihydro-1,2,4-oxadiazol-3-yl)phenyl)piperazin-1-ium chloride (7e)

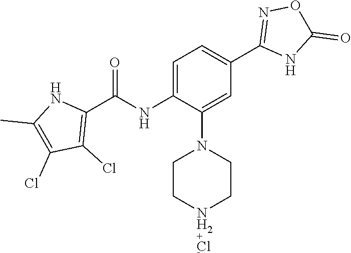

Step 1: Synthesis of tert-butyl 4-(5-cyano-2-nitrophenyl)piperazine-1-carboxylate (2e). Synthesised according to General procedure A from 3-fluoro-4-nitrobenzonitrile (2.00 g, 12.0 mmol) and piperazine-N₁-Boc protected (2.24 g, 12.0 mmol). Yield 100% (4.00 g); orange solid; mp 90-92° C. ¹H NMR (400 MHz, DMSO-d₆): δ 1.42 (s, 9H, tBu), 2.97-3.09 (m, 4H, 2×CH₂), 3.42 (t, 4H, J=4.8 Hz, 2×CH₂), 7.58 (dd, 1H, J=8.4, 1.6 Hz, ArH), 7.87 (d, 1H, J=1.6 Hz, ArH), 7.99 (d, 1H, J=8.4 Hz, ArH) ppm. MS (ESI) m/z=355.1 ([M+Na]+).

Step 2: Synthesis of tert-butyl 4-(5-(N-hydroxycarbamimidoyl)-2-nitrophenyl)piperazine-1-carboxylate (3e). Synthesised according to General procedure B from 2e (4.02 g, 12.1 mmol), hydroxylamine hydrochloride (3.60 g, 48.4 mmol) and sodium carbonate (3.85 g, 36.3 mmol). The product was used in the next step without purification with column chromatography. Yield 99% (4.389 g); orange solid; mp 140-143° C. ¹H NMR (400 MHz, DMSO-d₆): δ 1.42 (s, 9H, tBu), 2.94-3.06 (m, 4H, 2×CH₂), 3.44 (dd, 4H, J=6.6, 3.4 Hz, 2×CH₂), 6.04 (s, 2H, 2×NH), 7.44 (dd, 1H, J=8.6, 1.7 Hz, ArH), 7.55 (d, 1H, J=1.8 Hz, ArH), 7.85 (d, 1H, J=8.6 Hz, ArH), 10.01 (s, 1H, OH) ppm. MS (ESI) m/z=364.0 ([M−H]⁻).

Step 3: Synthesis of tert-butyl 4-(2-nitro-5-(5-oxo-4,5-dihydro-1,2,4-oxadiazol-3-yl)phenyl)piperazine-1-carboxylate (4e). Synthesised according to General procedure C from 3e (4.35 g, 11.9 mmol), 1,1′-carbonyldiimidazole (4.83 g, 29.8 mmol) and DBU (2.14 mL, 14.3 mmol). After purification with flash column chromatography using dichloromethane/methanol (15:1) as an eluent the residue was triturated with acetonitrile and the undissolved solid was filtered off to afford 4e (3.60 g) as orange solid. Yield 77% (3,600 g); orange solid; mp 165-169° C. ¹H NMR (400 MHz, DMSO-d₆): δ 1.42 (s, 9H, tBu), 2.97-3.09 (m, 4H, 2×CH₂), 3.46 (t, 4H, J=5.1 Hz, 2×CH₂), 7.53 (dd, 1H, J=8.5, 1.7 Hz, ArH), 7.68 (d, 1H, J=1.8 Hz, ArH), 8.03 (d, 1H, J=8.5 Hz, ArH), 13.18 (s, 1H, NH) ppm. MS (ESI) m/z=390.0 ([M−H]⁻).

Step 4: Synthesis of tert-butyl 4-(2-amino-5-(5-oxo-4,5-dihydro-1,2,4-oxadiazol-3-yl)phenyl)piperazine-1-carboxylate (5e). Synthesised according to General procedure D from 4e (0.846 g, 2.16 mmol). Yield 42% (0.324 g); pink solid; mp 190-193° C. ¹H NMR (400 MHz, DMSO-d₆): δ 1.43 (s, 9H, tBu), 2.68-2.82 (m, 4H, 2×CH₂), 3.51 (s, 4H, 2×CH₂), 5.63 (s, 2H, NH₂), 6.75-6.80 (m, 1H, ArH), 7.30 (d, 2H, J=7.9 Hz, 2×ArH), 12.51 (s, 1H, NH) ppm. MS (ESI) m/z=360.0 ([M−H]⁻).

Step 5: Synthesis of tert-butyl 4-(2-(3,4-dichloro-5-methyl-1H-pyrrole-2-carboxamido)-5-(5-oxo-4,5-dihydro-1,2,4-oxadiazol-3-yl)phenyl)piperazine-1-carboxylate (6e). Synthesised according to General procedure E from 3,4-dichloro-5-methyl-1H-pyrrole-2-carboxylic acid (0.129 g, 0.66 mmol) and 5e (0.200 g, 0.55 mmol). During the extraction the product precipitated and was filtered off. The crude product was triturated with water and the undissolved solid was filtered off. The product was then triturated with diethyl ether and the undissolved solid was filtered off to give 6e (0.131 g) as violet solid. Yield 44% (0.131 g); violet solid; mp>300° C. ¹H NMR (400 MHz, DMSO-d₆): δ 1.44 (s, 9H, tBu), 2.24 (s, 3H, CH₃), 2.83 (t, 4H, J=4.9 Hz, 2×CH₂), 3.53 (s, 4H, 2×CH₂), 7.68 (dd, 1H, J=8.6, 2.0 Hz, ArH), 7.78 (d, 1H, J=2.1 Hz, ArH), 8.55 (d, 1H, J=8.6 Hz, ArH), 9.76 (s, 1H, NH), 12.46 (s, 1H, NH), 12.86 (s, 1H, NH) ppm. HRMS for C₂₃H₂₇O₅N₆Cl₂: calculated 537.14145. found 537.13711. HPLC (30-90% ACN in 0.1% TFA in 10 min, UPLC): tᵣ 5.280 min (95.26% at 280 nm).

Step 6: Synthesis of 4-(2-(3,4-dichloro-5-methyl-1H-pyrrole-2-carboxamido)-5-(5-oxo-4,5-dihydro-1,2,4-oxadiazol-3-yl)phenyl)piperazin-1-ium chloride (7e). Synthesised according to General procedure F from 6e (0.074 g, 0.14 mmol). Yield 82% (0.053 g); light violet solid; mp>300° C. ¹H NMR (400 MHz, DMSO-d₆): δ 2.25 (s, 3H, CH₃), 3.10 (t, 4H, J=5.0 Hz, 2×CH₂), 3.28 (d, 4H, J=5.3 Hz, 2×CH₂), 7.71 (dd, 1H, J=8.6, 2.0 Hz, ArH), 7.76 (d, 1H, J=2.0 Hz, ArH), 8.53 (d, 1H, J=8.6 Hz, ArH), 9.08 (s, 2H, NH₂), 9.57 (s, 1H, NH), 12.51 (s, 1H, NH), 13.10 (s, 1H, NH) ppm. ¹³C NMR (100 MHz, DMSO-d₆): δ 11.24, 43.83, 49.23, 109.20, 110.71, 118.73, 119.19, 120.15, 120.35, 124.39, 130.43, 137.35, 141.53, 157.13, 157.34, 160.35 ppm. HRMS for Cl₈H₁₉O₃N₆Cl₂: calculated 437.08902. found 437.08848. HPLC (10-90% ACN in 0.1% TFA in 10 min, UPLC): tᵣ 3.283 min (95.16% at 280 nm).

### (Example 6) 3,4-Dichloro-5-methyl-N-(4-(5-oxo-4,5-dihydro-1,2,4-oxadiazol-3-yl)-2-(pyrrolidin-1-yl)phenyl)-1H-pyrrole-2-carboxamide (6f)

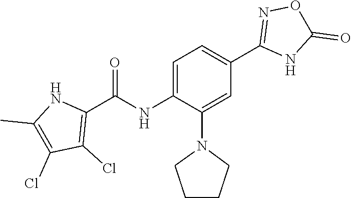 [SMILES: Cc1[nH]c(C(=O)Nc2ccc(-c3noc(=O)[nH]3)cc2N2CCCC2)c(Cl)c1Cl]

Step 1: Synthesis of 4-nitro-3-(pyrrolidin-1-yl)benzonitrile (2f). Synthesised according to General procedure A from 3-fluoro-4-nitrobenzonitrile (3.00 g, 18.1 mmol) and pyrrolidine (1.65 ml, 18.0 mmol). Yield 82% (3.23 g); orange solid; mp 98-100° C. ¹H NMR (400 MHz, DMSO-d₆): b 1.88-1.97 (m, 4H, 2×CH₂), 3.12-3.21 (m, 4H, 2×CH₂), 7.12 (dd, 1H, J=8.4, 1.6 Hz, ArH), 7.53 (d, 1H, J=1.6 Hz, ArH), 7.87 (d, 1H, J=8.4 Hz, ArH) ppm. MS (ESI) m/z=216.2 ([M−H]⁻).

Step 2: Synthesis of N-hydroxy-4-nitro-3-(pyrrolidin-1-yl)benzimidamide (3f). Synthesised according to General procedure B from 2f (3.17 g, 14.6 mmol), hydroxylamine hydrochloride (4.06 g, 58.4 mmol) and sodium carbonate (4.64 g, 43.8 mmol). The crude product after extraction was triturated with ethyl acetate, sonicated and the undissolved solid was filtered off to obtain 3f (3.61 g) as orange oil. Yield 99% (3.61 g); orange oil. ¹H NMR (400 MHz, DMSO-d₆): δ 1.86-1.98 (m, 4H, 2×CH₂), 3.16 (q, 4H, J=6.1, 5.0 Hz, 2×CH₂), 5.97 (s, 2H, 2×NH), 7.06 (dd, 1H, J=8.7, 1.7 Hz, ArH), 7.28 (d, 1H, J=1.7 Hz, ArH), 7.71 (d, 1H, J=8.6 Hz, ArH), 9.93 (s, 1H, OH) ppm. MS (ESI) m/z=249.2 ([M−H]⁻).

Step 3: Synthesis of 3-(4-nitro-3-(pyrrolidin-1-yl)phenyl)-1,2,4-oxadiazol-5(4H)-one (4f). Synthesised according to General procedure C from 3f (3.92 g, 15.7 mmol), 1,1′-carbonyldiimidazole (2.84 g, 17.5 mmol) and DBU (2.35 mL, 18.8 mmol). The crude product was triturated with ethyl acetate and the undissolved solid was filtered off to afford 4f (3.44 g) as orange solid. Yield 79% (3.44 g); orange solid; mp 182-184° C. ¹H NMR (400 MHz, DMSO-d₆): δ 1.90-1.98 (m, 4H, 2×CH₂), 3.13-3.24 (m, 4H, 2×CH₂), 7.15 (d, 1H, J=1.7 Hz, ArH), 7.43 (t, 1H, J=1.4 Hz, ArH), 7.89 (d, 1H, J=8.5 Hz, ArH), 13.24 (s, 1H, NH) ppm. MS (ESI) m/z=274.9 ([M−H]⁻).

Step 4: Synthesis of 3-(4-amino-3-(pyrrolidin-1-yl)phenyl)-1,2,4-oxadiazol-5(4H)-one (5f). Synthesised according to General procedure D from 4f (2.80 g, 10.1 mmol). The crude product was purified with flash column chromatography using dichloromethane/methanol (40:1) as an eluent to afford 5f (0.740 g) as grey solid. Yield 30% (0.740 g); grey solid; mp 159-161° C. ¹H NMR (400 MHz, DMSO-d₆): δ 1.83-1.94 (m, 4H, 2×CH₂), 2.94-3.04 (m, 4H, 2×CH₂), 5.43 (s, 2H, NH₂), 6.72 (d, 1H, J=8.3 Hz, ArH), 7.22 (dd, 1H, J=8.3, 2.0 Hz, ArH), 7.27 (d, 1H, J=2.0 Hz, ArH), 12.53 (s, 1H, NH) ppm. MS (ESI) m/z=245.0 ([M−H]⁻).

Step 5: Synthesis of 3,4-dichloro-5-methyl-N-(4-(5-oxo-4,5-dihydro-1,2,4-oxadiazol-3-yl)-2-(pyrrolidin-1-yl)phenyl)-1H-pyrrole-2-carboxamide (6f). Synthesised according to General procedure E from 3,4-dichloro-5-methyl-1H-pyrrole-2-carboxylic acid (0.189 g, 0.97 mmol) and 5f (0.200 g, 0.81 mmol). During the extraction the product precipitated and was filtered off. The crude product was triturated with methanol and the undissolved solid was filtered off to give 6f (0.253 g) as grey solid. Yield 74% (0.253 g); grey solid; mp 159-161° C. ¹H NMR (400 MHz, DMSO-d₆): δ 1.88-1.98 (m, 4H, 2×CH₂), 2.24 (s, 3H, CH₃), 3.04-3.13 (m, 4H, 2×CH₂), 7.53 (dd, 1H, J=8.5, 1.9 Hz, ArH), 7.65 (d, 1H, J=2.0 Hz, ArH), 8.32 (d, 1H, J=8.6 Hz, ArH), 9.55 (s, 1H, NH), 12.41 (s, 1H, NH), 12.91 (s, 1H, NH) ppm. HRMS for Cl₈H₁₈O₃N₆Cl₂: calculated 422.07812. found 422.07951. HPLC (30-90% ACN in 0.1% TFA in 10 min, UPLC): tᵣ 5.360 min (97.34% at 254 nm, 98.57% at 280 nm).

### (Example 7) 3,4-Dichloro-5-methyl-N-(4-(5-oxo-4,5-dihydro-1,2,4-oxadiazol-3-yl)-2-(4-phenylpiperazin-1-yl)phenyl)-1H-pyrrole-2-carboxamide (6g)

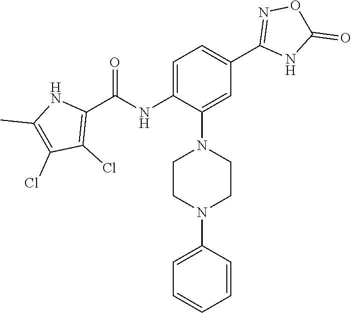 [SMILES: Cc1[nH]c(C(=O)Nc2ccc(-c3noc(=O)[nH]3)cc2N2CCN(c3ccccc3)CC2)c(Cl)c1Cl]

Step 1: Synthesis of 4-nitro-3-(4-phenylpiperazin-1-yl)benzonitrile (2g). Synthesised according to General procedure A from 3-fluoro-4-nitrobenzonitrile (2.50 g, 15.1 mmol), potassium carbonate (4.16 g, 30.1 mmol) and 1-phenylpiperazine (2.30 mL, 15.1 mmol). Yield 99% (4.61 g); orange solid; mp 109-111° C. ¹H NMR (400 MHz, DMSO-d₆): δ 3.17-3.29 (m, 8H, 4×CH₂), 6.78-6.86 (m, 1H, ArH), 6.95-7.01 (m, 2H, 2×ArH), 7.19-7.29 (m, 2H, 2×ArH), 7.56 (dd, 1H, J=8.3, 1.6 Hz, ArH), 7.90 (d, 1H, J=1.6 Hz, ArH), 7.99 (d, 1H, J=8.3 Hz, ArH) ppm. MS (ESI) m/z=309.2 ([M+H]⁺).

Step 2: Synthesis of N-hydroxy-4-nitro-3-(4-phenylpiperazin-1-yl)benzimidamide (3g). Synthesised according to General procedure B from 2g (3.48 g, 11.3 mmol), hydroxylamine hydrochloride (3.14 g, 45.1 mmol) and sodium carbonate (3.59 g, 33.9 mmol). The crude product was purified with flash column chromatography using ethyl acetate/petroleum ether (1/2) as an eluent to afford 3g (3.82 g) as red oil. Yield 99% (3.82 g); red oil. ¹H NMR (400 MHz, DMSO-d₆): δ 3.15-3.24 (m, 4H, 2×CH₂), 3.24-3.30 (m, 4H, 2×CH₂), 6.06 (s, 2H, 2×NH), 6.82 (tt, 1H, J=7.3, 1.0 Hz, ArH), 6.96-7.03 (m, 2H, 2×ArH), 7.21-7.28 (m, 2H, 2×ArH), 7.43 (dd, 1H, J=8.6, 1.7 Hz, ArH), 7.59 (d, 1H, J=1.7 Hz, ArH), 7.86 (d, 1H, J=8.6 Hz, ArH), 10.03 (s, 1H, OH) ppm. MS (ESI) m/z=342.3 ([M+H]⁺).

Step 3: Synthesis of 3-(4-nitro-3-(4-phenylpiperazin-1-yl)phenyl)-1,2,4-oxadiazol-5(4H)-one (4g). Synthesised according to General procedure C from 3g (4.40 g, 12.9 mmol), 1,1′-carbonyldiimidazole (6.27 g, 38.7 mmol) and DBU (2.31 mL, 15.5 mmol). The crude product was triturated with diethyl ether and then purified with flash column chromatography using dichloromethane/methanol (30/1) as an eluent to afford 4g (4.53 g) as red solid. Yield 96% (4.53 g); red solid; mp 135-138° C. ¹H NMR (400 MHz, DMSO-d₆): δ 3.20 (dd, 4H, J=6.4, 3.1 Hz, 2×CH₂), 3.28 (dd, 4H, J=6.6, 3.0 Hz, 2×CH₂), 6.78-6.85 (m, 1H, ArH), 6.95-7.04 (m, 2H, 2×ArH), 7.21-7.28 (m, 2H, 2×ArH), 7.51 (dd, 1H, J=8.5, 1.7 Hz, ArH), 7.70 (d, 1H, J=1.7 Hz, ArH), 7.95 (d, 1H, J=8.4 Hz, ArH) ppm. Signal for NH proton not seen. MS (ESI) m/z=366.4 ([M−H]⁻).

Step 4: Synthesis of 3-(4-amino-3-(4-phenylpiperazin-1-yl)phenyl)-1,2,4-oxadiazol-5(4H)-one (5g). Synthesised according to General procedure D from 4g (2.60 g, 7.08 mmol). Yield 22% (0.533 g); pink solid; mp 198-200° C. ¹H NMR (400 MHz, DMSO-d₆): δ 2.96 (t, 4H, J=4.8 Hz, 2×CH₂), 3.33 (m, 4H, 2×CH₂), 5.61 (s, 2H, NH₂), 6.77-6.84 (m, 2H, 2×ArH), 6.98-7.03 (m, 2H, 2×ArH), 7.22-7.28 (m, 2H, 2×ArH), 7.32 (dd, 1H, J=8.3, 2.0 Hz, ArH), 7.37 (d, 1H, J=2.0 Hz, ArH), 12.57 (s, 1H, NH) ppm. MS (ESI) m/z=336.0 ([M−H]⁻).

Step 5: Synthesis of 3,4-dichloro-5-methyl-N-(4-(5-oxo-4,5-dihydro-1,2,4-oxadiazol-3-yl)-2-(4-phenylpiperazin-1-yl)phenyl)-1H-pyrrole-2-carboxamide (6g). Synthesised according to General procedure E from 3,4-dichloro-5-methyl-1H-pyrrole-2-carboxylic acid (0.138 g, 0.71 mmol) and 5g (0.200 g, 0.59 mmol). During the extraction the product precipitated and was filtered off. The crude product was sequentially triturated with acetonitrile, methanol and THE and the undissolved solid was filtered off to give 6g (0.203 g) as grey solid. Yield 67% (0.203 g); grey solid; mp>300° C. ¹H NMR (400 MHz, DMSO-d₆): δ 2.23 (s, 3H, CH₃), 3.04 (t, 4H, J=4.9 Hz, 2×CH₂), 3.36 (t, 4H, J=4.9 Hz, 2×CH₂), 6.80-6.88 (m, 1H, ArH), 6.99-7.06 (m, 2H, 2×ArH), 7.23-7.30 (m, 2H, 2×ArH), 7.69 (dd, 1H, J=8.7, 2.0 Hz, ArH), 7.83 (d, 1H, J=2.0 Hz, ArH), 8.56 (d, 1H, J=8.6 Hz, ArH), 9.83 (s, 1H, NH), 12.46 (s, 1H, NH), 12.91 (s, 1H, NH) ppm. HRMS for C₂₄H₂₃O₃N₆Cl₂: calculated 513.12032. found 513.11975. HPLC (10-90% ACN in 0.1% TFA in 10 min, UPLC): tᵣ 6.097 min (95.49% at 254 nm).

### (Example 8) 3,4-Dichloro-5-methyl-N-(2-morpholino-4-(5-oxo-4,5-dihydro-1,2,4-oxadiazol-3-yl)phenyl)-1H-pyrrole-2-carboxamide (6h)

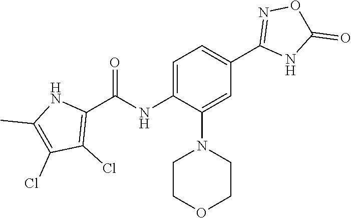 [SMILES: Cc1[nH]c(C(=O)Nc2ccc(-c3noc(=O)[nH]3)cc2N2CCOCC2)c(Cl)c1Cl]

Step 1: Synthesis of 3-morpholino-4-nitrobenzonitrile (2h). Synthesised according to General procedure A from 3-fluoro-4-nitrobenzonitrile (2.00 g, 12.0 mmol), potassium carbonate (2.32 g, 16.8 mmol)) and morpholine (1.04 mL, 12.0 mmol). The crude product was triturated with methanol and diethyl ether (2:1, 8 mL) and filtered off to afford 2h (1.85 g) as orange solid. Yield 66% (1.85 g); orange solid; mp 116-118° C. ¹H NMR (400 MHz, DMSO-d₆): δ 3.04-3.06 (m, 4H, 2×NCH₂), 3.67-3.69 (m, 4H, 2×OCH₂), 7.56 (dd, 1H, J=8.0, 1.6 Hz, Ar—H-6), 7.85 (d, 1H, J=1.6 Hz, Ar—H-2), 7.97 (d, 1H, J=8.0 Hz, Ar—H-5) ppm. HRMS for C₁₁H₁₀N₃O₃: calculated 232.0959. found 232.0956.

Step 2: Synthesis of N-hydroxy-3-morpholino-4-nitrobenzimidamide (3h). Synthesised according to General procedure B from 2h (1.28 g, 5.48 mmol), hydroxylamine hydrochloride (1.52 g, 21.9 mmol) and sodium carbonate (1.74 g, 16.4 mmol). Yield 95% (1.37 g); orange solid; mp 128-130° C. ¹H NMR (400 MHz, DMSO-d₆): δ 3.01-3.04 (m, 4H, 2×NCH₂), 3.69-3.72 (m, 4H, 2×OCH₂), 6.06 (br s, 2H, 2×NH), 7.43 (dd, 1H, J=8.5, 1.6 Hz, Ar—H-6), 7.54 (d, 1H, J=1.6 Hz, Ar—H-2), 7.84 (d, 1H, J=8.5 Hz, Ar—H-5), 10.02 (s, 1H, OH) ppm. MS (ESI) m/z=264.80 ([M−H]⁻). HRMS for C₁₁H₁₃N₄O₄: calculated 264.8038. found 264.8041.

Step 3: Synthesis of 3-(3-morpholino-4-nitrophenyl)-1,2,4-oxadiazol-5(4H)-one (4h). Synthesised according to General procedure C from 3h (1.31 g, 4.92 mmol), 1,1′-carbonyldiimidazole (1.99 g, 12.27 mmol) and DBU (0.88 mL, 5.90 mmol). The crude product was triturated with methanol (8 mL) and diethyl ether (2 mL) and the undissolved solid was filtered off to give 4h (1.10 g) as orange solid. Yield 77% (1.10 g); orange solid; mp 217-220° C. ¹H NMR (400 MHz, DMSO-d₆): δ 3.04-3.06 (m, 4H, 2×NCH₂), 3.70-3.72 (m, 4H, 2×OCH₂), 7.51 (dd, 1H, J=8.5, 1.7 Hz, Ar—H-6), 7.65 (d, 1H, J=1.7 Hz, Ar—H-2), 8.00 (d, 1H, J=8.5 Hz, Ar—H-5) ppm. Signal for NH not seen. MS (ESI) m/z=290.90 ([M−H]⁻). HRMS for C₁₁H₁₁N₄O₅: calculated 290.9122. found 290.9134.

Step 4: Synthesis of 3-(4-amino-3-morpholinophenyl)-1,2,4-oxadiazol-5(4H)-one (5h). To a solution of 4 h (0.570 g, 1.95 mmol) in EtOAc (25 mL), SnCl₂·2H₂O (1.62 g, 7.20 mmol) was added. The mixture was refluxed for 12 h and after the completion of reaction NaHCO₃ solution was added dropwise to reach pH 7. The mixture was filtered through Celite® and the pH of mother liquor was adjusted to pH 2. EtOAc (20 mL) was added to the aqueous layer and the phases were separated. The organic phase was washed with brine (2×20 mL), dried over Na₂SO₄, filtered and the solvent removed under reduced pressure to give 5h (0.310 g) as light yellow solid. Yield 61% (0.310 g); light yellow solid; mp 208-212° C. ¹H NMR (400 MHz, DMSO-d₆): δ 2.79-2.81 (m, 4H, 2×NCH₂), 3.76-3.78 (m, 4H, 2×OCH₂), 5.60 (br s, 2H, NH₂), 6.76 (d, 1H, J=8.5 Hz, Ar—H-5), 7.28-7.33 (m, 2H, Ar—H-2,6) ppm. Signal for NH not seen. MS (ESI) m/z=260.90 ([M−H]⁻). HRMS for C₁₂H₁₃N₄O₃: calculated 260.9066. found 260.9071.

Step 5: Synthesis of 3,4-dichloro-5-methyl-N-(2-morpholino-4-(5-oxo-4,5-dihydro-1,2,4-oxadiazol-3-yl)phenyl)-1H-pyrrole-2-carboxamide (6h). Synthesised according to General procedure E from 3,4-dichloro-5-methyl-1H-pyrrole-2-carboxylic acid (225 mg, 1.16 mmol) and 5h (254 mg, 0.967 mmol). During the extraction the product precipitated and was filtered off. The crude product was recrystallized from THF/DCM (2:1, 6 mL) to afford 6h (65 mg) as light brown solid. Yield 15% (0.065 g); light brown solid; mp>300° C. ¹H NMR (400 MHz, DMSO-d₆): δ 2.25 (s, 3H, CH₃), 2.87-2.89 (m, 4H, 2×NCH₂), 3.79-3.82 (m, 4H, 2×OCH₂), 7.68 (dd, 1H, J=8.6, 1.9 Hz, Ar—H-5), 7.80 (d, 1H, J=1.9 Hz, Ar—H-3), 8.55 (d, 1H, J=8.6 Hz, Ar—H-6), 9.78 (s, 1H, NH), 12.47 (s, 1H, NH), 12.93 (br s, 1H, NH) ppm. ¹³C NMR (100 MHz, DMSO-d₆): 10.73, 52.24, 66.49, 108.65, 109.88, 118.09, 118.66, 119.48, 119.92, 123.44, 130.01, 137.06, 141.80, 156.57, 156.88, 159.88 ppm. MS (ESI) m/z=436.06 ([M−H]⁻).

HRMS for Cl₈H₁₆Cl₂N₅O₄: calculated 436.0588. found 436.0574. HPLC: Phenomenex Luna C18 column (5 μm, 4.6×250 mm); mobile phase: 10-70% of acetonitrile in phosphate buffer (pH=6.8) in 15 min, 70% acetonitrile to 25 min; flow rate 1.0 mL/min; injection volume: 10 μL; t_{R}: 11.623 min (96.7% at 254 nm).

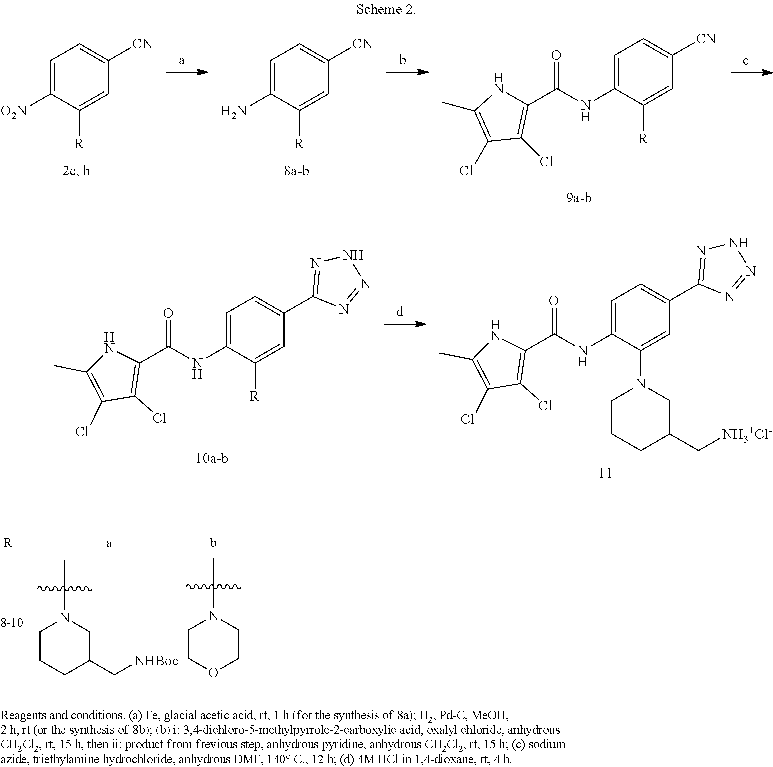

### (Example 9) (1-(2-(3,4-Dichloro-5-methyl-1H-pyrrole-2-carboxamido)-5-(2H-tetrazol-5-yl)phenyl)piperidin-3-yl)methanaminium chloride (11)

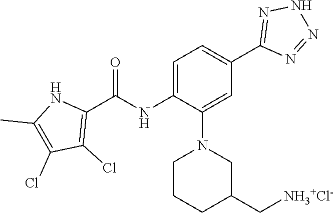

Step 1: Synthesis of tert-butyl ((1-(2-amino-5-cyanophenyl)piperidin-3-yl)methyl)carbamate (8a). Compound 2c (1.60 g, 4.44 mmol) was dissolved in 60 mL of glacial acetic acid. Iron powder (2.48 g, 44.4 mmol) was added after which the mixture turned slowly dark green within 3 min. The mixture was stirred for 1 h at rt upon which a white suspension formed. Water was added to the mixture that was then filtered over Celite. After the addition of water a suspension formed, so methanol was added until the product was dissolved. After filtration, MeOH was removed under reduced pressure. EtOAc was added to the flask and the phases were separated. The organic layer was washed with brine, dried over Na₂SO₄, filtered and evaporated. Residues of acetic acid were removed with azeptropic distillation with toluene. The crude product was purified with flash column chromatography using ethyl acetate/hexane (gradient from 1:3 to 3:1) as an eluent to afford 8 (1.58 g) as beige solid. Yield 100% (1.58 g); beige solid; mp 86-88° C. ¹H NMR (400 MHz, DMSO-d₆): δ 0.94-1.08 (m, 1H, CH), 1.36 (s, 9H, tBu), 1.69 (d, 3H, J=11.6 Hz, 3×CH), 1.74-1.86 (m, 1H, CH), 2.24 (t, 1H, J=10.6 Hz, CH), 2.35-2.45 (m, 1H, CH), 2.82 (dt, 1H, J=13.6, 6.7 Hz, CH), 2.91 (dt, 3H, J=12.6, 6.6 Hz, 3×CH), 5.69 (s, 2H, NH₂), 6.72 (d, 1H, J=8.3 Hz, ArH), 6.88 (t, 1H, J=5.9 Hz, NH), 7.16 (d, 1H, J=1.9 Hz, ArH), 7.21 (dd, 1H, J=8.3, 1.9 Hz, ArH) ppm. MS (ESI) m/z=353.4 ([M+Na]+).

Step 2: Synthesis of tert-butyl ((1-(5-cyano-2-(3,4-dichloro-5-methyl-1H-pyrrole-2-carboxamido)phenyl)piperidin-3-yl)methyl)carbamate (9a). Synthesised according to General procedure E from 3,4-dichloro-5-methyl-1H-pyrrole-2-carboxylic acid (0.282 g, 1.45 mmol) and 8 (0.400 g, 1.21 mmol). The crude product was purified with flash chromatography column using ethyl acetate/hexane (gradient from 1:3 to 3:1) as eluent to give 9 (0.290 g) as light yellow solid. Yield 47% (0.290 g); light yellow solid; mp 157-159° C. ¹H NMR (400 MHz, DMSO-d₆): δ 0.94-1.07 (m, 1H, CH), 1.57-1.72 (m, 1H, CH), 1.76 (dt, 2H, J=9.9, 5.3 Hz, 2×CH), 1.82-1.94 (m, 1H, CH), 2.23 (s, 3H, CH₃), 2.38 (d, 1H, J=10.8 Hz, CH), 2.73 (dd, 2H, J=14.1, 7.2 Hz, 2×CH), 2.79-2.99 (m, 3H, 3×CH), 6.88 (t, 1H, J=5.9 Hz, NH), 7.67 (dd, 1H, J=8.6, 1.9 Hz, ArH), 7.81 (d, 1H, J=1.9 Hz, ArH), 8.54 (d, 1H, J=8.6 Hz, ArH), 9.85 (s, 1H, NH), 12.48 (s, 1H, NH) ppm. HRMS for C₂₄H₃₀O₃N₅Cl₂: calculated 506.17202. found 506.17143. HPLC (30-90% ACN in 0.1% TFA in 10 min, UPLC): tᵣ 6.453 min (95.00% at 280 nm).

Step 3: Synthesis of tert-butyl ((1-(2-(3,4-dichloro-5-methyl-1H-pyrrole-2-carboxamido)-5-(1H-tetrazol-5-yl)phenyl)piperidin-3-yl)methyl)carbamate (10a). To a solution of 9 (0.107 g, 0.21 mmol) in dry DMF (6 mL), triethylamine hydrochloride (0.175 g, 1.27 mmol) and sodium azide (0.082 g, 1.27 mmol) were added. The mixture was heated at 140° C. for 15 h. The resulting precipitate was filtered off and discarded. The mother liquor was evaporated under reduced pressure and EtOAc and H₂O were added to the residue. The pH of the water phase was adjusted to 3 with HCl 1 M solution, the phases were separated, the organic phase was washed with brine, dried over Na₂SO₄, filtered and the solvent removed under reduced pressure. The crude product was triturated with methanol, sonicated and the undissolved solid was filtered off to give 10 (0.033 g) as brown solid. Yield 28% (0.033 g); brown solid; mp 186-188° C. ¹H NMR (400 MHz, DMSO-d₆): δ 0.98-1.12 (m, 1H, CH), 1.61-1.75 (m, 1H, CH), 1.75-1.86 (m, 2H, 2×CH), 1.86-1.98 (m, 1H, CH), 2.24 (s, 3H, CH₃), 2.41 (d, 1H, J=10.5 Hz, CH), 2.75-2.83 (m, 2H, 2×CH), 2.95 (dt, 3H, J=16.3, 10.6 Hz, 3×CH), 5.76 (s, 1H, NH), 6.91 (s, 1H, NH), 7.87 (dd, 1H, J=8.6, 1.8 Hz, ArH), 7.97-8.06 (m, 1H, ArH), 8.58 (d, 1H, J=8.6 Hz, ArH), 9.82 (s, 1H, NH), 12.44 (s, 1H, NH) ppm. MS (ESI−) m/z=546.7 ([M−H]⁻). HRMS for C₂₄H₃₁O₃N₈Cl₂: calculated 549.18907. found 549.18843. HPLC (30-90% ACN in 0.1% TFA in 10 min, UPLC): tᵣ 5.280 min (95.01% at 254 nm, 95.04% at 280 nm).

Step 4: Synthesis of (1-(2-(3,4-dichloro-5-methyl-1H-pyrrole-2-carboxamido)-5-(2H-tetrazol-5-yl)phenyl)piperidin-3-yl)methanaminium chloride (11). Compound 10 (0.026 g, 0.05 mmol) was dissolved in HCl 4 M solution in dioxane (3 mL). The mixture was stirred for 2 h. After the completion of the reaction, the solvent was removed under reduced pressure and the solid was washed with acetonitrile and subsequently with methanol to obtain 11 (0.010 g) as brown solid. Yield 43% (0.010 g); brown solid; mp 240-243° C. ¹H NMR (400 MHz, DMSO-d₆): δ 1.16 (q, 1H, J=11.2, 10.2 Hz, CH), 1.71 (dd, 1H, J=25.3, 12.4 Hz, CH), 1.79-1.98 (m, 2H, 2×CH), 2.02-2.14 (m, 1H, CH), 2.54-2.59 (m, 1H, CH), 2.67-2.71 (m, 1H, CH), 2.75-2.86 (m, 2H, 2×CH), 2.94 (d, 1H, J=11.4 Hz, CH), 3.08-3.15 (m, 1H, CH), 7.80 (s, 3H, NH₃), 7.91 (dd, 1H, J=8.6, 2.0 Hz, ArH), 8.08 (d, 1H, J=2.0 Hz, ArH), 8.59 (d, 1H, J=8.6 Hz, ArH), 9.82 (s, 1H, NH), 12.48 (s, 1H, NH) ppm. Signal for one NH proton not seen. HRMS for C₁₉H₂₃ON₈Cl₂: calculated 449.13664. found 449.13592. HPLC (10-90% ACN in 0.1% TFA in 10 min, UPLC): tᵣ 3.350 min (96.23% at 254 nm, 96.37% at 280 nm).

### (Example 10) 3,4-Dichloro-5-methyl-N-(2-morpholino-4-(2H-tetrazol-5-yl)phenyl)-1H-pyrrole-2-carboxamide (10b)

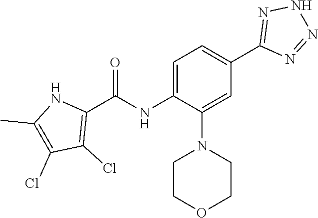 [SMILES: Cc1[nH]c(C(=O)Nc2ccc(-c3nn[nH]n3)cc2N2CCOCC2)c(Cl)c1Cl]

Step 1: Synthesis of 4-amino-3-morpholinobenzonitrile (8b). Compound 2h (0.500 g, 2.14 mmol) was dissolved in MeOH (20 mL) and the mixture was stirred for 30 min under argon atmosphere. Pd—C (100 mg) was added and the reaction mixture was stirred under hydrogen atmosphere for 2 h. The catalyst was filtered off and the solvent removed under reduced pressure to obtain 8b (430 mg) as beige crystals. Yield 99% (430 mg); beige crystals; mp 118-122° C. ¹H NMR (400 MHz, DMSO-d₆): δ 2.77-2.79 (m, 4H, 2×NCH₂), 3.73-3.76 (m, 4H, 2×OCH₂), 5.83 (br s, 2H, NH₂) 6.73 (d, 1H, J=8.7 Hz, Ar—H-5), 7.22-7.24 (m, 2H, Ar—H-2,6) ppm. MS (ESI) m/z=201.80 ([M−H]⁻). HRMS for C₁₁H₁₂N₃O: calculated 201.8078. found 201.8066.

Step 2: Synthesis of 3,4-dichloro-N-(4-cyano-2-morpholinophenyl)-5-methyl-1H-pyrrole-2-carboxamide (9b). Synthesised according to General procedure E from 3,4-dichloro-5-methyl-1H-pyrrole-2-carboxylic acid (388 mg, 1.17 mmol) and 8b (198 mg, 0.975 mmol). During extraction, a part of the product precipitated and was filtered off. The mother liquor was extracted with ethyl acetate and the filtered part of the compound was combined with the part after extraction. To the crude product diethyl ether (8 mL) was added, the obtained suspension was sonicated, filtered off and dried. The solid was further purified with flash column chromatography using dichloromethane/methanol (50/1) as an eluent to afford 9b (130 mg) as light brown solid. Yield 19% (130 mg); light brown solid; mp 250-253° C. ¹H NMR (400 MHz, DMSO-d₆): δ 2.24 (s, 3H, Pyrrole-CH₃), 2.87-2.89 (m, 4H, 2×NCH₂), 3.77-3.79 (m, 4H, 2×OCH₂), 7.69 (dd, 1H, ³J=8.6 Hz, ⁴J=1.8 Hz, Ar—H-5), 7.90 (d, 1H, ⁴J=1.8 Hz, Ar—H-3), 8.55 (d, 1H, ³J=8.6 Hz, Ar—H-6), 9.84 (s, 1H, NHAr), 12.51 (s, 1H, Pyrrole-CH₃) ppm. MS (ESI) m/z=377.06 ([M−H]⁻). HRMS for C₁₇H₁₅Cl₂N₄O₂: calculated 377.0566. found 377.0580. HPLC: Phenomenex Luna C18 column (5 μm, 4.6×250 mm); mobile phase: 10-70% of acetonitrile in phosphate buffer (pH=6.8) in 15 min, 70% acetonitrile to 25 min; flow rate 1.0 mL/min; injection volume: 10 μL; t_{R}: 19.310 min (97.5% at 254 nm).

Step 3: Synthesis of 3,4-dichloro-5-methyl-N-(2-morpholino-4-(1H-tetrazol-5-yl)phenyl)-1H-pyrrole-2-carboxamide (10b). To a solution of 9b (95 mg, 0.25 mmol) in anhydrous DMF (5 mL), triethylamine hydrochloride (103 mg, 0.75 mmol) and sodium azide (49 mg, 0.75 mmol) were added. The mixture was heated at 140° C. for 12 h. The solvent was evaporated under reduced pressure and HCl 1 M solution was added dropwise to reach pH 1. The resulting precipitate was filtered off, triturated with methanol (5 mL) and diethyl ether (1 mL) and the undissolved solid was filtered off to give 10b (21 mg) as brown solid. Yield 20% (21 mg); brown solid; mp 261-264° C. ¹H NMR (400 MHz, DMSO-d₆): δ 2.25 (s, 3H, CH₃), 2.91-2.94 (m, 4H, 2×NCH₂), 3.81-3.83 (m, 4H, 2×OCH₂), 7.90 (dd, 1H, J=8.6, 1.9 Hz, Ar—H-5), 8.02 (d, 1H, J=1.9 Hz, Ar—H-3), 8.59 (d, 1H, J=8.6 Hz, Ar—H-6), 9.77 (s, 1H, NHAr), 12.46 (s, 1H, CH₃) ppm. Signal for tetrazole NH not seen. ¹³C NMR (100 MHz, DMSO-d₆): δ 10.75, 52.31, 66.52, 108.62, 109.78, 118.75, 119.77, 120.51, 120.53, 124.40, 129.89, 136.25, 142.03, 156.57 ppm. Signal for one carbon not seen. MS (ESI) m/z=420.08 ([M−H]⁻). HRMS for C₁₇H₁₆Cl₂N₇O₂: calculated 420.0752. found 420.0737. HPLC: Phenomenex Luna C18 column (5 μm, 4.6×250 mm); mobile phase: 10-70% of acetonitrile in phosphate buffer (pH=6.8) in 15 min, 70% acetonitrile to 25 min; flow rate 1.0 mL/min; injection volume: 10 μL; t_{R}: 11.367 min (97.1% at 254 nm).

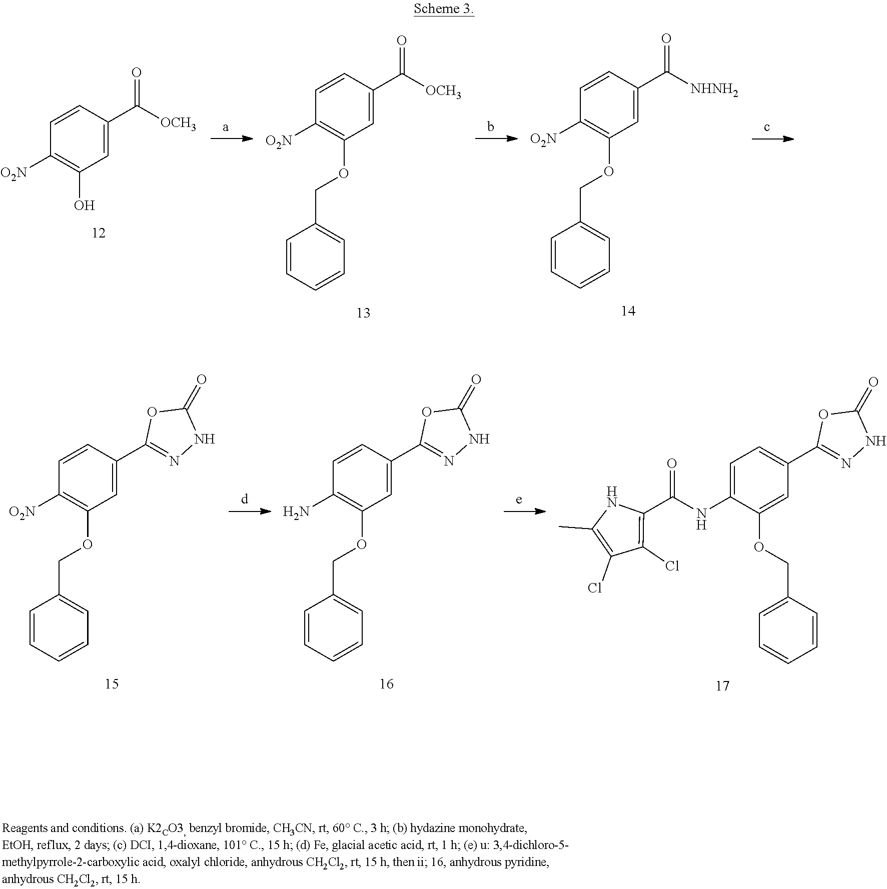 [SMILES: COC(=O)c1ccc([N+](=O)[O-])c(O)c1 ; COC(=O)c1ccc([N+](=O)[O-])c(OCc2ccccc2)c1 ; NNC(=O)c1ccc([N+](=O)[O-])c(OCc2ccccc2)c1 ; O=c1[nH]nc(-c2ccc([N+](=O)[O-])c(OCc3ccccc3)c2)o1 ; Nc1ccc(-c2n[nH]c(=O)o2)cc1OCc1ccccc1 ; Cc1[nH]c(C(=O)Nc2ccc(-c3n[nH]c(=O)o3)cc2OCc2ccccc2)c(Cl)c1Cl]

### (Example 11)N-(2-(Benzyloxy)-4-(5-oxo-4,5-dihydro-1,3,4-oxadiazol-2-yl)phenyl)-3,4-dichloro-5-methyl-1H-pyrrole-2-carboxamide (17)

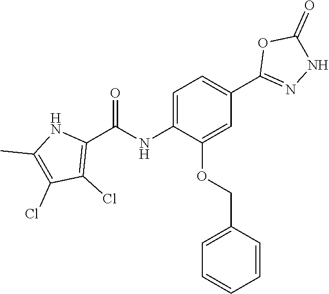 [SMILES: Cc1[nH]c(C(=O)Nc2ccc(-c3n[nH]c(=O)o3)cc2OCc2ccccc2)c(Cl)c1Cl]

Step 1: Synthesis of methyl 3-(benzyloxy)-4-nitrobenzoate (13). To a stirred suspension of methyl 3-hydroxy-4-nitrobenzoate (12, 500 mg, 2.53 mmol) and potassium carbonate (699 mg, 5.06 mmol) in acetonitrile (10 mL) benzyl bromide (0.30 mL, 2.53 mmol) was added and the mixture was stirred at 50° C. for 3 h. The solvent was removed under reduced pressure and to the residue ethyl acetate (20 mL) and water (20 mL) were added, and separated. The organic phase was washed with brine (2×20 mL), dried over Na₂SO₄, filtered and the solvent removed under reduced pressure to afford 13 (620 mg) as yellow solid. Yield 85% (620 mg); yellow solid; mp 90-93° C. ¹H NMR (400 MHz, DMSO-d₆): δ 3.92 (s, 3H, COOCH₃), 5.41 (s, 2H, CH₂), 7.35-7.48 (m, 5H, 5×Ar—H), 7.70 (dd, 1H, J=8.4, 1.2 Hz, Ar—H), 7.90 (d, 1H, J=1.2 Hz, Ar—H), 8.03 (d, 1H, J=8.4 Hz, Ar—H) ppm. MS (ESI) m/z=310.1 ([M+Na]+).

Step 2: Synthesis of 3-(benzyloxy)-4-nitrobenzohydrazide (14). To a solution of 13 (4.60 g, 16.3 mmol) in MeOH (100 mL) and THE (100 mL), hydrazine monohydrate 80% solution in water (7.95 mL, 163 mmol) was added. The reaction mixture was stirred at 65° C. overnight and the solvent was removed under reduced pressure. The residue was suspended in ethanol and flask left in the fridge for 1 h. Precipitate was filtered off, suspended in water, the suspension was sonicated, the solid filtered off and dried to give 1.44 g of 14. Ethanol from the mother liquor of the previous filtration was removed and the residue was purified with flash column chromatography (dichloromethane/methanol 10/1) to give 2.09 of 14. Yield: 77% (3.54 g). ¹H NMR (400 MHz, DMSO-d₆): δ 4.63 (s, 2H, NH₂), 5.37 (s, 2H, CH₂), 7.34-7.38 (m, 1H, ArH), 7.40-7.48 (m, 4H, 4×ArH), 7.54 (dd, 1H, J=8.4, 1.6 Hz, ArH), 7.83 (d, 1H, J=1.5 Hz, ArH), 7.97 (d, 1H, J=8.4 Hz, ArH), 10.06 (s, 1H, NH) ppm. MS (ESI) m/z=285.9 ([M−H]⁻).

Step 3: Synthesis of 5-(3-(benzyloxy)-4-nitrophenyl)-1,3,4-oxadiazol-2(3H)-one (15). To a solution of compound 14 (3.50 g, 12.2 mmol) in 1,4-dioxane (175 mL) 1,1′-carbonyldiimidazole (2.96 g, 18.3 mmol) was added and the reaction mixture was stirred at 101° C. overnight. The solvent was removed under reduce pressure, the residue was suspended in methanol, the suspension was sonicated, heated and the solid filtered off to give 3.22 g of 15. Mother liquor was evaporated and purified with flash column chromatography (dichloromethane/methanol 30/1) and the purified product was combined with 15. Yield: 98% (3.74 g). ¹H NMR (400 MHz, DMSO-d₆): δ 5.43 (s, 2H, CH₂), 7.33-7.50 (m, 5H, 5×ArH), 7.54 (dd, 1H, J=8.4, 1.6 Hz, ArH), 7.73 (d, 1H, J=1.6 Hz, ArH), 8.06 (d, 1H, J=8.4 Hz, ArH), 12.91 (s, 1H, NH) ppm. MS (ESI) m/z=312.0 ([M−H]⁻).

Step 4: Synthesis of 5-(4-amino-3-(benzyloxy)phenyl)-1,3,4-oxadiazol-2(3H)-one (16). Compound 15 (523 mg, 1.67 mmol) was suspended in acetic acid (25 mL), iron (932 mg, 16.7 mmol) was added and the reaction mixture was stirred at rt for 90 min. Water was added and iron was filtered over Celite. The flask was left on an ice bath for 1 h upon which the product in the mother liquor crystalized. The product was filtered off and dried. Yield: 57% (269 mg). ¹H NMR (400 MHz, DMSO-d₆): δ 5.18 (s, 2H, CH₂), 5.54 (s, 2H, NH₂), 6.73 (d, 1H, J=8.2 Hz, ArH), 7.16 (dd, 1H, J=8.2, 1.8 Hz, ArH), 7.21 (d, 1H, J=1.8 Hz, ArH), 7.33 (s, 1H, ArH), 7.40 (s, 2H, 2×Ar—H), 7.51 (d, 2H, J=7.0 Hz, 2×Ar—H), 12.22 (s, 1H, NH) ppm. MS (ESI) m/z=283.9 ([M+H]⁺).

Step 5: Synthesis of N-(2-(benzyloxy)-4-(5-oxo-4,5-dihydro-1,3,4-oxadiazol-2-yl)phenyl)-3,4-dichloro-5-methyl-1H-pyrrole-2-carboxamide (17). Synthesised according to General procedure E from 3,4-dichloro-5-methyl-1H-pyrrole-2-carboxylic acid (0.123 g, 0.71 mmol) and 16 (0.150 g, 0.53 mmol). During the extraction the product precipitated and was filtered off. The crude product was sequentially triturated with acetonitrile, methanol, diethyl ether and the undissolved solid was filtered off. The crude product was purified by crystallization from DMF to afford 17 (0.113 g) as white solid. Yield 47% (0.113 g); white solid; mp 293-295° C. ¹H NMR (400 MHz, DMSO-d₆): δ 2.20 (s, 3H, CH₃), 5.30 (s, 2H, CH₂), 7.42-7.49 (m, 4H, 4×Ar—H), 7.51-7.62 (m, 3H, 3×Ar—H), 8.56 (d, 1H, J=8.5 Hz, ArH), 9.20 (s, 1H, NH), 12.42 (s, 1H, NH), 12.59 (s, 1H, NH) ppm. HRMS for C₂₁H₁₅O₄N₄Cl₂: calculated 457.04758. found 457.04776. HPLC (30-90% ACN in 0.1% TFA in 10 min, UPLC): tᵣ 5.430 min (98.87% at 254 nm, 98.66% at 280 nm).

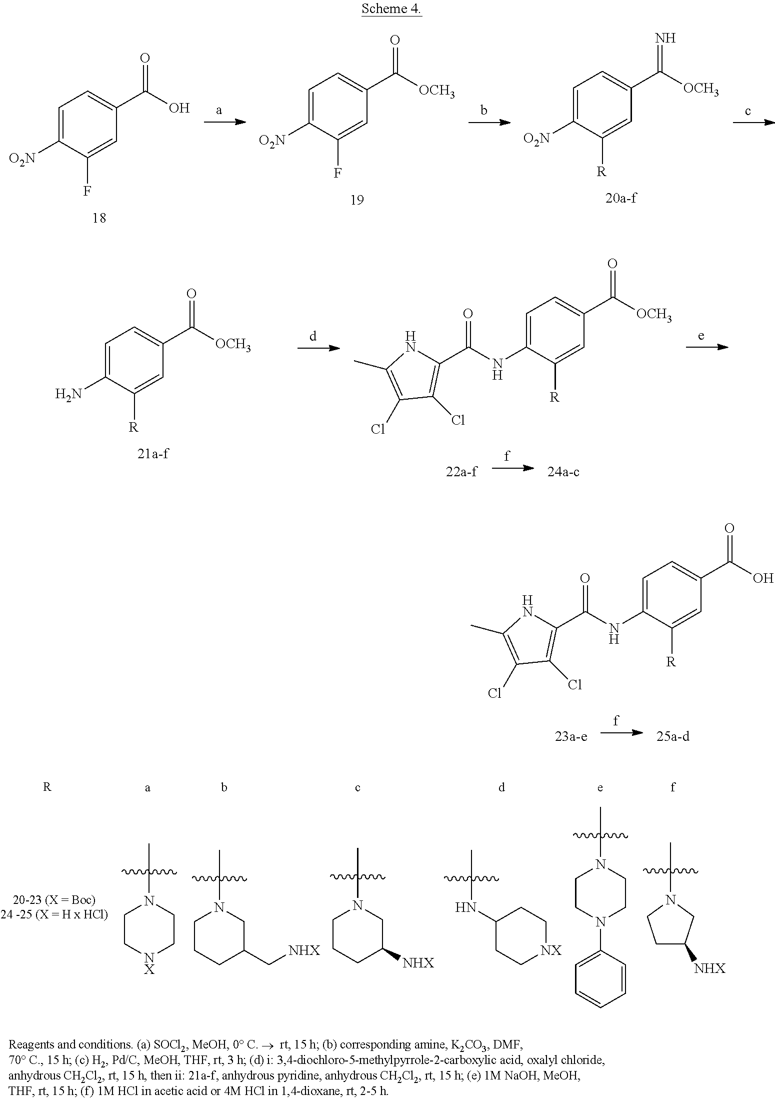

### (Example 12) 4-(5-Carboxy-2-(3,4-dichloro-5-methyl-1H-pyrrole-2-carboxamido)phenyl)piperazin-1-ium chloride (25a)

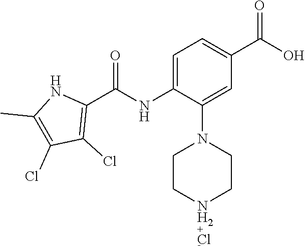

Step 1: Synthesis of methyl 3-fluoro-4-nitrobenzoate (19). To the solution of 3-fluoro-4-nitrobenzoic acid (5.00 g, 27.0 mmol) in methanol (150 mL) cooled on an ice bath thionyl chloride (5.88 mL, 81.0 mmol) was added dropwise and the reaction mixture was heated at 650 for 15 h. The solvent was removed under reduced pressure, to the residue petroleum ether was added, the obtained suspension was sonicated and white precipitate was filtered off and dried to obtain 19 (4.92 g). Yield 95% (4.92 g); yellow solid; mp 40-42° C. ¹H NMR (400 MHz, DMSO-d₆): δ 4.01 (s, 3H, CH₃), 7.97-8.00 (m, 2H, 2×Ar—H), 8.12-8.16 (m, 1H, Ar—H) ppm.

Step 2: Synthesis of tert-butyl 4-(5-(methoxycarbonyl)-2-nitrophenyl)piperazine-1-carboxylate (20a). Synthesised according to General procedure A from 19 (5.00 g, 25.0 mmol), piperazine N1-Boc protected (4.68 g, 25.0 mmol) and K₂CO₃ (4.16 g, 30.0 mmol). To the crude product petroleum ether was added, the obtained suspension was sonicated and yellow precipitate was filtered off to obtain 20a as yellow solid (7.01 g). Yield 83% (7.56 g); yellow solid; mp 88-90° C. ¹H NMR (400 MHz, DMSO-d₆): δ 1.43 (s, 9H, tBu), 2.98-3.08 (m, 4H, 2×CH₂), 3.40-3.50 (m, 4H, 2×CH₂), 3.90 (s, 3H, CH₃), 7.68 (dd, 1H, J=8.4, 1.7 Hz, ArH), 7.80 (d, 1H, J=1.6 Hz, ArH), 7.96 (d, 1H, J=8.4 Hz, ArH).

Step 3: Synthesis of tert-butyl 4-(2-amino-5-(methoxycarbonyl)phenyl)piperazine-1-carboxylate (21a). To the solution of compound 20a (5.44 g, 16.0 mmol) in a mixture of methanol (200 mL) and tetrahydrofuran (90 mL) under an argon atmosphere Pd—C (1.00 g) was added, the mixture was saturated with hydrogene and stirred under a hydrogene atmosphere at rt for 4 h. The catalyst was filtered off and the solvent was removed under reduced pressure to afford 21a (4.87 g) as white solid. Yield 90% (4.87 g); white solid; mp 135-137° C. ¹H NMR (400 MHz, DMSO-d₆): δ 1.43 (s, 9H, tBu), 2.68-2.84 (m, 4H, 2×CH₂), 3.44-3.59 (m, 4H, 2×CH₂), 3.74 (s, 3H, CH₃), 5.73 (br s, 2H, NH₂), 6.70 (d, 1H, J=8.4 Hz, ArH), 7.44 (d, 1H, J=1.9 Hz, ArH), 7.49 (dd, 1H, J=8.4, 1.9 Hz, ArH) ppm. MS (ESI) m/z=336.2 ([M+H]⁺). HRMS for C₁₇H₂₆O₃N₄: calculated 336.1923. found 336.1924.

Step 4: Synthesis of tert-butyl 4-(2-(3,4-dichloro-5-methyl-1H-pyrrole-2-carboxamido)-5-(methoxycarbonyl)phenyl)piperazine-1-carboxylate (22a). Synthesised according to General procedure E from 3,4-dichloro-5-methyl-1H-pyrrole-2-carboxylic acid (0.850 g, 4.38 mmol) and 21a (1.62 g, 4.82 mmol). During the extraction the product precipitated and was filtered off. The crude product was sequentially triturated with methanol and tetrahydrofuran, and the undissolved solid was filtered off and dried, to obtain 22a (0.589 g) as white solid. Yield 25% (0.589 g); white solid; mp 132-136° C. ¹H NMR (400 MHz, DMSO-d₆): δ 1.44 (s, 9H, tBu), 2.24 (s, 3H, CH₃), 2.78-2.87 (m, 4H, 2×CH₂), 3.52 (br s, 4H, 2×CH₂), 3.84 (s, 3H, CH₃), 7.78-7.89 (m, 2H, 2×ArH), 8.51 (d, 1H, J=8.6 Hz, ArH), 9.79 (s, 1H, NH), 12.45 (s, 1H, NH). MS (ESI) m/z=511.2 ([M+H]⁺). HRMS for C₂₃H₂₉N₄O₅Cl₂: calculated 511.1515. found 511.1524. HPLC (30-90% ACN in 0.1% TFA in 16 min, 90% ACN to 20 min, Agilent Eclipse Plus C18: 5 μm, 4,6×150 mm): tᵣ 16.803 min (98.3% at 280 nm).

Step 5: Synthesis of 3-(4-(tert-butoxycarbonyl)piperazin-1-yl)-4-(3,4-dichloro-5-methyl-1H-pyrrole-2-carboxamido)benzoic acid (23a). To a solution of compound 22a (201 mg, 0.391 mmol) in a mixture of methanol (10 mL) and tetrahydrofuran (7 mL) 1 M NaOH (1.56 mL, 1.56 mmol) was added and the mixture was stirred at rt for 15 h. The solvent was removed under reduced pressure, to the residue water (10 mL) was added and the mixture was acidified to pH 4 with 1 M HCl. The water phase was extracted with ethyl acetate (20 mL), the organic phase was washed with brine (10 mL), dried over Na₂SO₄ and the solvent was removed under reduced pressure. The crude product was triturated with diethyl ether and the undissolved solid was filtered off and dried to obtain 23a as pale pink solid (70 mg). Yield 35% (70 mg); pale pink solid; mp 260-262° C. ¹H NMR (400 MHz, DMSO-d₆): δ 1.44 (s, 9H, tBu), 2.24 (s, 3H, CH₃), 2.75-2.91 (m, 4H, 2×CH₂), 3.42-3.63 (m, 4H, 2×CH₂), 7.77-7.88 (m, 2H, 2×ArH), 8.49 (d, 1H, J=8.6 Hz, ArH), 9.78 (s, 1H, NH), 12.45 (s, 1H, NH), 12.87 (s, 1H, COOH) ppm. MS (ESI) m/z=495.1 ([M−H]⁻). HRMS for C₂₂H₂₅N₄O₅Cl₂: calculated 495.1202. found 495.1206. HPLC (30-90% ACN in 0.1% TFA in 16 min, 90% ACN to 20 min, Agilent Eclipse Plus C18: 5 μm, 4,6×150 mm): tᵣ 14.030 min (96.3% at 280 nm).

Step 6: Synthesis of 4-(2-(3,4-dichloro-5-methyl-1H-pyrrole-2-carboxamido)-5-(methoxycarbonyl)phenyl)piperazin-1-ium chloride (24a). Compound 22a (102 mg, 0.200 mmol) was dissolved in 1 M HCl solution in acetic acid (5 mL) and the mixture was stirred at rt for 5 h. The solvent was removed under reduced pressure, the solid residue was sequentially triturated with diethyl ether and water, and the undissolved solid was filtered off and dried to obtain 24a (71 mg) as white solid. Yield 35% (71 mg); white solid; mp 244-248° C. ¹H NMR (400 MHz, DMSO-d₆): b 2.25 (s, 3H, CH₃), 3.05-3.14 (m, 4H, 2×CH₂), 3.22-3.31 (m, 4H, 2×CH₂), 3.86 (s, 3H, COOCH₃), 7.82 (d, 1H, J=1.9 Hz, ArH), 7.87 (dd, 1H, J=8.6, 1.9 Hz, ArH), 8.53 (d, 1H, J=8.6 Hz, ArH), 9.07 (s, 2H, NH₂+), 9.67 (s, 1H, NH), 12.51 (s, 1H, NH) ppm. MS (ESI) m/z=411.1 ([M−H]⁻). HRMS for C₁₈H₂₁N₄O₃Cl₂: calculated 411.0991. found 411.0998. HPLC (30-90% ACN in 0.1% TFA in 16 min, 90% ACN to 20 min, Agilent Eclipse Plus C18: 5 μm, 4,6×150 mm): tᵣ 6.367 min (100% at 280 nm).

Step 7: Synthesis of 4-(5-carboxy-2-(3,4-dichloro-5-methyl-1H-pyrrole-2-carboxamido)phenyl)piperazin-1-ium chloride (25a). Compound 23a (50 mg, 0.100 mmol) was dissolved in a mixture of 1 M HCl solution in acetic acid (5 mL), tetrahydrofuran (2 mL) and dichloromethan (4 mL) and the mixture was stirred at rt for 5 h. The solvent was removed under reduced pressure, the solid residue was sequentially triturated with diethyl ether and water, and the undissolved solid was filtered off and dried to obtain 25a (16 mg) as pale pink solid. Yield 32% (16 mg); pale pink solid; mp 258-261° C. ¹H NMR (400 MHz, DMSO-d₆): δ 2.25 (s, 3H, CH₃), 3.03-3.11 (m, 4H, 2×CH₂), 3.22-3.29 (m, 4H, 2×CH₂), 7.76-7.92 (m, 2H, 2×ArH), 8.50 (d, 1H, J=8.4 Hz, ArH), 9.68 (s, 1H, NH), 12.50 (s, 1H, NH) ppm. Signal for NH₂⁺ is overlapping with the signal for water. Signal for COOH proton not seen. MS (ESI) m/z=395.1 ([M−H]⁻). HRMS for C₁₇H₁₇N₄O₃Cl₂: calculated 395.0678. found 395.0670. HPLC (30-90% ACN in 0.1% TFA in 16 min, 90% ACN to 20 min, Agilent Eclipse Plus C18: 5 μm, 4,6×150 mm): tᵣ 4.769 min (98.1% at 280 nm).

### (Example 13) (1-(5-Carboxy-2-(3,4-dichloro-5-methyl-1H-pyrrole-2-carboxamido)phenyl)piperidin-3-yl)methanaminium chloride (25b)

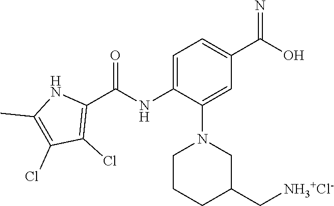

Step 1: Synthesis of ethyl 3-(3-(((tert-butoxycarbonyl)amino)methyl)piperidin-1-yl)-4-nitrobenzoate (20b). Synthesised according to General procedure A from 19 (1.50 g, 7.53 mmol), 3-(Boc-aminomethyl)piperidine (1.61 g, 7.53 mmol) and potassium carbonate (1.46 g, 10.54 mmol). The crude product was triturated with dietyl ether, the undissolved solid was filtered off and dried to afford 20b (2.10 g) as orange solid. Yield 71% (2.10 g); orange solid; mp 88-89° C. ¹H NMR (400 MHz, CDCl₃): δ 1.16-1.28 (m, 1H, CH), 1.46 (s, 9H, tBu), 1.67-1.87 (m, 3H, 3×CH), 1.88-2.00 (m, 1H, CH), 2.63-2.68 (m, 1H, CH), 2.83-2.89 (m, 1H, CH), 3.05-3.14 (m, 2H, 2×CH), 3.18-3.27 (m, 2H, 2×CH), 3.96 (s, 3H, COOCH₃), 4.68 (t, 1H, J=5.3 Hz, NHBoc), 7.64 (dd, 1H, J=8.4, 1.5 Hz, Ar—H-6), 7.75 (d, 1H, J=8.4 Hz Ar—H-5), 7.81 (d, 1H, J=1.5 Hz Ar—H-2) ppm. MS (ESI) m/z=394.0 ([M+H]⁺).

Step 2: Synthesis of methyl 4-amino-3-(3-(((tert-butoxycarbonyl)amino)methyl)piperidin-1-yl)benzoate (21b). To the solution of compound 20b (2.10 g, 5.34 mmol) in methanol (150 mL) under an argon atmosphere Pd—C (210 mg) was added, the mixture was saturated with hydrogene and stirred under a hydrogene atmosphere at rt for 4 h. The catalyst was filtered off and the solvent was removed under reduced pressure. The crude product was purified with flash column chromatography using ethyl acetate/petroleum ether (1/2) as an eluent to give 21b (1.60 g) as pink solid. Yield 82% (1.60 g); pink solid; mp 119-121° C. ¹H NMR (400 MHz, CDCl₃): δ 1.04-1.23 (m, 1H, CH), 1.43 (s, 9H, tBu), 1.59-1.72 (m, 1H, CH), 1.73-1.95 (m, 3H, CH), 2.33-2.50 (m, 1H, CH), 2.51-2.,66 (m, 1H, CH), 2.95-3.19 (m, 4H, CH), 3.85 (s, 3H, COOCH₃), 4.41 (s, 2H, NH₂), 4.66 (t, 1H, J=5.9 Hz, NHBoc), 6.68 (d, 1H, J=8.4 Hz Ar—H-5), 7.64 (dd, 1H, J=8.4 Hz, J=1.9 Hz, Ar—H-6), 7.67 (d, 1H, J=1.9 Hz Ar—H-2) ppm. MS (ESI) m/z=364.22 ([M+H]⁺).

Step 3: Synthesis of methyl 3-(3-(((tert-butoxycarbonyl)amino)methyl)piperidin-1-yl)-4-(3,4-dichloro-5-methyl-1H-pyrrole-2-carboxamido)benzoate (22b). Synthesised according to General procedure E from 3,4-dichloro-5-methyl-1H-pyrrole-2-carboxylic acid (695 mg, 3.58 mmol) and 21b (1.24 g, 3.41 mmol). The solid was purified with flash column chromatography using dichloromethane/methanol (50/1) as an eluent to give 22b (0.450 g) as white solid. Yield 24% (0.450 g); white solid; mp 192-194° C. ¹H NMR (400 MHz, DMSO-d₆): δ 1.05-1.13 (m, 1H, CH), 1.36 (s, 9H, tBu), 1.62-1.75 (m, 1H, CH), 1.77-1.86 (m, 2H, 2×CH), 1.87-1.99 (m, 1H, CH), 2.28 (s, 3H, CH₃), 2.39-2.45 (m, 1H, CH), 2.64-2.69 (m, 1H, CH), 2.76-2.85 (m, 1H, CH), 2.87-3.02 (m, 3H, 3×CH), 3.89 (s, 3H, COOCH₃), 6.93 (t, 1H, J=5.7 Hz, NHBoc), 7.85 (dd, 1H, J=8.4, 1.9 Hz, Ar—H-6), 7.91 (d, 1H, J=1.9 Hz Ar—H-2), 8.56 (d, 1H, J=8.4 Hz, Ar—H-5), 9.91 (s, 1H, NH), 12.47 (s, 1H, NH) ppm. MS (ESI) m/z=539.18 ([M+H]⁺). HRMS for C₂₅H₃₃Cl₂N4O₅: calculated 539.1815. found 539.1823. HPLC: Agilent Zorbax 80 Å Extend-C18 (3.5 μm, 4.6×150 mm); mobile phase: 5% acetonitrile in 0.1% TFA to 8 min, 5-95% of acetonitrile from 8 to 15 min, 95% acetonitrile from 15 to 16 min, 95-5% of acetonitrile from 16 to 18 min, 5% of acetonitrile from 18 to 21 min; flow rate 1.0 mL/min; injection volume: 10 μL; t_{R}: 15.577 min (99.6% at 254 nm).

Step 4: Synthesis of 3-(3-(((tert-butoxycarbonyl)amino)methyl)piperidin-1-yl)-4-(3,4-dichloro-5-methyl-1H-pyrrole-2-carboxamido)benzoic acid (23b). To a solution of compound 22b (180 mg, 0.33 mmol) in a mixture of methanol (10 mL) and tetrahydrofuran (10 mL) 1 M NaOH (1.32 mL, 1.32 mmol) was added and the mixture was stirred at rt for 15 h. The solvent was removed under reduced pressure, EtOAc (10 mL) and 1 M HCl (10 mL) were added to the residue. The phases were separated, the organic phase was washed with brine (2×10 mL), dried over Na₂SO₄, filtered and the solvent removed under reduced pressure to afford 23b (160 mg) as white solid. Yield 92% (160 mg); white solid; mp 221-224° C. ¹H NMR (400 MHz, DMSO-d₆): δ 0.99-1.09 (m, 1H, CH), 1.31 (s, 9H, tBu), 1.59-1.71 (m, 1H, CH), 1.72-1.82 (m, 2H, 2×CH), 1.83-1.93 (m, 1H, CH), 2.24 (s, 3H, CH₃), 2.35-2.40 (m, 1H, CH), 2.59-2.69 (m, 1H, CH), 2.69-2.80 (m, 1H, CH), 2.81-2.98 (m, 3H, 3×CH), 6.90 (t, 1H, J=5.8 Hz, NHBoc), 7.78 (dd, 1H, J=8.4, 1.8 Hz, Ar—H-6), 7.86 (d, 1H, J=1.8 Hz Ar—H-2), 8.49 (d, 1H, J=8.4 Hz, Ar—H-5), 9.86 (s, 1H, NHAr), 12.44 (s, 1H, NH), 12.86 (br s, 1H, COOH) ppm. MS (ESI) m/z=525.17 ([M+H]⁺). HRMS for C₂₄H₃₁Cl₂N₄O₅: calculated 525.1666. found 525.1662. HPLC: Agilent Zorbax 80 Å Extend-C18 (3.5 μm, 4.6×150 mm); mobile phase: 5% acetonitrile in 0.1% TFA to 8 min, 5-95% of acetonitrile from 8 to 15 min, 95% acetonitrile from 15 to 16 min, 95-5% of acetonitrile from 16 to 18 min, 5% of acetonitrile from 18 to 21 min; flow rate 1.0 mL/min; injection volume: 10 μL; t_{R}: 13.587 min (99.4% at 254 nm).

Step 5: Synthesis of (1-(5-carboxy-2-(3,4-dichloro-5-methyl-1H-pyrrole-2-carboxamido)phenyl)piperidin-3-yl)methanaminium chloride (24b). Compound 22c (59 mg, 0.11 mmol) was dissolved in HCl 4 M solution in dioxane (8 mL). The mixture was stirred for 12 h. The solvent was removed under reduced pressure and the solid was washed with diethyl ether (2×5 mL) to obtain 24b (49 mg) as white solid. Yield 96% (49 mg); white solid; mp 234-238° C. ¹H NMR (400 MHz, DMSO-d₆): δ 1.09-1.19 (m, 1H, CH), 1.63-1.73 (m, 1H, CH), 1.75-1.83 (m, 1H, CH), 1.85-1.95 (m, 1H, CH), 2.02-2.13 (m, 1H, CH), 2.25 (s, 3H, CH₃), 2.54-2.63 (m, 3H, J=9.0 Hz, 3×CH), 2.71-2.80 (m, 2H, 2×CH), 2.83-2.92 (m, 1H, CH), 3.03-3.10 (m, 1H, CH), 7.81 (dd, 1H, J=8.4, 1.8 Hz, Ar—H-4), 7.88 (d, 1H, J=1.8 Hz Ar—H-6), 7.97 (br s, 3H, NH₃+) 8.50 (d, 1H, J=8.4 Hz, Ar—H-3), 9.87 (s, 1H, NH), 12.48 (s, 1H, NH) ppm. Signal for COOH not seen. MS (ESI) m/z=425.11 ([M+H]⁺). HRMS for C₁₉H₂₃Cl₂N₄O₃: calculated 425.1142. found 425.1137. HPLC: Agilent Zorbax 80 Å Extend-C18 (3.5 μm, 4.6×150 mm); mobile phase: 5% acetonitrile in 0.1% TFA to 8 min, 5-95% of acetonitrile from 8 to 15 min, 95% acetonitrile from 15 to 16 min, 95-5% of acetonitrile from 16 to 18 min, 5% of acetonitrile from 18 to 21 min; flow rate 1.0 mL/min; injection volume: 10 μL; t_{R}: 8.557 min (99.2% at 254 nm).

Step 6: Synthesis of (1-(5-carboxy-2-(3,4-dichloro-5-methyl-1H-pyrrole-2-carboxamido)phenyl)piperidin-3-yl)methanaminium chloride (25b). Compound 23b (100 mg, 0.851 mmol) was dissolved in HCl 4 M solution in dioxane (10 mL) and the mixture was stirred for 4 h. After the completion of the reaction the solvent was removed under reduced pressure and the obtained solid was washed with diethyl ether (2×5 mL) to obtain 25b (70 mg) as beige solid. Yield 79% (70 mg); beige solid; mp 194-198° C. ¹H NMR (400 MHz, DMSO-d₆): δ 1.16-1.25 (m, 1H, CH), 1.68-1.79 (m, 1H, CH), 1.80-1.89 (m, 1H, CH), 1.90-2.00 (m, 1H, CH), 2.07-2.19 (m, 1H, CH), 2.30 (s, 3H, CH₃), 2.58-2.68 (m, 2H, 2×CH), 2.76-2.87 (m, 2H, 2×CH), 2.89-2.98 (m, 1H, CH), 3.08-3.14 (m, 1H, CH), 3.91 (s, 3H, COOCH₃), 7.89 (dd, 1H, J=8.4, 1.8 Hz, Ar—H-4), 7.94 (d, 1H, J=1.8 Hz Ar—H-6), 8.02 (br s, 3H, NH₃+), 8.58 (d, 1H, J=8.4 Hz, Ar—H-3), 9.93 (s, 1H, NH), 12.53 (s, 1H, NH) ppm. ¹³C NMR (100 MHz, DMSO-d₆): δ 10.76, 25.01, 27.09, 34.93, 41.84, 52.05, 53.46, 55.54, 108.66, 110.00, 118.50, 118.72, 122.65, 124.33, 127.14, 130.07, 138.36, 142.07, 156.62, 165.67 ppm. MS (ESI) m/z=439.13 ([M+H]⁺). HRMS for C₂₀H₂₅Cl₂N₄O₃: calculated 439.1293. found 439.1298. HPLC: Agilent Zorbax 80 Å Extend-C18 (3.5 μm, 4.6×150 mm); mobile phase: 5% acetonitrile in 0.1% TFA to 8 min, 5-95% of acetonitrile from 8 to 15 min, 95% acetonitrile from 15 to 16 min, 95-5% of acetonitrile from 16 to 18 min, 5% of acetonitrile from 18 to 21 min; flow rate 1.0 mL/min; injection volume: 10 μL; t_{R}: 9.373 min (95.6% at 254 nm).

### (Example 14) 4-(5-Carboxy-2-(3,4-dichloro-5-methyl-1H-pyrrole-2-carboxamido)phenyl)piperazin-1-ium chloride (25c)

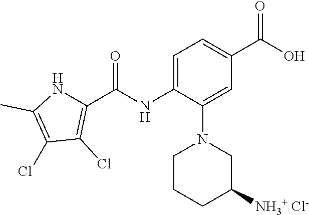

Step 1: Synthesis of methyl (S)-3-(3-((tert-butoxycarbonyl)amino)piperidin-1-yl)-4-nitrobenzoate (20c). Synthesised according to General procedure A from 19 (1.07 g, 5.39 mmol), (3S)-3-aminopiperidine 3-Boc protected (1.00 g, 5.35 mmol) and K₂CO₃ (0.894 g, 6.47 mmol). Yield 87% (1.78 g); orange solid; mp 120-124° C. ¹H NMR (400 MHz, CDCl₃): δ 1.48 (s, 9H, tBu), 1.64-1.80 (m, 3H, CH, CH₂), 1.84-1.93 (m, 1H, CH), 2.92-3.12 (m, 3H, CH, CH₂), 3.26-3.30 (m, 1H, CH), 3.88-3.97 (m. 1H, CH), 3.97 (s, 3H, CH₃), 5.05-5.15 (m, 1H, NH), 7.71 (dd, 1H, J=8.4, 1.5 Hz, ArH-6), 7.77 (d, 1H, J=8.4 Hz, Ar—H-5), 7.83 (d, 1H, J=1.5 Hz, Ar—H-2) ppm.

Step 2: Synthesis of methyl (S)-4-amino-3-(3-((tert-butoxycarbonyl)amino)piperidin-1-yl)benzoate (21c). To the solution of compound 20c (1.78 g, 4.69 mmol) in a mixture of methanol (65 mL) and tetrahydrofuran (25 mL) under an argon atmosphere Pd—C (500 mg) was added, the mixture was saturated with hydrogene and stirred under a hydrogene atmosphere at rt for 4 h. The catalyst was filtered off and the solvent was removed under reduced pressure to afford 21c (1.56 g) as white solid. Yield 88% (1.56 g); white solid; mp 112-114° C. ¹H NMR (400 MHz, CDCl₃): δ 1.27-1.37 (m, 1H, CH), 1.39 (s, 9H, tBu), 1.57-1.75 (m, 1H, CH), 1.71-1.87 (m, 1H, CH₂), 2.36-2.50 (m, 1H, CH₂), 2.70-2.81 (m, 1H, CH), 2.96-3.00 (m, 1H, CH), 3.60-3.69 (m, 1H, CH), 3.74 (s, 3H, CH₃), 5.71 (br s, 2H, NH₂), 6.68 (d, 1H, J=8.3 Hz, Ar—H-5), 6.93-7.05 (m, 1H, NH), 7.42 (d, J=1.9 Hz, Ar—H-2), 7.47 (dd, J=8.3, 1.9 Hz, Ar—H-6) ppm. MS (ESI) m/z=350.2 ([M+H]⁺). HRMS for C₁₈H₂₈N3O₄: calculated 350.2080. found 350.2072.

Step 3: Synthesis of methyl (S)-3-(3-((tert-butoxycarbonyl)amino)piperidin-1-yl)-4-(3,4-dichloro-5-methyl-1H-pyrrole-2-carboxamido)benzoate (22c). Synthesised according to General procedure E from 3,4-dichloro-5-methyl-1H-pyrrole-2-carboxylic acid (0.590 g, 3.07 mmol) and 21c (1.00 g, 2.98 mmol). During the extraction part of the product precipitated and was filtered off. The crude product was sequentially triturated with diethyl ether and methanol, and the undissolved solid was filtered off and dried, to obtain 22c (0.965 g) as white solid. Yield 64% (0.965 g); white solid; mp 188-191° C. ¹H NMR (400 MHz, DMSO-d₆): δ 1.22-1.30 (m, 1H, CH), 1.35 (s, 9H, tBu), 1.60-1.73 (m, 1H, CH), 1.76-1.94 (m, 1H, CH₂), 2.25 (s, 3H, CH₃), 2.53-2.62 (m, 2H, CH₂), 2.78-2.88 (m, 1H, CH), 2.91-3.01 (m, 1H, CH), 3.54-3.66 (m, 1H, CH), 3.85 (s, 3H, CH₃), 6.88 (d, 1H, J=7.2 Hz, NH), 7.81-7.83 (m, 2H, 2×ArH), 8.51 (d, 1H, J=8.9 Hz, ArH), 9.83 (s, 1H, NH), 12.45 (s, 1H, NH) ppm. [α]²⁰_{D}=+0.30° (c 0.375, THF). MS (ESI) m/z=525.2 ([M+H]⁺). HRMS for C₂₄H₃₁N₄O₅Cl₂: calculated 525.1672. found 525.1681. HPLC (30-90% ACN in 0.1% TFA in 16 min, 90% ACN to 20 min, Agilent Eclipse Plus C18: 5 μm, 4,6×150 mm): tᵣ 16.404 min (98.7% at 280 nm).

Step 4: Synthesis of (S)-3-(3-((tert-butoxycarbonyl)amino)piperidin-1-yl)-4-(3,4-dichloro-5-methyl-1H-pyrrole-2-carboxamido)benzoic acid (23c). To a solution of compound 22c (203 mg, 0.391 mmol) in a mixture of methanol (5 mL) and tetrahydrofuran (4 mL) 1 M NaOH (3.13 mL, 3.13 mmol) was added and the mixture was stirred at rt for 15 h. The solvent was removed under reduced pressure, to the residue water (10 mL) was added and the mixture was acidified to pH 4 with 1 M HCl. The water phase was extracted with ethyl acetate (20 mL), the organic phase was washed with brine (10 mL), dried over Na₂SO₄ and the solvent was removed under reduced pressure to obtain 23c (101 mg) as pale yellow solid. Yield 50% (101 mg); pale yellow solid; mp 231-233° C. ¹H NMR (400 MHz, DMSO-d₆): δ 1.23-1.30 (m, 1H, CH), 1.36 (s, 9H, tBu), 1.60-1.74 (m, 1H, CH), 1.75-1.96 (m, 2H, CH₂), 2.25 (s, 3H, CH₃), 2.53-2.62 (m, 2H, CH₂), 2.78-2.88 (m, 1H, CH), 2.91-3.03 (m, 1H, CH), 3.55-3.67 (m, 1H, CH), 6.80-6.99 (m, 1H, NHBoc), 7.75-7.91 (m, 2H, 2×ArH), 8.49 (d, 1H, J=8.5 Hz, ArH-5), 9.83 (s, 1H, NH), 12.46 (s, 1H, NH), 12.89 (br s, 1H, COOH) ppm. [α]²⁰_{D}=+0.44° (c 0,297, THF). MS (ESI) m/z=511.2 ([M+H]⁺). HRMS for C₂₃H₂₉N₄O₅Cl₂: calculated 511.1515. found 511.1512. HPLC (30-90% ACN in 0.1% TFA in 16 min, 90% ACN to 20 min, Agilent Eclipse Plus C18: 5 μm, 4,6×150 mm): tᵣ 13.706 min (97.7% at 280 nm).

Step 5: Synthesis of (S)-1-(2-(3,4-dichloro-5-methyl-1H-pyrrole-2-carboxamido)-5-(methoxycarbonyl)phenyl)piperidin-3-aminium chloride (24c). Compound 22c (102 mg, 0.209 mmol) was dissolved in 1 M HCl solution in acetic acid (5 mL) and the mixture was stirred at rt for 15 h. The solvent was removed under reduced pressure, the solid residue was triturated with diethyl ether, and the undissolved solid was filtered off and dried to obtain 24c (79 mg) as brown solid. Yield 74% (79 mg); brown solid; mp 201-203° C. ¹H NMR (400 MHz, DMSO-d₆): δ 1.43-1.58 (m, 1H, CH), 1.63-1.79 (m, 1H, CH), 1.85-1.92 (m, 1H, CH), 2.05-2.16 (m, 1H, CH), 2.25 (s, 3H, CH₃), 2.65-2.79 (m, 2H, CH₂), 2.86-2.94 (m, 1H, CH), 3.14-3.23 (m, 1H, CH), 3.26-3.36 (m, 1H, CH), 3.86 (s, 3H, CH₃), 7.79-7.90 (m, 2H, 2×ArH), 8.16 (br s, 3H, NH₃+), 8.47 (d, J=8.5 Hz, ArH), 9.64 (s, 1H, NH), 12.55 (s, 1H, NH) ppm. [α]²⁰_{D}=−0.72° (c 0,337, THF). MS (ESI) m/z=425.1 ([M+H]⁺). HRMS for C₁₉H₂₃N₄O₃Cl₂: calculated 425.1147. found 425.1145. HPLC (30-90% ACN in 0.1% TFA in 16 min, 90% ACN to 20 min, Agilent Eclipse Plus C18: 5 μm, 4,6×150 mm): tᵣ 7.370 min (98.9% at 280 nm).

Step 6: Synthesis of (S)-1-(5-carboxy-2-(3,4-dichloro-5-methyl-1H-pyrrole-2-carboxamido)phenyl)piperidin-3-aminium chloride (25c). Compound 23c (70 mg, 0.14 mmol) was dissolved in 1 M HCl solution in acetic acid (5 mL) and the mixture was stirred at rt for 15 h. The solvent was removed under reduced pressure, the solid residue was triturated with diethyl ether, and the undissolved solid was filtered off and dried to obtain 25c (64 mg) as pale yellow solid. Yield 91% (64 mg); pale yellow solid; mp 240-244° C. ¹H NMR (400 MHz, DMSO-d₆): δ 1.44-1.58 (m, 1H, CH), 1.63-1.76 (m, 1H, CH), 1.84-1.92 (m, 1H, CH), 2.04-2.14 (m, 1H, CH), 2.25 (s, 3H, CH₃), 2.64-2.78 (m, 2H, CH₂), 2.85-2.93 (m, 1H, CH), 3.14-3.21 (m, 1H, CH), 3.28-3.33 (m, 1H, CH), 7.77-7.85 (m, 2H, 2×ArH), 8.45 (d, J=9.1 Hz, ArH), 9.63 (s, 1H, NH), 12.54 (br s, 1H, NH) ppm. Signals for COOH and NH₃⁺ protons not seen. ¹³C NMR (100 MHz, DMSO-d₆): b 10.74, 23.64, 27.54, 47.55, 52.34, 54.82, 108.69, 110.27, 118.69, 119.24, 122.24, 125.84, 127.13, 129.81, 137.40, 141.18, 156.62, 166.69 ppm. [α]²⁰_{D}=−0.55° (c 0.264, THF). MS (ESI) m/z=411.1 ([M+H]⁺). HRMS for C₈₁H₂₁N₄O₃Cl₂: calculated 411.0991. found 411.1002. HPLC (30-90% ACN in 0.1% TFA in 16 min, 90% ACN to 20 min, Agilent Eclipse Plus C18: 5 μm, 4,6×150 mm): tᵣ 5.947 min (99.7% at 280 nm).

### (Example 15) 4-((5-Carboxy-2-(3,4-dichloro-5-methyl-1H-pyrrole-2-carboxamido)phenyl)amino)piperidin-1-ium chloride (25d)

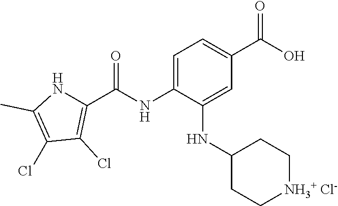

Step 1: Synthesis of ethyl 3-(3-(((tert-butoxycarbonyl)amino)methyl)piperidin-1-yl)-4-nitrobenzoate (20c). Synthesised according to General procedure A from 19 (400 mg, 2.01 mmol), tert-butyl 4-aminopiperidine-1-carboxylate (483 mg, 2.41 mmol) and K₂CO₃ (555 mg, 4.02 mmol). The crude product was purified with flash column chromatography using ethyl acetate/hexane (1:6) as eluent, to obtain 20d (212 mg) as red solid. Yield 28% (212 mg); red solid; mp 94-97° C. ¹H NMR (400 MHz, DMSO-d₆): δ 1.42 (s, 9H, tBu), 1.43-1.55 (m, 2H, CH₂), 1.90-1.99 (m, 2H, CH₂), 3.03 (s, 2H, CH₂), 3.83-3.99 (m, 6H, CH, CH₂, CH₃), 7.17 (dd, 1H, J=1.7, 8.8 Hz, ArH), 7.60 (d, 1H, J=1.7 Hz, ArH), 7.89 (d, 1H, J=7.9 Hz, NH), 8.19 (d, 1H, J=8.8 Hz, ArH) ppm. MS (ESI) m/z=401.9 ([M+Na]+).

Step 2: Synthesis of tert-butyl 4-((2-amino-5-(methoxycarbonyl)phenyl)amino)piperidine-1-carboxylate (21d). Compound 20d (200 mg, 0.527 mmol) was dissolved in methanol (50 mL) and flushed with argon. Pd/C (10%, 20 mg) was added and the reaction mixture was then stirred at rt under hydrogen atmosphere for 1 h. The catalyst was filtered off and the solvent removed in vacuo. Yield 100% (184 mg); light brown solid; mp: 140-143° C. ¹H NMR (400 MHz, DMSO-d₆): δ 1.20-1.34 (m, 2H, CH₂), 1.41 (s, 9H, tBu), 1.85-1.97 (m, 2H, CH₂), 2.95 (s, 2H, CH₂), 3.39-3.51 (m, 1H, CH), 3.72 (s, 3H, CH₃), 3.89 (d, 2H, J=13.0 Hz, CH₂), 4.40 (d, 1H, J=7.6 Hz, NH), 5.45 (s, 2H, NH₂), 6.55 (d, 1H, J=8.1 Hz, ArH), 7.05 (d, 1H, J=1.9 Hz, ArH), 7.14 (dd, 1H, J=1.9, 8.1 Hz, ArH) ppm. MS (ESI) m/z=372.0 ([M+Na]+).

Step 3: Synthesis of tert-butyl 4-((2-(3,4-dichloro-5-methyl-1H-pyrrole-2-carboxamido)-5-(methoxycarbonyl)phenyl)amino)piperidine-1-carboxylate (22d). To a suspension of 3,4-dichloro-5-methyl-1H-pyrrole-2-carboxylic acid (113 mg, 0.584 mmol) in anhydrous dichloromethane (10 mL), oxalyl chloride (209 μL, 2.43 mmol) was added dropwise and the reaction mixture was stirred at rt under argon atmosphere overnight. The solvent was evaporated under reduced pressure, 21d (170 mg, 0.487 mmol), anhydrous pyridine (2 mL) and anhydrous dichloromethane (10 mL) were added and the reaction mixture was stirred at rt under argon atmosphere overnight. The solvent was removed in vacuo, to the residue ethyl acetate and water were added and the precipitate formed which was filtered off and dried. Yield 63% (160 mg); white solid; mp 210-212° C. ¹H NMR (400 MHz, DMSO-d₆): δ 1.23-1.37 (m, 2H, CH₂), 1.40 (s, 9H, tBu), 1.80-1.94 (m, 2H, CH₂), 2.24 (s, 3H, CH₃), 2.97 (s, 2H, CH₂), 3.38-3.48 (m, 2H, CH₂), 3.83 (s, 3H, CH₃), 3.87 (s, 1H, CH), 5.11 (d, 1H, J=6.9 Hz, NH), 7.32-7.47 (m, 2H 2×ArH), 7.84 (d, 1H, J=8.2 Hz, ArH), 8.94 (s, 1H, NH), 12.30 (s, 1H, NH) ppm. MS (ESI) m/z=522.9 ([M−H]⁻).

Step 4: Synthesis of 3-((1-(tert-butoxycarbonyl)piperidin-4-yl)amino)-4-(3,4-dichloro-5-methyl-1H-pyrrole-2-carboxamido)benzoic acid (23d). To a solution of 22d (40 mg, 0.076 mmol) in methanol, 1 M NaOH (0.761 mL, 0.761 mmol) was added and the reaction mixture was stirred at 60° C. for 24 h. The solvent was evaporated in vacuo and the residue was neutralised with 1M HCl to pH 7. The precipitate that formed was filtered off and dried. Yield 77% (30 mg); light brown solid. ¹H NMR (400 MHz, DMSO-d₆): δ 1.25-1.38 (m, 2H, CH₂), 1.40 (s, 9H, tBu), 1.88 (d, 2H, J=12.7 Hz, CH₂), 2.24 (s, 3H, CH₃), 2.97 (s, 2H, CH₂), 3.48 (d, 1H, J=9.7 Hz, CH), 3.86 (d, 2H, J=13.6 Hz, CH₂), 5.07 (d, 1H, J=6.9 Hz, NH), 7.33-7.43 (m, 2H, 2×ArH), 7.80 (d, 1H, J=8.3 Hz, ArH), 8.93 (s, 1H, NH), 12.30 (s, 1H, NH), 12.60 (br s, 1H, COOH) ppm.

Step 5: Synthesis of 4-((5-carboxy-2-(3,4-dichloro-5-methyl-1H-pyrrole-2-carboxamido)phenyl)amino)piperidin-1-ium chloride (25d). To a suspension of 23d (30 mg, 0.059 mmol) in 1,4-dioxane (1 mL), 4 M HCl in 1,4-dioxane (4 mL) was added and the reaction mixture was stirred at rt for 4 h. The precipitate that formed was filtered off, washed with diethyl ether and dried. Yield 94% (24.7 mg); pale yellow solid; mp 260-265° C. ¹H NMR (400 MHz, DMSO-d₆): δ 1.60-1.75 (m, 2H, CH₂), 2.05 (d, 2H, J=13.9 Hz, CH₂), 2.25 (s, 3H, CH₃), 3.05 (q, 2H, J=10.6, 11.3 Hz, CH₂), 3.25-3.35 (m, 3H, CH, CH₂), 3.64 (1H, overlapped with the signal for water, CH), 7.35-7.44 (m, 2H, 2×ArH), 7.83 (d, 1H, J=8.1 Hz, ArH), 8.65 (s, 1H, NH), 8.79 (s, 1H, NH), 9.14 (s, 1H, NH), 12.52 (s, 2H, NH, COOH) ppm. Signal for one NH proton not seen. HRMS (ESI⁺) m/z for C₁₈H₂₁Cl₂N₄O₃ ([M+H]⁺): calculated 411.0985. found 411.0980.

### (Example 16) 4-(3,4-Dichloro-5-methyl-1H-pyrrole-2-carboxamido)-3-(4-phenylpiperazin-1-yl)benzoic acid (23e)

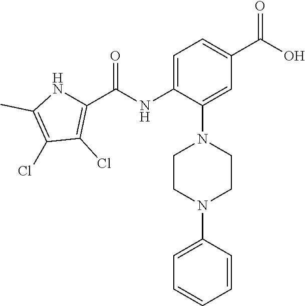 [SMILES: Cc1[nH]c(C(=O)Nc2ccc(C(=O)O)cc2N2CCN(c3ccccc3)CC2)c(Cl)c1Cl]

Step 1: Synthesis of methyl 4-nitro-3-(4-phenylpiperazin-1-yl)benzoate (20e). Synthesised according to General procedure A from 19 (500 mg, 2.51 mmol), 1-phenylpiperazine (460 μL, 2.92 mmol) and potassium carbonate (0.694 g, 5.02 mmol). Yield: 76% (650 mg); orange solid; mp 86-88° C. ¹H NMR (400 MHz, DMSO-d₆): δ 3.17-3.22 (m, 4H, 2×CH₂), 3.22-3.30 (m, 4H, 2×CH₂), 3.90 (s, 3H, CH₃), 6.78-6.86 (m, 1H, ArH), 6.94-7.00 (m, 2H, 2×ArH), 7.20-7.29 (m, 2H, 2×ArH), 7.66 (dd, J=1.7, 8.4 Hz, 1H, ArH), 7.82 (d, 1H, J=1.7 Hz, ArH), 7.96 (d, 1H, J=8.4 Hz, ArH) ppm. MS (ESI) m/z=342.0 ([M+H]⁺).

Step 2: Synthesis of methyl 4-amino-3-(4-phenylpiperazin-1-yl)benzoate (21e). Compound 20e (0.600 g, 1.93 mmol) was dissolved in methanol and tetrahydrofuran (3:1, 80 mL) and flushed with argon. Pd/C (10%, 60 mg) was added and the reaction mixture was then stirred at rt under hydrogen atmosphere for 3 h. The catalyst was filtered off and the solvent removed in vacuo. The crude product was suspended in diethyl ether, sonicated, filtered off and dried. Yield 59% (320 mg); grey solid; mp 124-129° C. ¹H NMR (400 MHz, DMSO-d₆): δ 2.95 (t, 4H, J=4.9 Hz, 2×CH₂), 3.32 (4H, overlapped with the signal for water, 2×CH₂), 3.75 (s, 3H, CH₃), 5.69 (s, 2H, NH₂), 6.73 (d, 1H, J=8.8 Hz, ArH), 6.76-6.85 (m, 1H, ArH), 6.95-7.03 (m, 2H, 2×ArH), 7.19-7.29 (m, 2H, 2×ArH), 7.47-7.52 (m, 2H, 2×ArH) ppm. MS (ESI) m/z=312.0 ([M+H]⁺).

Step 3: Synthesis of methyl 4-(3,4-dichloro-5-methyl-1H-pyrrole-2-carboxamido)-3-(4-phenylpiperazin-1-yl)benzoate (22e). To a suspension of 3,4-dichloro-5-methyl-1H-pyrrole-2-carboxylic acid (224 mg, 1.16 mmol) in anhydrous dichloromethane (15 mL), oxalyl chloride (413 μL, 4.82 mmol) was added dropwise and the reaction mixture was stirred at rt under argon atmosphere overnight. The solvent was evaporated under reduced pressure, 21e (300 mg, 0.963 mmol), anhydrous pyridine (2 mL) and anhydrous dichloromethane (15 mL) were added and the reaction mixture was stirred at rt under argon atmosphere overnight. The solvent was removed in vacuo, to the residue ethyl acetate and water were added and the precipitate formed which was filtered off, washed with methanol and dried. Yield 32% (150 mg); light grey solid; mp 218-221° C. ¹H NMR (400 MHz, DMSO-d₆): δ 2.24 (s, 3H, CH₃), 3.04 (t, 4H, J=4.9 Hz, 2×CH₂), 3.40 (4H, overlapped with the signal for water, 2×CH₂), 3.86 (s, 3H, CH₃), 6.84 (t, 1H, J=7.2 Hz, ArH), 6.99-7.05 (m, 2H, 2×ArH), 7.23-7.30 (m, 2H, 2×ArH), 7.85 (dd, 1H, J=2.0, 8.6 Hz, ArH), 7.92 (d, 1H, J=2.0 Hz, ArH), 8.54 (d, 1H, J=8.6 Hz, ArH), 9.88 (s, 1H, NH), 12.48 (s, 1H, NH) ppm.

Step 4: Synthesis of 4-(3,4-dichloro-5-methyl-1H-pyrrole-2-carboxamido)-3-(4-phenylpiperazin-1-yl)benzoic acid (23e). To a solution of 22e (30 mg, 0.062 mmol) in methanol, 1 M NaOH (0.616 mL, 0.616 mmol) was added and the reaction mixture was stirred at 50° C. overnight. The solvent was evaporated in vacuo and the residue was neutralised with 1 M HCl to pH 7. The precipitate that formed was filtered off. Yield 99% (28.8 mg); light brown solid; mp>300° C. ¹H NMR (400 MHz, DMSO-d₆): δ 2.24 (s, 3H, CH₃), 3.04 (s, 4H, 2×CH₂), 3.33 (4H, overlapped with the signal for water, 2×CH₂) 6.84 (s, 1H, ArH), 7.03 (s, 2H, 2×ArH), 7.27 (s, 2H, 2×ArH), 7.84 (s, 1H, ArH), 7.91 (s, 1H, ArH), 8.51 (d, 1H, J=8.8 Hz, ArH), 9.87 (s, 1H, NH), 12.47 (s, 1H, NH), 12.90 (s, 1H, COOH) ppm. HRMS (ESI⁺) m/z for C₂₃H₂₃Cl₂N₄O₃ ([M+H]⁺): calculated 473.1142. found 473.1138.

### (Example 17) Methyl (S)-3-(3-((tert-butoxycarbonyl)amino)pyrrolidin-1-yl)-4-(3,4-dichloro-5-methyl-1H-pyrrole-2-carboxamido)benzoate (22f)

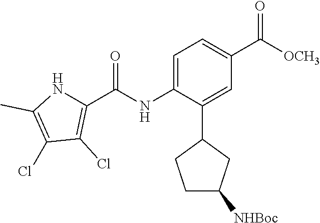 [SMILES: COC(=O)c1ccc(NC(=O)c2[nH]c(C)c(Cl)c2Cl)c(C2CC[C@H](NC(=O)OC(C)(C)C)C2)c1]

Step 1: Synthesis of methyl (S)-3-(3-((tert-butoxycarbonyl)amino)pyrrolidin-1-yl)-4-nitrobenzoate (20f). Synthesised according to General procedure A from 19 (1.00 g, 5.03 mmol), (3S)-Boc-3-aminopyrrolidine (0.938 g, 5.03 mmol) and K₂CO₃ (0.835 g, 6.04 mmol). Yield 84% (1.62 g); pale orange solid; mp 120-124° C. ¹H NMR (400 MHz, DMSO-d₆): δ 1.38 (s, 9H), 1.88-1.95 (m, 1H, CH), 2.07-2.16 (m, 1H, CH), 2.87-2.92 (m, 1H, CH), 3.27-3.33 (m, 2H, 2×CH), 3.39-3.45 (m, 1H, CH), 3.89 (s, 3H, CH₃), 4.07-4.14 (m, 1H, CH), 7.24 (d, 1H, J=6.4 Hz, NH), 7.28 (dd, 1H, J=8.4, 1.7 Hz, ArH), 7.51 (d, 1H, J=1.7 Hz, ArH), 7.84 (d, 1H, J=8.4 Hz, ArH) ppm. [α]_{D}²⁵ 0.54 (c 0,287 DMF). MS (ESI) m/z=388.2 ([M+Na]+).

Step 2: Synthesis of methyl (S)-4-amino-3-(3-((tert-butoxycarbonyl)amino)pyrrolidin-1-yl)benzoate (21f). To the solution of compound 20f (1.62 g, 4.43 mmol) in a mixture of methanol (65 mL) and tetrahydrofuran (15 mL) under an argon atmosphere Pd—C (500 mg) was added, the mixture was saturated with hydrogene and stirred under a hydrogene atmosphere at rt for 4 h. The catalyst was filtered off and the solvent was removed under reduced pressure. The crude product was purified with flash column chromatography (ethyl acetate/petroleum ether=1:3 to 1:1) to afford 21f (1.78 g) as pink solid. Yield 87% (1.78 g); pink solid; mp 105-107° C. ¹H NMR (400 MHz, CDCl₃): δ 1.39 (s, 9H, tBu), 1.63-1.70 (m, 1H, CH), 2.15-2.27 (m, 1H, CH), 2.78-2.82 (m, 2H, 2×CH), 3.04-3.13 (m, 2H, 2×CH), 3.73 (s, 3H, CH₃), 4.07-4.15 (m, 1H, CH), 5.69 (s, 2H, NH₂), 6.64 (d, 1H, J=8.2 Hz, ArH), 7.22 (d, 1H, J=7.9 Hz, NHBoc), 7.37-7.49 (m, 2H, 2×ArH) ppm. [α]_{D}²⁵ 0.073 (c 0,418, DMF). MS (ESI) m/z=336.3 ([M+H]⁺).

Step 3: Synthesis of methyl (S)-3-(3-((tert-butoxycarbonyl)amino)pyrrolidin-1-yl)-4-(3,4-dichloro-5-methyl-1H-pyrrole-2-carboxamido)benzoate (22f). Synthesised according to General procedure E from 3,4-dichloro-5-methyl-1H-pyrrole-2-carboxylic acid (0.446 g, 2.30 mmol) and 21f (0.700 g, 2.09 mmol). The crude product obtained after the extraction was triturated with methanol, and the undissolved solid was filtered off and dried, to obtain 22f (391 mg) as brown solid. Yield 60% (0.965 g); white solid; mp 113-116° C. ¹H NMR (400 MHz, DMSO-d₆): δ 1.40 (s, 9H, tBu), 1.78-1.89 (m, 1H, CH), 2.14-2.23 (m, 1H, CH), 2.24 (s, 3H, CH₃), 2.88-2.92 (m, 1H, CH), 3.01-3.07 (m, 1H, CH), 3.12-3.19 (m, 1H, CH), 3.29-3.34 (m, 1H, CH), 3.85 (s, 3H, CH₃), 4.07-4.15 (m, 1H, CH), 7.19 (d, 1H, J=6.7 Hz, NHBoc), 7.74 (dd, 1H, J=8.5, 2.0 Hz, ArH), 7.81 (d, 1H, J=2.0 Hz, ArH), 8.36 (d, 1H, J=8.5 Hz, ArH), 9.60 (s, 1H, NH), 12.41 (s, 1H, NH) ppm. [α]_{D}²⁵0.953 (c 0,270, DMF). MS (ESI) m/z=511.2 ([M+H]⁺).

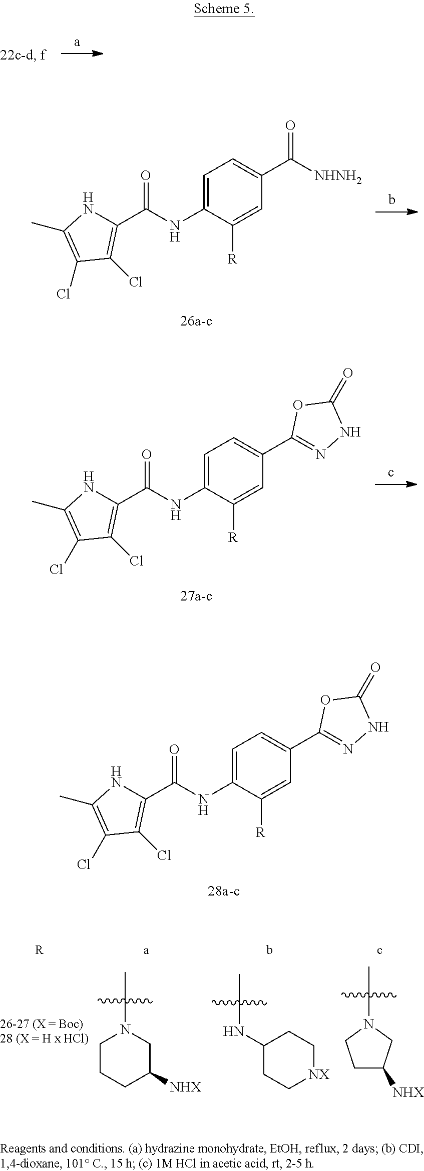

### (Example 18) (S)-1-(2-(3,4-Dichloro-5-methyl-1H-pyrrole-2-carboxamido)-5-(5-oxo-4,5-dihydro-1,3,4-oxadiazol-2-yl)phenyl)piperidin-3-aminium chloride (28a)

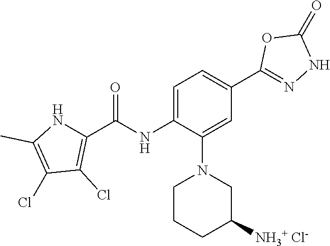

Step 1: Synthesis of tert-butyl (S)-(1-(2-(3,4-dichloro-5-methyl-1H-pyrrole-2-carboxamido)-5-(hydrazinecarbonyl)phenyl)piperidin-3-yl)carbamate (26a). To a solution of 22c (295 mg, 0.561 mmol) in a mixture of MeOH (6 mL) and THE (5 mL) in a high-pressure tube hydrazine hydrate (64%, 2.72 mL, 56.1 mmol) was added. The tube was sealed and reaction mixture was stirred at 120° C. overnight. The tube was cooled down to rt, the solvent was removed under reduced pressure and the solid residue was triturated with methanol, the undissolved solid was filtered off and dried, to obtain 26a (295 mg) as yellow solid. Yield 66% (295 mg); yellow solid; mp 224-228° C. ¹H NMR (400 MHz, DMSO-d₆): δ 1.20-1.29 (m, 1H, CH), 1.36 (s, 9H, tBu), 1.59-1.73 (m, 1H, CH), 1.74-1.94 (m, 2H, CH₂), 2.24 (s, 3H, CH₃), 2.55-2.64 (m, 1H, CH), 2.76-2.84 (m, 1H, CH), 2.90-2.99 (m, 1H, CH), 3.57-3.68 (m, 1H, CH), 4.47 (s, 2H, NH₂), 6.91 (d, J=7.5 Hz, 1H, NHBoc), 7.68 (dd, J=8.6, 2.0 Hz, 1H, ArH), 7.79 (d, J=2.0 Hz, 1H, ArH), 8.42 (d, J=8.6 Hz, 1H, ArH), 9.75 (s, 2H, 2×NH) ppm. Signals for one CH and one NH proton not seen. [α]²⁰_{D}=−0.70° (c 0,321, THF). MS (ESI) m/z=523.2 ([M−H]⁻). HRMS for C₂₃H₂₉N₆O₄Cl₂: calculated 523.1627. found 523.1627. HPLC (30-90% ACN in 0.1% TFA in 16 min, 90% ACN to 20 min, Agilent Eclipse Plus C18: 5 μm, 4,6×150 mm): tᵣ 7.883 min (95.8% at 254 nm).

Step 2: Synthesis of tert-butyl (S)-(1-(2-(3,4-dichloro-5-methyl-1H-pyrrole-2-carboxamido)-5-(5-oxo-4,5-dihydro-1,3,4-oxadiazol-2-yl)phenyl)piperidin-3-yl)carbamate (27a). To a solution of compound 26a (240 mg, 0.449 mmol) in a mixture of DMF (5 mL) and 1,4-dioxane (10 mL) 1,1′-carbonyldiimidazole (219 mg, 1.35 mmol) was added and the reaction mixture was stirred at 101° C. overnight. The solvent was removed under reduce pressure, the crude product was triturated with a mixture of acetonitrile and methanol (20 mL, 1:1), the undissolved solid was filtered off and dried to obtain 27a (202 mg) as brown solid. Yield 84% (202 mg); brown solid; mp 260-263° C. ¹H NMR (400 MHz, DMSO-d₆): δ 1.17-1.27 (m, 1H, CH), 1.36 (s, 9H, tBu), 1.58-1.74 (m, 1H, CH), 1.75-1.99 (m, 2H, CH₂), 2.24 (s, 3H, CH₃), 2.54-2.70 (m, 2H, CH₂), 2.77-2.91 (m, 1H, CH), 2.91-3.05 (m, 1H, CH), 3.54-3.70 (m, 1H, CH), 6.89 (d, J=7.5 Hz, 1H, NHBoc), 7.62-7.64 (m, 2H, 2×ArH), 8.53 (d, J=8.6 Hz, 1H, ArH), 9.76 (s, 1H, NH), 12.44 (s, 1H, NH), 12.55 (s, 1H, NH) ppm. [α]²⁰_{D}=−0.73° (c 0,318, THF). MS (ESI) m/z=549.1 ([M−H]⁻). HRMS for C₂₄H₂₇N₆O₅Cl₂: calculated 549.1420. found 549.1436. HPLC (30-90% ACN in 0.1% TFA in 16 min, 90% ACN to 20 min, Agilent Eclipse Plus C18: 5 μm, 4,6×150 mm): tᵣ 11.960 min (95.1% at 280 nm).

Step 3: Synthesis of (S)-1-(2-(3,4-dichloro-5-methyl-1H-pyrrole-2-carboxamido)-5-(5-oxo-4,5-dihydro-1,3,4-oxadiazol-2-yl)phenyl)piperidin-3-aminium chloride (28a). To a solution of compound 27a (133 mg, 0.241 mmol) in DMF (2 mL) 4 M HCl in 1,4-dioxane (6 mL) was added and the reaction mixture was stirred at rt for 15 h. The solvent was removed under reduced pressure and the solid residue was sequentially triturated with diethyl ether and acetonitrile, the undissolved solid was filtered off and dried, to obtain 28a (71 mg) as off-white solid. Yield 60% (71 mg); off-white solid; mp>230° C. ¹H NMR (400 MHz, DMSO-d₆): δ 1.44-1.62 (m, 1H, CH), 1.62-1.77 (m, 1H, CH), 1.83-1.93 (m, 1H, CH), 2.05-2.13 (m, 1H, CH), 2.25 (s, 3H, CH₃), 2.65-2.81 (m, 2H, CH₂), 2.83-2.94 (m, 1H, CH), 3.16-3.25 (m, 1H, CH), 3.26-3.40 (s, 1H, CH), 7.63-7.66 (m, 2H, 2×ArH), 8.27 (br s, 3H, NH₃+), 8.46 (d, J=8.4 Hz, 1H, ArH), 9.58 (s, 1H, NH), 12.57 (s, 1H, NH), 12.64 (s, 1H, NH) ppm. ¹³C NMR (100 MHz, DMSO-d₆): δ 10.76, 23.55, 27.52, 47.50, 52.13, 54.70, 108.70, 110.32, 118.20, 118.66, 119.19, 120.27, 122.82, 129.78, 136.01, 141.93, 153.38, 154.40, 156.58 ppm. MS (ESI) m/z=449.1 ([M−H]⁻). HRMS for C₁₉H₁₉N₆O₃Cl₂: calculated 449.0896. found 449.0890. HPLC (30-90% ACN in 0.1% TFA in 16 min, 90% ACN to 20 min, Agilent Eclipse Plus C18: 5 μm, 4,6×150 mm): tᵣ 4.633 min (95.8% at 280 nm).

### (Example 19) 4-((2-(3,4-Dichloro-5-methyl-1H-pyrrole-2-carboxamido)-5-(5-oxo-4,5-dihydro-1,3,4-oxadiazol-2-yl)phenyl)amino)piperidin-1-ium chloride (28b)

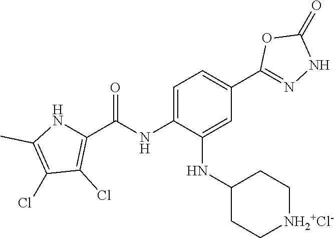

Step 1: Synthesis of tert-butyl 4-((2-(3,4-dichloro-5-methyl-1H-pyrrole-2-carboxamido)-5-(hydrazinecarbonyl)phenyl)amino)piperidine-1-carboxylate (26b). To a suspension of 22d (110 mg, 0.209 mmol) in a mixture of methanol and THE (2:1, 15 mL), hydrazine monohydrate (419 μL, 8.37 mmol) was added and the reaction mixture was stirred in a pressure tube at 115° C. for 48 h. The precipitate that was formed was filtered off and dried. Yield 64% (70 mg); white solid; mp 247-250° C. ¹H NMR (400 MHz, DMSO-d₆): b 1.32 (t, 2H, J=11.7 Hz, CH₂), 1.40 (s, 9H, tBu), 1.84-1.96 (m, 2H, CH₂), 2.23 (s, 3H, CH₃), 2.96 (s, 2H, CH₂), 2.42 (s, 1H, CH), 3.89 (d, 2H, J=13.4 Hz, CH₂), 4.45 (s, 2H, NH₂), 5.02 (d, 1H, J=7.2 Hz, NH), 7.20 (dd, 1H, J=1.9, 8.3 Hz, ArH), 7.27 (d, 1H, J=1.9 Hz, ArH), 7.63 (d, 1H, J=8.3 Hz, ArH), 8.80 (s, 1H, NH), 9.69 (s, 1H, NH), 12.25 (s, 1H, NH) ppm. HRMS (ESI⁺) m/z for C₁₈H₂₃Cl₂N₆O₂ ([M+H]⁺): calculated 425.1254. found 425.1259.

Step 2: Synthesis of tert-butyl 4-((2-(3,4-dichloro-5-methyl-1H-pyrrole-2-carboxamido)-5-(5-oxo-4,5-dihydro-1,3,4-oxadiazol-2-yl)phenyl)amino)piperidine-1-carboxylate (27b). To a suspension of 26b (60 mg, 0.114 mmol) in a mixture of 1,4-dioxane and DMF (2:1, 15 mL), 1,1′-carbonyldiimidazole (55.6 mg, 0.343 mmol) was added and the reaction mixture was stirred 101° C. for 24 h. The solvent was evaporated in vacuo, the residue was suspended in acetonitrile, sonicated, filtered off and dried. Yield 56% (35 mg); yellow solid; mp 168-171° C. ¹H NMR (400 MHz, DMSO-d₆) δ 1.25-1.37 (m, 2H, CH₂), 1.40 (s, 9H, tBu), 1.89 (d, 2H, J=9.7 Hz, CH₂), 2.23 (s, 3H, CH₃), 2.93 (m, 2H, CH₂), 3.53 (s, 1H, CH), 3.79-3.92 (m, 2H, CH₂), 5.19 (d, 1H, J=7.1 Hz, NH), 7.14-7.19 (m, 2H, 2×ArH), 7.74-7.78 (m, 1H, ArH), 8.84 (s, 1H, NH), 12.26 (s, 1H, NH), 12.49 (s, 1H, NH) ppm.

Step 3: Synthesis of 4-((2-(3,4-dichloro-5-methyl-1H-pyrrole-2-carboxamido)-5-(5-oxo-4,5-dihydro-1,3,4-oxadiazol-2-yl)phenyl)amino)piperidin-1-ium chloride (28b). To a suspension of 27b (25 mg, 0.045 mmol) in 1,4-dioxane (7 mL), 4 M HCl in 1,4-dioxane (3 mL) was added and the reaction mixture was stirred at rt for 3 h. The precipitate that formed was filtered off, washed with diethyl ether and dried. Yield 100% (22 mg); pale yellow solid; mp 212-217° C. ¹H NMR (400 MHz, DMSO-d₆): δ 1.65-1.72 (m, 2H, CH₂), 2.06 (dd, 2H, J=13.2, 2.8 Hz, CH₂), 2.24 (s, 3H, CH₃), 3.01-3.11 (m, 2H, CH₂), 3.25-3.35 (m, 2H, CH₂), 3.67-3.72 (m, 1H, CH), 7.17 (m, 2H, 2×ArH), 7.79 (d, 1H, J=8.2 Hz, ArH), 8.72 (s, 2H, NH₂+), 9.10 (s, 1H, NH), 12.53 (s, 1H, NH), 12.55 (s, 1H, NH) ppm. Signal for one NH proton not seen. HRMS (ESI⁺) m/z for C₁₉H₂₁Cl₂N₆O₃ ([M+H]⁺): calculated 451.1047. found 451.1040.

### (Example 20) (S)-1-(2-(3,4-Dichloro-5-methyl-1H-pyrrole-2-carboxamido)-5-(5-oxo-4,5-dihydro-1,3,4-oxadiazol-2-yl)phenyl)pyrrolidin-3-aminium chloride (28c)

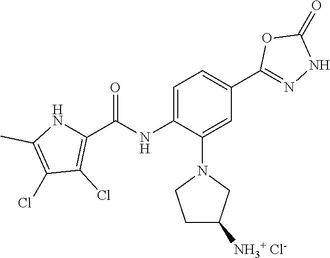

Step 1: Synthesis of tert-butyl (S)-(1-(2-(3,4-dichloro-5-methyl-1H-pyrrole-2-carboxamido)-5-(hydrazinecarbonyl)phenyl)pyrrolidin-3-yl)carbamate (26c). To a solution of 22f (540 mg, 1.06 mmol) in a mixture of MeOH (11 mL) and THE (8 mL) in a high-pressure tube hydrazine hydrate (64%, 5.14 mL, 0.106 mol) was added. The tube was sealed and reaction mixture was stirred at 120° C. overnight. The tube was cooled down to rt, the solvent was removed under reduced pressure and the solid residue was triturated with methanol, the undissolved solid was filtered off and dried, to obtain 26c (390 mg) as white solid. Yield 72% (390 mg); white solid; mp 224-228° C. ¹H NMR (400 MHz, DMSO-d₆): δ 1.39 (s, 9H, tBu), 1.80-1.88 (m, 1H, CH), 2.14-2.21 (m, 1H, CH), 2.23 (s, 3H, CH₃), 2.80-2.92 (m, 1H, CH), 2.99-3.05 (m, 1H, CH), 3.12-3.19 (m, 1H, CH), 3.27-3.33 (m, 1H, CH), 4.05-4.14 (m, 1H, CH), 4.46 (s, 2H, NH₂), 7.17 (d, 1H, J=6.6 Hz, NHBoc), 7.56 (dd, 1H, J=8.5, 2.0 Hz, ArH), 7.71 (d, 1H, J=2.0 Hz, ArH), 8.22 (d, 1H, J=8.5 Hz, ArH), 9.47 (s, 1H, NH), 9.72 (s, 1H, NH), 12.37 (s, 1H, NH) ppm. [α]_{D}²⁵−0.314 (c 0,373, DMF). MS (ESI) m/z=511.3 ([M+H]⁺).

Step 2: Synthesis of tert-butyl (S)-(1-(2-(3,4-dichloro-5-methyl-1H-pyrrole-2-carboxamido)-5-(5-oxo-4,5-dihydro-1,3,4-oxadiazol-2-yl)phenyl)pyrrolidin-3-yl)carbamate (27c). To a solution of compound 26c (100 mg, 0.192 mmol) in a mixture of DMF (2.5 mL) and 1,4-dioxane (5 mL) 1,1′-carbonyldiimidazole (94 mg, 0.576 mmol) was added and the reaction mixture was stirred at 101° C. overnight. The solvent was removed under reduce pressure, the crude product was sequentially triturated with acetonitrile and methanol, the undissolved solid was filtered off and dried to obtain 27c (24 mg) as pale yellow solid. Yield 23% (24 mg); pale yellow solid; mp 260-263° C. ¹H NMR (400 MHz, DMSO-d₆): δ 1.40 (s, 9H, tBu), 1.80-1.90 (m, 1H, CH), 2.16-2.22 (m, 1H, CH), 2.25 (s, 3H, CH₃), 2.91-2.96 (m, 1H, CH), 3.04-3.12 (m, 1H, CH), 3.17-3.24 (m, 1H, CH), 3.24-3.33 (m, 1H, CH), 4.07-4.15 (m, 1H, CH), 7.20 (d, 1H, NHBoc), 7.53 (d, 1H, J=8.6 Hz, ArH), 7.60 (s, 1H, ArH), 8.32 (d, 1H, J=8.6 Hz, ArH), 9.50 (s, 1H, NH), 12.40 (br s, 2H, 2×NH) ppm. [α]²⁰_{D}=0.035 (c 0,255, DMF). HRMS for C₂₃H₂₇Cl₂N₆O₅: calculated 537.1414. found 537.1416.

Step 3: Synthesis of (S)-1-(2-(3,4-dichloro-5-methyl-1H-pyrrole-2-carboxamido)-5-(5-oxo-4,5-dihydro-1,3,4-oxadiazol-2-yl)phenyl)pyrrolidin-3-aminium chloride (28c). To a solution of compound 27c (24 mg, 0.045 mmol) in DMF (1 mL) 4 M HCl in 1,4-dioxane (3 mL) was added and the reaction mixture was stirred at rt for 15 h. The solvent was removed under reduced pressure and the solid residue was sequentially triturated with diethyl ether and acetonitrile, the undissolved solid was filtered off and dried, to obtain 28c (13 mg) as off-white solid. Yield 60% (13 mg); off-white solid; mp>230° C. ¹H NMR (400 MHz, DMSO-d₆): δ 1.98-2.06 (m, 1H, CH), 2.25 (s, 3H, CH₃), 2.26-2.32 (m, 1H, CH), 3.02-3.14 (m, 2H, 2×CH), 3.37-345 (m, 1H, CH), 3.50-3.57 (m, 1H, CH), 3.87-3.97 (s, 1H, CH), 7.55 (dd, 1H, J=8.5, 1.9 Hz, ArH), 7.61 (d, 1H, J=1.9 Hz, ArH), 8.25 (d, 1H, J=8.5 Hz, ArH), 8.32 (br s, 3H, NH₃+), 9.50 (s, 1H, NH), 12.58 (s, 1H, NH), 12.63 (s, 1H, NH) ppm. [α]_{D}²⁵ 1.25 (c 0,271, DMF). HRMS for C₁₈H₁₉Cl₂N₆O₃: calculated 437.0890. found 437.0890. HPLC (30-90% ACN in 0.1% TFA in 16 min, 90% ACN to 20 min, Agilent Eclipse Plus C18: 5 μm, 4,6×150 mm): tᵣ 3.523 min (96.05% at 254 nm).

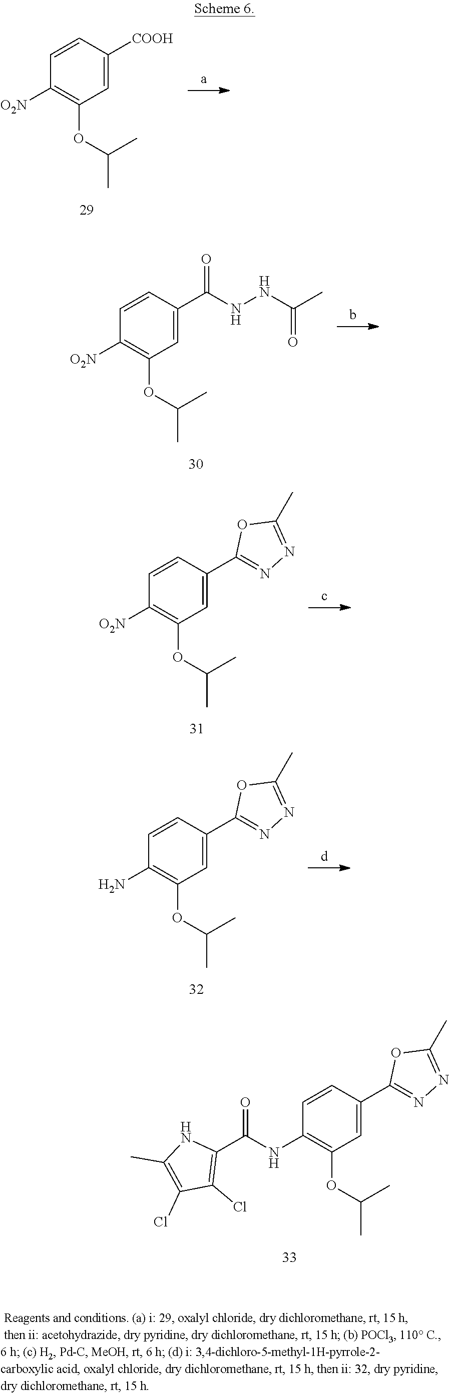

### (Example 21) 3,4-Dichloro-N-(2-isopropoxy-4-(5-methyl-1,3,4-oxadiazol-2-yl)phenyl)-5-methyl-1H-pyrrole-2-carboxamide (29)

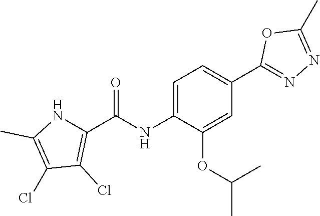

Step 1: Synthesis of N′-acetyl-3-isopropoxy-4-nitrobenzohydrazide (30). To a solution of 29 (1.20 g, 5.32 mmol) in anhydrous dichloromethane (70 mL) oxalyl chloride (1.83 mL, 21.28 mmol) was added dropwise and the solution was stirred at rt for 15 h under argon atmosphere. The solvent was evaporated under reduced pressure, fresh anhydrous dichloromethane (35 mL), acetohydrazide (0.357 g, 4.82 mmol) and pyridine (17 mL) were added and the reaction mixture was stirred under argon atmosphere at rt for 15 h. Solvent was removed under reduced pressure, the residue dissolved in ethyl acetate (15 mL) and washed with HCl 1 M solution (15 mL) and brine (2×15 mL). The organic phase was dried over Na₂SO₄, filtered and the solvent removed under reduced pressure. The crude product was purified with flash column chromatography using dichloromethane/methanol (10/1) as an eluent to afford 81 (0.950 g) as white solid. Yield 70% (0.950 g); white solid; mp 165-167° C. ¹H NMR (400 MHz, CDCl₃) δ 1.38 (d, 6H, ³J=6.0 Hz, CH(CH₃)₂), 2.18 (s, 3H, CH₃), 4.70 (spt, 1H, ³J=6.0 Hz, CH(CH₃)₂), 7.37 (dd, 1H, ³J=8.0 Hz, ⁴J=1.6 Hz, Ar—H-6), 7.53 (d, 1H, ⁴J=1.6 Hz, Ar—H-2), 7.71 (d, 1H, ³J=8.0 Hz, Ar—H-5), 8.82 (d, 1H, ³J=4.2 Hz, NHCOCH₃), 10.06 (d, 1H, ³J=4.2 Hz, NHCOAr) ppm. HRMS for Cl₂H₁₄N₃O₅: calculated 280.0933. found 280.0937.

Step 2: Synthesis of 2-(3-Isopropoxy-4-nitrophenyl)-5-methyl-1,3,4-oxadiazole (31). A solution of 30 (0.940 g, 3.34 mmol) in phosphorous oxychloride (32.9 mL, 354 mmol) was stirred at 110° C. for 6 h under argon atmosphere. The solvent was evaporated under reduced pressure, NaOH 1 M solution (15 mL) was added dropwise to quench the reaction mixture. EtOAc (20 mL) was added to the mixture and the layers were separated. The organic phase was washed with brine (2×20 mL), dried over Na₂SO₄, filtered and the solvent removed under reduced pressure. The solid was then purified with flash column chromatography using EtOAc as an eluent to afford 31 (410 mg) as yellow solid. Yield 47% (410 mg); yellow solid; mp 104-106° C. ¹H NMR (400 MHz, DMSO-d₆) δ 1.33 (d, 6H, J=6.0 Hz, CH(CH₃)₂), 2.62 (s, 3H, CH₃), 4.98 (spt, 1H, J=6.0 Hz, CH(CH₃)₂), 7.66 (dd, 1H, J=8.0, 1.6 Hz, Ar—H-6), 7.82 (d, 1H, J=1.6 Hz, Ar—H-2), 8.03 (d, 1H, J=8.4 Hz, Ar—H-5) ppm. MS (ESI) m/z=264.10 ([M+H]⁺). HRMS for Cl₂H₁₄N₃O₄: calculated 264.0984. found 264.0980.

Step 3: Synthesis of 2-isopropoxy-4-(5-methyl-1,3,4-oxadiazol-2-yl)aniline (32). To a solution of compound 31 (362 mg, 1.137 mmol) in MeOH (10 mL) unden argon atmosphere Pd—C (50 mg) was added and the reaction mixture was stirred under hydrogen atmosphere for 6 h. The catalyst was filtered off and the solvent removed under reduced pressure. The crude product was purified with flash column chromatography using dichloromethane/methanol (20/1) as an eluent as an eluent to afford 32 (270 mg) as brown oil. Yield 85% (0.270 g), brown oil. ¹H NMR (400 MHz, CDCl₃) δ 1.39 (d, 6H, J=6.0 Hz, CH(CH₃)₂), 2.57 (s, 3H, CH₃), 4.04 (br s, 2H, NH₂), 4.67 (spt, 1H, J=6.0 Hz, CH(CH₃)₂), 6.74 (d, 1H, J=8.2 Hz, Ar—H-6), 7.41 (dd, 1H, J=8.2, 1.6 Hz, Ar—H-5), 7.46 (d, 1H, J=1.6 Hz, Ar—H-3) ppm. MS (ESI) m/z=234.10 ([M+H]⁺). HRMS for C₁₂H₁₆N₃O₄: calculated 234.1243. found 234.1244.

Step 4: Synthesis of 3,4-dichloro-N-(2-isopropoxy-4-(5-methyl-1,3,4-oxadiazol-2-yl)phenyl)-5-methyl-1H-pyrrole-2-carboxamide (33). Synthesised according to General procedure E from 3,4-dichloro-5-methyl-1H-pyrrole-2-carboxylic acid (258 mg, 1.33 mmol) and 32 (259 mg, 1.11 mmol). During extraction the product precipitated and was filtered off. The crude product was purified with flash column chromatography using dichloromethane/methanol (20/1) as an eluent as an eluent to afford 33 (280 mg) as white crystals. Yield 59% (0.280 g); white crystals; mp 230-234° C. ¹H NMR (400 MHz, DMSO-d₆) δ 1.39 (d, 6H, J=6.0 Hz, CH(CH₃)₂), 2.25 (s, 3H, CH₃), 2.59 (s, 3H, CH₃), 4.92 (spt, 1H, J=6.0 Hz, CH(CH₃)₂), 7.58-7.61 (m, 1H, Ar—H-3,5), 8.59 (d, 1H, J=8.8 Hz, Ar—H-6), 9.29 (s, 1H, NHAr), 12.48 (s, 1H, NH) ppm. ¹³C NMR (100 MHz, DMSO-d₆) δ 10.75, 21.69 (3×CH₃), 71.49 CH(CH₃)₂, 108.64, 109.84, 109.94, 118.32, 118.56, 119.00, 119.32, 129.91, 131.35, 145.72, 156.20, 163.63, 163.73 ppm. Signal for one aliphatic carbon not seen. MS (ESI) m/z=407.10 ([M−H]⁻). HRMS for C₁₈H₁₇Cl₂N₄O₃: calculated 407.0678. found 407.0676. HPLC: Phenomenex Luna C18 column (5 μm, 4.6×250 mm); mobile phase: 10-70% of acetonitrile in phosphate buffer (pH=6.8) in 15 min, 70% acetonitrile to 25 min; flow rate 1.0 mL/min; injection volume: 10 μL; t_{R}: 22.320 min (99.8% at 254 nm).

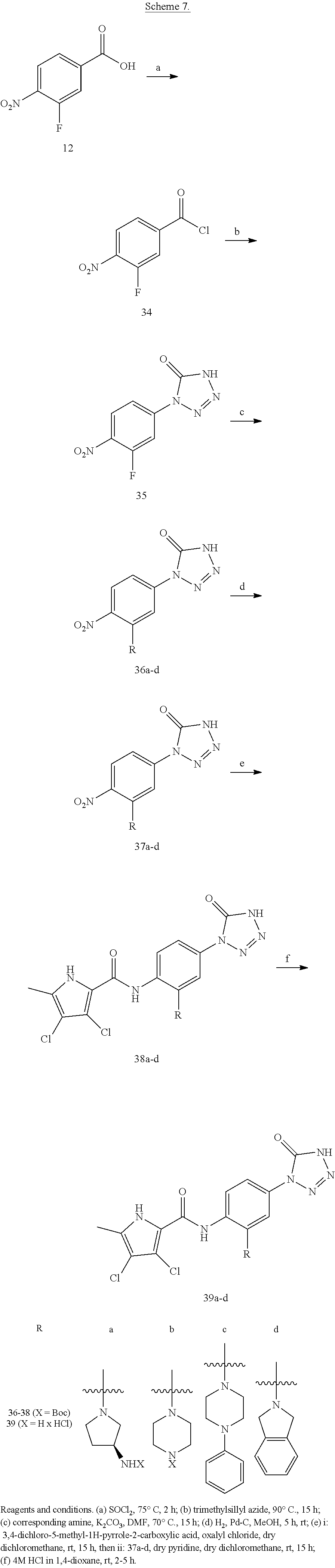

### (Example 22) (S)-1-(2-(3,4-Dichloro-5-methyl-1H-pyrrole-2-carboxamido)-5-(5-oxo-4,5-dihydro-1H-tetrazol-1-yl)phenyl)pyrrolidin-3-aminium chloride (39a)

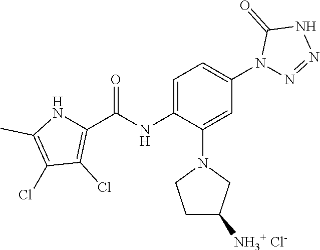

Step 1: Synthesis of 3-fluoro-4-nitrobenzoyl chloride (34). 3-Fluoro-4-nitrobenzoic acid (12, 10.0 g, 54.0 mmol) was dissolved in thionyl chloride (61 mL) and stirred at 75° C. for 2 h. Excess thionyl chloride was evaporated in vacuo. To remove traces of thionyl chloride toluene was added to the residue and evaporated in vacuo. Yield 91% (10.0 g); brown oil. ¹H NMR (400 MHz, CDCl₃): δ 8.05-8.12 (m, 2H, 2×ArH), 8.17-8.24 (m, 1H, ArH) ppm.

Step 2: Synthesis of 1-(3-fluoro-4-nitrophenyl)-1H-tetrazol-5(4H)-one (35). A mixture of 34 (5.00 g, 24.6 mmol) and trimethylsilyl azide (17.8 mL, 135 mmol) was stirred at 90° C. overnight. The reaction mixture was cooled on ice bath and the precipitate formed was filtered off, washed with diethyl ether and dried. Yield 76% (4.22 g); yellow solid; mp 155-160° C. ¹H NMR (400 MHz, DMSO-d₆): δ 8.01 (ddd, 1H, J=1.2, 2.2, 9.1 Hz, ArH), 8.11 (dd, 1H, J=2.2, 12.6 Hz, ArH), 8.40 (dd, 1H, J=8.5, 9.1 Hz, ArH), 15.08 (s, 1H, NH) ppm. MS (ESI) m/z=223.9 ([M−H]⁻).

Step 3: Synthesis of (S)-tert-butyl (1-(2-nitro-5-(5-oxo-4,5 dihydro-1H-tetrazol-1-yl)phenyl)pyrrolidin-3-yl)carbamate (36a). To a suspension of 35 (300 mg, 1.33 mmol) and K₂CO₃ (368 mg, 2.67 mmol) in DMF (15 mL), (S)-tert-butyl pyrrolidin-3-ylcarbamate (298 mg, 1.60 mmol) was added and the reaction mixture was stirred at 70° C. overnight. The solvent was evaporated in vacuo, to the residue ethyl acetate (80 mL) was added and the solution was washed with 1% citric acid (50 mL) and brine (50 mL). The organic phase was dried over Na₂SO₄, filtered and the solvent removed in vacuo. Yield 98% (0.512 g); yellow solid; mp 100-105° C. ¹H NMR (400 MHz, DMSO-d₆): δ 1.38 (s, 9H, tBu), 1.86-1.97 (m, 1H, CH), 2.06-2.17 (m, 1H, CH), 2.86-2.97 (m, 1H, CH), 3.26-3.31 (m, 1H, CH), 3.38-3.47 (m, 2H, 2×CH), 4.07-4.16 (m, 1H, CH), 7.23-7.33 (m, 2H, NH, ArH), 7.61 (d, 1H, J=2.1 Hz, ArH), 7.95 (d, 1H, J=9.0 Hz, ArH), 14.97 (s, 1H, NH) ppm. MS (ESI) m/z=390.0 ([M−H]⁻).

Step 4: Synthesis of tert-butyl (S)-(1-(2-amino-5-(5-oxo-4,5-dihydro-1H-tetrazol-1-yl)phenyl)pyrrolidin-3-yl)carbamate (37a). 36a (500 mg, 1.28 mmol) was dissolved in methanol (50 mL) and flushed with argon. Pd/C (10%, 50 mg) was added and the reaction mixture was then stirred at rt under hydrogen atmosphere for 5 h. The catalyst was filtered off and the solvent was removed in vacuo. Yield 87% (400 mg); light brown solid; mp 96-99° C. ¹H NMR (400 MHz, DMSO-d₆): δ 1.40 (s, 9H, tBu), 1.69 (td, 1H, J=12.6, 7.7 Hz, CH), 2.14-2.27 (m, 1H, CH), 2.79-2.89 (m, 2H, 2×CH), 3.07-3.12 (m, 1H, CH), 3.13-3.19 (m, 1H, CH), 4.04-4.17 (m, 1H, CH), 5.08 (s, 2H, NH₂), 6.72 (d, 1H, J=8.4 Hz, ArH), 7.12 (dd, 1H, J=8.4, 2.4 Hz, ArH), 7.18 (d, 1H, J=2.4 Hz, ArH), 7.22 (d, 1H, J=7.8 Hz, NH) ppm. Signal for one NH not seen. MS (ESI) m/z=360.1 ([M−H]⁻).

Step 5: Synthesis of (S)-tert-butyl (1-(2-(3,4-dichloro-5-methyl-1H-pyrrole-2-carboxamido)-5-(5-oxo-4,5-dihydro-1H-tetrazol-1-yl)phenyl)pyrrolidin-3-yl)carbamate (38a). To a suspension of 3,4-dichloro-5-methyl-1H-pyrrole-2-carboxylic acid (124 mg, 0.639 mmol) in anhydrous dichloromethane (15 mL), oxalyl chloride (274 μL, 3.20 mmol) was added dropwise and the reaction mixture was stirred at rt under argon atmosphere overnight. The solvent was evaporated under reduced pressure, 37a (200 mg, 0.553 mmol), anhydrous pyridine (2 mL) and anhydrous dichloromethane (10 mL) were added and the reaction mixture was stirred at rt under argon atmosphere overnight. The solvent was removed in vacuo, to the residue ethyl acetate and 1% citric acid were added. The precipitate formed was filtered off, washed with diethyl ether and methanol and dried. Yield 17% (50 mg); grey solid. ¹H NMR (400 MHz, DMSO-d₆): δ 1.38 (s, 9H, tBu), 1.80-1.88 (m, 1H, CH), 2.12-2.21 (m, 1H, CH), 2.24 (s, 3H, CH₃), 2.96 (dd, 1H, J=5.5, 9.5 Hz, CH), 3.11 (q, 1H, J=7.5 Hz, CH), 3.23 (q, 1H, J=7.8 Hz, CH), 3.38 (1H, overlapped with the signal for water, CH), 4.02-4.16 (m, 1H, CH), 7.19 (d, 1H, J=6.6 Hz, NH), 7.46 (dd, 1H, J=2.4, 8.6 Hz, ArH), 7.61 (d, 1H, J=2.4 Hz, ArH), 8.09 (d, 1H, J=8.6 Hz, ArH), 9.23 (s, 1H, NH), 12.33 (s, 1H, NH), 14.76 (s, 1H, NH) ppm.

Step 6: Synthesis of (S)-1-(2-(3,4-dichloro-5-methyl-1H-pyrrole-2-carboxamido)-5-(5-oxo-4,5-dihydro-1H-tetrazol-1-yl)phenyl)pyrrolidin-3-aminium chloride (39a). To a suspension of 38a (25 mg, 0.047 mmol) in 1,4-dioxane (1 mL), 4 M HCl in 1,4-dioxane (3 mL) was added and the reaction mixture was stirred at rt for 4 h. The precipitate that was formed was filtered off, washed with diethyl ether and methanol and dried. Yield 91% (20 mg). ¹H NMR (400 MHz, DMSO-d₆): δ 1.99 (ddd, 1H, J=17.7, 7.9, 4.6 Hz, CH), 2.24 (s, 3H, CH₃), 2.26-2.35 (m, 1H, CH), 3.10 (td, 2H, J=8.3, 3.7 Hz, 2×CH), 3.55 (dd, 2H, J=10.5, 6.9 Hz, 2×CH), 3.83-3.96 (m, 1H, CH), 7.51 (dd, 1H, J=8.8, 2.4 Hz, ArH), 7.65 (d, 1H, J=2.4 Hz, ArH), 8.07 (d, 1H, J=8.8 Hz, ArH), 8.16 (d, 3H, J=3.3 Hz, NH₃), 9.25 (s, 1H, NH), 12.47 (s, 1H, NH), 14.81 (s, 1H, NH) ppm.

### (Example 23) 1-(2-(3,4-Dichloro-5-methyl-1H-pyrrole-2-carboxamido)-5-(5-oxo-4,5-dihydro-1H-tetrazol-1-yl)phenyl)piperidin-4-aminium chloride (39b)

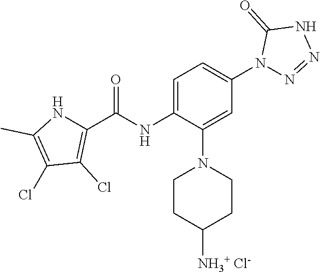

Step 1: Synthesis of tert-butyl (1-(2-nitro-5-(5-oxo-4,5-dihydro-1H-tetrazol-1-yl)phenyl)piperidin-4-yl)carbamate (36b). To a suspension of 35 (324 mg, 1.44 mmol) and K₂CO₃ (398 mg, 2.88 mmol) in DMF (15 mL), tert-butyl piperidin-4-ylcarbamate (346 mg, 1.73 mmol) was added and the reaction mixture was stirred at 70° C. overnight. The solvent was evaporated in vacuo, to the residue ethyl acetate (80 mL) was added and the solution was washed with 1% citric acid (50 mL) and brine (50 mL). The organic phase was dried over Na₂SO₄, filtered and the solvent removed in vacuo. The crude product was purified with flash column chromatography using dichloromethane/methanol (15:1) as eluent. Yield: 51% (300 mg); yellow solid. ¹H NMR (400 MHz, DMSO-d₆): δ 1.39 (s, 9H, tBu), 1.52 (qd, 2H, J=12.5, 3.6 Hz, CH₂), 1.82 (d, 2H, J=11.0 Hz, CH₂), 2.90 (t, 2H, J=11.1 Hz, CH₂), 3.20 (t, 2H, J=12.4 Hz, CH₂), 3.45 (m, 1H, CH), 6.95 (d, 1H, J=7.7 Hz, NH), 7.56 (dd, 1H, J=8.9, 2.1 Hz, ArH), 7.80 (d, 1H, J=2.1 Hz, ArH), 8.04 (d, 1H, J=8.9 Hz, ArH), 14.97 (s, 1H, NH) ppm. MS (ESI) m/z=404.0 ([M−H]⁻).

Step 2: Synthesis of tert-butyl (1-(2-amino-5-(5-oxo-4,5-dihydro-1H-tetrazol-1-yl)phenyl)piperidin-4-yl)carbamate (37b). 36b (250 mg, 0.617 mmol) was dissolved in a mixture of methanol and tetrahydrofuran (4:1, 50 mL) and flushed with argon. Pd/C (10%, 25 mg) was added and the reaction mixture was then stirred at rt under hydrogen atmosphere for 7 h. The catalyst was filtered off and the solvent removed in vacuo. Yield: 76% (175 mg); light brown solid. ¹H NMR (400 MHz, DMSO-d₆): δ 1.40 (s, 9H, tBu), 1.57 (dd, 2H, J=21.2, 9.7 Hz, CH₂), 1.83 (d, 2H, J=9.7 Hz, CH₂), 2.54 (2H, overlapped with the signal for DMSO-d₅, CH₂), 3.05 (d, 2H, J=11.9 Hz, CH₂), 3.35 (1H, overlapped with the signal for water, CH), 4.99 (s, 2H, NH₂), 6.77 (d, 1H, J=8.5 Hz, 1H), 6.92 (d, 1H, J=7.4 Hz, NH), 7.16 (dd, 1H, J=8.5, 2.3 Hz, ArH), 7.25 (d, 1H, J=2.3 Hz, ArH), 14.53 (s, 1H, NH) ppm.

Step 3: Synthesis of tert-butyl (1-(2-(3,4-dichloro-5-methyl-1H-pyrrole-2-carboxamido)-5-(5-oxo-4,5-dihydro-1H-tetrazol-1-yl)phenyl)piperidin-4-yl)carbamate (38b). To a suspension of 3,4-dichloro-5-methyl-1H-pyrrole-2-carboxylic acid (93 mg, 0.479 mmol) in anhydrous dichloromethane (10 mL), oxalyl chloride (172 μL, 2.01 mmol) was added dropwise and the reaction mixture was stirred at rt under argon atmosphere overnight. The solvent was evaporated under reduced pressure, 37b (150 mg, 0.400 mmol), anhydrous pyridine (2 mL) and anhydrous dichloromethane (10 mL) were added and the reaction mixture was stirred at rt under argon atmosphere overnight. The solvent was removed in vacuo, to the residue ethyl acetate and 1% citric acid were added. The phases were separated, organic phase was dried over Na₂SO₄, filtered and solvent removed in vacuo. The crude product was purified with flash column chromatography using dichloromethane/methanol (15:1) as eluent. Yield 25% (56 mg); light brown solid. ¹H NMR (400 MHz, DMSO-d₆): δ 1.40 (s, 9H, tBu), 1.58-1.72 (m, 2H, CH₂), 1.86 (d, 2H, J=10.8 Hz, CH₂), 2.24 (s, 3H, CH₃), 2.74 (t, 2H, J=10.7 Hz, CH₂), 2.97 (d, 2H, J=11.6 Hz, CH₂), 4.11 (dd, 1H, J=10.1, 5.0 Hz, CH), 7.11 (d, 1H, J=7.7 Hz, NH), 7.63 (dd, 1H, J=8.9, 2.2 Hz, ArH), 7.82 (d, 1H, J=2.2 Hz, ArH), 8.54 (d, 1H, J=8.9 Hz, ArH), 9.66 (s, 1H, NH), 12.40 (s, 1H, NH), 14.75 (s, 1H, NH) ppm.

Step 4: Synthesis of 1-(2-(3,4-dichloro-5-methyl-1H-pyrrole-2-carboxamido)-5-(5-oxo-4,5-dihydro-1H-tetrazol-1-yl)phenyl)piperidin-4-aminium chloride (39b). To a suspension of 38b (40 mg, 0.073 mmol) in 1,4-dioxane (1 mL), 4 M HCl in 1,4-dioxane (4 mL) was added and the reaction mixture was stirred at rt overnight. The precipitate that was formed was filtered off, washed with diethyl ether and dried. Yield 93% (33 mg); light grey solid; mp>300° C. ¹H NMR (400 MHz, DMSO-d₆): δ 1.71-1.87 (m, 2H, CH₂), 2.03-2.12 (m, 2H, CH₂), 2.25 (s, 3H, CH₃), 2.82 (t, 2H, J=11.3 Hz, CH₂), 2.98-3.10 (m, 2H, CH₂), 3.19 (s, 1H, CH), 7.66 (dd, 1H, J=2.4, 8.9 Hz, ArH), 7.85 (d, 1H, J=2.4 Hz, ArH), 8.26 (d, 3H, J=5.3 Hz, NH₃), 8.57 (d, 1H, J=8.9 Hz, ArH), 9.59 (s, 1H, NH), 12.43 (s, 1H, NH), 14.86 (s, 1H, NH) ppm.

### (Example 24) 3,4-Dichloro-5-methyl-N-(4-(5-oxo-4,5-dihydro-1H-tetrazol-1-yl)-2-(4-phenylpiperazin-1-yl)phenyl)-1H-pyrrole-2-carboxamide (38c)

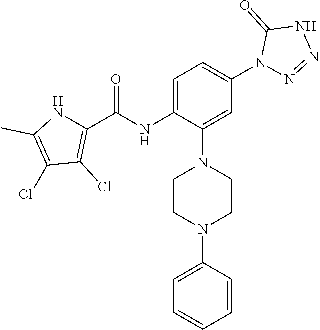 [SMILES: Cc1[nH]c(C(=O)Nc2ccc(-n3nn[nH]c3=O)cc2N2CCN(c3ccccc3)CC2)c(Cl)c1Cl]

Step 1: Synthesis of 1-(4-nitro-3-(4-phenylpiperazin-1-yl)phenyl)-1H-tetrazol-5(4H)-one (36c). To a suspension of 35 (0.610 g, 2.71 mmol) and K₂CO₃ (0.749 g, 5.42 mmol) in DMF (15 mL), 1-phenylpiperazine (469 μL, 2.98 mmol) was added and the reaction mixture was stirred at 70° C. overnight. The solvent was evaporated in vacuo, to the residue ethyl acetate (150 mL) was added and the solution was washed with 1% citric acid (100 mL) and brine (100 mL). The organic phase was dried over Na₂SO₄, filtered and the solvent removed in vacuo. The crude product was purified with flash column chromatography using dichloromethane/methanol (50:1) as eluent. Yield: 32% (320 mg); yellow solid; mp 115-120° C. ¹H NMR (400 MHz, DMSO-d₆): δ 3.20-3.26 (m, 4H, 2×CH₂), 3.28-3.32 (m, 4H, 2×CH₂), 6.82 (tt, 1H, J=1.0, 7.3 Hz, ArH), 6.99 (dd, 2H, J=1.1, 8.8 Hz, 2×ArH), 7.25 (dd, 2H, J=7.2, 8.7 Hz, 2×ArH), 7.63 (dd, 1H, J=2.2, 8.9 Hz, ArH), 7.87 (d, 1H, J=2.2 Hz, ArH), 8.10 (d, 1H, J=9.0 Hz, ArH), 14.98 (br s, 1H, NH) ppm. MS (ESI) m/z=366.1 ([M−H]⁻).

Step 2: Synthesis of 1-(4-amino-3-(4-phenylpiperazin-1-yl)phenyl)-1H-tetrazol-5(4H)-one (37c). 36c (300 mg, 0.817 mmol) was dissolved in a mixture of methanol and tetrahydrofuran (4:1, 50 mL) and flushed with argon. Pd/C (10%, 30 mg) was added and the reaction mixture was then stirred at rt under hydrogen atmosphere for 7 h. The catalyst was filtered off and the solvent removed in vacuo. Yield 25% (70 mg); light brown solid. ¹H NMR (400 MHz, DMSO-d₆): δ 2.98 (s, 4H, 2×CH₂), 3.33 (s, 4H, 2×CH₂), 5.08 (s, 2H, NH₂), 6.75-6.84 (m, 2H, 2×ArH), 7.00 (d, 2H, J=8.1 Hz, 2×ArH), 7.21-7.27 (m, 3H, 3×ArH), 7.33 (d, 1H, J=2.4 Hz, ArH) ppm. Signal for one NH proton not seen. MS (ESI) m/z=336.1 ([M−H]⁻).

Step 3: Synthesis of 3,4-dichloro-5-methyl-N-(4-(5-oxo-4,5-dihydro-1H-tetrazol-1-yl)-2-(4-phenylpiperazin-1-yl)phenyl)-1H-pyrrole-2-carboxamide (38c). A mixture of 3,4-dichloro-5-methyl-1H-pyrrole-2-carboxylic acid (35 mg, 0.180 mmol) and thionyl chloride (1 mL, 13.9 mmol) was stirred at 75° C. under argon atmosphere for 2 h. Excess thionyl chloride was evaporated in vacuo. To the residue 37c (50 mg, 0.148 mmol), anhydrous pyridine (2 mL) and anhydrous dichloromethane (10 mL) were added and the reaction mixture was stirred at rt under argon atmosphere overnight. The solvent was removed in vacuo, to the residue ethyl acetate and 1% citric acid were added. The phases were separated, organic phase was dried over Na₂SO₄, filtered and solvent removed in vacuo. The crude product was purified with flash column chromatography using dichloromethane/methanol (20:1) as eluent. Yield 16% (12.3 mg); light brown solid. ¹H NMR (400 MHz, DMSO-d₆) δ 2.24 (s, 3H, CH₃), 2.98-3.08 (m, 4H, 2×CH₂), 3.35-3.41 (m, 4H, 2×CH₂), 6.80-6.86 (m, 1H, ArH), 6.99-7.05 (m, 2H, 2×ArH), 7.23-7.30 (m, 2H, 2×ArH), 7.68 (dd, 1H, J=2.4, 8.9 Hz, ArH), 7.88 (d, 1H, J=2.4 Hz, ArH), 8.54 (d, 1H, J=8.9 Hz, ArH), 9.68 (s, 1H, NH), 12.45 (s, 1H, NH), 14.78 (s, 1H, NH) ppm.

### (Example 25) 3,4-Dichloro-N-(2-(isoindolin-2-yl)-4-(5-oxo-4,5-dihydro-1H-tetrazol-1-yl)phenyl)-5-methyl-1H-pyrrole-2-carboxamide (38d)

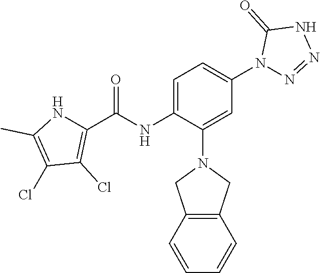 [SMILES: Cc1[nH]c(C(=O)Nc2ccc(-n3nn[nH]c3=O)cc2N2Cc3ccccc3C2)c(Cl)c1Cl]

Step 1: Synthesis of 1-(3-(isoindolin-2-yl)-4-nitrophenyl)-1H-tetrazol-5(4H)-one (36d). To a suspension of 35 (400 mg, 1.78 mmol) and K₂CO₃ (491 mg, 3.55 mmol) in DMF (15 mL), isoindolin-2-ium chloride (332 mg, 2.13 mmol) was added and the reaction mixture was stirred at 70° C. overnight. The solvent was evaporated in vacuo, to the residue ethyl acetate (100 mL) was added and the solution was washed with 1% citric acid (70 mL) and brine (70 mL). The organic phase was dried over Na₂SO₄, filtered and the solvent removed in vacuo. The crude product was purified with flash column chromatography using dichloromethane/methanol (30:1) as eluent. Yield 59% (340 mg); yellow solid; mp 180-183° C. ¹H NMR (400 MHz, DMSO-d₆): δ 4.66 (s, 4H, 2×CH₂), 7.33 (dd, 2H, J=3.1, 5.6 Hz, 2×ArH), 7.38 (dd, 1H, J=2.1, 8.9 Hz, ArH), 7.44 (dd, 2H, J=3.2, 5.5 Hz, 2×ArH), 7.76 (d, 1H, J=2.1 Hz, ArH), 7.97 (d, 1H, J=8.9 Hz, ArH), 14.97 (s, 1H, NH) ppm. MS (ESI) m/z=323.0 ([M−H]⁻).

Step 2: Synthesis of 1-(4-amino-3-(isoindolin-2-yl)phenyl)-1H-tetrazol-5(4H)-one (37d). 36d (321 mg, 0.990 mmol) was dissolved in a mixture of methanol and tetrahydrofuran (4:1, 50 mL) and flushed with argon. Pd/C (10%, 32 mg) was added and the reaction mixture was stirred at rt under hydrogen atmosphere for 7 h. The catalyst was filtered off and the solvent removed in vacuo. Yield 54% (156 mg); light brown solid; mp 105-109° C. ¹H NMR (400 MHz, DMSO-d₆): δ 4.45 (s, 4H, 2×CH₂), 5.12 (s, 2H, NH₂), 6.81 (d, 1H, J=8.5 Hz, ArH), 7.14 (dd, 1H, J=2.3, 8.4 Hz, ArH), 7.23-7.31 (m, 2H, 2×ArH), 7.31-7.39 (m, 2H, 2×ArH), 7.42 (d, 1H, J=2.3 Hz, ArH), 14.54 (br s, 1H, NH) ppm.

Step 3: Synthesis of 3,4-dichloro-N-(2-(isoindolin-2-yl)-4-(5-oxo-4,5-dihydro-1H-tetrazol-1-yl)phenyl)-5-methyl-1H-pyrrole-2-carboxamide (38d). To a suspension of 3,4-dichloro-5-methyl-1H-pyrrole-2-carboxylic acid (103 mg, 0.530 mmol) in anhydrous dichloromethane (10 mL), oxalyl chloride (189 μL, 2.21 mmol) was added dropwise and the reaction mixture was stirred at rt under argon atmosphere overnight. The solvent was evaporated under reduced pressure, 37d (130 mg, 0.442 mmol), anhydrous pyridine (2 mL) and anhydrous dichloromethane (10 mL) were added and the reaction mixture was stirred at rt under argon atmosphere overnight. The solvent was removed in vacuo, to the residue ethyl acetate and 1% citric acid were added. The phases were separated, organic phase was dried over Na₂SO₄, filtered and solvent removed in vacuo. The crude product was purified with flash column chromatography using dichloromethane/methanol (15:1) as eluent. Yield 8% (17 mg); light brown solid; mp 260° C. ¹H NMR (400 MHz, DMSO-d₆): δ 2.23 (s, 3H, CH₃), 4.59 (s, 4H, 2×CH₂), 7.27-7.33 (m, 2H, 2×ArH), 7.33-7.39 (m, 2H, 2×ArH), 7.50 (dd, 1H, J=2.3, 8.7 Hz, ArH), 7.79 (d, 1H, J=2.4 Hz, ArH), 8.07 (d, 1H, J=8.7 Hz, ArH), 9.52 (s, 1H, NH), 12.33 (s, 1H, NH), 14.77 (s, 1H, NH) ppm.

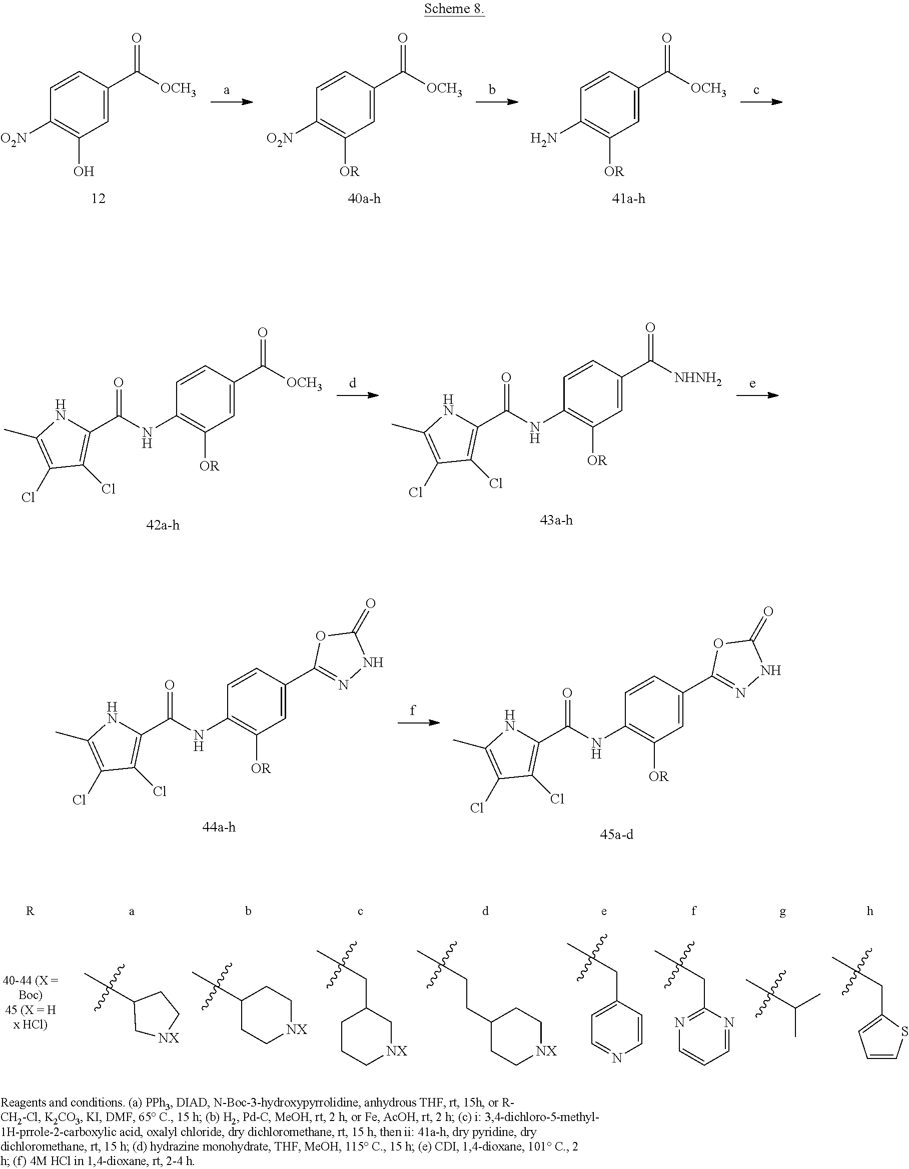

### (Example 26) 3-(2-(3,4-Dichloro-5-methyl-1H-pyrrole-2-carboxamido)-5-(5-oxo-4,5-dihydro-1,3,4-oxadiazol-2-yl)phenoxy)pyrrolidin-1-ium chloride (45a)

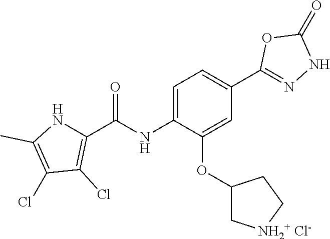

Step 1: Synthesis of tert-butyl 3-(5-(methoxycarbonyl)-2-nitrophenoxy)pyrrolidine-1-carboxylate (40a). General procedure H. To a stirred solution of methyl 3-hydroxy-4-nitrobenzoate (12, 2.00 g, 10.1 mmol) and triphenylphosphine (3.46 g, 13.2 mmol) in anhydrous tetrahydrofuran (130 mL) tert-butyl 3-hydroxypyrrolidine-1-carboxylate (2.28 g, 12.2 mmol) was added and the mixture was stirred at rt for 10 min. Diisopropyl azodicarboxylate (2.4 mL, 12.2 mmol) was added dropwise and the mixture was stirred at rt for 15 h under argon atmosphere. The solvent was evaporated under reduced pressure and the crude product was purified with two consecutive flash column chromatographies using ethyl acetate/petroleum ether (1:2) and dichloromethane/methanol (75:1) as eluents. Yield 3.66 g (41%); light yellow solid. ¹H NMR (400 MHz, DMSO-d₆): δ 1.40 (d, 9H, J=7.8 Hz, tBu), 2.00-2.26 (m, 2H, 2×CH), 3.23-3.32 (m, 1H, CH), 3.38-3.48 (m, 2H, 2×CH), 3.50-3.63 (m, 1H, CH), 3.91 (s, 3H, CH₃), 5.39 (br s, 1H, CH), 7.70 (dd, 1H, J=8.3, 1.5 Hz, ArH), 7.83 (d, 1H, J=1.5 Hz, ArH), 8.00 (d, 1H, J=8.3 Hz, ArH) ppm.

Step 2: Synthesis of tert-butyl 3-(2-amino-5-(methoxycarbonyl)phenoxy)pyrrolidine-1-carboxylate (41a). General procedure I. Compound 40a (1.50 g, 4.08 mmol) was dissolved in MeOH (40 mL) and the mixture was stirred at rt for 10 min under argon atmosphere. Pd/C (10%, 149 mg) was added and the reaction mixture was stirred under hydrogen atmosphere for 1 h. The catalyst was filtered off and the solvent was removed under reduced pressure. Yield 1.38 g (92%); light brown oil. ¹H NMR (400 MHz, DMSO-d₆): δ 1.40 (d, 9H, J=6.4 Hz, tBu), 2.02-2.14 (m, 2H, 2×CH), 3.38-3.46 (m, 3H, 3×CH), 3.50-3.60 (m, 1H, CH), 3.75 (s, 3H, CH₃), 4.98 (s, 1H, CH), 5.66 (s, 2H, NH₂), 6.66 (d, 1H, J=8.3 Hz, ArH), 7.26 (d, 1H, J=1.5 Hz, ArH), 7.39 (dd, 1H, J=8.3, 1.5 Hz, ArH) ppm.

Step 3: Synthesis of tert-butyl 3-(2-(3,4-dichloro-5-methyl-1H-pyrrole-2-carboxamido)-5-(methoxycarbonyl)phenoxy)pyrrolidine-1-carboxylate (42a). General procedure J. To a dry 3,4-dichloro-5-methyl-1H-pyrrole-2-carboxylic acid (863 mg, 4.45 mmol) anhydrous SOCl₂ (7.5 mL) was added under argon atmosphere. The solution was stirred at 75° C. for 40 min under argon atmosphere. The solvent was evaporated and dried, using a water pump and rotavapor. tert-Butyl 3-(2-amino-5-(methoxycarbonyl)phenoxy)pyrrolidine-1-carboxylate (1.25 g, 3.71 mmol), fresh anhydrous CH₂Cl₂ (12 mL) and pyridine (12 mL) were added and the reaction mixture was stirred under argon atmosphere at rt for 15 hours. The solvent was removed under reduced pressure, the residue was suspended in ethyl acetate and washed with 1 M HCl (15 mL), then washed again with methanol, ethyl acetate and diethyl ether. Yield 1.22 g (64%); light brown solid. ¹H NMR (400 MHz, DMSO-d₆): δ 1.43 (d, 9H, J=7.0 Hz, tBu), 2.02-2.21 (m, 2H, 2×CH), 2.23 (s, 3H, CH₃), 3.46 (t, 2H, J=10.0 Hz, 2×CH), 3.52-3.70 (m, 2H, 2×CH), 3.85 (s, 3H, CH₃), 5.31 (d, 1H, J=4.5 Hz, CH), 7.58 (d, 1H, J=8.7 Hz, ArH), 7.66 (dd, 1H, J=8.5, 1.8 Hz, ArH), 8.54 (dd, 1H, J=8.5, 2.4 Hz, ArH), 9.22 (d, 1H, J=7.8 Hz, NH), 12.47 (s, 1H, NH) ppm.

Step 4: Synthesis of tert-butyl 3-(2-(3,4-dichloro-5-methyl-5-methyl-1H-pyrrole-2-carboxamido)-5-(hydrazinecarbonyl)phenoxy)pyrrolidine-1-carboxylate (43a). General procedure K. To a solution of 42a (200 mg, 0.390 mmol) in methanol (5 mL) and THE (3 mL) in a pressure tube hydrazine monohydrate (0.8 mL, 27.3 mmol) was added. The reaction mixture was stirred at 115° C. overnight. The obtained suspension was cooled in an ice bath and the precipitate was washed with methanol, filtered off and dried. Yield 0.142 g (71%); white solid. ¹H NMR (400 MHz, DMSO-d₆): δ 1.43 (d, 9H, J=5.8 Hz, tBu), 2.08-2.20 (m, 2H, 2×CH), 2.23 (s, 3H, CH₃), 3.35-3.40 (m, 1H, CH), 3.46 (t, 1H, J=9.8 Hz, CH), 3.52-3.69 (m, 2H, 2×CH), 4.48 (s, 2H, NH₂), 5.25 (s, 1H, CH), 7.52 (d, 1H, J=8.5 Hz, ArH), 7.56 (d, 1H, J=5.5 Hz, NH), 8.45 (d, 1H, J=8.8 Hz, ArH), 9.14 (d, 1H, J=8.2 Hz, ArH), 9.76 (s, 1H, NH), 12.44 (s, 1H, NH) ppm.

Step 5: Synthesis of tert-butyl 3-(2-(3,4-dichloro-5-methyl-1H-pyrrole-2-carboxamido)-5-(5-oxo-4,5-dihydro-1,3,4-oxadiazol-2-yl)phenoxy)pyrrolidine-1-carboxylate (44a). General procedure L. To a solution of 43a (200 mg, 0,390 mmol) in 1,4-dioxane (12 mL) and DMF (6 mL), carbonyldiimidazole (127 mg, 0.781 mmol) was added and the reaction mixture was stirred at 101° C. for 15 h. The solvent was evaporated and the solid was washed with acetonitrile and methanol. Yield 189 mg (90%); white solid. ¹H NMR (400 MHz, DMSO-d₆): δ 1.43 (d, 9H, J=6.6 Hz, tBu), 2.05-2.22 (m, 2H, 2×CH), 2.23 (s, 3H, CH₃), 3.46 (t, 1H, J=9.7 Hz, CH), 3.53-3.70 (m, 2H, 2×CH), 5.36 (s, 1H, CH), 7.43 (d, 1H, J=9.5 Hz, ArH), 7.47 (dd, 1H, J=8.5, 1.8 Hz, ArH), 8.56 (dd, 1H, J=8.4, 2.3 Hz, ArH), 9.19 (d, 1H, J=7.7 Hz, NH), 12.46 (s, 1H, NH), 12.58 (s, 1H, NH) ppm. Signal for one CH is under the peak for solvent.

Step 6: Synthesis of 3-(2-(3,4-dichloro-5-methyl-1H-pyrrole-2-carboxamido)-5-(5-oxo-4,5-dihydro-1,3,4-oxadiazol-2-yl)phenoxy)pyrrolidin-1-ium chloride (45a). General procedure M. Compound 44a (148 mg, 0.275 mmol) was suspended in 4 M HCl in 1,4-dioxane (19.9 mL). The mixture was stirred at rt for 1 h. The solid was filtered off and washed with diethyl ether and methanol. Yield 112 mg (86%); light yellow solid. ¹H NMR (400 MHz, DMSO-d₆): δ 2.25 (s, 3H, CH₃), 2.26-2.32 (m, 1H, CH), 2.32-2.41 (m, 1H, CH), 3.37-3.49 (m, 3H, 3×CH), 3.52-3.60 (m, 1H, CH), 5.45 (s, 1H, CH), 7.40 (d, 1H, J=1.8 Hz, ArH), 7.50 (dd, 1H, J=8.5, 1.8 Hz, ArH), 8.53 (d, 1H, J=8.6 Hz, ArH), 9.10 (s, 1H, Ha from NH₂), 9.30 (s, 1H, Hb from NH₂), 9.37 (s, 1H, NH), 12.52 (s, 1H, NH), 12.64 (s, 1H, NH) ppm. HRMS (ESI) m/z for Cl₈H₁₃O₄N₅Cl₂ calculated 438.0730. found 438.0730 ([M+H]⁺). HPLC (30-90% ACN in 0.1% TFA in 21 min—UPLC): tᵣ 3.677 (96.66% at 254 nm).

### (Example 27) 4-(2-(3,4-Dichloro-5-methyl-1H-pyrrole-2-carboxamido)-5-(5-oxo-4,5-dihydro-1,3,4-oxadiazol-2-yl)phenoxy)piperidin-1-ium chloride (45b)

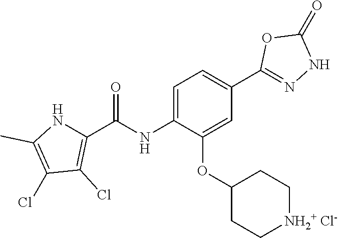

Step 1: Synthesis of tert-butyl 4-(5-(methoxycarbonyl)-2-nitrophenoxy)piperidine-1-carboxlate (40b). Sinthesized according to General procedure H using tert-butyl 4-hydroxypiperidine-1-carboxylate (2.45 g, 12.2 mmol) as reagent. Yield 0.554 g (25%); white solid. ¹H NMR (400 MHz, DMSO-d₆): δ 1.40 (s, 9H, tBu), 1.55-1.66 (m, 2H, CH₂), 1.83-1.93 (m, 2H, CH₂), 3.34-3.40 (m, 2H, CH₂), 3.41-3.52 (m, 2H, CH₂), 3.90 (s, 3H, CH₃), 4.97-5.04 (m, 1H, CH), 7.67 (dd, 1H, J=8.4, 1.6 Hz, ArH), 7.85 (d, 1H, J=1.5 Hz, ArH), 7.99 (d, 1H, J=8.4 Hz, ArH) ppm.

Step 2: Synthesis of tert-butyl 4-(2-amino-5-(methoxycarbonyl)phenoxy)piperidine-1-carboxylate (41b) Sinthesized according to General procedure I using 40b (554 mg, 1.47 mmol) as reagent. Yield 155 mg (30%); white solid. ¹H NMR (400 MHz, DMSO-d₆): δ 1.40 (s, 9H, tBu), 1.54-1.65 (m, 2H, CH₂), 1.80-1.65 (m, 2H, CH₂), 3.18-3.30 (m, 2H, CH₂), 3.58-3.68 (m, 2H, CH₂), 3.74 (s, 3H, CH₃), 4.49-4.56 (m, 1H, CH), 5.66 (s, 2H, NH₂), 6.65 (d, 1H, J=8.2 Hz, ArH), 7.34 (d, 1H, J=1.9 Hz, ArH), 7.37 (d, 1H, J=8.2 Hz, ArH) ppm.

Step 3: Synthesis of tert-butyl 4-(2-(3,4-dichloro-5-methyl-1H-pyrrole-2-carboxamido)-5-(methoxycarbonyl)phenoxy)piperidine-1-carboxylate (42b). Sinthesized according to General procedure J using 41b (300 mg, 0.856 mmol) as reagent. Yield 230 mg (51%); light brown solid. ¹H NMR (400 MHz, DMSO-d₆): δ 1.41 (s, 9H, tBu), 1.48-1.61 (m, 2H, CH₂), 1.97-2.09 (m, 2H, CH₂), 2.24 (s, 3H, CH₃), 3.01-3.22 (m, 2H, CH₂), 3.74-3.84 (m, 2H, CH₂), 3.85 (s, 3H, CH₃), 4.78-4.89 (m, 1H, CH), 7.64-7.66 (m, 2H, 2×ArH), 8.52 (d, 1H, J=8.8 Hz, ArH), 9.22 (s, 1H, NH), 12.48 (s, 1H, NH) ppm.

Step 4: Synthesis of tert-butyl 4-(2-(3,4-dichloro-5-methyl-1H-pyrrole-2-carboxamido)-5-(hydrazinecarbonyl)phenoxy)piperidine-1-carboxylate (43b). Sinthesized according to General procedure K using 42b (230 mg, 0.437 mmol) as reagent. Yield 0.178 mg (77%); white solid. ¹H NMR (400 MHz, DMSO-d₆): δ 1.42 (s, 9H, tBu), 1.49-1.63 (m, 2H, CH₂), 2.01-2.11 (m, 2H, CH₂), 2.24 (s, 3H, CH₂), 3.01-3.16 (m, 2H, CH₂), 3.77-3.90 (m, 2H, CH₂), 4.48 (s, 2H, NH₂), 4.72-4.83 (m, 1H, CH), 7.50 (d, 1H, J=8.6 Hz, ArH), 7.60 (s, 1H, ArH), 8.43 (d, 1H, J=8.5 Hz, ArH), 9.13 (s, 1H, NH), 9.74 (s, 1H, NH), 12.46 (s, 1H, NH) ppm.

Step 5: Synthesis of tert-butyl 4-(2-(3,4-dichloro-5-methyl-1H-pyrrole-2-carboxamido)-5-(5-oxo-4,5dihydro-1,3,4-oxadiazol-2-yl)phenoxy)piperidine-1-carboxylate (44b). Sinthesized according to General procedure L using 43b (158 mg, 0.300 mmol) as reagent. Yield 144 mg (87%); white solid. ¹H NMR (400 MHz, DMSO-d₆): δ 1.41 (s, 9H, tBu), 1.49-1.63 (m, 2H, CH₂), 1.98-2.11 (m, 2H, CH₂), 2.24 (s, 3H, CH₃), 3.02-3.20 (m, 2H, CH₂), 3.75-3.82 (m, 2H, CH₂), 4.82-4.92 (m, 1H, CH), 7.45 (dd, 1H, J=8.5, 1.7 Hz, ArH), 7.50 (d, 1H, J=1.7 Hz, ArH), 8.54 (d, 1H, J=8.5 Hz, ArH), 9.18 (s, 1H, NH), 12.47 (s, 1H, NH), 12.55 (s, 1H, NH) ppm.

Step 6: Synthesis of 4-(2-(3,4-dichloro-5-methyl-1H-pyrrole-2-carboxamido)-5-(5-oxo-4,5-dihydro-1,3,4-oxadiazol-2-yl)phenoxy)piperidin-1-ium-chloride (45b). Sinthesized according to General procedure M using 44b (124 mg, 0.225 mmol) as reagent. Yield 84 mg (76%); white solid. ¹H NMR (400 MHz, DMSO-d₆): δ 1.82-2.01 (m, 2H, CH₂), 2.16-2.24 (m, 2H, CH₂), 2.24 (s, 3H, CH₃), 3.03-3.21 (m, 2H, CH₂), 3.29-3.33 (m, 2H, CH₂), 5.91-5.03 (m, 1H, CH), 7.48 (dd, 1H, J=8.5, 1.7 Hz, ArH), 7.51 (d, 1H, J=1.9 Hz, ArH), 8.53 (d, 1H, J=8.5 Hz, ArH), 8.71 (s, 1H, Ha from NH₂), 8.86 (s, 1H, Hb from NH₂), 9.19 (s, 1H, HN), 12.51 (s, 1H, NH), 12.63 (s, 1H, NH) ppm. ¹³C NMR (100 MHz, DMSO-d₆): δ 11.25, 28.03, 41.62, 71.63, 109.16, 109.70, 110.62, 119.13, 119.45, 119.51, 120.06, 130.41, 131.49, 145.67, 153.99, 154.94, 156.84 ppm. HRMS for C₁₉H₂₀Cl₂N₅O₄: calculated 452.0887; found 452.0876. HPLC (10-90% ACN in 0.1% TFA in 11 min—UPLC): tᵣ 3.453 min (99.11% at 254 nm; 98.20% at 280 nm).

### (Example 28) 3-((2-(3,4-Dichloro-5-methyl-1H-pyrrole-2-carboxamido)-5-(5-oxo-4,5-dihydro-1,3,4-oxadiazol-2-yl)phenoxy)methyl)piperidin-1-ium chloride (45c)

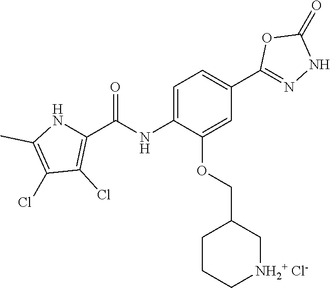

Step 1: Synthesis of tert-butyl 3-((5-(methoxycarbonyl)-2-nitrophenoxy)-methyl)piperidine-1-carboxylate (40c). Sinthesized according to General procedure H using tert-butyl 4-(hydroxymethyl)piperidine-1-carboxylate (2.62 g, 10.1 mmol) as reagent. Yield 3.66 g (72%); light yellow solid. 1H NMR (400 MHz, DMSO-d₆): δ 1.19-1.45 (m, 11H, tBu, CH₂), 1.61-1.69 (m, 1H, CH), 1.75-1.85 (m, 1H, CH), 1.84-1.96 (m, 1H, CH), 2.81 (ddd, 2H, J=13.5, 10.5, 2.9 Hz, CH₂), 3.67-3.82 (m, 1H, CH), 3.91 (s, 3H, CH₃), 3.94-4.12 (m, 2H, CH₂), 4.12-4.20 (m, 1H, CH), 7.67 (dd, 1H, J=8.3, 1.6 Hz, ArH), 7.77 (d, 1H, J=1.6 Hz, ArH), 8.01 (d, 1H, J=8.3 Hz, ArH) ppm.

Step 2: Synthesis of tert-butyl 3-((2-amino-5-(methoxycarbonyl)phenoxy)methyl)-piperidine-1-carboxylate (41c). Sinthesized according to General procedure/using 40c (2.90 g, 7.35 mmol) as reagent. Yield 329 mg (12%); white solid. ¹H NMR (400 MHz, DMSO-d₆): b 1.28-1.48 (s, 11H, tBu, CH₂), 1.57-1.70 (m, 1H, CH), 1.77-1.88 (m, 1H, CH), 1.87-1.98 (m, 1H, CH), 2.71-3.12 (m, 2H, CH₂), 3.61-3.74 (m, 1H, CH), 3.74 (s, 3H, CH₃), 3.78-4.06 (m, 3H, 3×CH), 5.60 (s, 2H, NH₂), 6.65 (d, 1H, J=8.2 Hz, ArH), 7.27 (d, 1H, J=1.8 Hz, ArH), 7.38 (dd, 1H, J=8.2, 1.8 Hz, ArH) ppm.

Step 3: Synthesis of tert-butyl 3-((2-(3,4-dichloro-5-methyl-1H-pyrrole-2-carboxamido)-5-(methoxycarbonyl)phenoxy)methyl)piperidine-1-carboxylate (42c). Sinthesized according to General procedure J using 41c (300 mg, 0.823 mmol) as reagent. Yield 260 mg (58%); white solid. ¹H NMR (400 MHz, DMSO-d₆): δ 1.11-1.50 (m, 11H, tBu, CH₂), 1.62-1.72 (m, 1H, CH), 1.80-1.94 (m, 1H, CH), 1.95-2.05 (m, 1H, CH), 2.24 (s, 3H, CH₃), 2.88-3.07 (m, 2H, CH₂), 3.58-3.77 (m, 1H, CH), 3.85 (s, 4H, CH₃, CH), 4.02-4.16 (m, 2H, CH₂), 7.57 (d, 1H, J=1.8 Hz, ArH), 7.66 (dd, 1H, J=8.5, 1.8 Hz, ArH), 8.53 (d, 1H, J=8.5 Hz, ArH), 9.26 (s, 1H, NH), 12.49 (s, 1H, NH) ppm.

Step 4: Synthesis of tert-butyl 3-((2-(3,4-dichloro-5-methyl-1H-pyrrole-2-carboxamido)-5-(hydrazinecarbonyl)phenoxy)methyl)piperidine-1-carboxylate (43c). Sinthesized according to General procedure K using 42c (230 mg, 0.426 mmol) as reagent. Yield 132 mg (58%); white solid. ¹H NMR (400 MHz, DMSO-d₆): δ 1.09-1.52 (m, 11H, tBu, CH₂), 1.60-1.74 (m, 1H, CH), 1.81-1.93 (m, 1H, CH), 1.95-2.09 (m, 1H, CH), 2.23 (s, 3H, CH₃), 2.98-3.08 (m, 2H, CH₂), 3.61-3.74 (m, 1H, CH), 3.80-3.94 (m, 1H, CH), 4.01-4.12 (m, 2H, CH₂), 4.48 (s, 2H, NH₂), 7.44-7.53 (m, 1H, ArH), 7.56 (s, 1H, ArH), 8.44 (d, 1H, J=8.4 Hz, ArH), 9.20 (s, 1H, NH), 9.73 (s, 1H, NH) ppm. Signal for one NH proton not seen.

Step 5: Synthesis of tert-butyl 3-((2-(3,4-dichloro-5-methyl-1H-pyrrole-2-carboxamido)-5-(5-oxo-4,5-dihydro-1,3,4-oxadiazol-2-yl)phenoxy)methylpiperidine-1-carboxylate (44c). Sinthesized according to General procedure L using 43c (100 mg, 0.185 mmol) as reagent. Yield 60 mg (57%); white solid. ¹H NMR (400 MHz, DMSO-d₆): b 1.17-1.43 (m, 11H, tBu, CH₂), 1.61-1.74 (m, 1H, CH), 1.79-1.94 (m, 1H, CH), 1.95-2.10 (m, 1H, CH), 2.24 (s, 3H, CH₃), 2.83-3.11 (m, 2H, CH₂), 3.58-3.78 (m, 1H, CH), 3.83-4.00 (m, 1H, CH), 4.02-4.17 (m, 2H, CH₂), 7.42 (d, 1H, J=1.8 Hz, ArH), 7.46 (dd, 1H, J=8.4, 1.8 Hz, ArH), 8.55 (d, 1H, J=8.5 Hz, ArH), 9.22 (s, 1H, NH), 12.48 (s, 1H, NH), 12.58 (s, 1H, NH) ppm.

Step 6: Synthesis of 3-((2-(3,4-dichloro-5-methyl-1H-pyrrole-2-carboxamido)-5-(5-oxo-4,5-dihydro-1,3,4-oxadiazol-2-yl)phenoxy)methyl)piperidin-1-ium chloride (45c). Sinthesized according to General procedure Musing 44c (45 mg, 0.079 mmol) as reagent. Yield 25 mg (63%); white solid. ¹H NMR (400 MHz, DMSO-d₆): δ 1.33-1.46 (m, 1H, CH), 1.62-1.77 (m, 1H, CH), 1.81-1.91 (m, 1H, CH), 1.91-2.00 (m, 1H, CH), 2.24 (s, 3H, CH₃), 2.30-2.38 (m, 1H, CH), 2.74-2.89 (m, 2H, CH₂), 3.27 (d, 1H, J=12.4 Hz, CH), 3.45 (m, 1H, CH), 4.09-4.22 (m, 2H, CH₂), 7.41 (d, 1H, J=1.8 Hz, ArH), 7.48 (dd, 1H, J=8.5, 1.8 Hz, ArH), 8.54 (d, 1H, J=8.5 Hz, ArH), 8.68 (q, 1H, J=11.6, 10.7 Hz, Ha from NH₂), 8.81 (d, 1H, J=11.3 Hz, Hb from NH₂), 9.12 (s, 1H, NH), 12.50 (s, 1H, NH), 12.62 (s, 1H, NH) ppm. ¹³C NMR (100 MHz, DMSO-d₆): δ 11.25, 21.50, 25.30, 33.57, 43.97, 45.77, 70.87, 108.28, 109.17, 110.49, 119.05, 119.14, 119.45, 119.84, 130.45, 130.59, 147.46, 153.99, 154.91, 156.86 ppm. HRMS (ESI) m/z for C₂₀H₂₂O₄N₅Cl₂ calculated 466.1043. found 466.1037. HPLC (10-90% ACN in 0.1% TFA in 11 min—UPLC): tᵣ 3.570 min (96.10% at 254 nm, 95.30% at 280 nm).

### (Example 29) 4-(2-(2-(3,4-Dichloro-5-methyl-1H-pyrrole-2-carboxamido)-5-(5-oxo-4,5-dihydro-1,3,4-oxadiazol-2-yl)phenoxy)ethyl)piperidin-1-ium chloride (45d)

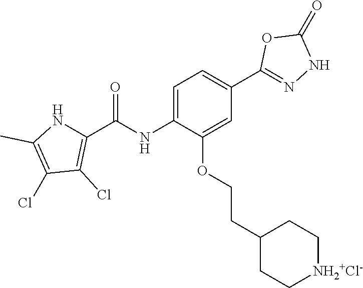

Step 1: Synthesis of tert-butyl 4-(2-(5-(methoxycarbonyl)-2-nitrophenoxy)ethyl)piperidine-1-carboxylate (40d). Sinthesized according to General procedure H using tert-butyl 4-(2-hydroxyethyl)piperidine-1-carboxylate (2.79 g, 12.2 mmol) as reagent. Yield 1.49 g (37%); white solid. ¹H NMR (400 MHz, DMSO-d₆): δ 0.96-1.11 (m, 2H, CH₂), 1.39 (s, 9H, tBu), 1.58-1.66 (m, 2H, CH₂), 1.66-1.72 (m, 3H, CH, CH₂), 2.69 (s, 2H, CH₂), 3.91 (s, 5H, CH₂, CH₃), 4.28 (t, 2H, J=6.2 Hz, CH₂), 7.66 (dd, 1H, J=8.4, 1.6 Hz, ArH), 7.79 (d, 1H, J=1.6 Hz, ArH), 7.98 (d, 1H, J=8.3 Hz, ArH) ppm.

Step 2: Synthesis of tert-butyl 4-(2-(2-amino-5-(methoxycarbonyl)phenoxy)-ethylpiperidine-1-carboxylate (41d). Sinthesized according to General procedure/using 40d (1.49 g, 3.66 mmol) as reagent. Yield 1.36 g (98%); white solid. ¹H NMR (400 MHz, DMSO-d₆): δ 0.97-1.12 (m, 2H, CH₂), 1.39 (s, 9H, tBu), 1.64-1.78 (m, 5H, 2×CH₂, CH), 3.74 (s, 3H, CH₃), 3.86-3.98 (m, 2H, CH₂), 4.01 (t, 2H, J=5.8 Hz, CH₂), 5.58 (s, 2H, NH₂), 6.64 (d, 1H J=8.2 Hz, ArH), 7.28 (d, 1H, J=1.7 Hz, ArH), 7.37 (dd, 1H, J=8.2, 1.8 Hz, ArH) ppm.

Step 3: Synthesis of tert-butyl 4-(2-(2-(3,4-dichloro-5-methyl-1H-pyrrole-2-carboxamido)-5-(methoxycarbonyl)phenoxy)ethyl)piperidine-1-carboxylate (42d). Sinthesized according to General procedure J using 41d (1.30 g, 3.44 mmol) as reagent. Yield 703 mg (37%); white solid. ¹H NMR (400 MHz, DMSO-d₆): δ 1.01-1.16 (m, 2H, CH₂), 1.39 (s, 9H, tBu), 1.63-1.82 (m, 5H, 2×CH₂, CH), 2.24 (s, 3H, CH₃), 2.57-2.78 (m, 2H, CH₂), 3.85 (s, 3H, CH₃), 3.88-3.99 (m, 2H, CH₂), 4.23 (t, 2H, J=6.2 Hz, CH₂), 7.59 (d, 1H, J=1.7 Hz, ArH), 7.65 (dd, 1H, J=8.5, 1.7 Hz, ArH), 8.51 (d, 1H, J=8.5 Hz, ArH), 9.23 (s, 1H, NH), 12.48 (s, 1H, NH) ppm.

Step 4: Synthesis of tert-butyl 4-(2-(2-(3,4-dichloro-5-methyl-1H-pyrrole-2-carboxamido)-5-(hydrazinecarbonyl)phenoxy)ethyl)piperidine-1-carboxylate (43d). Sinthesized according to General procedure K using 42d (503 mg, 0.907 mmol) as reagent. Yield 362 mg (72%); white solid. ¹H NMR (400 MHz, DMSO-d₆) δ 1.09 (q, 2H, J=11.5 Hz, CH₂), 1.39 (s, 9H, tBu), 1.64-1.81 (m, 5H, 2×CH₂, CH), 2.23 (s, 3H, CH₃), 2.67 (s, 2H, CH₂), 3.94 (d, 2H, J=11.1 Hz, CH₂), 4.20 (t, 2H, J=6.1 Hz, CH₂), 4.49 (s, 2H, NH₂), 7.49 (d, 1H, J=8.4 Hz, ArH), 7.56 (s, 1H, ArH), 8.41 (d, 1H, J=8.4 Hz, ArH), 9.15 (s, 1H, NH), 9.71 (s, 1H, NH), 12.45 (s, 1H, NH) ppm.

Step 5: Synthesis of tert-butyl 4-(2-(2-(3,4-dichloro-5-methyl)-1H-pyrrole-2-carboxamido)-5-(5-oxo-4,5-dihydro-1,3,4-oxadiazol-2-yl)phenoxy)ethylpiperidine-1-carboxylate (44d). Sinthesized according to General procedure L using 43d (200 mg, 0.361 mmol) as reagent. Yield 171 mg (82%); white solid. ¹H NMR (400 MHz, DMSO-d₆): δ 1.08 (q, 2H, J=11.2 Hz, CH₂), 1.39 (s, 9H, tBu), 1.81-1.62 (m, 5H, 2×CH₂, CH), 2.23 (s, 3H, CH₃), 2.78-2.62 (m, 2H, CH₂), 3.94 (d, 2H, J=10.6 Hz, CH₂), 4.23 (t, 2H, J=6.1 Hz, CH₂), 7.40 (d, 1H, J=1.7 Hz, ArH), 7.43 (dd, 1H, J=8.4, 1.7 Hz, ArH), 8.52 (d, 1H, J=8.4 Hz, ArH), 9.17 (s, 1H, NH), 12.46 (s, 1H, NH), 12.55 (s, 1H, NH) ppm.

Step 6: Synthesis of 4-(2-(2-(3,4-dichloro-5-methy-1H-pyrrole-2-carboxamido)-5-(5-oxo-4,5-dihydro-1,3,4-oxadiazol-2-yl)phenoxy)ethyl)piperidin-1-ium chloride (45d). Sinthesized according to General procedure Musing 44d (142 mg, 0.245 mmol) as reagent. Yield 100 mg (79%); white solid. ¹H NMR (400 MHz, DMSO-d₆): b 1.29-1.48 (m, 2H, CH₂), 1.94-1.73 (m, 5H, 2×CH₂, CH), 2.24 (s, 3H, CH₃), 2.83 (q, 2H, J=12.4 Hz, CH₂), 3.26 (d, 2H, J=12.3 Hz, CH₂), 4.25 (t, 2H, J=5.8 Hz, CH₂), 7.42 (d, 1H, J=1.9 Hz, ArH), 7.46 (dd, 1H, J=8.4, 1.8 Hz, ArH), 8.43 (d, 1H, J=11.4 Hz, Ha from NH₂), 8.53 (d, 1H, J=8.4 Hz, ArH), 8.69 (d, 1H, J=11.1 Hz, Hb from NH₂), 9.20 (s, 1H, NH), 12.50 (s, 1H, NH), 12.61 (s, 1H, NH) ppm. ¹³C NMR (100 MHz, DMSO-d₆): δ 10.18, 27.68, 29.52, 34.05, 42.48, 65.70, 107.22, 108.07, 109.27, 117.91, 117.99, 118.32, 118.38, 129.40, 129.61, 146.55, 152.99, 153.84, 155.70 ppm. HRMS (ESI) m/z for C₂₁H₂₄O₄N₅Cl₂ ([M+H]⁺): calculated 480.1205. found 480.1192. HPLC (10-90% ACN in 0.1% TFA in 11 min—UPLC): tᵣ 3.770 min (99,10% at 254 nm, 98,88% at 280 nm).

### (Example 30) 3,4-Dichloro-5-methyl-N-(4-(5-oxo-4,5-dihydro-1,3,4-oxadiazol-2-yl)-2-(pyridin-4-ylmethoxy)phenyl)-1H-pyrrole-2-carboxamide (44e)

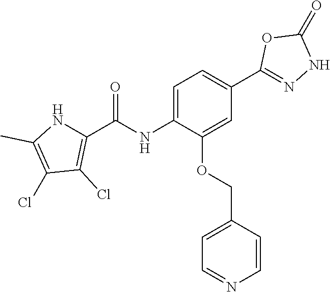 [SMILES: Cc1[nH]c(C(=O)Nc2ccc(-c3n[nH]c(=O)o3)cc2OCc2ccncc2)c(Cl)c1Cl]

Step 1: Synthesis of methyl 4-nitro-3-(pyridin-4-ylmethoxy)benzoate (40e). To a solution of methyl 3-hydroxy-4-nitrobenzoate (12, 2.00 g, 10.1 mmol) in acetonitrile (90 mL) K₂CO₃ (5.60 g, 40.6 mmol), 4-(chloromethyl)pyridine hydrochloride (1.83 g, 11.6 mmol) and KI (20%) were added and the reaction mixture was stirred at 60° C. overnight. The solvent was removed under reduced pressure to the residue ethyl acetate and water were added and the phases were separated. Organic phase was washed with brine, dried over Na₂SO₄, filtered and the solvent removed in vacuo. The crude product was resuspended in methanol, filtered off and dried. Yield 1.08 g (37%); beige solid. ¹H NMR (400 MHz, DMSO-d₆): δ 3.91 (s, 3H, CH₃), 5.50 (s, 2H, CH₂), 7.45 (d, 2H, J=5.5 Hz, 2×ArH), 7.72 (dd, 1H, J=8.4, 1.5 Hz, ArH), 7.87 (d, 1H, J=1.3 Hz, ArH), 8.08 (d, 1H, J=8.4 Hz, ArH), 8.62 (d, 2H, J=3.7 Hz, 2×ArH) ppm. MS (ESI⁺): 289.0 [M+H⁺].

Step 2: Synthesis of methyl-4-amino-3-(pyridin-4-ylmethoxy)benzoate (41e). Compound 40e (350 mg, 1.24 mmol) was dissolved in glacial acetic acid (15 mL). Iron powder (0.678 g, 12.4 mmol) was added and the mixture was stirred for 3 h at rt. Water (30 mL) was added to the mixture that was filtered over celite. The solution was neutralized with 100 mL of saturated solution of NaHCO₃ and the compound precipitated at pH 7. The precipitate was filtered off and washed with water. The solid was dissolved in ethyl acetate, organic phase was washed with water, dried over Na₂SO₄, filtered and the solvent removed in vacuo. Yield 233 mg (73%); light yellow solid. ¹H NMR (400 MHz, DMSO-d₆): δ 3.74 (s, 3H, CH₃), 5.22 (s, 2H, CH₂), 5.81 (s, 2H, NH₂), 6.69 (d, 1H, J=8.2 Hz, ArH), 7.36 (d, 1H, J=1.8 Hz, ArH), 7.41 (dd, 1H, J=8.2, 1.8 Hz, ArH), 7.49-7.59 (m, 2H, 2×ArH), 8.54-8.63 (m, 2H, 2×ArH) ppm.

Step 3: Synthesis of methyl 4-(3,4-dichloro-5-methyl-1H-pyrrole-2-carboxamido)-3-(pyridin-4-ylmethoxy)benzoate (42e). Sinthesized according to General procedure J using 41e (200 mg, 0.774 mmol) as reagent. Yield 159 mg (47%); white solid. ¹H NMR (400 MHz, DMSO-d₆): δ 2.22 (s, 3H, CH₃), 3.85 (s, 3H, CH₃), 5.37 (s, 2H, CH₂), 7.52-7.59 (m, 2H, 2×ArH), 7.67-7.71 (m, 2H, 2×ArH), 8.54 (d, 1H, J=8.9 Hz, ArH), 8.61-8.68 (m, 2H, 2×ArH), 9.24 (s, 1H, NH), 12.46 (s, 1H, NH) ppm.

Step 4: Synthesis of 3,4-dichloro-N-(4-(hydrazinecarbonyl)-2-(pyridin-4-ylmethoxy)phenyl)-5-methyl-1H-pyrrole-2-carboxamide (43e). Sinthesized according to General procedure K using 42e (130 mg, 0.299 mmol) as reagent. Yield 90 mg (69%); light brown solid. ¹H NMR (400 MHz, DMSO-d₆): δ 2.22 (s, 3H, CH₃), 4.49 (s, 2H, CH₂), 5.33 (s, 2H, NH₂), 7.50-7.59 (m, 3H, 3×ArH), 7.66 (d, 1H, J=1.8 Hz, ArH), 8.44 (d, 1H, J=8.5 Hz, ArH), 8.61-8.68 (m, 2H, 2×ArH), 9.20 (s, 1H, NH), 9.74 (s, 1H, NH) ppm.

Step 5: Synthesis of 3,4-dichloro-5-methyl-N-(4-(5-oxo-4,5-dihydro-1,3,4-oxadiazol-2-yl)-2-(pyridin-4-ylmethoxy)phenyl)-1H-pyrrole-2-carboxamide (44e). Sinthesized according to General procedure L using 43e (70 mg, 0.161 mmol) as reagent. Yield 50 mg (68%); light brown solid. ¹H NMR (400 MHz, DMSO-d₆): δ 2.22 (s, 3H, CH₃), 5.39 (s, 2H, CH₂), 7.46-7.59 (m, 4H, 4×ArH), 8.56 (d, 1H, J=8.5 Hz, ArH), 8.61-8.68 (m, 2H, 2×ArH), 9.21 (s, 1H, NH), 12.45 (s, 1H, NH), 12.58 (s, 1H, NH) ppm. HRMS (ESI) m/z for C₂₀H₁₆O₄N₅Cl₂ calculated 460.0574. found 460.0565. HPLC (10-90% ACN in 0.1% TFA in 11 min—UPLC) tᵣ 3.553 min (96.11% at 254 nm; 95.83% at 280 nm).

### (Example 31) 3,4-Dichloro-5-methyl-N-(4-(5-oxo-4,5-dihydro-1,3,4-oxadiazol-2-yl)-2-(pyrimidin-2-ylmethoxy)phenyl)-1H-pyrrole-2-carboxamide (44f)

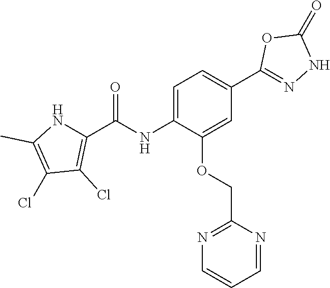 [SMILES: Cc1[nH]c(C(=O)Nc2ccc(-c3n[nH]c(=O)o3)cc2OCc2ncccn2)c(Cl)c1Cl]

Step 1: Synthesis of methyl 4-nitro-3-(pyrimidin-2-ylmethoxy)benzoate (40f). To a solution of methyl 3-hydroxy-4-nitrobenzoate (12, 326 mg, 1.68 mmol) in DMF (17 mL) 2-(chloromethyl)-pirimidine (360 mg, 2.18 mmol) and K₂CO₃ (464 mg, 3.36 mmol) were added and the solution was stirred at 65° C. overnight. The reaction mixture was evaporated, the solid was dissolved in ethyl acetate and the organic phase was washed with water and brine, dried over Na₂SO₄, filtered and the solvent removed in vacuo. Yield 337 mg (69%); yellow solid. ¹H NMR (400 MHz, DMSO-d₆): δ 3.86 (s, 3H, CH₃), 5.60 (s, 2H, CH₂), 7.49 (t, 1H, J=4.9 Hz, ArH), 7.67 (dd, 1H, J=8.3, 1.6 Hz, ArH), 7.77 (d, 1H, J=1.6 Hz, ArH), 8.01 (d, 1H, J=8.3 Hz, ArH), 8.84 (d, 2H, J=4.9 Hz, 2×ArH) ppm.

Step 2: Synthesis of methyl 4-amino-3-(pyrimidin-2-ylmethoxy)benzoate (41f). Sinthesized according to General procedure I using 40f (301 mg, 1.07 mmol) as reagent. Yield 203 mg (73%); light brown solid. ¹H NMR (400 MHz, DMSO-d₆): δ 3.71 (s, 3H, CH₃), 5.29 (s, 2H, CH₂), 5.72 (s, 2H, NH₂), 6.67 (d, 1H, J=8.3 Hz, ArH), 7.30 (d, 1H, J=1.7 Hz, ArH), 7.38 (dd, 1H, J=8.2, 1.8 Hz, ArH), 7.48 (t, 1H, J=4.9 Hz, ArH), 8.85 (d, 2H, J=4.9 Hz, 2×ArH) ppm.

Step 3: Synthesis of methyl 4-(3,4-dichloro-5-methyl-1H-pyrrole-2-carboxamido)-3-(pyrimidin-2-ylmethoxy)benzoate (42f). Sinthesized according to General procedure J using 41f (185 mg, 0.700 mmol) as reagent. Yield 112 mg (37%); light brown solid. ¹H NMR (400 MHz, DMSO-d₆): δ 2.23 (s, 3H, CH₃), 3.83 (s, 3H, CH₃), 5.51 (s, 2H, CH₂), 7.53 (t, 1H, J=4.9 Hz, ArH), 7.68-7.65 (m, 1H, ArH), 7.69 (d, 1H, J=1.7 Hz, ArH), 8.54 (d, 1H, J=8.9 Hz, ArH), 8.87 (d, 2H, J=4.9 Hz, 2×ArH), 9.46 (s, 1H, NH), 12.46 (s, 1H, NH) ppm.

Step 4: Synthesis of 3,4-dichloro-N-(4-(hydrazinecarbonyl)-2-(pyrimidin-2-ylmethoxy)phenyl)-5-methyl-1H-pyrrole-2-carboxamide (43f). Sinthesized according to General procedure K using 42f (100 mg, 0.230 mmol) as reagent. Yield 87 mg (88%); white solid. ¹H NMR (400 MHz, DMSO-d₆): δ 2.22 (s, 3H, CH₃), 4.46 (s, 2H, NH₂), 5.48 (s, 2H, CH₂), 7.48-7.57 (m, 2H, 2×ArH), 7.65 (d, 1H, J=1.6 Hz, ArH), 8.45 (d, 1H, J=8.5 Hz, ArH), 8.87 (d, 2H, J=4.9 Hz, 2×ArH), 9.39 (s, 1H, NH), 9.71 (s, 1H, NH) ppm.

Step 5: Synthesis of 3,4-dichloro-N-(2-isopropoxy-4-(5-oxo-4,5-dihydro-1,3,4-oxadiazol-2-yl)phenyl)-5-methyl-1H-pyrrole-2-carboxamide (44f). Sinthesized according to General procedure L using 43f (70 mg, 0.161 mmol) as reagent. Yield 10 mg (13%); white solid. ¹H NMR (400 MHz, DMSO-d₆): δ 2.23 (s, 3H, CH₃), 5.53 (s, 2H, CH₂), 7.48 (dd, 1H, J=8.5, 1.8 Hz, ArH), 7.50-7.57 (m, 2H, 2×ArH), 8.56 (d, 1H, J=8.5 Hz, ArH), 8.87 (d, 2H, J=4.9 Hz, 2×ArH), 9.42 (s, 1H, NH), 12.46 (s, 1H, NH), 12.55 (s, 1H, NH) ppm. HRMS (ESI) m/z for C₁₉H₁₅O₄N₆Cl₂ calculated 461.0526. found 461.0525. HPLC (10-90% ACN in 0.1% TFA in 11 min—UPLC): tᵣ 5.077 min (95.02% at 254 nm, 95.16% at 280 nm).

### (Example 32) 3,4-Dichloro-N-(2-isopropoxy-4-(5-oxo-4,5-dihydro-1,3,4-oxadiazol-2-yl)phenyl)-5-methyl-1H-pyrrole-2-carboxamide (44g)

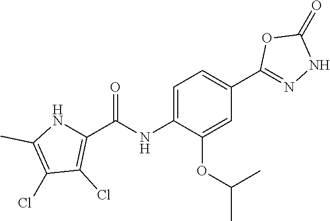 [SMILES: Cc1[nH]c(C(=O)Nc2ccc(-c3n[nH]c(=O)o3)cc2OC(C)C)c(Cl)c1Cl]

Step 1: Synthesis of methyl 3-isopropoxy-4-nitrobenzoate (40g). Sinthesized according to General procedure H using 2-propanol (366 mg, 6.08 mmol) as reagent. Yield 1.05 g (87%); yellow solid. ¹H NMR (400 MHz, DMSO-d₆): δ 1.30 (d, 6H, J=6.0 Hz, 2×CH₃), 3.90 (s, 3H, CH₃), 4.92 (hept, 1H, J=6.0 Hz, CH), 7.64 (dd, 1H, J=8.3, 1.6 Hz, ArH), 7.78 (d, 1H, J=1.5 Hz, ArH), 7.95 (d, 1H, J=8.3 Hz, ArH) ppm.

Step 2: Synthesis of methyl 4-amino-3-isopropoxybenzoate (41g). Sinthesized according to General procedure I using 40g (680 mg, 2.84 mmol) as reagent. Yield 348 mg (59%); white solid. ¹H NMR (400 MHz, DMSO-d₆): δ 1.28 (d, 6H, J=6.0 Hz, 2×CH₃), 3.74 (s, 3H, CH₃), 4.53 (hept, 1H, J=6.0 Hz, CH), 5.55 (s, 2H, NH₂), 6.65 (d, 1H, J=8.3 Hz, ArH), 7.30 (d, 1H, J=1.7 Hz, ArH), 7.37 (dd, 1H, J=8.2, 1.8 Hz, ArH) ppm.

Step 3: Synthesis of methyl 4-(3,4-dichloro-5-methyl-1H-pyrrole-2-carboxamido)-3-isopropoxybenzoate (42g) Sinthesized according to General procedure J using 41g (330 mg, 1.58 mmol) as reagent. Yield 238 mg (39%); white solid. ¹H NMR (400 MHz, DMSO-d₆): δ 1.36 (d, 6H, J=6.0 Hz, 2×CH₃), 2.24 (s, 3H, CH₃), 3.85 (s, 3H, CH₃), 4.86 (hept, 1H, J=6.0 Hz, CH), 7.58 (d, 1H, J=1.8 Hz, ArH), 7.63 (dd, 1H, J=8.5, 1.8 Hz, ArH), 8.52 (d, 1H, J=8.4 Hz, ArH), 9.31 (s, 1H, NH), 12.48 (s, 1H, NH) ppm.

Step 4: Synthesis of 3,4-dichloro-N-(4-(hydrazinecarbonyl)-2-isopropoxyphenyl)-5-methyl-1H-pyrrole-2-carboxamide (43g). Sinthesized according to General procedure K using 42g (220 mg, 0.574 mmol) as reagent. Yield 149 mg (67%); white solid. ¹H NMR (400 MHz, DMSO-d₆) δ 1.36 (d, 6H, J=6.0 Hz, 2×CH₃), 2.24 (s, 3H, CH₃), 4.50 (s, 2H, NH₂), 4.91-4.75 (m, 1H, CH), 7.48 (dd, 1H, J=8.5, 1.7 Hz, ArH), 7.56 (d, 1H, J=1.7 Hz, ArH), 8.43 (d, 1H, J=8.5 Hz, ArH), 9.22 (s, 1H, NH), 9.74 (s, 1H, NH), 12.45 (s, 1H, NH) ppm.

Step 5: Synthesis of 3,4-dichloro-N-(2-isopropoxy-4-(5-oxo-4,5-dihydro-1,3,4-oxadiazol-2-yl)phenyl)-5-methyl-1H-pyrrole-2-carboxamide (44g). Sinthesized according to General procedure L using 43g (80 mg, 0.208 mmol) as reagent. Yield 74 mg (87%); white solid. ¹H NMR (400 MHz, DMSO-d₆): δ 1.36 (d, 6H, J=6.0 Hz, 2×CH₃), 2.24 (s, 3H, CH₃), 4.89 (hept, 1H, J=6.0 Hz, CH), 7.39-7.46 (m, 2H, 2×ArH), 8.54 (d, 1H, J=8.3 Hz, ArH), 9.26 (s, 1H, NH), 12.47 (s, 1H, NH), 12.54 (s, 1H, NH) ppm. ¹³C NMR (100 MHz, DMSO-d₆): δ 11.25, 22.17, 71.99, 109.14, 109.44, 110.33, 118.86, 119.08, 119.30, 119.42, 130.41, 131.63, 146.19, 154.09, 154.94, 156.69 ppm. HRMS (ESI) m/z for C₁₇H₁₇O₄N₄C₂ calculated 411.0621. found 411.0616. HPLC (10-90% ACN in 0.1% TFA in 11 min—UPLC): tᵣ 6.577 min (99.37% at 254 nm; 98.77% at 280 nm).

### (Example 33) 3,4-Dichloro-5-methyl-N-(4-(5-oxo-4,5-dihydro-1,3,4-oxadiazol-2-yl)-2-(thiophen-2-ylmethoxy)phenyl)-1H-pyrrole-2-carboxamide (44h)

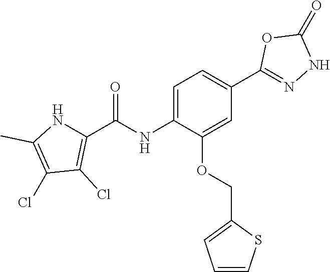 [SMILES: Cc1[nH]c(C(=O)Nc2ccc(-c3n[nH]c(=O)o3)cc2OCc2cccs2)c(Cl)c1Cl]

Step 1: Synthesis of methyl 4-nitro-3-(thiophen-3-ylmethoxy)benzoate (40h). Sinthesized according to General procedure H using thiophen-2-ylmethanol (1.46 g, 12.2 mmol) as reagent. Yield 1.59 g (54%); light yellow solid. ¹H NMR (400 MHz, DMSO-d₆): δ 3.91 (s, 3H, CH₃), 5.60 (s, 2H, CH₂), 7.06 (dd, 1H, J=5.1, 3.5 Hz, ArH), 7.24 (d, 1H, J=3.4 Hz, ArH), 7.60 (dd, 1H, J=5.1, 1.2 Hz, ArH), 7.69 (dd, 1H, J=8.4, 1.5 Hz, ArH), 7.96 (d, 1H, J=1.5 Hz, ArH), 8.00 (d, 1H, J=8.3 Hz, ArH) ppm.

Step 2: Synthesis of methyl 4-amino-3-(thiophen-2-ylmethoxy)benzoate (41h). Sinthesized according to General procedure/using 40h (600 mg, 2.05 mmol) as reagent. Yield 295 mg (55%); white solid. ¹H NMR (400 MHz, DMSO-d₆) δ 3.75 (s, 3H, CH₃), 5.32 (s, 2H, NH₂), 5.59 (s, 2H, CH₂), 6.67 (d, 1H, J=8.2 Hz, ArH), 7.05 (dd, 1H, J=5.1, 3.5 Hz, ArH), 7.19-7.27 (m, 1H, ArH), 7.40 (dd, 1H, J=8.2, 1.8 Hz, ArH), 7.44 (d, 1H, J=1.8 Hz, ArH), 7.56 (dd, 1H, J=5.1, 1.3 Hz, ArH) ppm.

Step 3: Synthesis of methyl 4-(3,4-dichloro-5-methyl-1H-pyrrole-2-carboxamido)-3-(thiophen-2-ylmethoxy)benzoate (42h). Sinthesized according to General procedure J using 41h (265 mg, 1.01 mmol) as reagent. Yield 300 mg (68%); light brown solid. ¹H NMR (400 MHz, DMSO-d₆): δ 2.21 (s, 3H, CH₃), 3.86 (s, 3H, CH₃), 5.51 (s, 2H, CH₂), 7.10 (ddd, 1H, J=5.8, 3.8, 2.1 Hz, ArH), 7.28-7.34 (m, 1H, ArH), 7.63-7.72 (m, 2H, 2×ArH), 7.76 (t, 1H, J=2.0 Hz, ArH), 8.53 (dd, 1H, J=8.5, 2.2 Hz, ArH), 9.24 (s, 1H, NH), 12.43 (s, 1H, NH) ppm.

Step 4: Synthesis of 3,4-dichloro-N-(4-(hydrazinecarbonyl)-2-(thiophen-2-ylmethoxy)phenyl)-5-methyl-1H-pyrrole-2-carboxamide (43h). Sinthesized according to General procedure K using 42h (270 mg, 0.614 mmol) as reagent. Yield 201 mg (75%); white solid. ¹H NMR (400 MHz, DMSO-d₆): δ 2.20 (s, 3H, CH₃), 4.50 (s, 2H, NH₂), 5.47 (s, 2H, CH₂), 7.09 (dd, 1H, J=5.1, 3.5 Hz, ArH), 7.32 (dd, 1H, J=3.5, 1.2 Hz, ArH), 7.52 (dd, 1H, J=8.4, 1.8 Hz, ArH), 7.66 (dd, 1H, J=5.1, 1.3 Hz, ArH), 7.74 (d, 1H, J=1.8 Hz, ArH), 8.43 (d, 1H, J=8.4 Hz, ArH), 9.16 (s, 1H, NH), 9.73 (s, 1H, NH), 12.26 (s, 1H, NH) ppm.

Step 5: Synthesis of 3,4-dichloro-5-methyl-N-(4-(5-oxo-4,5-dihydro-1,3,4-oxadiazol-2-yl)-2-(thiophen-2-ylmethoxy)phenyl)-1H-pyrrole-2-carboxamide (44h). Sinthesized according to General procedure L using 43h (100 mg, 0.228 mmol) as reagent. Yield 77 mg (73%); white solid. ¹H NMR (400 MHz, DMSO-d₆): δ 2.21 (s, 3H, CH₃), 5.53 (s, 2H, CH₂), 7.10 (dd, 1H, J=5.1, 3.5 Hz, ArH), 7.32 (dd, 1H, J=3.5, 1.2 Hz, ArH), 7.48 (dd, 1H, J=8.4, 1.8 Hz, ArH), 7.61 (d, 1H, J=1.8 Hz, ArH), 7.66 (dd, 1H, J=5.1, 1.3 Hz, ArH), 8.55 (d, 1H, J=8.4 Hz, ArH), 9.21 (s, 1H, NH), 12.43 (s, 1H, NH), 12.57 (s, 1H, NH) ppm. HRMS (ESI) m/z for C₁₉H₁₅O₄N₅Cl₂S: calculated 465.0186. found 465.0179 ([M+H]⁺). HPLC (10-90% ACN in 0.1% TFA in 11 min—UPLC): tᵣ 6.510 min (95.12% at 254 nm, 93.30% at 280 nm).

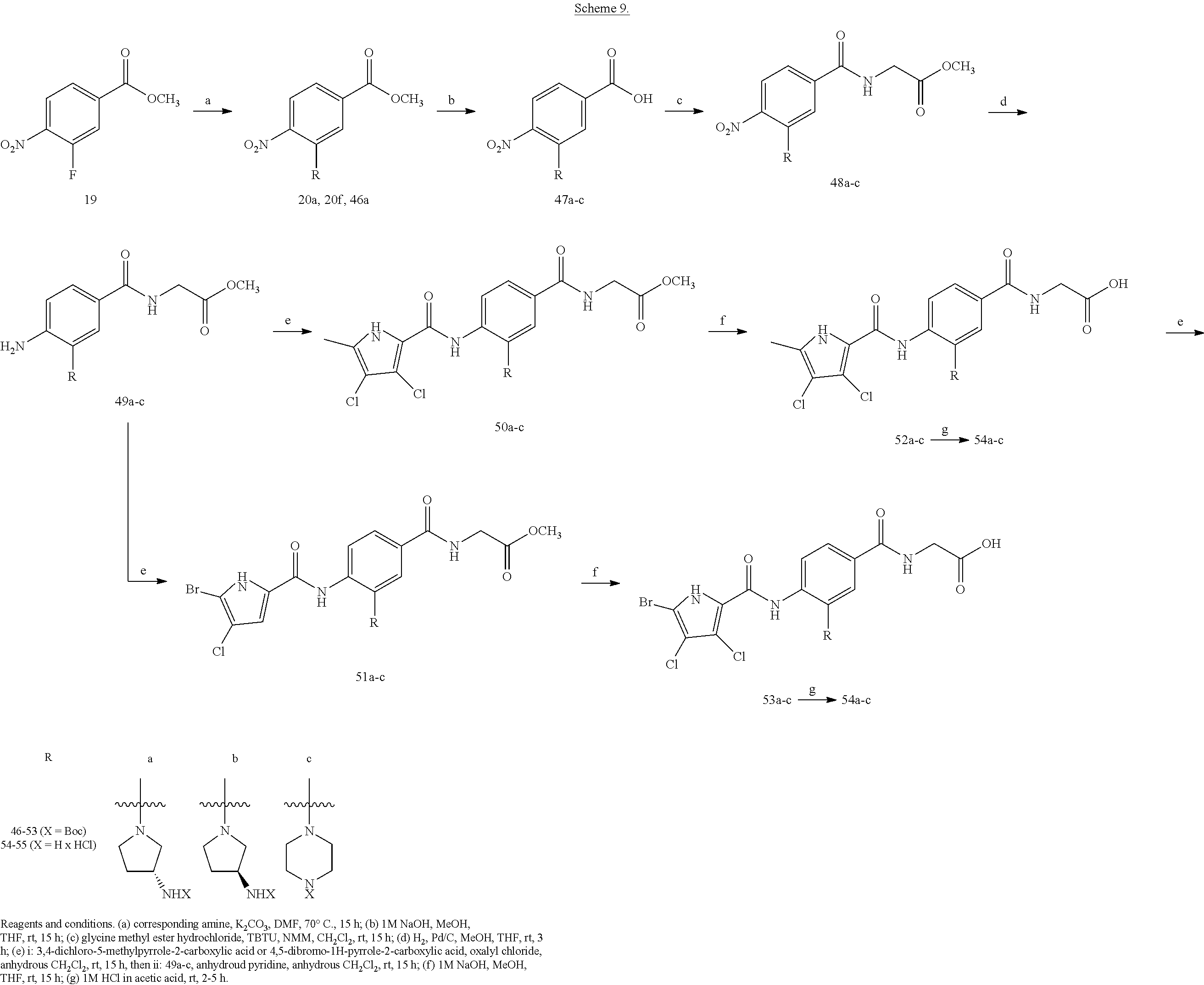

### (Example 34) (R)-1-(5-((Carboxymethyl)carbamoyl)-2-(3,4-dichloro-5-methyl-1H-pyrrole-2-carboxamido)phenyl)pyrrolidin-3-aminium chloride (54a)

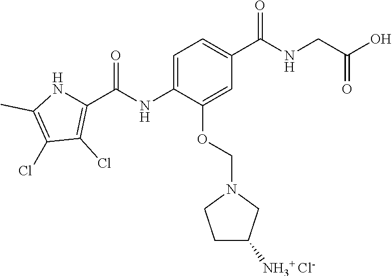

Step 1: Synthesis of methyl (R)-3-(3-((tert-butoxycarbonyl)amino)pyrrolidin-1-yl)-4-nitrobenzoate (46a). Synthesized according to General procedure A from 19 (1.07 g, 5.4 mmol), (3R)-3-aminopyrrolidine 3-Boc protected (1.00 g, 5.4 mmol) and K₂CO₃ (0.890 g, 6.4 mmol). The crude product was purified with flash column chromatography using ethyl acetate/petroleum ether (a1.64 g) as yellow solid. Yield 84% (1.64 g); yellow solid; mp 113-118′° C. ¹H NMR (400 MHz, D MSO-d₆): δ 1.47 (s, 9H, tBu), 1.97-2.07 (m, 1H, CH), 2.24-2.32 (m, 1H, CH), 3.11-3.15 (m, 1H, CH), 3.27-3.36 (m, 1H, CH), 3.47-3.53 (m, 2H, 2×CH), 3.96 (s, 3H, CH₃), 4.32-4.43 (m, 1H, CH/NH), 4.63-4.77 (m, 1H, CH/NH), 7.40 (dd, 1H, J=8.5, 1.6 Hz, Ar—H-6), 7.61 (d, J=1.6 Hz, 1H, Ar—H-2), 7.77 (d, 1H, J=8.5 Hz Ar—H-5) ppm. ¹³C NMR (100 MHz, DMSO-d₆): δ 28.15, 30.21, 47.95, 49.89, 52.65, 55.27, 77.95, 115.39, 117.14, 126.70, 133.26, 138.43, 141.59, 155.19, 165.31 ppm. [α]_{D}²⁵−4.2 (c 0.208, MeOH). MS (ESI) m/z=388.04 ([M+Na]⁺).

Step 2: Synthesis of (R)-3-(3-((tert-butoxycarbonyl)amino)pyrrolidin-1-yl)-4-nitrobenzoic acid (47a). To the solution of 46a (1.49 g, 4.0 mmol) in a mixture of methanol (50 mL) and tetrahydrofuran (10 mL) NaOH 1 M (8.00 mL, 8.0 mmol) was added and the reaction mixture was stirred overnight at rt. HCl 1 M was added dropwise until pH 7 and solvent was removed under reduce pressure. To the crude residue were added HCl 1 M until pH 4, water (100 mL) and ethyl acetate (100 mL). The phases were separated and organica phase was washed with water (100 mL) and brine (100 mL), dried over Na₂SO₄, filtered and evaporated, to obtain 47a (1.35 g) as orange crystals. Yield 1.35 g (94%); orange crystals; mp 158-162° C. ¹H NMR (400 MHz, DMSO-d₆): δ 1.38 (s, 9H, tBu), 1.87-1.95 (m, 1H, CH), 2.07-2.15 (m, 1H, CH), 2.86-2.90 (m, 1H, CH), 3.27-3.44 (m, 3H, 3×CH, overlapping with the signal for water), 4.08-4.12 (m, 1H, CH), 7.23-7.28 (m, 2H, Ar—H-6, NH), 7.52 (d, 1H, J=1.6 Hz, Ar—H-2), 7.81 (d, 1H, J=8.5 Hz Ar—H-5), 13.47 (br s, 1H, COOH) ppm. ¹³C NMR (100 MHz, DMSO-d₆): δ 28.15, 30.23, 47.91, 49.91, 55.26, 77.94, 115.67, 117.28, 126.53, 134.55, 138.29, 141.62, 155.20, 166.34 ppm. [α]_{D}²⁵−11.7 (c 0.180, MeOH). MS (ESI) m/z=374.01 ([M+Na]+).

Step 3: Synthesis of methyl (R)-(3-(3-((tert-butoxycarbonyl)amino)pyrrolidin-1-yl)-4-nitrobenzoyl)glycinate (48a) To the solution of 47a (1.26 g, 3.6 mmol) and TBTU (1.50 g, 4.7 mmol) in DMF (30 mL) NMM (1.19 mL, 10.8 mmol) was added and the solution was stirred for 30 min at rt. Glycine methyl ester hydrochloride (496 mg, 4.0 mmol) was added and reaction mixture was stirred at rt for 20 h. Solvent was removed under reduced pressure, the residue was dissolved in ethyl acetate (50 mL) and washed with water (2×20 mL), saturated solutions of NaHCO₃ (2×20 mL) and brine (20 mL), then organic phase was dried over Na₂SO₄, filtered and evaporated. The crude product was purified with flash column chromatography using ethyl acetate/petroleum ether (1:1) as mobile phase to obtain 48a as yellow solid (1.22 g). Yield 1.22 g (80%); yellow solid, mp 130-134° C. ¹H NMR (400 MHz, DMSO-d₆): δ 1.38 (s, 9H, tBu), 1.89-1.96 (m, 1H, CH), 2.06-2.15 (m, 1H, CH), 2.90-2.94 (m, 1H, CH), 3.24-3.36 (m, 3H, 3×CH, overlapping with the signal for water), 3.67 (s, 3H, CH₃), 4.04 (d, 2H, J=5.8 Hz, CH₂), 4.08-4.13 (m, 1H, CH), 7.20 (dd, 1H, J=8.5, 1.6 Hz, ArH-6), 7.26 (d, 1H, J=6.0 Hz, NH), 7.45 (d, 1H, J=1.6 Hz, ArH-2), 7.82 (d, 1H, J=8.5 Hz, ArH-5), 9.17 (t, 1H, J=5.8 Hz, NH) ppm. [α]_{D}²⁵−6.4 (c 0.220, MeOH). MS (ESI) m/z=444.99 ([M+Na]+).

Step 4: Synthesis of methyl (R)-(4-amino-3-(3-((tert-butoxycarbonyl)amino)pyrrolidin-1-yl)benzoyl)glycinate (49a) The solution ob 48a (1.20 g, 2.8 mmol) in methanol (40 mL) was stirred for 15 min under an argon atmosphere. Pd/C (239 mg) was added, the solution was saturated with hydrogen and the reaction mixture was stirred for 4 h under hydrogen atmosphere. The catalyst was filtered off and the solvent was evaporated. The crude product was purified with flash column chromatography using ethyl acetate/petroleum ether (3:1) as mobile phase, to obtain 49a (1.10 g) as pink crystals. Yield 1.10 g (99%); pink crystals; mp 77-80° C. ¹H NMR (400 MHz, DMSO-d₆): δ 1.39 (s, 9H, tBu), 1.65-1.73 (m, 1H, CH), 2.17-2.25 (m, 1H, CH), 2.77-2.85 (m, 2H, 2×CH), 3.06-3.17 (m, 2H, 2×CH), 3.63 (s, 3H, CH₃), 3.93 (d, 1H, J=5.8 Hz, CH₂), 4.06-4.15 (m, 1H, CH), 5.44 (br s, 2H, NH₂), 6.64 (d, 1H, J=8.3 Hz, ArH-3), 7.22 (d, 1H, J=7.9 Hz, NH), 7.36 (dd, 1H, J=8.3, 1.9 Hz, ArH-4), 7.39 (d, 1H, J=1.9 Hz, ArH), 8.52 (t, 1H, J=5.8 Hz, NH) ppm. [α]_{D}²⁵+18.7 (c 0.167, MeOH). MS (ESI) m/z=415.06 ([M+Na]+).

Step 5: Synthesis of methyl (R)-(3-(3-((tert-butoxycarbonyl)amino)pyrrolidin-1-yl)-4-(3,4-dichloro-5-methyl-1H-pyrrole-2-carboxamido)benzoyl)glycinate (50a). Synthesised according to General procedure E from 49a (450 mg, 1.1 mmol), 3,4-dichloro-5-methyl-1H-pyrrole-2-carboxylic acid (0.266 g, 1.4 mmol) and oxalyl chloride (0.47 mL, 5.5 mmol). Yield 470 mg (72%); grey solid; mp 204-208° C. ¹H NMR (400 MHz, DMSO-d₆): δ 1.39 (s, 9H, tBu), 1.81-1.89 (m, 1H, CH), 2.15-2.24 (m, 4H, CH, CH₃), 2.89-2.94 (m, 1H, CH), 3.00-3.06 (m, 1H, CH), 3.14-3.20 (m, 1H, CH), 3.29-3.33 (m, 1H, CH, overlapping with the signal for water), 3.67 (s, 3H, CH₃), 4.01 (d, 2H, J=5.6 Hz, CH₂), 4.05-4.13 (m, 1H, CH), 7.19 (d, 1H, J=6.4 Hz, NH), 7.63 (dd, 1H, J=8.5, 1.8 Hz, ArH-6), 7.76 (d, 1H, J=1.8 Hz, ArH-2), 8.27 (d, 1H, J=8.5 Hz, ArH-5), 8.94 (t, 1H, J=5.6 Hz, NH), 9.49 (s, 1H, NH), 12.38 (s, 1H, NH) ppm. [α]_{D}²⁵+14.2 (c 0.120, MeOH). MS (ESI) m/z=566.0 ([M−H]⁻). HRMS for C₂₅H₃₀Cl₂N₅O₆: calculated 566.1573. found 566.1576.

Step 6: Synthesis of (R)-(3-(3-((tert-butoxycarbonyl)amino)pyrrolidin-1-yl)-4-(3,4-dichloro-5-methyl-1H-pyrrole-2-carboxamido)benzoyl)glycine (52a). To the solution of 50a (200 mg, 0.35 mmol) in a mixture of methanol (8 mL) and tetrahydrofuran (3 mL) 1 M NaOH (1.41 mL, 1.4 mmol) was added and the mixture was stirred at rt for 15 h. To the mixture water (10 mL) was added and the pH was adjusted to 4 with 1 M HCl upon which the precipitate formed that was filtered off and dried to obtain 52a (91 mg) as grey solid. Yield 91 mg (47%); grey solid; mp 210-214° C. ¹H NMR (400 MHz, DMSO-d₆): δ 1.39 (s, 9H, tBu), 1.81-1.89 (m, 1H, CH), 2.15-2.24 (m, 4H, CH, CH₃), 2.89-2.94 (m, 1H, CH), 3.00-3.06 (m, 1H, CH), 3.14-3.19 (m, 1H, CH), 3.27-3.33 (m, 1H, CH, overlapping with the signal for water), 3.92 (d, 2H, J=5.8 Hz, CH₂), 4.07-4.14 (m, 1H, CH), 7.19 (d, 1H, J=6.5 Hz, NH), 7.63 (dd, 1H, J=8.5, 1.8 Hz, ArH-6), 7.76 (d, 1H, J=1.8 Hz, ArH-2), 8.27 (d, 1H, J=8.5 Hz, ArH-5), 8.81 (t, 1H, J=5.8 Hz, NH), 9.50 (s, 1H, NH), 12.38 (s, 1H, NH), 12.57 (br s, 1H, COOH) ppm. [α]_{D}²⁵+11.8 (c 0.187, MeOH). MS (ESI) m/z=552.1 ([M−H]⁻).

Step 7: Synthesis of (R)-1-(5-((carboxymethyl)carbamoyl)-2-(3,4-dichloro-5-methyl-1H-pyrrole-2-carboxamido)phenyl)pyrrolidin-3-aminium chloride (54a). Solution of 52a (35 mg, 0.063 mmol) in 4 M HCl in 1,4-dioxane (5 mL) was stirred at rt for 2 h. The solvent was removed, to the residue diethyl ether was added, the obtained suspension was sonicated and the undissolved solid was filtered off and dried to give 54a (22 mg) as grey solid. Yield 22 mg (71%); grey solid; mp 217-221° C. ¹H NMR (400 MHz, DMSO-d₆): δ 1.96-2.06 (m, 1H, CH), 2.24-2.34 (m, 4H, CH, CH₃), 2.95-3.00 (m, 1H, CH), 3.08-3.12 (m, 1H, CH), 3.36-3.42 (m, 1H, CH), 3.47-3.52 (m, 1H, CH, overlapping with the signal for water), 3.66-3.71 (m, 1H, CH, overlapping with the signal for water), 3.92 (d, 2H, J=5.8 Hz, CH₂), 7.65 (dd, 1H, J=8.5, 1.8 Hz, ArH-4), 7.79 (d, 1H, J=1.8 Hz, ArH-6), 8.17 (d, 1H, J=8.5 Hz, ArH-3), 8.43 (s, 3H, NH₃+), 8.92 (t, 1H, J=5.8 Hz, NH), 9.47 (s, 1H, NH), 12.6 (s, 1H, NH) ppm. Signal for COOH not seen. ¹³C NMR (100 MHz, DMSO-d₆): δ 10.75, 29.57, 41.18, 49.00, 50.28, 55.10, 108.73, 111.15, 118.70, 119.23, 120.96, 122.90, 129.07, 129.47, 134.78, 139.31, 156.69, 165.83, 171.35 ppm. [α]_{D}²⁵+11.0 (c 0.100, MeOH). MS (ESI) m/z=452.1 ([M−H]⁻). HRMS for C₁₉H₂₀Cl₂N₅O₄: calculated 452.0892. found 452.0898. HPLC: Agilent Eclipse Plus C18 column (5 μm, 4.6×150 mm); mobile phase: 20-40% of acetonitrile in phosphate buffer (pH=6.8) in 16 min, 40% acetonitrile to 20 min; flow rate 1.0 mL/min; injection volume: 10 μL; t_{R}: 9.252 min (99.4% at 280 nm).

### (Example 35) (S)-1-(5-((Carboxymethyl)carbamoyl)-2-(3,4-dichloro-5-methyl-1H-pyrrole-2-carboxamido)phenyl)pyrrolidin-3-aminium chloride (54b)

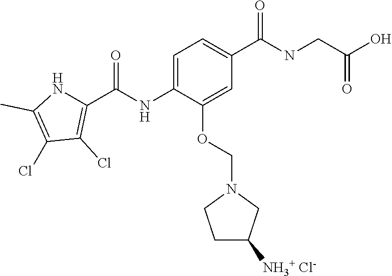

Step 1: Synthesis of (S)-3-(3-((tert-butoxycarbonyl)amino)pyrrolidin-1-yl)-4-nitrobenzoic acid (47b). To the solution of 20f (6.90 g, 18.9 mmol) in a mixture of methanol (200 mL) and tetrahydrofuran (40 mL) NaOH 1 M (28.4 mL, 28 mmol) was added and the reaction mixture was stirred overnight at rt. HCl 1 M was added dropwise until pH 7 and solvent was removed under reduce pressure. To the crude residue were added HCl 1 M until pH 4, water (100 mL) and ethyl acetate (100 mL). The phases were separated and organica phase was washed with water (50 mL) and brine (50 mL), dried over Na₂SO₄, filtered and evaporated, to obtain 47b (6.61 g) as orange crystals. Yield 6.61 g (99%); orange crystals; mp 153-163° C. ¹H NMR (400 MHz, DMSO-d₆): δ 1.38 (s, 9H, tBu), 1.87-1.95 (m, 1H, CH), 2.07-2.15 (m, 1H, CH), 2.86-2.90 (m, 1H, CH), 3.27-3.44 (m, 3H, 3×CH, overlapping with the signal for water), 4.08-4.12 (m, 1H, CH), 7.23-7.28 (m, 2H, ArH-6, NH), 7.52 (d, 1H, J=1.6 Hz, ArH-2), 7.81 (d, 1H, J=8.5 Hz ArH-5), 13.47 (br s, 1H, COOH) ppm. [α]_{D}²⁵+12.8 (c 0.133, MeOH). MS (ESI) m/z=374.03 ([M+Na]+).

Step 2: Synthesis of methyl (S)-(3-(3-((tert-butoxycarbonyl)amino)pyrrolidin-1-yl)-4-nitrobenzoyl)glycinate (48b). To the solution of 47b (3.00 g, 8.5 mmol) and TBTU (3.57 g, 11.1 mmol) in DMF (100 mL) NMM (2.82 mL, 25.6 mmol) was added and the solution was stirred for 30 min at rt. Glycine methyl ester hydrochloride (1.18 g, 9.4 mmol) was added and reaction mixture was stirred at rt for 20 h. Solvent was removed under reduced pressure, the residue was dissolved in ethyl acetate (50 mL) and washed with water (2×20 mL), saturated solutions of NaHCO₃ (2×20 mL) and brine (20 mL), then organic phase was dried over Na₂SO₄, filtered and evaporated. The crude product was recrystallized from ethyl acetate. The solvent of the mother liquor was evaporated and the residue was recrystallized from ethanol. The pure products were combined to obtain 48b as yellow solid (2.98 g). Yield 2.98 g (84%); yellow solid, mp 133-138° C. ¹H NMR (400 MHz, DMSO-d₆): δ 1.38 (s, 9H, tBu), 1.89-1.96 (m, 1H, CH), 2.06-2.15 (m, 1H, CH), 2.90-2.94 (m, 1H, CH), 3.24-3.36 (m, 3H, 3×CH, overlapping with the signal for water), 3.67 (s, 3H, CH₃), 4.04 (d, 2H, J=5.8 Hz, CH₂), 4.08-4.13 (m, 1H, CH), 7.20 (dd, 1H, J=8.5, 1.6 Hz, ArH-6), 7.26 (d, 1H, J=6.0 Hz, NH), 7.45 (d, 1H, J=1.6 Hz, ArH-2), 7.82 (d, 1H, J=8.5 Hz, ArH-5), 9.17 (t, 1H, J=5.8 Hz, NH) ppm. [α]_{D}²⁵+5.3 (c 0.133, MeOH). MS (ESI) m/z=445.00 ([M+Na]+).

Step 3: Synthesis of methyl (S)-(4-amino-3-(3-((tert-butoxycarbonyl)amino)pyrrolidin-1-yl)benzoyl)glycinate (49b). The solution ob 48b (2.41 g, 5.7 mmol) in methanol (90 mL) was stirred for 15 min under an argon atmosphere. Pd/C (483 mg) was added, the solution was saturated with hydrogen and the reaction mixture was stirred for 5 h under hydrogen atmosphere. The catalyst was filtered off and the solvent was evaporated. The crude product was purified with flash column chromatography using ethyl acetate/petroleum ether (2:1) as mobile phase, to obtain 49b (2.23 g) as grey crystals. Yield 2.23 g (99%); grey crystals; mp 78-81° C. ¹H NMR (400 MHz, DMSO-d₆): δ 1.39 (s, 9H, tBu), 1.65-1.73 (m, 1H, CH), 2.17-2.25 (m, 1H, CH), 2.77-2.85 (m, 2H, 2×CH), 3.06-3.17 (m, 2H, 2×CH), 3.63 (s, 3H, CH₃), 3.93 (d, 1H, J=5.8 Hz, CH₂), 4.06-4.15 (m, 1H, CH), 4.91-5.80 (br s, 2H, NH₂), 6.64 (d, 1H, J=8.3 Hz, ArH-5), 7.22 (d, 1H, J=7.9 Hz, NH), 7.36 (dd, 1H, J=8.3, 1.9 Hz, ArH-6), 7.39 (d, 1H, J=1.9 Hz, ArH-2), 8.52 (t, 1H, J=5.8 Hz, NH) ppm. [α]_{D}²⁵−18.3 (c 0.131, MeOH). MS (ESI) m/z=415.05 ([M+Na]+).

Step 4: Synthesis of methyl (S)-(3-(3-((tert-butoxycarbonyl)amino)pyrrolidin-1-yl)-4-(3,4-dichloro-5-methyl-1H-pyrrole-2-carboxamido)benzoyl)glycinate (50b). Synthesised according to General procedure E from 49b (300 mg, 0.76 mmol), 3,4-dichloro-5-methyl-1H-pyrrole-2-carboxylic acid (178 mg, 0.92 mmol) and oxalyl chloride (0.31 mL, 3.7 mmol). The crude product was triturated with diethyl ether and the undissolved solid was filtered off to give 50b as beige solid (245 mg). Yield 245 mg (57%); beige solid; mp 206-209° C. ¹H NMR (400 MHz, DMSO-d₆): δ 1.39 (s, 9H, tBu), 1.81-1.89 (m, 1H, CH), 2.15-2.24 (m, 4H, CH, CH₃), 2.89-2.94 (m, 1H, CH), 3.00-3.06 (m, 1H, CH), 3.14-3.20 (m, 1H, CH), 3.29-3.33 (m, 1H, CH, overlapping with the signal for water), 3.67 (s, 3H, CH₃), 4.01 (d, 2H, J=5.6 Hz, CH₂), 4.05-4.13 (m, 1H, CH), 7.19 (d, 1H, J=6.4 Hz, NH), 7.63 (dd, 1H, J=8.5, 1.8 Hz, ArH-6), 7.76 (d, 1H, J=1.8 Hz, ArH-2), 8.27 (d, 1H, J=8.5 Hz, ArH-5), 8.94 (t, 1H, J=5.6 Hz, NH), 9.49 (s, 1H, NH), 12.38 (s, 1H, NH) ppm. [α]_{D}²⁵−15.7 (c 0.153, MeOH). MS (ESI) m/z=566.0 ([M−H]⁻). HRMS for C₂₅H₃₀Cl₂N₅O₆: calculated 566.1573. found 566.1573.

Step 5: Synthesis of (S)-(3-(3-((tert-butoxycarbonyl)amino)pyrrolidin-1-yl)-4-(3,4-dichloro-5-methyl-1H-pyrrole-2-carboxamido)benzoyl)glycine (52b). To the solution of 50b (155 mg, 0.27 mmol) in a mixture of methanol (10 mL) and tetrahydrofuran (2 mL) 1 M NaOH (1.09 mL, 1.09 mmol) was added and the mixture was stirred at rt for 15 h. To the mixture 1M HCl was added to reach pH 7 and methanol was removed under reduced pressure. 1M HCl was added to the aqueous residue to reach pH 4 upon which ethyl acetate (15 mL) was added. The undissolved precipitate was filtered off and dried to give 52b (81 mg). Mother liquid was poured into a separating funnel and the two phases were separated. Organic phase was washed with brine (2×10 mL), dried with Na₂SO₄, filtered and evaporated. Diethyl ether was added to the residue, the obtained suspension was sonicated and the precipitate filtered off and dried (26 mg). The pure products were combined to obtain 52b (107 mg) as brown crystals. Yield 107 mg (71%); brown crystals; mp 210-215° C. ¹H NMR (400 MHz, DMSO-d₆): δ 1.39 (s, 9H, tBu), 1.81-1.89 (m, 1H, CH), 2.15-2.24 (m, 4H, CH, CH₃), 2.89-2.94 (m, 1H, CH), 3.00-3.06 (m, 1H, CH), 3.14-3.19 (m, 1H, CH), 3.27-3.33 (m, 1H, CH, overlapping with the signal for water), 3.92 (d, 2H, J=5.8 Hz, CH₂), 4.07-4.14 (m, 1H, CH), 7.19 (d, 1H, J=6.5 Hz, NH), 7.63 (dd, 1H, J=8.5, 1.8 Hz, Ar—H-6), 7.76 (d, 1H, J=1.8 Hz, Ar—H-2), 8.27 (d, 1H, J=8.5 Hz, Ar—H-5), 8.82 (t, 1H, J=5.8 Hz, NH), 9.50 (s, 1H, NH), 12.38 (s, 1H, NH), 12.57 (br s, 1H, COOH) ppm. [α]_{D}²⁵−12.0 (c 0.125, MeOH). MS (ESI) m/z=552.0 ([M−H]⁻). HRMS for C₂₄H₂₈Cl₂N₅O₆: calculated 552.1417. found 552.1412.

Step 6: Synthesis of (S)-1-(5-((carboxymethyl)carbamoyl)-2-(3,4-dichloro-5-methyl-1H-pyrrole-2-carboxamido)phenyl)pyrrolidin-3-aminium chloride (54b). The solution of 52b (40 mg, 0.072 mmol) in a mixture of 4 M HCl in 1,4-dioxane (1 mL) and tetrahidrofuran (1 mL) was stirred at rt for 2 h. The solvent was removed, to the residue diethyl ether was added, the obtained suspension was sonicated and the undissolved solid was filtered off and dried to give 54b (34 mg) as beige solid. Yield 24 mg (97%); beige solid; mp 216-220° C. ¹H NMR (400 MHz, DMSO-d₆): δ 1.96-2.06 (m, 1H, CH), 2.24-2.34 (m, 4H, CH, CH₃), 2.95-3.00 (m, 1H, CH), 3.08-3.12 (m, 1H, CH), 3.36-3.42 (m, 1H, CH), 3.47-3.52 (m, 1H, CH, overlapping with the signal for water), 3.66-3.71 (m, 1H, CH, overlapping with the signal for water), 3.92 (d, 2H, J=5.8 Hz, CH₂), 7.65 (dd, 1H, J=8.5, 1.8 Hz, ArH-4), 7.79 (d, 1H, J=1.8 Hz, ArH-6), 8.17 (d, 1H, J=8.5 Hz, ArH-3), 8.43 (s, 3H, NH₃+), 8.92 (t, 1H, J=5.8 Hz, NH), 9.47 (s, 1H, NH), 12.6 (s, 1H, NH) ppm. Signal for COOH not seen. ¹³C NMR (100 MHz, DMSO-d₆): δ 10.75, 29.57, 41.18, 49.00, 50.28, 55.10, 108.73, 111.15, 118.70, 119.23, 120.96, 122.90, 129.07, 129.47, 134.78, 139.31, 156.69, 165.83, 171.35 ppm. [α]_{D}²⁵−11.9 (c 0.117, MeOH). MS (ESI) m/z=452.0 ([M−H]⁻). HRMS for C₁₉H₂₀Cl₂N₅O₄: calculated 452.0892. found 452.0881. HRMS for C₁₉H₂₀N₅O₄Cl₂: calculated 452.0892. found 452.0881 HPLC: Agilent Eclipse Plus C18 column (5 μm, 4.6×150 mm); mobile phase: 20-40% of acetonitrile in phosphate buffer (pH=6.8) in 16 min, 40% acetonitrile to 20 min; flow rate 1.0 mL/min; injection volume: 10 μL; t_{R}: 9.252 min (99.3% at 280 nm).

### (Example 36) 4-(5-((Carboxymethyl)carbamoyl)-2-(3,4-dichloro-5-methyl-1H-pyrrole-2-carboxamido)phenyl)piperazin-1-ium chloride (54c)

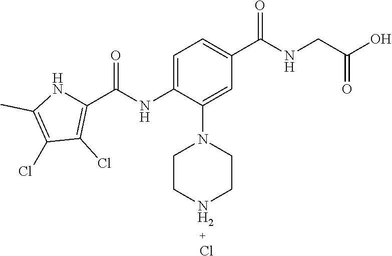 [SMILES: Cc1[nH]c(C(=O)Nc2ccc(C(=O)NCC(=O)O)cc2N2CCNCC2)c(Cl)c1Cl ; Cl]

Step 1: Synthesis of 3-(4-(tert-butoxycarbonyl)piperazin-1-yl)-4-nitrobenzoic acid (47c). To the solution of 20c (200 mg, 0.55 mmol) in methanol (10 mL) NaOH 1 M (0.81 mL, 0.82 mmol) was added and the reaction mixture was stirred overnight at rt. HCl 1 M was added dropwise until pH 7 and solvent was removed under reduce pressure. To the crude residue were added HCl 1 M until pH 4, water (20 mL) and ethyl acetate (25 mL). The phases were separated and organica phase was washed with water (20 mL) and brine (20 mL), dried over Na₂SO₄, filtered and evaporated, to obtain 171 mg of product as orange crystals. Yield 171 mg (89%); orange crystals; mp 170-175° C. ¹H NMR (400 MHz, DMSO-d₆): δ 1.42 (s, 9H, tBu), 2.93-2.95 (m, 4H, 2×CH₂), 3.41-3.47 (m, 4H, 2×CH₂), 7.58 (dd, 1H, J=6.2, 1.4 Hz, ArH-6), 7.73-7.75 (m, 2H, ArH-2, ArH-5) ppm. MS (ESI) m/z=350.13 ([M−H]⁻).

Step 2: Synthesis of tert-butyl 4-(5-((2-methoxy-2-oxoethyl)carbamoyl)-2-nitrophenyl)piperazine-1-carboxylate (48c). To the solution of 47c (0.900 g, 2.4 mmol) and TBTU (1.01 g, 3.1 mmol) in DMF (20 mL) NMM (0.53 mL, 4.8 mmol) was added and the solution was stirred for 30 min at rt. Glycine methyl ester hydrochloride (333 mg, 2.7 mmol) was added and reaction mixture was stirred at rt for 20 h. Solvent was removed under reduced pressure, the residue was dissolved in ethyl acetate (20 mL) and washed with water (2×20 mL), saturated solutions of NaHCO₃ (2×20 mL) and brine (20 mL), then organic phase was dried over Na₂SO₄, filtered and evaporated. The crude product was purified with flash column chromatography using ethyl acetate/petroleum ether 1:1 as mobile phase to obtain 48c as orange oil (0.852 g). Yield 0.852 g (84%); orange oil. ¹H NMR (400 MHz, DMSO-d₆): δ 1.42 (s, 9H, tBu), 3.00-3.03 (m, 4H, 2×CH₂), 3.43-3.48 (m, 4H, 2×CH₂, overlapped with the signal of water), 3.67 (s, 3H, CH₃), 4.05 (d, 2H, J=5.8 Hz, CH₂), 7.59 (dd, 1H, J=8.5, 1.7 Hz, ArH-6), 7.76 (d, 1H, J=1.7 Hz, ArH-2), 7.94 (d, 1H, J=8.5 Hz, ArH-5), 9.22 (t, 1H, J=5.8 Hz, NH) ppm. MS (ESI) m/z=445.01 ([M+Na]+).

Step 3: Synthesis of tert-butyl 4-(2-amino-5-((2-methoxy-2-oxoethyl)carbamoyl)phenyl)piperazine-1-carboxylate (49c). The solution ob 48c (2.51 g, 5.9 mmol) in methanol (90 mL) was stirred for 15 min under an argon atmosphere. Pd/C (0.500 g) was added, the solution was saturated with hydrogen and the reaction mixture was stirred for 4 h under hydrogen atmosphere. The catalyst was filtered off and the solvent was evaporated. The crude product was purified with flash column chromatography using ethyl acetate/petroleum ether 2:1 as mobile phase, to obtain 2.08 g of 49c as white crystals. Yield 2.08 g (90%); white crystals; mp 80-84° C. ¹H NMR (400 MHz, DMSO-d₆): δ 1.49 (s, 9H, tBu), 2.75-2.85 (m, 4H, 2×CH₂), 3.51-3.63 (m, 4H, 2×CH₂, overlapped with the signal of water), 3.69 (s, 3H, CH₃), 3.99 (d, 2H, J=5.8 Hz, CH₂), 4.58-6.07 (br s, 2H, NH₂), 6.74 (d, 1H, J=8.4 Hz, ArH-3), 7.48 (dd, 1H, J=8.4, 1.9 Hz, ArH-4), 7.53 (d, 1H, J=1.9 Hz, ArH-6), 8.59 (t, 1H, J=5.8 Hz, NH) ppm. MS (ESI) m/z=415.07 ([M+Na]+).

Step 4: Synthesis of tert-butyl 4-(2-(3,4-dichloro-5-methyl-1H-pyrrole-2-carboxamido)-5-((2-methoxy-2-oxoethyl)carbamoyl)phenyl)piperazine-1-carboxylate (50c). Synthesised according to General procedure E from 49c (0.300 g, 0.76 mmol), 3,4-dichloro-5-methyl-1H-pyrrole-2-carboxylic acid (0.178 g, 0.92 mmol) and oxalyl chloride (0.31 mL, 3.7 mmol). Yield 246 mg (57%); grey solid; mp 194-197° C. ¹H NMR (400 MHz, DMSO-d₆): δ 1.44 (s, 9H, tBu), 2.24 (s, 3H, CH₃), 2.82-2.85 (m, 4H, 2×CH₂), 3.50-3.57 (m, 4H, 2×CH₂), 3.67 (s, 3H, CH₃), 4.02 (d, 2H, J=5.7 Hz, CH₂), 7.75 (dd, 1H, J=8.6, 1.9 Hz, ArH-4), 7.91 (d, 1H, J=1.9 Hz, ArH-6), 8.48 (d, 1H, J=8.4 Hz, ArH-3), 8.93 (t, 1H, J=5.7 Hz, NH), 9.79 (s, 1H, NH), 12.43 (s, 1H, NH) ppm. MS (ESI) m/z=566 ([M−H]⁻).

Step 5: Synthesis of (3-(4-(tert-butoxycarbonyl)piperazin-1-yl)-4-(3,4-dichloro-5-methyl-1H-pyrrole-2-carboxamido)benzoyl)glycine (52c). To the solution of 50c (0.160 g, 0.28 mmol) in methanol (10 mL) 1 M NaOH (0.84 mL, 0.84 mmol) was added and the mixture was stirred at rt for 15 h. The mixture was neutralized with 1 M HCl and methanol was removed under reduced pressure. The pH was adjusted to 4 with 1 M HCl, ethyl acetate was added and the precipitate that formed was filtered off and dried to obtain 51c as grey solid. Yield 138 mg (89%); grey solid; mp>300° C. ¹H NMR (400 MHz, DMSO-d₆): δ 1.44 (s, 9H, tBu), 2.24 (s, 3H, CH₃), 2.82-2.85 (m, 4H, 2×CH₂), 3.50-3.58 (m, 4H, 2×CH₂), 3.93 (d, 2H, J=5.7 Hz, CH₂), 7.75 (dd, 1H, J=8.6, 1.9 Hz, ArH-6), 7.91 (d, 1H, J=1.9 Hz, ArH-2), 8.48 (d, 1H, J=8.6 Hz, ArH-5), 8.81 (t, 1H, J=5.7 Hz, NH), 9.79 (s, 1H, NH), 12.43 (s, 1H, NH), 12.6 (br s, 1H, COOH) ppm. MS (ESI) m/z=552.0 ([M−H]⁻).

Step 6: Synthesis of 4-(5-((carboxymethyl)carbamoyl)-2-(3,4-dichloro-5-methyl-1H-pyrrole-2-carboxamido)phenyl)piperazin-1-ium chloride (54c). Compound 52c (50 mg, 0.09 mmol) was suspended in 4 M HCl in 1,4-dioxane (5 mL) and THE (2 mL), and the mixture was stirred at rt for 1 h. The solvents were evaporated, to the solid residue diethyl ether was added, the obtained suspension was sonicated and the solid was filtered off to give 54c (39 mg) as grey solid. Yield 39 mg (89%); grey solid; mp 262-265° C. ¹H NMR (400 MHz, DMSO-d₆): δ 2.24 (s, 3H, CH₃), 3.09-3.12 (m, 4H, 2×CH₂), 3.24-3.31 (m, 4H, 2×CH₂), 3.93 (d, 2H, J=5.7 Hz, CH₂), 7.79 (dd, 1H, J=8.6, 1.8 Hz, ArH-4), 7.83 (d, 1H, J=1.8 Hz, ArH-6), 8.45 (d, 1H, J=8.6 Hz, ArH-3), 8.96 (t, 1H, J=5.7 Hz, NH), 9.23 (br s, 2H, NH₂+), 9.59 (s, 1H, NH), 12.49 (s, 1H, NH), 12.59 (br s, 1H, COOH) ppm. ¹³C NMR (100 MHz, DMSO-d₆): δ 10.75, 41.17, 43.42, 48.85, 108.62, 109.99, 118.81, 118.86, 120.70, 125.27, 128.85, 129.74, 136.35, 140.31, 156.60, 165.54, 171.37 ppm. MS (ESI) m/z=452.0 ([M−H]⁻). HRMS for C₁₉H₂₀Cl₂N₅O₄: calculated 452.0892. found 452.0891. HPLC: Agilent Eclipse Plus C18 column (5 μm, 4.6×150 mm); mobile phase: 20-40% of acetonitrile in phosphate buffer (pH=6.8) in 16 min, 40% acetonitrile to 20 min; flow rate 1.0 mL/min; injection volume: 10 μL; t_{R}: 9.087 min (99.6% at 280 nm).

### (Example 37) (R)-1-(5-((Carboxymethyl)carbamoyl)-2-(4,5-dibromo-1H-pyrrole-2-carboxamido)phenyl)pyrrolidin-3-aminium chloride (55a)

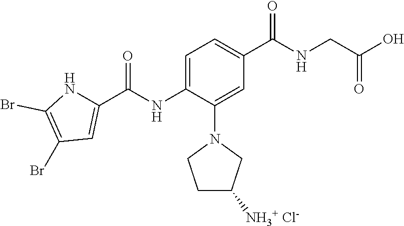

Step 1: Synthesis of methyl (R)-(3-(3-((tert-butoxycarbonyl)amino)pyrrolidin-1-yl)-4-(4,5-dibromo-1H-pyrrole-2-carboxamido)benzoyl)glycinate (51a). Synthesised according to General procedure E from 49a (0.500 g, 1.3 mmol), 4,5-dibromo-1H-pyrrole-2 carboxylic acid (411 mg, 1.5 mmol) and oxalyl chloride (0.52 mL, 6.1 mmol). The precipitate that was formed during the extraction was filtered off to obtain crude product 1. The two phases of the mother liquor were separated and organic phase was washed with water (20 mL), saturated solution of NaHCO₃ (2×20 mL) and brine (2×20 mL), dried over Na₂SO₄, filtered and the solvent evaporated to obtain crude product 2. The combined crude products were triturated with diethyl ether and the undissolved solid was filtered off and dried to give 51a (778 mg) as grey solid. Yield 778 mg (93%); brown solid; mp 203-208° C. ¹H NMR (400 MHz, DMSO-d₆): δ 1.36 (s, 9H, tBu), 1.75-1.85 (m, 1H, CH), 2.03-2.10 (m, 1H, CH), 3.04-3.07 (m, 1H, CH), 3.21-3.27 (m, 1H, CH), 3.31-3.45 (m, 2H, 2×CH, overlapping with the signal for water), 3.66 (s, 3H, CH₃), 3.99-4.06 (m, 3H, CH, CH₂), 7.14-7.17 (m, 2H, NH, ArH), 7.29-7.35 (m, 3H, 3×ArH), 8.94 (t, 1H, J=5.8 Hz, NH), 9.53 (s, 1H, NH), 12.89 (d, 1H, J=2.7 Hz, NH) ppm. [α]_{D}²⁵+25.0 (c 0.120, MeOH). MS (ESI) m/z=640.0 ([M−H]⁻). HRMS for C₂₄H₂₈Br₂N₅O₆: calculated 640.0406. found 640.0403.

Step 2: Synthesis of (R)-(3-(3-((tert-butoxycarbonyl)amino)pyrrolidin-1-yl)-4-(4,5-dibromo-1H-pyrrole-2-carboxamido)benzoyl)glycine (53a). To the solution of 51a (300 mg, 0.47 mmol) in a mixture of methanol (10 mL) and tetrahydrofuran (1 mL) 1 M NaOH (1.86 mL, 1.8 mmol) was added and the mixture was stirred at rt for 15 h. The mixture was neutralized with 1 M HCl and methanol was removed under reduced pressure. Water (10 mL) was added and the pH was adjusted to 4 with 1 M HCl. The parcipitate was filtered off and dried to obtain 53a (234 mg) as grey solid. Yield 234 mg (80%); grey solid; mp 218-223° C. ¹H NMR (400 MHz, DMSO-d₆): δ 1.37 (s, 9H, tBu), 1.75-1.84 (m, 1H, CH), 2.03-2.10 (m, 1H, CH), 3.04-3.07 (m, 1H, CH), 3.20-3.26 (m, 1H, CH), 3.39-3.44 (m, 2H, 2×CH, overlapping with the signal for water), 3.91 (d, 2H, J=5.8 Hz, CH₂), 4.01-4.06 (m, 1H, CH), 7.13-7.20 (m, 2H, NH, ArH), 7.29-7.35 (m, 3H, 3×ArH), 8.80 (t, 1H, J=5.8 Hz, NH), 9.52 (s, 1H, NH), 12.47 (br s, 1H, COOH), 12.89 (s, 1H, NH) ppm. [α]_{D}²⁵+35.3 (c 0.133, MeOH). MS (ESI) m/z=626.0 ([M−H]⁻). HRMS for C₂₃H₂₆Br₂N₅O₆: calculated 626.0250. found 626.0252.

Step 3: Synthesis of (R)-1-(5-((carboxymethyl)carbamoyl)-2-(4,5-dibromo-1H-pyrrole-2-carboxamido)phenyl)pyrrolidin-3-aminium chloride (55a). Solution of 53a (70 mg, 0.11 mmol) in a mixture of 1,4-dioxane (10 mL) and 4 M HCl in 1,4-dioxane (5 mL) was stirred at rt for 2 h. The solvent was removed under reduced pressure, to the residue diethyl ether was added, the obtained suspension was sonicated, the percipitate was filtered off, washed with diethyl ether and dried to obtain 55a (62 mg) as grey solid. Yield 62 mg (98%); grey solid; mp 209-214° C. ¹H NMR (400 MHz, DMSO-d₆): δ 1.94-2.04 (m, 1H, CH), 2.20-2.29 (m, 1H, CH), 3.01-3.07 (m, 1H, CH), 3.20-3.23 (m, 1H, CH), 3.49-3.53 (m, 2H, 2×CH), 3.74-3.94 (m, 3H, CH, CH₂ overlapping with the signal for water), 7.36 (d, 1H, J=2.4 Hz, ArH), 7.41-7.43 (m, 2H, 2×ArH), 7.50-7.52 (m, 1H, ArH), 8.22 (s, 3H, NH₃+) 8.84 (t, 1H, J=5.8 Hz, NH), 9.61 (s, 1H, NH), 12.38 (br s, 1H, COOH), 13.01 (d, 1H, J=2.4 Hz, NH) ppm. ¹³C NMR (100 MHz, DMSO-d₆): δ 28.89, 41.21, 47.86, 49.36, 53.36, 98.78, 106.42, 113.99, 115.77, 119.00, 127.15, 127.34, 129.22, 131.11, 143.03, 157.89, 167.55, 171.72 ppm. [α]_{D}²⁵+10.8 (c 0.120, MeOH). MS (ESI) m/z=526.0 ([M−H]⁻). HRMS for C₁₈H₁₈Br₂N₅O₄: calculated 525.9726. found 525.9727. HPLC: Agilent Eclipse Plus C18 column (5 μm, 4.6×150 mm); mobile phase: 20-40% of acetonitrile in phosphate buffer (pH=6.8) in 16 min, 40% acetonitrile to 20 min; flow rate 1.0 mL/min; injection volume: 10 μL; t_{R}: 5.908 min (95.1% at 280 nm).

### (Example 38) (S)-1-(5-((Carboxymethyl)carbamoyl)-2-(4,5-dibromo-1H-pyrrole-2-carboxamido)phenyl)pyrrolidin-3-aminium chloride (55b)

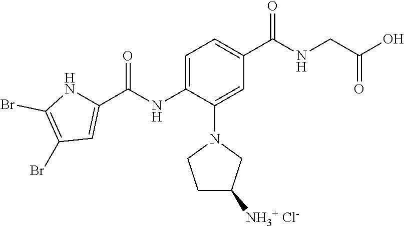

Step 1: Synthesis of methyl (S)-(3-(3-((tert-butoxycarbonyl)amino)pyrrolidin-1-yl)-4-(4,5-dibromo-1H-pyrrole-2-carboxamido)benzoyl)glycinate (51b). Synthesised according to General procedure E from 49b (0.600 g, 1.5 mmol), 4,5-dibromo-1H-pyrrole-2 carboxylic acid (493 mg, 1.8 mmol) and oxalyl chloride (0.629 mL, 7.3 mmol). The precipitate that was formed during the extraction was filtered off to obtain crude product 1. The two phases of the mother liquor were separated and organic phase was washed with water (20 mL), saturated solution of NaHCO₃ (2×20 mL) and brine (2×20 mL), dried over Na₂SO₄, filtered and the solvent evaporated to obtain crude product 2. The combined crude products were triturated with diethyl ether and the undissolved solid was filtered off and dried to give 51b (782 mg) as grey solid. Yield 797 mg (81%); brown solid; mp 213-217° C. ¹H NMR (400 MHz, DMSO-d₆): δ 1.36 (s, 9H, tBu), 1.76-1.84 (m, 1H, CH), 2.03-2.11 (m, 1H, CH), 3.04-3.08 (m, 1H, CH), 3.21-3.27 (m, 1H, CH), 3.31-3.43 (m, 2H, 2×CH, overlapping with the signal for water), 3.66 (s, 3H, CH₃), 3.99-4.07 (m, 3H, CH, CH₂), 7.14-7.17 (m, 2H, NH, ArH), 7.29-7.35 (m, 3H, 3×ArH), 8.91 (t, 1H, J=5.8 Hz, NH), 9.53 (s, 1H, NH), 12.89 (d, 1H, J=2.7 Hz, NH) ppm. [α]_{D}²⁵−26.5 (c 0.147, MeOH). MS (ESI) m/z=640 ([M−H]⁻). HRMS for C₂₄H₂₈Br₂N₅O₆: calculated 640.0406. found 640.0414.

Step 2: Synthesis of (S)-(3-(3-((tert-butoxycarbonyl)amino)pyrrolidin-1-yl)-4-(4,5-dibromo-1H-pyrrole-2-carboxamido)benzoyl)glycine (53b). To the solution of 51b (250 mg, 0.38 mmol) in methanol (10 mL) 1 M NaOH (1.52 mL, 1.52 mmol) was added and the mixture was stirred at rt for 15 h. The mixture was neutralized with 1 M HCl and methanol was removed under reduced pressure. The pH was adjusted to 4 with 1 M HCl, ethyl acetate was added, and the organic phase was washed with water (10 mL) and brine (2×10 mL), dried over Na₂SO₄, filtered and the solvent removed. To the residue diethyl ether was added, the obtained suspension was sonicated and precipitate was filtered to obtain 53b (188 mg) as grey solid. Yield 188 mg (77%); grey solid; mp 219-223° C. ¹H NMR (400 MHz, DMSO-d₆): δ 1.37 (s, 9H, tBu), 1.75-1.84 (m, 1H, CH), 2.03-2.11 (m, 1H, CH), 3.04-3.08 (m, 1H, CH), 3.21-3.27 (m, 1H, CH), 3.31-3.43 (m, 2H, 2×CH, overlapping with the signal for water), 3.91 (d, 2H, J=5.8 Hz, CH₂), 4.01-4.06 (m, 1H, CH), 7.13-7.20 (m, 1H, ArH), 7.29-7.35 (m, 3H, 3×ArH), 8.80 (t, 1H, J=5.8 Hz, NH), 9.52 (s, 1H, NH), 12.47 (br s, 1H, COOH), 12.89 (s, 1H, NH) ppm. [α]_{D}²⁵−37.3 (C 0.126, MeOH). MS (ESI) m/z=626.0 ([M−H]⁻). HRMS for C₂₃H₂₆Br₂N₅O₆: calculated 626.0250. found 626.0239.

Step 3: Synthesis of (S)-1-(5-((carboxymethyl)carbamoyl)-2-(4,5-dibromo-1H-pyrrole-2-carboxamido)phenyl)pyrrolidin-3-aminium chloride (55a). Suspension of 53a (70 mg, 0.11 mmol) in 4 M HCl in 1,4-dioxane (5 mL) was stirred at rt for 2 h. The percipitate was filtered off and washed with diethyl ether and dried to obtain 55a (48 mg) as grey solid. Yield 48 mg (76%); grey solid; mp 209-212° C. ¹H NMR (400 MHz, DMSO-d₆): δ 1.94-2.04 (m, 1H, CH), 2.20-2.29 (m, 1H, CH), 3.01-3.07 (m, 1H, CH), 3.20-3.23 (m, 1H, CH), 3.49-3.53 (m, 2H, 2×CH), 3.88 (s, 1H, CH, overlapping with the signal for water), 3.91 (d, 2H, J=5.8 Hz, CH₂), 7.36 (d, 1H, J=2.4 Hz, ArH), 7.41-7.43 (m, 2H, 2×ArH), 7.50-7.52 (m, 1H, ArH), 8.22 (s, 3H, NH₃+) 8.84 (t, 1H, J=5.8 Hz, NH), 9.61 (s, 1H, NH), 12.38 (br s, 1H, COOH), 13.01 (d, 1H, J=2.4 Hz, NH) ppm. ¹³C NMR (100 MHz, DMSO-d₆): δ 29.08, 41.19, 47.74, 49.36, 53.59, 98.36, 105.84, 114.39, 115.89, 119.01, 127.03, 127.82, 129.74, 131.07, 142.56, 157.35, 166.15, 171.37 ppm. [α]_{D}²⁵−11.1 (c 0.189, MeOH). MS (ESI) m/z=526.0 ([M−H]⁻). HRMS for C₁₈H₁₈Br₂N₅O₄: calculated 525.9726. found 525.9726. HPLC: Agilent Eclipse Plus C18 column (5 μm, 4.6×150 mm); mobile phase: 20-40% of acetonitrile in phosphate buffer (pH=6.8) in 16 min, 40% acetonitrile to 20 min; flow rate 1.0 mL/min; injection volume: 10 μL; t_{R}: 5.908 min (95.2% at 280 nm).

### (Example 39) 4-(5-((Carboxymethyl)carbamoyl)-2-(4,5-dibromo-1H-pyrrole-2-carboxamido)phenyl)piperazin-1-ium chloride (55c)

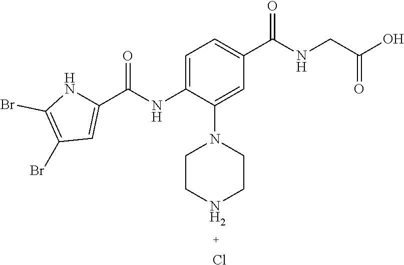 [SMILES: O=C(O)CNC(=O)c1ccc(NC(=O)c2cc(Br)c(Br)[nH]2)c(N2CCNCC2)c1 ; Cl]

Step 1: Synthesis of tert-butyl 4-(2-(4,5-dibromo-1H-pyrrole-2-carboxamido)-5-((2-methoxy-2-oxoethyl)carbamoyl)phenyl)piperazine-1-carboxylate (51c). Synthesised according to General procedure E from 49c (0.600 g, 1.5 mmol), 4,5-dibromo-1H-pyrrole-2 carboxylic acid (493 mg, 1.8 mmol) and oxalyl chloride (0.629 mL, 7.3 mmol). The precipitate that was formed during the extraction was filtered off to obtain crude product 1. The two phases of the mother liquor were separated and organic phase was washed with water (20 mL), saturated solution of NaHCO₃ (2×20 mL) and brine (2×20 mL), dried over Na₂SO₄, filtered and the solvent evaporated to obtain crude product 2. The combined crude products were triturated with diethyl ether and the undissolved solid was filtered off and dried to give 51c (801 mg) as grey solid. Yield 801 mg (83%); grey solid; mp 140-143° C. ¹H NMR (400 MHz, DMSO-d₆): δ 1.43 (s, 9H, tBu), 2.81-2.83 (m, 4H, 2×CH₂), 3.52-3.57 (m, 4H, 2×CH₂), 3.67 (s, 3H, CH₃), 4.01 (d, 2H, J=5.7 Hz, CH₂), 7.17 (d, 1H, J=2.7 Hz, ArH), 7.68 (dd, 1H, J=8.4, 1.9 Hz, ArH-4), 7.75 (d, 1H, J=1.9 Hz, ArH-6), 8.10 (d, 1H, J=8.4 Hz, ArH-3), 8.93 (t, 1H, J=5.7 Hz, NH), 9.19 (s, 1H, NH), 13.08 (d, 1H, J=2.7 Hz, NH) ppm. MS (ESI) m/z=640.0 ([M−H]⁻). HRMS for C₂₄H₂₈Br₂N₅O₆: calculated 640.0406. found 640.0403.

Step 2: Synthesis of (3-(4-(tert-butoxycarbonyl)piperazin-1-yl)-4-(4,5-dibromo-1H-pyrrole-2-carboxamido)benzoyl)glycine (53c). To the solution of 51c (200 mg, 0.32 mmol) in methanol (10 mL) 1 M NaOH (1.24 mL, 1.2 mmol) was added and the mixture was stirred at rt for 15 h. The mixture was neutralized with 1 M HCl and methanol was removed under reduced pressure. Water (10 mL) was added, the pH was adjusted to 4 with 1 M HCl, ethyl acetate (20 mL) was added, and the organic phase was washed with water (10 mL) and brine (2×10 mL), dried over Na₂SO₄, filtered and the solvent removed. To the residue diethyl ether was added, the obtained suspension was sonicated and precipitate was filtered to obtain 53c (115 mg) as grey solid. Yield 115 mg (59%); grey solid; mp>300° C. ¹H NMR (400 MHz, DMSO-d₆): δ 1.43 (s, 9H, tBu), 2.81-2.83 (m, 4H, 2×CH₂), 3.51-3.57 (m, 4H, 2×CH₂), 3.93 (d, 2H, J=5.8 Hz, CH₂), 7.17 (d, 1H, J=2.8 Hz, ArH), 7.68 (dd, 1H, J=8.5, 1.9 Hz, ArH-6), 7.76 (d, 1H, J=1.9 Hz, ArH-2), 8.10 (d, 1H, J=8.5 Hz, ArH-5), 8.81 (t, 1H, J=5.8 Hz, NH), 9.20 (s, 1H, NH), 12.59 (br s, 1H, COOH), 13.08 (s, 1H, NH) ppm. MS (ESI) m/z=626.0 ([M−H]⁻). HRMS for C₂₃H₂₆Br₂N₅O₆: calculated 626.0250. found 626.0255.

Step 3: Synthesis of 4-(5-((carboxymethyl)carbamoyl)-2-(4,5-dibromo-1H-pyrrole-2-carboxamido)phenyl)piperazin-1-ium chloride (55c). Compound 53c (50 mg, 0.079 mmol) was dissolved in 4 M HCl in 1,4-dioxane (4 mL) and THE (1 mL), and the solution was stirred at rt for 2 h. The solvents were evaporated, to the solid residue diethyl ether was added, the obtained suspension was sonicated and the solid was filtered off to give 55c (45 mg) as beige solid. Yield 45 mg (100%); beige solid; mp 225-228° C. ¹H NMR (400 MHz, DMSO-d₆): δ 3.08-3.10 (m, 4H, 2×CH₂), 3.27-3.36 (m, 4H, 2×CH₂), 3.93 (d, 2H, J=5.8 Hz, CH₂), 7.29 (d, 1H, J=2.8 Hz, ArH), 7.69-7.72 (m, 2H, ArH-4,6), 8.06 (d, 1H, J=9.0 Hz, ArH-3), 8.95 (t, 1H, J=5.8 Hz, NH), 9.19 (s, 1H, NH), 9.24 (br s, 2H, NH₂+) 12.52 (br s, 1H, COOH), 13.19 (d, 1H, J=2.8 Hz, NH) ppm. ¹³C NMR (100 MHz, DMSO-d₆): δ 41.18, 43.03, 48.23, 98.64, 106.66, 114.02, 119.54, 121.96, 123.85, 127.52, 129.71, 134.87, 142.22, 156.99, 165.67, 171.36 ppm. MS (ESI) m/z=526.0 ([M−H]⁻). HRMS for C₁₈H₁₈Br₂N₅O₄: calculated 525.9726. found 525.9727. HPLC: Agilent Eclipse Plus C18 column (5 μm, 4.6×150 mm); mobile phase: 20-40% of acetonitrile in phosphate buffer (pH=6.8) in 16 min, 40% acetonitrile to 20 min; flow rate 1.0 mL/min; injection volume: 10 μL; t_{R}: 6.884 min (98.4% at 280 nm).

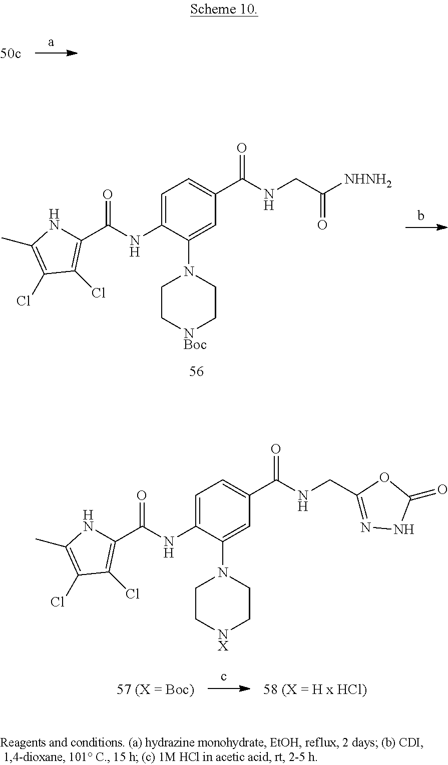

### (Example 40) 4-(2-(3,4-Dichloro-5-methyl-1H-pyrrole-2-carboxamido)-5-(((5-oxo-4,5-dihydro-1,3,4-oxadiazol-2-yl)methyl)carbamoyl)phenyl)piperazin-1-ium chloride (58)

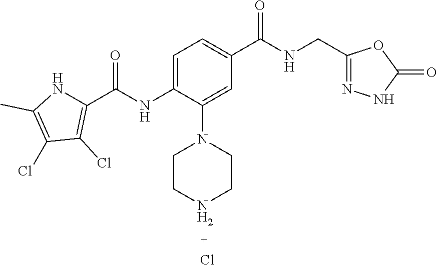 [SMILES: Cc1[nH]c(C(=O)Nc2ccc(C(=O)NCc3n[nH]c(=O)o3)cc2N2CCNCC2)c(Cl)c1Cl ; Cl]

Step 1: Synthesis of tert-butyl 4-(2-(3,4-dichloro-5-methyl-1H-pyrrole-2-carboxamido)-5-((2-hydrazineyl-2-oxoethyl)carbamoyl)phenyl)piperazine-1-carboxylate (56). To the solution of compound 50c (0.589 g, 1.0 mmol) in a mixture of methanol (10 mL) and THE (10 mL) hydrazine hydrate solution (80%, 0.50 mL, 10.2 mmol) was added and the mixture was stirred under reflux for 20 h. The solvent was removed under reduced pressure, to the residue ethanol was added and the obtained suspension was sonicated, the undissolved solid was filtered off and dried to obtain 56 (430 mg) as white solid. Yield 430 mg (76%); white solid; mp 156-160° C. ¹H NMR (400 MHz, DMSO-d₆): δ 1.44 (s, 9H, tBu), 2.24 (s, 3H, CH₃), 2.82-2.85 (m, 4H, 2×CH₂), 3.49-3.57 (m, 4H, 2×CH₂), 3.84 (d, 2H, J=5.8 Hz, CH₂), 4.67 (br s, 2H, NH₂), 7.75 (dd, 1H, J=8.6, 1.8 Hz, ArH-4), 7.92 (d, 1H, J=1.8 Hz, ArH-6), 8.46 (d, 1H, J=8.6 Hz, ArH-3), 8.71 (t, 1H, J=5.8 Hz, NH), 9.23 (br s, 1H, NH), 9.79 (s, 1H, NH), 12.43 (s, 1H, NH) ppm. MS (ESI) m/z=566.0 ([M−H]⁻). HRMS for C₂₄H₃₀Cl₂N₇O₅: calculated 566.1685. found 566.1682.

Step 2: Synthesis of tert-butyl 4-(2-(3,4-dichloro-5-methyl-1H-pyrrole-2-carboxamido)-5-(((5-oxo-4,5-dihydro-1,3,4-oxadiazol-2-yl)methyl)carbamoyl)phenyl)piperazine-1-carboxylate (57). The solution of 56 (392 mg, 0.69 mmol) and CDI (224 mg, 1.38 mmol) in a mixture of 1,4-dioxane (15 mL) and DMF (5 mL) was stirred at 101° C. for 20 h. The solvent was removed and the residue was purified with flash column chromatography using dichloromethane/methanol (10:1) as the eluent, to obtain 57 (70 mg) as white solid. Yield 70 mg (17%); white solid; mp 147-151° C. ¹H NMR (400 MHz, DMSO-d₆): δ 1.43 (s, 9H, tBu), 2.24 (s, 3H, CH₃), 2.81-2.85 (m, 4H, 2×CH₂), 3.45 (4H, 2×CH₂, overlapping with the signal for water), 4.39 (d, 2H, J=5.6 Hz, CH₂), 7.76 (dd, 1H, J=8.6, 1.9 Hz, ArH-4), 7.90 (d, 1H, J=1.9 Hz, ArH-6), 8.47 (d, 1H, J=8.6 Hz, ArH-3), 9.03 (t, 1H, J=5.8 Hz, NH), 9.79 (s, 1H, NH), 12.29 (s, 1H, NH), 12.43 (s, 1H, NH) ppm. MS (ESI) m/z=592.0 ([M−H]⁻). HRMS for C₂₅H₂₈Cl₂N₇O₆: calculated 592.1478. found 592.1474.

Step 3: Synthesis of 4-(2-(3,4-dichloro-5-methyl-1H-pyrrole-2-carboxamido)-5-(((5-oxo-4,5-dihydro-1,3,4-oxadiazol-2-yl)methyl)carbamoyl)phenyl)piperazin-1-ium chloride (58). The solution of 57 (65 mg, 0.12 mmol) in a mixture of 1,4-dioxane (10 mL) and 4 M HCl in 1,4-dioxane (6 mL) was stirred for 2 h at rt. The percipitate was filtered off and dried to obtain 58 (46 mg) as grey solid. Yield 46 mg (79%); grey solid; mp 258-262° C. ¹H NMR (400 MHz, DMSO-d₆): δ 2.24 (s, 3H, CH₃), 3.06-3.12 (m, 4H, 2×CH₂), 3.24-3.31 (m, 4H, 2×CH₂), 4.39 (d, 2H, J=5.6 Hz, CH₂), 7.78 (dd, 1H, J=8.6, 1.8 Hz, ArH-4), 7.84 (d, 1H, J=1.9 Hz, ArH-6), 8.46 (d, 1H, J=8.6 Hz, ArH-3), 9.02 (br s, 2H, NH₂+), 9.18 (t, 1H, J=5.6 Hz, NH) 9.60 (s, 1H, NH), 12.34 (s, 1H, NH), 12.46 (s, 1H, NH) ppm. ¹³C NMR (100 MHz, DMSO-d₆): δ 10.72, 35.06, 43.53, 48.83, 108.66, 110.02, 118.76, 118.86, 120.91, 125.41, 128.31, 129.87, 136.63, 140.31, 154.42, 154.87, 156.60, 165.69 ppm. MS (ESI) m/z=492.1 ([M−H]⁻). HRMS for C₂₀H₂₀Cl₂N₇O₄: calculated 492.0954. found 492.0959. HPLC: Agilent Eclipse Plus C18 column (5 μm, 4.6×150 mm); mobile phase: 20-40% of acetonitrile in phosphate buffer (pH=6.8) in 16 min, 40% acetonitrile to 20 min; flow rate 1.0 mL/min; injection volume: 10 μL; t_{R}: 13.668 min (96.7% at 280 nm).

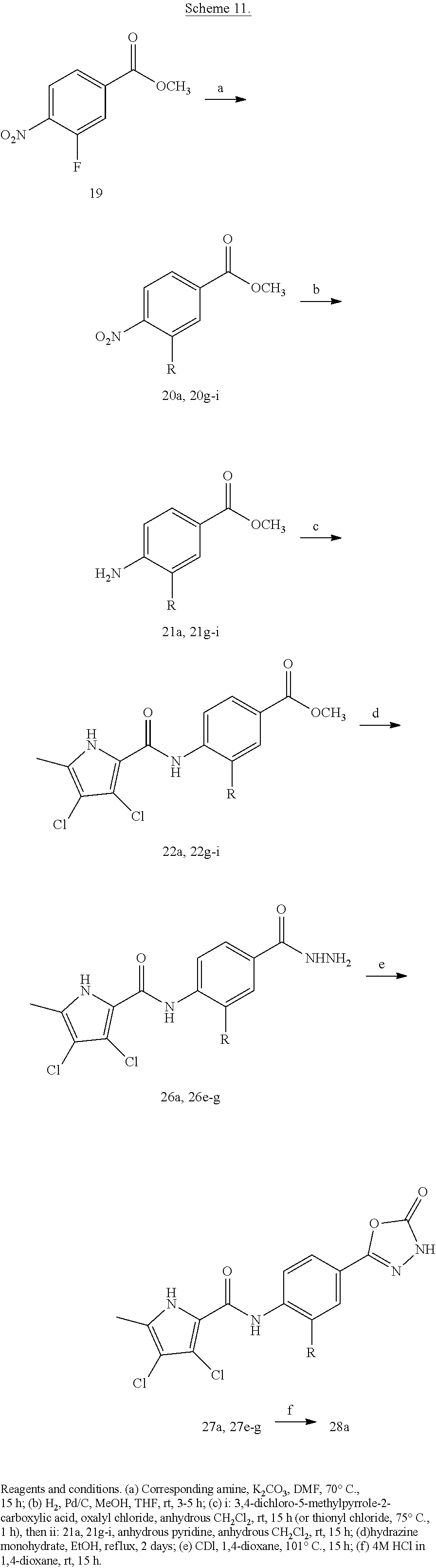

### (Example 41) 4-(2-(3,4-Dichloro-5-methyl-1H-pyrrole-2-carboxamido)-5-(5-oxo-4,5-dihydro-1,3,4-oxadiazol-2-yl)phenyl)piperazin-1-ium chloride (28a)

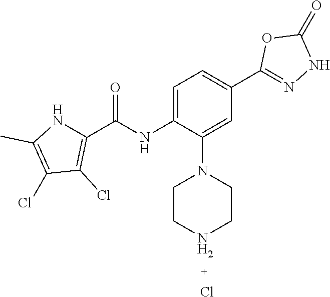 [SMILES: Cc1[nH]c(C(=O)Nc2ccc(-c3n[nH]c(=O)o3)cc2N2CCNCC2)c(Cl)c1Cl ; Cl]

Step 1: Synthesis of tert-butyl 4-(2-(3,4-dichloro-5-methyl-1H-pyrrole-2-carboxamido)-5-(hydrazinecarbonyl)phenyl)piperazine-1-carboxylate (26a). To the solution of 22a (0.71 g, 1.38 mmol) in a mixture of MeOH (20 mL) and THE (10 mL) in a high-pressure tube hydrazine hydrate (64%, 4.71 mL, 96.8 mmol) was added. The tube was sealed and reaction mixture was stirred at 120° C. overnight. The tube was cooled down to rt and the precipitate was filtered off and dried, to obtain 26a (0.579 g) as white solid. Yield 82% (0.579 g); white solid; mp 277-280° C. ¹H NMR (400 MHz, DMSO-d₆): δ 1.44 (s, 9H, tBu), 2.24 (s, 3H, CH₃), 2.77-2.85 (m, 4H, 2×CH₂), 3.47-3.57 (m, 4H, 2×CH₂), 4.47 (s, 2H, NH₂), 7.70 (d, 1H, J=8.6H, ArH), 7.84 (s, 1H, ArH), 8.43 (d, 1H, J=8.6 Hz, ArH), 9.71 (s, 1H, NH), 9.74 (s, 1H, NH), 12.42 (s, 1H, NH) ppm.

Step 2: Synthesis of tert-butyl 4-(2-(3,4-dichloro-5-methyl-1H-pyrrole-2-carboxamido)-5-(5-oxo-4,5-dihydro-1,3,4-oxadiazol-2-yl)phenyl)piperazine-1-carboxylate (27a). To a solution of compound 26a (0.500 g, 0.980 mmol) in a mixture of DMF (30 mL) and 1,4-dioxane (15 mL) 1,1′-carbonyldiimidazole (0.640 g, 3.93 mmol) was added and the reaction mixture was stirred at 101° C. overnight. The solvent was removed under reduce pressure, the crude product was sequentially triturated with acetonitrile, water and THF, the undissolved solid was filtered off and dried. The crude product was crystallized from DMF and dried to obtain 27a (226 mg) as a white solid. Yield 43% (226 mg); white solid; mp 260-263° C. ¹H NMR (400 MHz, DMSO-d₆): δ 1.44 (s, 9H, tBu), 2.24 (s, 3H, CH₃), 2.80-2.88 (m, 4H, 2×CH₂), 3.52 (s, 4H, 2×CH₂), 7.64 (d, 1H, J=8.5 Hz, ArH), 7.69 (s, 1H, ArH), 8.53 (d, 1H, J=8.6 Hz, ArH), 9.73 (s, 1H, NH), 12.45 (s, 1H, NH), 12.50 (s, 1H, NH) ppm. ¹³C NMR (100 MHz, DMSO-d₆): δ 11.24, 28.54, 44.32, 52.22, 79.56, 109.12, 110.33, 119.22, 119.53, 120.07, 123.49, 130.42, 136.92, 142.34, 153.97, 154.24, 154.93, 157.02 ppm. Peaks of two aromatic carbons overlapping. HRMS for C₂₃H₂₇O₅N₆Cl₂ ([M+H]⁺): calculated 537.14099. found 537.14145. HPLC (10-90% ACN in 0.1% TFA in 11 min, UPLC): tᵣ 6.560 min (97.45% at 224 nm, 96.79% at 280 nm).

Step 3: Synthesis of 4-(2-(3,4-dichloro-5-methyl-1H-pyrrole-2-carboxamido)-5-(5-oxo-4,5-dihydro-1,3,4-oxadiazol-2-yl)phenyl)piperazin-1-ium chloride (28a). To a solution of 27a (60.0 mg, 0.110 mmol) in DMF (6 mL) 4 M HCl in 1,4-dioxane (6 mL) was added and the reaction mixture was stirred at rt for 15 h. The solvent was removed under reduced pressure and the solid residue was triturated with acetonitrile, the undissolved solid was filtered off and dried, to obtain 28a (47.4 mg) as a white solid. Yield 91% (47.4 mg); white solid; mp 287-289° C. ¹H NMR (400 MHz, DMSO-d₆): δ 2.25 (s, 3H, CH₃), 3.08-3.15 (m, 4H, 2×CH₂), 3.23-3.31 (m, 4H, 2×CH₂), 7.62 (s, 1H, ArH), 7.68 (d, 1H, J=8.5 Hz, ArH), 8.53 (dd, 1H, J=8.7, 1.3 Hz, ArH), 9.17 (s, 2H, NH₂+), 9.60 (s, 1H, NH), 12.51 (s, 1H, NH), 12.63 (s, 1H, NH) ppm. ¹³C NMR (100 MHz, DMSO-d₆): δ 11.24, 43.89, 49.15, 109.17, 110.65, 118.76, 119.22, 119.58, 120.42, 123.88, 130.36, 136.88, 141.54, 153.80, 154.88, 157.08 ppm. HRMS for C18H19O3N6Cl2 ([M+H]⁺): calculated 437.08880. found 437.08902. HPLC (10-90% ACN in 0.1% TFA in 11 min, UPLC): tᵣ 3.107 min (96.56% at 224 nm, 95.85% at 280 nm).

### (Example 42) 3,4-Dichloro-5-methyl-N-(2-morpholino-4-(5-oxo-4,5-dihydro-1,3,4-oxadiazol-2-yl)phenyl)-1H-pyrrole-2-carboxamide (27e)

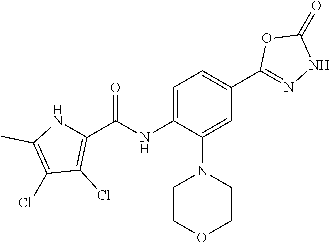 [SMILES: Cc1[nH]c(C(=O)Nc2ccc(-c3n[nH]c(=O)o3)cc2N2CCOCC2)c(Cl)c1Cl]

Step 1: Synthesis of methyl 3-morpholino-4-nitrobenzoate (20g). Synthesised according to General procedure A from 19 (3.30 g, 16.6 mmol), morpholine (1.45 mL, 16.6 mmol) and K₂CO₃ (2.75 g, 19.9 mmol). Yield 94% (4.15 g); orange solid; mp 72-78° C. ¹H NMR (400 MHz, DMSO-d₆): δ 3.00-3.06 (m, 4H, 2×CH₂), 3.67-3.72 (m, 4H, 2×CH₂), 3.89 (s, 3H, CH₃), 7.66 (dd, 1H, J=8.4, 1.7 Hz, ArH), 7.78 (d, J=1.7 Hz, 1H, ArH), 7.94 (d, 1H, J=8.4 Hz, ArH) ppm.

Step 2: Synthesis of methyl 4-amino-3-morpholinobenzoate (21g). To the solution of compound 20g (4.00 g, 15.0 mmol) in a mixture of methanol (190 mL) and tetrahydrofuran (80 mL) under an argon atmosphere Pd—C (400 mg) was added, the mixture was saturated with hydrogene and stirred under a hydrogene atmosphere at rt for 3 h. The catalyst was filtered off and the solvent was removed under reduced pressure to obtain 21g (3.51 g) as white solid. Yield 99% (3.51 g); white solid; mp 122-124° C. ¹H NMR (400 MHz, DMSO-d₆): δ 2.74-2.82 (m, 4H, 2×CH₂), 3.75-3.81 (mz, 7H, 2×CH₂, CH₃), 5.68 (s, 2H, NH₂), 6.70 (d, 1H, J=8.3 Hz, ArH), 7.45 (d, 1H, J=2.0 Hz, ArH), 7.48 (dd, 1H, J=8.2, 1.9 Hz, ArH) ppm.

Step 3: Synthesis of methyl 4-(3,4-dichloro-5-methyl-1H-pyrrole-2-carboxamido)-3-morpholinobenzoate (22g). To the 3,4-dichloro-5-methyl-1H-pyrrole-2-carboxylic acid (0.65 g, 3.38 mmol) SOCl₂ (8.28 mL, 114.15 mmol) was added and the mixture was stirred at 75° C. for 1 h under argon atmosphere. The solvent was evaporated using the water pump, then toluene (50 mL) and 21g (0.66 g, 2.81 mmol) were added. The reaction mixture was stirred overnight at 130° C. under argon atmosphere. The solvent was evaporated under reduced pressure, the crude residue was suspended in EtOAc and filtered off to obtain a brown solid. The solid was suspended again in MeOH, filtered off and dried to obtain 22g as a light-brown solid. Yield 70% (0.811 g); light-brown solid; mp 274-280° C. ¹H NMR (400 MHz, DMSO-d₆): δ 2.25 (s, 3H, CH₃), 2.87 (t, 4H, J=4.5 Hz, 2×CH₂), 3.79 (t, 4H, J=4.4 Hz, 2×CH₂), 3.85 (s, 3H, CH₃), 7.80-7.87 (m, 1H, ArH), 7.87-7.93 (m, 1H, ArH), 8.52 (d, 1H, J=8.6 Hz, ArH), 9.82 (s, 1H, NH), 12.45 (s, 1H, NH) ppm.

Step 4: Synthesis of 3,4-dichloro-N-(4-(hydrazinecarbonyl)-2-morpholinophenyl)-5-methyl-1H-pyrrole-2-carboxamide (26e). To the solution of 22g (0.70 g, 1.70 mmol) in a mixture of MeOH (15 mL) and THE (10 mL) in a high-pressure tube hydrazine hydrate (64%, 5.77 mL, 119 mmol) was added. The tube was sealed and the reaction mixture was stirred at 120° C. overnight. The tube was cooled to rt and the precipitate was filtered off and dried. The crude product was triturated with methanol, the undissolved solid was filtered off and dried, to obtain 26e (0.519 g) as an off-white solid. Yield 74% (0.519 g); off-white solid; mp 281-283° C.

¹H NMR (400 MHz, DMSO-d₆): δ 2.24 (s, 3H, CH₃), 2.84-2.89 (m, 4H, 2×CH₂), 3.77-3.82 (m, 4H, 2×CH₂), 4.47 (s, 2H, NH₂), 7.69 (dd, 1H, J=8.5, 1.9 Hz, ArH), 7.85 (d, 1H, J=1.9 Hz, ArH), 8.44 (d, 1H, J=8.5 Hz, ArH), 9.74 (s, 1H, NH), 9.76 (s, 1H, NH), 12.42 (s, 1H, NH) ppm.

Step 5: Synthesis of 3,4-dichloro-5-methyl-N-(2-morpholino-4-(5-oxo-4,5-dihydro-1,3,4-oxadiazol-2-yl)phenyl)-1H-pyrrole-2-carboxamide (27e). To a solution of compound 26e (400 mg, 0.970 mmol) in a mixture of DMF (20 mL) and 1,4-dioxane (20 mL) 1,1′-carbonyldiimidazole (310 mg, 1.94 mmol) was added and the reaction mixture was stirred at 101° C. overnight. The solvent was removed under reduce pressure, the crude product was sequentially triturated with acetonitrile, water and MeOH, and the undissolved solid was filtered off and dried. The crude product was crystallized from DMF and dried to obtain 27e (42.9 mg) as a white solid. Yield 11% (42.9 mg); white solid; mp 304-305° C. ¹H NMR (400 MHz, DMSO-d₆): δ 2.24 (s, 3H, CH₃), 2.84-2.95 (m, 4H, 2×CH₂), 3.72-2.84 (m, 4H, 2×CH₂), 7.64 (dd, 1H, J=8.5, 1.9 Hz, ArH), 7.70 (d, 1H, J=2.0 Hz, ArH), 8.54 (d, 1H, J=8.6 Hz, ArH), 9.75 (s, 1H, NH), 12.44 (s, 1H, NH), 12.54 (s, 1H, NH) ppm. ¹³C NMR (100 MHz, DMSO-d₆): δ 11.24, 52.64, 67.00, 109.11, 110.28, 118.95, 119.23, 119.57, 120.09, 123.39, 130.42, 136.97, 142.35, 153.97, 154.93, 157.02 ppm. HRMS for C₁₈H₁₈O₄N₅Cl₂ ([M+H]⁺): calculated 438.07237. found 438.07304. HPLC (10-90% ACN in 0.1% TFA in 11 min, UPLC): tr 5.333 min (97.38% at 224 nm, 97.45% at 280 nm).

### (Example 43) 3,4-Dichloro-5-methyl-N-(2-(2-methylmorpholino)-4-(5-oxo-4,5-dihydro-1,3,4-oxadiazol-2-yl)phenyl)-1H-pyrrole-2-carboxamide (27f)

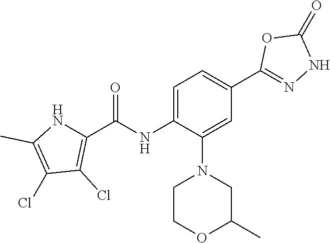 [SMILES: Cc1[nH]c(C(=O)Nc2ccc(-c3n[nH]c(=O)o3)cc2N2CCOC(C)C2)c(Cl)c1Cl]

Step 1: Synthesis of methyl 3-(2-methylmorpholino)-4-nitrobenzoate (20h). Synthesised according to General procedure A from 19 (1.77 g, 8.90 mmol), 2-methylmorpholine (0.90 mL, 8.90 mmol) and K₂CO₃ (1.48 g, 10.7 mmol). Yield 93% (2.32 g); orange solid; mp 62-65° C. ¹H NMR (400 MHz, DMSO-d₆): δ 1.11 (d, 3H, J=6.2 Hz, CH₃), 2.62-2.68 (m, 1H, CH), 2.89-2.96 (m, 1H, CH), 2.99-3.05 (m, 1H, CH), 3.07-3.13 (m, 1H, CH), 3.55-3.67 (m, 2H, 2×CH), 3.81-3.88 (m, 1H, CH), 3.90 (s, 3H, CH₃), 7.65 (d, 1H, J=8.4 Hz, ArH), 7.77 (s, 1H, ArH), 7.94 (d, 1H, J=8.4 Hz, ArH) ppm.

Step 2: Synthesis of methyl 4-amino-3-(2-methylmorpholino)benzoate (21h). To the solution of compound 20h (2.10 g, 7.49 mmol) in a mixture of methanol (90 mL) and tetrahydrofuran (40 mL) under an argon atmosphere Pd—C (210 mg) was added, the mixture was saturated with hydrogene and stirred under a hydrogene atmosphere at rt for 5 h. The catalyst was filtered off and the solvent was removed under reduced pressure obtain 21h (1.80 g) as white solid. Yield 96% (1.80 g); white solid; mp 111-114° C. ¹H NMR (400 MHz, DMSO-d₆): δ 1.10 (d, 3H, J=6.2 Hz, CH₃), 2.32 (t, 1H, J=10.5 Hz, CH), 2.57 (td, 1H, J=11.3, 3.3 Hz, CHz), 2.84-2.94 (m, 2H, 2×CH), 3.74 (s, 5H, 2×CH, CH₃), 3.80-3.87 (m, 1H, CH), 5.68 (s, 2H, NH₂), 6.70 (d, 1H, J=8.3 Hz, ArH), 7.44 (s, 1H, ArH), 7.46-7.52 (m, 1H, ArH) ppm.

Step 3: Synthesis of methyl 4-(3,4-dichloro-5-methyl-1H-pyrrole-2-carboxamido)-3-(2-methylmorpholino)benzoate (22h). Synthesised according to General procedure E from 3,4-dichloro-5-methyl-1H-pyrrole-2-carboxylic acid (0.82 g, 4.20 mmol) and 21h (0.700 g, 2.80 mmol). The suspension formed in the reaction mixture was filtered off and the solvent of the filtrate was evaporated under reduced pressure. The crude product was suspended in EtOAc (6 mL) and water (6 mL), filtered off and washed with EtOAc to obtain 22h (227 mg) as a pale-brown solid. Yield 19% (227 mg); white solid; mp 232-235° C. ¹H NMR (400 MHz, DMSO-d₆): δ 1.12 (d, 3H, J=6.1 Hz, CH₃), 2.24 (s, 3H, CH₃), 2.52-2.58 (m, 1H, CH), 2.76-2.85 (m, 2H, 2×CH), 2.89 (d, 1H, J=11.1 Hz, CH), 3.68-3.80 (m, 2H, 2×CH), 3.85 (s, 3H, CH₃), 3.90 (d, 1H, J=11.3 Hz, CH), 7.80-7.89 (m, 2H, 2×ArH), 8.53 (d, 1H, J=8.6 Hz, ArH), 9.83 (s, 1H, NH), 12.45 (s, 1H, NH) ppm.

Step 4: Synthesis of 3,4-dichloro-N-(4-(hydrazinecarbonyl)-2-(2-methylmorpholino)phenyl)-5-methyl-1H-pyrrole-2-carboxamide (26f). To the solution of 22h (420 mg, 0.985 mmol) in a mixture of MeOH (15 mL) and THE (10 mL) in a high-pressure tube hydrazine hydrate (64%, 3.34 mL, 68.6 mmol) was added. The tube was sealed and the reaction mixture was stirred at 120° C. overnight. The tube was cooled to rt and the precipitate was filtered off and dried, to obtain 26f (311 mg) as a white solid. Yield 74% (311 mg); white solid; mp 282-284° C. ¹H NMR (400 MHz, DMSO-d₆): δ 1.12 (d, 3H, J=6.1 Hz, CH₃), 2.24 (s, 3H, CH₃), 2.53-2.60 (m, 1H, CH), 2.74-2.92 (m, 3H, 3×CH), 3.69-3.79 (m, 2H, 2×CH), 3.87-3.98 (m, 1H, CH), 4.47 (s, 2H, NH₂), 7.69 (d, 1H, J=8.6 Hz, ArH), 7.84 (s, 1H, ArH), 8.44 (d, 1H, J=8.6 Hz, ArH), 9.74 (s, 1H, NH), 9.76 (s, 1H, NH), 12.42 (s, 1H, NH) ppm.

Step 5: Synthesis of 3,4-dichloro-5-methyl-N-(2-(2-methylmorpholino)-4-(5-oxo-4,5-dihydro-1,3,4-oxadiazol-2-yl)phenyl)-1H-pyrrole-2-carboxamide (27f). To a solution of compound 26f (310 mg, 0.703 mmol) in a mixture of DMF (20 mL) and 1,4-dioxane (10 mL) 1,1′-carbonyldiimidazole (456 mg, 2.81 mmol) was added and the reaction mixture was stirred at 101° C. overnight. The solvent was removed under reduce pressure, the solid residue was sequentially triturated with acetonitrile, water and THF, and the undissolved solid was filtered off and dried. The crude product was crystallized from DMF and dried to obtain 27f (169 mg) as a white solid. Yield 53% (169 mg); white solid; mp 302-303° C. ¹H NMR (400 MHz, DMSO-d₆): δ 1.12 (d, 3H, J=6.2 Hz, CH₃), 2.24 (s, 3H, CH₃), 2.55-2.61 (m, 1H, CH), 2.78-2.94 (m, 3H, 3×CH), 3.69-3.81 (m, 2H, 2×CH), 3.88-3.93 (m, 1H, CH), 7.64 (dd, 1H, J=8.6, 2.0 Hz, ArH), 7.68 (d, 1H, J=2.0 Hz, ArH), 8.55 (d, 1H, J=8.6 Hz, ArH), 9.75 (s, 1H, NH), 12.44 (s, 1H, NH), 12.56 (s, 1H, NH) ppm. ¹³C NMR (100 MHz, DMSO-d₆): δ 11.24, 19.26, 52.09, 58.59, 66.74, 72.15, 109.10, 110.26, 119.03, 119.24, 119.53, 120.00, 123.41, 130.41, 137.05, 142.15, 153.97, 154.93, 157.03 ppm. HRMS for C₁₉H₁₈O4N₅Cl₂ ([M−H]⁻): calculated 450.07428. found 450.07413. HPLC (10-90% ACN in 0.1% TFA in 11 min, UPLC): tr 5.703 min (95.50% at 224 nm, 95.08% at 280 nm).

### (Example 44) 3,4-Dichloro-N-(2-(2,6-dimethylmorpholino)-4-(hydrazinecarbonyl)phenyl)-5-methyl-1H-pyrrole-2-carboxamide (27g)

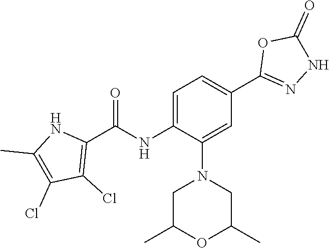 [SMILES: Cc1[nH]c(C(=O)Nc2ccc(-c3n[nH]c(=O)o3)cc2N2CC(C)OC(C)C2)c(Cl)c1Cl]

Step 1: Synthesis of methyl 3-(2,6-dimethylmorpholino)-4-nitrobenzoate (20i). Synthesised according to General procedure A from 19 (2.00 g, 10.0 mmol), 2,6-dimethylmorpholine (1.24 mL, 10.0 mmol) and K₂CO₃ (1.66 g, 12.1 mmol). Yield 99% (2.91 g); orange solid; mp 72-77° C. ¹H NMR (400 MHz, DMSO-d₆): δ 1.11 (d, 6H, J=6.2 Hz, 2×CH₃), 2.54-2.62 (m, 2H, 2×CH), 3.07-3.11 (m, 2H, 2×CH), 3.62-3.75 (m, 2H, 2×CH), 3.90 (s, 3H, CH₃), 7.64 (dd, 1H, J=8.3, 1.7 Hz, ArH), 7.77 (d, 1H, J=1.8 Hz, ArH), 7.95 (d, 1H, J=8.5 Hz, ArH) ppm.

Step 2: Synthesis of methyl 4-amino-3-(2,6-dimethylmorpholino)benzoate (21i). To the solution of compound 20i (2.50 g, 8.49 mmol) in a mixture of methanol (90 mL) and tetrahydrofuran (40 mL) under an argon atmosphere Pd—C (250 mg) was added, the mixture was saturated with hydrogene and stirred under a hydrogene atmosphere at rt for 3 h. The catalyst was filtered off and the solvent was removed under reduced pressure and the crude product was recrystallized from EtOAc to obtain 21i (0.628 g) as white solid. Yield 28% (0.628 g); white solid; mp 137-142° C. ¹H NMR (400 MHz, DMSO-d₆): δ 1.10 (d, 6H, J=6.2 Hz, 2×CH₃), 2.17-2.27 (m, 2H, 2×CH), 2.89-2.95 (m, 2H, 2×CH), 3.74 (s, 3H, CH₃), 3.77-3.85 (m, 2H, 2×CH), 5.66 (s, 2H, NH₂), 6.69 (d, 1H, J=8.3 Hz, ArH), 7.42 (d, 1H, J=1.9 Hz, ArH), 7.47 (dd, 1H, J=8.3, 1.9 Hz, ArH) ppm.

Step 3: Synthesis of methyl 4-(3,4-dichloro-5-methyl-1H-pyrrole-2-carboxamido)-3-(2,6-dimethylmorpholino)benzoate (22i). Synthesised according to General procedure E from 3,4-dichloro-5-methyl-1H-pyrrole-2-carboxylic acid (220 mg, 1.13 mmol) and 21i (200 mg, 0.760 mmol). The suspension formed in the reaction mixture was filtered off, washed with dichloromethan and dried to obtain 22i (288 mg) as a white solid. Yield 86% (288 mg); white solid; mp 277-279° C. ¹H NMR (400 MHz, DMSO-d₆): 1.12 (d, 6H, J=6.3 Hz, 2×CH₃), 2.24 (s, 3H, CH₃), 2.42-2.47 (m, 2H, 2×CH), 2.85-2.89 (m, 2H, 2×CH), 3.78-3.84 (m, 2H, 2×CH), 3.85 (s, 3H, CH₃), 7.83 (dd, 1H, J=8.6, 2.0 Hz, ArH), 7.85 (d, 1H, J=2.0 Hz, ArH), 8.54 (d, 1H, J=8.5 Hz, ArH), 9.84 (s, 1H, NH), 12.46 (s, 1H, NH) ppm.

Step 4: Synthesis of 3,4-dichloro-N-(4-(hydrazinecarbonyl)-2-morpholinophenyl)-5-methyl-1H-pyrrole-2-carboxamide (26g). To the solution of 22i (329 mg, 0.747 mmol) in a mixture of MeOH (10 mL) and THE (7 mL) in a high-pressure tube hydrazine hydrate (64%, 2.54 mL, 52.3 mmol) was added. The tube was sealed and the reaction mixture was stirred at 120° C. overnight. The tube was cooled to rt and the precipitate was filtered off and dried, to obtain 26g (244 mg) as a white solid. Yield 70% (244 mg); white solid; mp>300° C. ¹H NMR (400 MHz, DMSO-d₆): δ 1.12 (d, 6H, J=6.2 Hz, 2×CH₃), 2.24 (s, 3H, CH₃), 2.44-2.48 (m, 2H, 2×CH), 2.82-2.86 (m, 2H, 2×CH), 3.77-3.85 (m, 2H, 2×CH), 4.47 (s, 2H, NH₂), 7.69 (dd, 1H, J=8.7, 1.9 Hz, ArH), 7.83 (d, 1H, J=2.0 Hz, ArH), 8.46 (d, 1H, J=8.6 Hz, ArH), 9.73 (s, 1H, NH), 9.78 (s, 1H, NH), 12.37 (s, 1H, NH) ppm.

Step 5: Synthesis of 3,4-dichloro-N-(2-(2,6-dimethylmorpholino)-4-(5-oxo-4,5-dihydro-1,3,4-oxadiazol-2-yl)phenyl)-5-methyl-1H-pyrrole-2-carboxamide (27g). To the solution of compound 26g (217 mg, 0.492 mmol) in a mixture of DMF (8 mL) and 1,4-dioxane (15 mL) 1,1′-carbonyldiimidazole (319 mg, 1.97 mmol) was added and the reaction mixture was stirred at 101° C. overnight. The solvent was removed under reduce pressure, the solid residue was sequentially triturated with acetonitrile, MeOH and THF, and the undissolved solid was filtered off and dried. The crude product was crystallized from DMF and dried to obtain 27g (112 mg) as a white solid. Yield 49% (112 mg); white solid; mp 301-303° C. ¹H NMR (400 MHz, DMSO-d₆): δ 1.12 (d, 6H, J=6.1 Hz, 2×CH₃), 2.24 (s, 3H, CH₃), 2.43-2.48 (m, 2H, 2×CH), 2.86-2.93 (m, 2H, 2×CH), 3.77-3.86 (m, 2H, 2×CH), 7.61-7.71 (m, 2H, 2×ArH), 8.57 (d, 1H, J=8.6 Hz, ArH), 9.79 (s, 1H, NH), 12.45 (s, 1H, NH), 12.57 (s, 1H, NH) ppm. ¹³C NMR (100 MHz, DMSO-d₆): δ 11.24, 19.25, 58.08, 71.98, 109.08, 110.23, 119.13, 119.27, 119.50, 119.91, 123.45, 130.41, 137.14, 141.93, 153.97, 154.94, 157.04 ppm. HRMS for C₂₀H₂₂O₄N₅Cl₂ ([M+H]⁺): calculated 466.10399. found 466.10434. HPLC (10-90% ACN in 0.1% TFA in 11 min, UPLC): 6.077 min (98.33% at 224 nm, 98.27% at 280 nm).

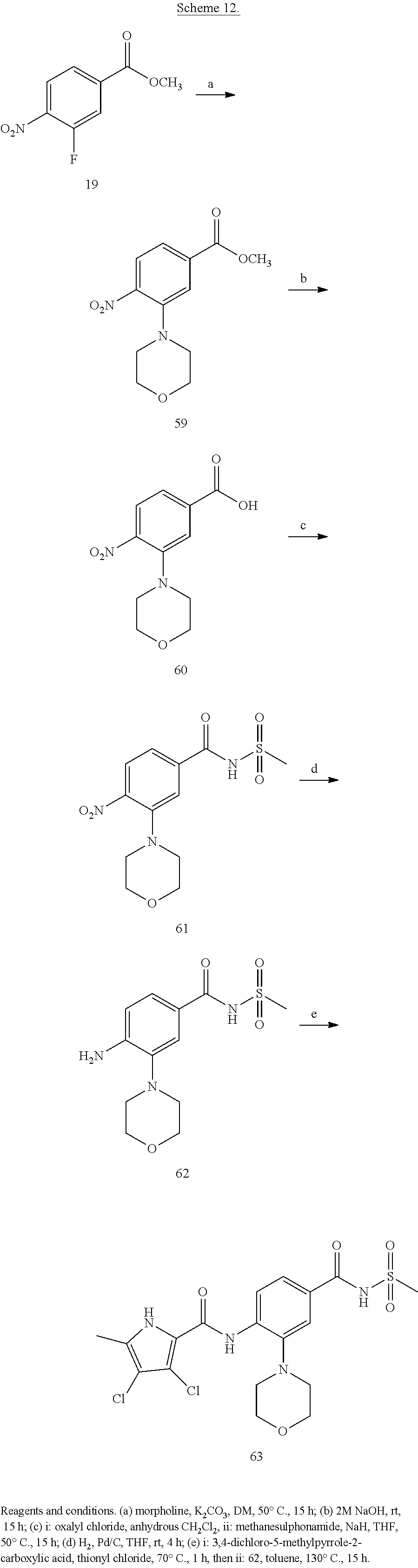

### (Example 45) 3,4-Dichloro-5-methyl-N-(4-((methylsulfonyl)carbamoyl)-2-morpholinophenyl)-1H-pyrrole-2-carboxamide (63)

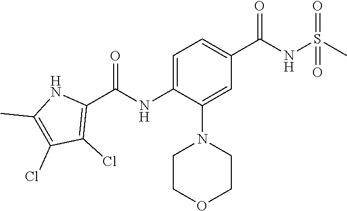

Step 1: Synthesis of methyl 3-morpholino-4-nitrobenzoate (59). To the solution of compound 19 (5.02 g, 25.2 mmol, 1 eq.) in DMF (100 mL) K₂CO₃ (6.93 g, 50.1 mmol, 2 eq.) and morpholine (2.4 mL, 27.7 mmol, 1.1 eq.) were added and the mixture was stirred at 50° C. overnight. The solvent was removed under reduced pressure and the residue was dissolved in EtOAc (140 mL) and water (100 mL). The phases were separated and the organic phase was washed with water (80 mL) and brine (2×80 mL), dried over Na₂SO₄, filtered and the solvent evaporated to obtain 59 (5.87 g) as orange solid. Yield 5.87 g (87%); orange solid; mp 75-77° C. ¹H NMR (400 MHz, DMSO-d₆): δ 3.00-3.07 (m, 4H, 2×CH₂), 3.66-3.74 (m, 4H, 2×CH₂), 3.90 (s, 3H, CH₃), 7.67 (dd, 1H, J=8.4, 1.7 Hz, ArH), 7.78 (d, 1H, J=1.7 Hz, ArH), 7.94 (d, 1H, J=8.4 Hz, ArH) ppm. MS (ESI) m/z=266.8 ([M+H]⁺).

Step 2: Synthesis of 3-morpholino-4-nitrobenzoic acid (60). To the solution of 59 (2.01 g, 7.55 mmol, 1 eq.) in MeOH (100 mL) 2 M NaOH (15.1 mL, 30.2 mmol, 4 eq) was added and the mixture was stirred overnight at rt. The solvent was removed under reduced pressure and to the residue water (70 mL) was added and the pH was adjusted to pH 2-3 with 2 M HCl (approximately 15 mL). The water phase was extracted with EtOAc (2×150 mL) and the combined organic phases were washed with brine (100 mL), dried over Na₂SO₄, filtered and the solvent evaporated to obtain 60 (1.68 g) as orange solid. Yield 1.68 g (88%); orange solid; mp 145-149° C. ¹H NMR (400 MHz, DMSO-d₆): δ 2.98-3.07 (m, 4H, 2×CH₂), 3.64-3.75 (m, 4H, 2×CH₂), 7.65 (dd, 1H, J=8.4, 1.7 Hz, ArH), 7.77 (d, 1H, J=1.7 Hz, ArH), 7.91 (d, 1H, J=8.4 Hz, ArH), 13.58 (s, 1H, COOH) ppm. MS (ESI) m/z=250.8 ([M−H]⁻).

Step 3: Synthesis of N-(methylsulfonyl)-3-morpholino-4-nitrobenzamide (61). To the solution of 60 (0.916 g, 3.63 mmol, 1 eq.) in anhydrous dichloromethane (40 mL) oxalyl chloride (1.27 mL, 14.5 mmol, 4 eq) was added dropwise and the mixture was stirred overnight at rt. The solvent was removed under reduced pressure and the obtained 3-morpholino-4-nitrobenzoyl chloride was dried. In a separate flask, methanesulphonamide (0.747 g, 7.85 mmol) was dissolved in anhydrous THE (11 mL) and to this solution, cooled on an ice bath, NaH (314 mg, 13.1 mmol, 60% dispersion in mineral oil) was added in small portions and the mixture was stirred for 30 min at 0° C. To this mixture, a solution of 3-morpholino-4-nitrobenzoyl chloride in anhydrous THE (15 mL) was added dropwise, and the obtained solution was stirred for 1 h at rt and then at 50° C. overnight. The solvent was removed under reduced pressure, and the residue was dissolved in EtOAc (50 mL) and 0.5 M HCl (15 mL) to reach pH=1-2. The phases were separated and the organic phase was washed with 0.5 M HCl (10 mL), brine (20 mL), dried over Na₂SO₄, filtered and the solvent evaporated. The crude product was triturated with diethyl ether and the undissolved solid was filtered off and dried to obtain 61 (0.554 g) as orange solid. Yield 0.554 g (43%); orange solid; mp 180-182° C. ¹H NMR (400 MHz, DMSO-d₆): δ 3.02-3.08 (m, 4H, 2×CH₂), 3.40 (s, 3H, CH₃), 3.67-3.74 (m, 4H, 2×CH₂), 7.61 (dd, 1H, J=8.4, 1.8 Hz, ArH), 7.81 (d, 1H, J=1.8 Hz, ArH), 7.93 (d, 1H, J=8.4 Hz, ArH), 12.44 (s, 1H, NH) ppm. MS (ESI) m/z=327.9 ([M−H]⁻).

Step 4: Synthesis of 4-amino-N-(methylsulfonyl)-3-morpholinobenzamide (62). The solution of 61 (0.644 g, 1.96 mmol) in THE (40 mL) was stirred for 15 min under an argon atmosphere. Pd/C (124 mg) was added, the solution was saturated with hydrogen and the reaction mixture was stirred for 4 h under hydrogen atmosphere. The catalyst was filtered off and the solvent was evaporated to obtain 62 (184 mg) as pale brown solid. Yield 184 mg (31%); pale brown solid; mp 215-219° C. ¹H NMR (400 MHz, DMSO-d₆): δ 2.75-2.84 (m, 4H, 2×CH₂), 3.32 (s, 3H), 3.73-3.80 (m, 4H, 2×CH₂), 5.75 (s, 2H, NH₂), 6.69 (d, 1H, J=8.4 Hz, ArH), 7.51 (dd, 1H, J=8.4, 2.1 Hz, ArH), 7.57 (d, 1H, J=2.1 Hz, ArH), 11.60 (s, 1H, NH) ppm. MS (ESI) m/z=297.9 ([M−H]⁻).

Step 5: Synthesis of 3,4-dichloro-5-methyl-N-(4-((methylsulfonyl)carbamoyl)-2-morpholinophenyl)-1H-pyrrole-2-carboxamide (63). A mixture of 3,4-dichloro-5-methyl-1H-pyrrole-2-carboxylic acid (99 mg, 0.510 mmol) and thionyl chloride (2 mL) was stirred at 70° C. under argon atmosphere for 1 h. Excess thionyl chloride was evaporated in vacuo. To the residue 62 (100 mg, 0.334 mmol) and toluene (10 mL) were added, and the reaction mixture was stirred at 130° C. overnight. The solvent was removed in vacuo, the solid residue was triturated with diethyl ether and the undissolved solid was filtered off and dried. The crude product was triturated with acetonitrile and the undissolved solid was filtered off and dried to obtain 63 (150 mg) as pale brown solid. Yield 150 mg (95%); pale brown solid; mp>250° C. ¹H NMR (400 MHz, DMSO-d₆): δ 2.25 (s, 3H, pyrrole-CH₃), 2.85-2.92 (m, 4H, 2×CH₂), 3.39 (s, 3H, CH₃), 3.74-3.84 (m, 4H, 2×CH₂), 7.84 (dd, 1H, J=8.7, 2.1 Hz, ArH), 8.05 (d, 1H, J=2.1 Hz, ArH), 8.51 (d, 1H, J=8.7 Hz, ArH), 9.86 (s, 1H, NH), 12.12 (s, 1H, NH), 12.50 (s, 1H, NH) ppm. ¹³C NMR (100 MHz, DMSO-d₆): δ 11.26, 41.89, 52.79, 67.03, 109.15, 110.47, 119.11, 119.21, 122.89, 126.66, 127.15, 130.55, 138.79, 141.52, 157.11, 165.82 ppm. HRMS for C₁₈H₂₁O₅N₄Cl₂S ([M+H]⁺): calculated 475.06042. found 475.06044. HPLC: Waters Acquity UPLC BEH C18 (1.7 μm, 2.1×50 mm); mobile phase: 10-90% of acetonitrile in TFA (0.1%) in 10 min; flow rate 0.4 mL/min; injection volume: 1.75 μL; t_{R}: 5.080 min (95.28% at 280 nm).

### Biological Data

### Enzyme Inhibition Assays

Inhibitory activities for compounds of the present invention were determined on E. coli and S. aureus DNA gyrase and topoisomerase IV in an assay from Inspiralis on streptavidin-coated 96-well microtiter plates from Thermo scientific Pierce. First, the plates were rehydrated with buffer (20 mM Tris-HCl with pH 7.6, 0.01% w/v BSA, 0.05% v/v Tween 20, 137 mM NaCl) and the biotinylated oligonucleotide was then immobilized. After washing off the unbound oligonucleotide, the enzyme test was performed. The reaction volume of 30 μL in buffer (35 mM Tris×HCl with pH 7.5, 4 mM MgCl₂, 24 mM KCl, 2 mM DTT, 1.8 mM spermidine, 1 mM ATP, 6.5% w/v glycerol, 0.1 mg/mL albumin for DNA gyrase assays or 40 mM HEPES KOH with pH 7.6, 100 mM potassium glutamate, 10 mM magnesium acetate, 10 mM DTT, 1 mM ATP, 0.05 mg/mL albumin for topoisomerase IV assays) contained 1.5 U of DNA gyrase or topoisomerase IV from E. coli or S. aureus, 0.75 μg of relaxed pNO1 plasmid, and 3 μL solution of the inhibitor in 10% DMSO and 0.008% Tween 20. Reaction solutions were incubated at 37° C. for 30 min. After that, the TF buffer (50 mM NaOAc with pH 5.0, 50 mM NaCl and 50 mM MgCl₂) was added to terminate the enzymatic reaction. After additional incubation for 30 min at rt, during which biotin-oligonucleotide-plasmid triplex was formed, the unbound plasmid was washed off using TF buffer and SybrGOLD in T10 buffer (10 mM Tris HCl with pH 8.0 and 1 mM EDTA) was added. The fluorescence was measured with a microplate reader (BioTek Synergy H4, excitation: 485 nm, emission: 535 nm). Initial screening was done at 100 or 10 nM concentration of inhibitors against E. coli DNA gyrase or 1 μM or 100 nM concentration against S. aureus DNA gyrase and E. coli and S. aureus topo IV. For the most active inhibitors IC₅₀ was determined using seven concentrations of tested compounds. GraphPad Prism 6.0 software was used to calculate the IC₅₀ values. The result is given as the average value of three independent measurements. As the positive control novobiocin (IC₅₀=0.168 μM and 0.041 μM (lit. 0.08 μM and 0.01 μM (Alt S. et. al. J. Antimicrob. Chemoth. 66, 2061-2096 (2011))) for E. coli and S. aureus gyrase and IC₅₀=11.1 μM and 26.7 μM (lit. 10 μM and 20 μM (Alt S. et. al. J. Antimicrob. Chemoth. 66, 2061-2096 (2011))) for E. coli and S. aureus topoisomerase IV) was used.

Inhibitory activities of compounds described in Examples 1-46 of the present invention against DNA gyrase from E. coli, DNA gyrase from S. aureus, DNA topoisomerase IV from E. coli and DNA topoisomerase IV from S. aureus (Table 1).

TABLE 1
IC₅₀^{a} or RA^{b} values of example compounds 1-40

| Example | E. coli | S. aureus | E. coli | S. aureus |
|---|---|---|---|---|
| No. | DNA gyrase | DNA gyrase | topo IV | topo IV |
| Example 1 | IC₅₀ = 0.253 nM | N.D. | RA (1 μM) = 33% | N.D. |
| | | | RA (0.1 μM) = 105% | |
| Example 2 | IC₅₀ = 2.49 nM | IC₅₀ = 55.8 nM | IC₅₀ = 0.145 μM | N.D. |
| Example 3 | IC₅₀ = 3.19 nM | IC₅₀ = 24.1 nM | IC₅₀ = 0.0418 μM | N.D. |
| Example 4 | IC₅₀ = 9.66 nM | IC₅₀ = 249 nM | IC₅₀ = 1.18 μM | N.D. |
| Example 5 | IC₅₀ = 6.07 nM | N.D. | N.D. | N.D. |
| Example 6 | IC₅₀ = 12.6 nM | N.D. | N.D. | N.D. |
| Example 7 | IC₅₀ = 109 nM | N.D. | N.D. | N.D. |
| Example 8 | IC₅₀ = 13.0 ± 5.9 nM | N.D. | N.D. | RA (1 μM) = 10% |
| | | | | RA (0.1 μM) = 51% |
| Example 9 | IC₅₀ = 2.16 nM | IC₅₀ = 35.0 nM | IC₅₀ = 0.0603 μM | N.D. |
| Example 10 | IC₅₀ = 44.5 nM | N.D. | N.D. | RA (1 μM) = 7% |
| | | | | RA (0.1 μM) = 101% |
| Example 11 | IC₅₀ = 886 nM | N.D. | RA (10 μM) = 106% | N.D. |
| Example 12 | RA (1 μM) = 67% | N.D. | RA (10 μM) = −18% | RA (10 μM) = −17% |
| Example 13 | IC₅₀ = 6.34 ± 4.49 nM | RA (0.1 μM) = 28% | N.D. | RA (0.1 μM) = 0% |
| | | RA (0.01 μM) = 125% | | |
| Example 14 | RA (1 μM) = −2% | N.D. | N.D. | N.D. |
| | RA (0.1 μM) = 52% | | | |
| Example 15 | IC₅₀ = 532 nM | N.D. | N.D. | N.D. |
| Example 16 | IC₅₀ = 199 nM | N.D. | N.D. | N.D. |
| Example 17 | N.D. | N.D. | N.D. | N.D. |
| Example 18 | IC₅₀ = 27.2 nM | RA (1 μM) = 11% | N.D. | IC₅₀ = 406 ± 79 nM |
| | | RA (0.1 μM) = 41% | | |
| Example 19 | IC₅₀ = 4.40 nM | N.D. | N.D. | N.D. |
| Example 20 | IC₅₀ = 2.80 ± 0.29 nM | RA (0.1 μM) = 39% | RA (1 μM) = 105% | RA (0.1 μM) = 99% |
| | | RA (0.01 μM) = 90% | | |
| Example 21 | RA (1 μM) = 95% | N.D. | N.D. | N.D. |
| Example 22 | IC₅₀ = 4.90 nM | IC₅₀ = 121 nM | RA (10 μM) = 26% | N.D. |
| | | | RA (1 μM) = 97% | |
| Example 23 | IC₅₀ = 3.96 nM | IC₅₀ = 216 nM | IC₅₀ = 11.5 μM | IC₅₀ = 6.20 μM |
| Example 24 | IC₅₀ = 7.80 nM | N.D. | RA (100 μM) = 91% | N.D. |
| Example 25 | RA (0.1 nM) = 26% | N.D. | RA (10 μM) = 105% | N.D. |
| | RA (0.01 nM) = 73% | | | |
| Example 26 | IC₅₀ = 3.54 nM | N.D. | N.D. | N.D. |
| Example 27 | IC₅₀ = 2.26 nM | N.D. | N.D. | N.D. |
| Example 28 | IC₅₀ = 0.750 nM | N.D. | N.D. | N.D. |
| Example 29 | RA (10 nM) = 1% | N.D. | RA (10 μM) = 103% | N.D. |
| Example 30 | IC₅₀ = 42.2 nM | N.D. | N.D. | N.D. |
| Example 31 | N.D. | N.D. | N.D. | N.D. |
| Example 32 | N.D. | N.D. | N.D. | N.D. |
| Example 33 | IC₅₀ = 3743 nM | N.D. | N.D. | N.D. |
| Example 34 | IC₅₀ = 21.3 nM | IC₅₀ = 1240 nM | IC₅₀ = 2.37 μM | IC₅₀ = 2.03 μM |
| Example 35 | IC₅₀ = 19.3 nM | IC₅₀ = 112 nM | IC₅₀ = 0.958 μM | IC₅₀ = 2.56 μM |
| Example 36 | IC₅₀ = 40.3 nM | IC₅₀ = 621 nM | RA (10 μM) = −43% | IC₅₀ = 4.19 μM |
| Example 37 | IC₅₀ = 2380 nM | RA (10 μM) = 78% | N.D. | N.D. |
| Example 38 | IC₅₀ = 35.7 nM | RA (10 μM) = 46% | RA (10 μM) = 84% | RA (10 μM) = 91% |
| Example 39 | IC₅₀ = 841 nM | N.D. | RA (10 μM) = 97% | RA (10 μM) = 73% |
| Example 40 | IC₅₀ = 22.4 nM | RA (1 μM) = 37% | IC₅₀ = 2.97 μM | IC₅₀ = 2.70 μM |
| Example 41 | IC₅₀ = 17.9 nM | N.D. | RA (10 μM) = 48% | N.D. |
| | | | RA (1 μM) = 71% | |
| Example 42 | IC₅₀ = 82.2 nM | N.D. | RA (10 μM) = 54% | N.D. |
| | | | RA (1 μM) = 66% | |
| Example 43 | IC₅₀ = 53.5 nM | N.D. | RA (10 μM) = 102% | N.D. |
| | | | RA (1 μM) = 103% | |
| Example 44 | IC₅₀ = 109 nM | N.D. | RA (10 μM) = 105% | N.D. |
| | | | RA (1 μM) = 105% | |
| Example 45 | IC₅₀ = 243 nM | N.D. | N.D. | N.D. |

^{a} Concentration of compound that inhibits the enzyme activity by 50%.
^{b} Residual activity of the enzyme at 1 μM of the compound.
^{c} Not determined.

Example compound 18 was additionally evaluated against DNA gyrase from Mycobacterium tuberculosis using a gel-based supercoiling assay. The determined IC₅₀ value was 26.5±2.1 nM.

### Antimicrobial Activity

The chemical entities described in the present invention were assessed for their antimicrobial activity using a microbroth dilution assay as recommended by the CLSI (Methods for Dilution Antimicrobial Susceptibility Tests for Bacteria That Grow Aerobically, and Methods for Antimicrobial Susceptibility Testing of Anaerobic Bacteria, Clinical and Laboratory Standards Institute, Document M07-A8, and M11-A8, Wayne, PA 19087, USA, 2012.). Compounds were dissolved by using 100% DMSO. Serial two-fold dilutions of each compound were made by using 100% DMSO as the diluent.

A library of Gram-negative and Gram-positive microbes and their drug- and multidrug-resistant variants were employed as test organisms. The MIC (minimum inhibitory concentration) was determined as the lowest concentration of an individual drug that lead to no visible growth.

The compounds of the invention have showed activity against a number of different bacterial pathogens and opportunistic bacterial pathogens (Table 2).

TABLE 2
MIC values of example compounds against selected Gram-positive and Gram-negative bacteria

| | MIC (μg/mL) | | | | | | | |
|---|---|---|---|---|---|---|---|---|
| | P. | A. | A. | K. | K. | | E. spp. | S. |
| | aeruginosa | baumannii | baumannii | pneumoniae | pneumoniae | E. coli | cloacae | aureus |
| | ATCC | ATCC | (ATCC | ATCC | (ATCC | ATCC | ATCC | ATCC |
| Example No. | 27853 | 17978 | 19606) | 10031 | 700603) | 25922 | 13047 | 29213 |
| Example 1 | ≥64 | ≥64 | N.D.^{a} | 64 | N.D. | ≥64 | ≥64 | 64 |
| Example 2 | >64 | >64 | N.D. | 16 | N.D. | >64 | >64 | >64 |
| Example 3 | >64 | >64 | N.D. | 2 | N.D. | >64 | >64 | 4 |
| Example 4 | >64 | >64 | N.D. | >64 | N.D. | >64 | >64 | >64 |
| Example 5 | >64 | >64 | N.D. | >64 | N.D. | >64 | >64 | >64 |
| Example 6 | >64 | >64 | N.D. | >64 | N.D. | >64 | >64 | >64 |
| Example 7 | >64 | >64 | N.D. | >64 | N.D. | >64 | >64 | 4 |
| Example 8 | N.D. | N.D. | N.D. | N.D. | N.D. | N.D. | N.D. | N.D. |
| Example 9 | >64 | >64 | N.D. | 64 | N.D. | >64 | >64 | 16 |
| Example 10 | ≥64 | ≥64 | ≥64 | ≥64 | ≥64 | ≥64 | ≥64 | ≥64 |
| Example 11 | ≥32 | ≥32 | N.D. | ≥32 | N.D. | ≥32 | ≥32 | ≥32 |
| Example 12 | ≥64 | N.D. | ≥64 | N.D. | ≥64 | ≥64 | N.D. | ≥64 |
| Example 13 | ≥64 | N.D. | ≥64 | N.D. | ≥64 | ≥64 | N.D. | 23 |
| Example 14 | ≥64 | N.D. | ≥64 | N.D. | ≥64 | ≥64 | N.D. | ≥64 |
| Example 15 | >64 | >64 | N.D. | >64 | N.D. | >64 | >64 | >64 |
| Example 16 | >64 | >64 | N.D. | >64 | N.D. | >64 | >64 | >64 |
| Example 17 | N.D. | N.D. | N.D. | N.D. | N.D. | N.D. | N.D. | N.D. |
| Example 18 | 64 | 16 | N.D. | 0.250 | N.D. | 16 | >64 | 2 |
| Example 19 | >64 | >64 | N.D. | 2 | N.D. | >64 | >64 | >64 |
| Example 20 | 32 | 8 | N.D. | 0.064 | N.D. | 8 | 32 | 2 |
| Example 21 | ≥64 | N.D. | ≥64 | N.D. | ≥64 | ≥64 | N.D. | ≥64 |
| Example 22 | ≥32 | ≥32 | N.D. | ≥32 | N.D. | ≥32 | ≥32 | ≥32 |
| Example 23 | ≥32 | ≥32 | N.D. | ≥32 | N.D. | ≥32 | ≥32 | ≥32 |
| Example 24 | ≥32 | ≥32 | N.D. | ≥32 | N.D. | ≥32 | ≥32 | ≥32 |
| Example 25 | >64 | >64 | N.D. | >64 | N.D. | >64 | >64 | 16 |
| Example 26 | ≥32 | ≥32 | N.D. | ≥32 | N.D. | ≥32 | ≥32 | ≥32 |
| Example 27 | ≥32 | ≥32 | N.D. | 0.125 | N.D. | ≥32 | ≥32 | 16 |
| Example 28 | >64 | >64 | N.D. | 0.5 | N.D. | >64 | >64 | >64 |
| Example 29 | ≥32 | ≥32 | N.D. | ≥32 | N.D. | ≥32 | ≥32 | ≥32 |
| Example 30 | N.D. | N.D. | N.D. | N.D. | N.D. | N.D. | N.D. | N.D. |
| Example 31 | N.D. | N.D. | N.D. | N.D. | N.D. | N.D. | N.D. | N.D. |
| Example 32 | N.D. | N.D. | N.D. | N.D. | N.D. | N.D. | N.D. | N.D. |
| Example 33 | N.D. | N.D. | N.D. | N.D. | N.D. | N.D. | N.D. | N.D. |
| Example 34 | ≥64 | N.D. | ≥64 | N.D. | ≥64 | ≥64 | N.D. | ≥64 |
| Example 35 | ≥64 | N.D. | ≥64 | N.D. | ≥64 | ≥64 | N.D. | ≥64 |
| Example 36 | ≥64 | N.D. | ≥64 | N.D. | ≥64 | ≥64 | N.D. | ≥64 |
| Example 37 | ≥64 | N.D. | ≥64 | N.D. | ≥64 | ≥64 | N.D. | ≥64 |
| Example 38 | ≥64 | N.D. | ≥64 | N.D. | ≥64 | ≥64 | N.D. | ≥64 |
| Example 39 | ≥64 | N.D. | ≥64 | N.D. | ≥64 | ≥64 | N.D. | ≥64 |
| Example 40 | ≥64 | N.D. | ≥64 | N.D. | ≥64 | ≥64 | N.D. | ≥64 |
| Example 41 | ≥64 | ≥64 | N.D. | 0.25 | N.D. | ≥64 | ≥64 | 2 |
| Example 42 | ≥64 | ≥64 | N.D. | ≥64 | N.D. | ≥64 | ≥64 | 0.25 |
| Example 43 | ≥64 | ≥64 | N.D. | ≥64 | N.D. | ≥64 | ≥64 | ≥64 |
| Example 44 | N.D. | N.D. | N.D. | N.D. | N.D. | N.D. | N.D. | N.D. |
| Example 45 | N.D. | N.D. | N.D. | N.D. | N.D. | N.D. | N.D. | N.D. |

| | MIC (μg/mL) | | | | | |
|---|---|---|---|---|---|---|
| | S. | S. | | | E. | |
| | aureus | aureus | E. | E. | faecium | E. |
| | ATCC | ATCC | faecalis | faecium | (ATCC | aerogenes |
| | 43300 | 700699 | ATCC | ATCC | 35667) | (ATCC |
| Example No. | (MRSA) | (VISA) | 29212 | 700221 | Päivi | 13408) |
| Example 1 | 32 | 16 | 32 | 32 | N.D. | N.D. |
| Example 2 | >64 | >64 | 8 | 8 | N.D. | N.D. |
| Example 3 | 4 | 2 | 0.25 | 2 | N.D. | N.D. |
| Example 4 | >64 | >64 | >64 | >64 | N.D. | N.D. |
| Example 5 | >64 | >64 | >64 | >64 | N.D. | N.D. |
| Example 6 | 0.5 | <0.03126 | <0.03125 | 0.0625 | N.D. | N.D. |
| Example 7 | 0.0625 | 2 | 0.125 | 4 | N.D. | N.D. |
| Example 8 | N.D. | N.D. | N.D. | N.D. | N.D. | N.D. |
| Example 9 | >64 | >64 | 8 | 16 | N.D. | N.D. |
| Example 10 | ≥64 | N.D. | N.D. | ≥64 | 10 | ≥64 |
| Example 11 | ≥32 | ≥32 | ≥32 | ≥32 | N.D. | N.D. |
| Example 12 | N.D. | N.D. | N.D. | N.D. | ≥64 | ≥64 |
| Example 13 | N.D. | N.D. | N.D. | N.D. | 23 | ≥64 |
| Example 14 | N.D. | N.D. | N.D. | N.D. | ≥64 | ≥64 |
| Example 15 | >64 | >64 | >64 | >64 | N.D. | N.D. |
| Example 16 | >64 | >64 | >64 | >64 | N.D. | N.D. |
| Example 17 | N.D. | N.D. | N.D. | N.D. | N.D. | N.D. |
| Example 18 | 4 | N.D. | 2 | N.D. | N.D. | N.D. |
| Example 19 | >64 | >64 | >64 | >64 | N.D. | N.D. |
| Example 20 | 2 | N.D. | 1 | N.D. | N.D. | N.D. |
| Example 21 | N.D. | N.D. | N.D. | N.D. | ≥64 | ≥64 |
| Example 22 | ≥32 | >64 | >64 | ≥32 | N.D. | N.D. |
| Example 23 | ≥32 | ≥32 | ≥32 | ≥32 | N.D. | N.D. |
| Example 24 | ≥32 | 0.5 | 0.5 | 0.5 | N.D. | N.D. |
| Example 25 | >64 | >64 | 0.25 | 0.5 | N.D. | N.D. |
| Example 26 | ≥32 | ≥32 | ≥32 | ≥32 | N.D. | N.D. |
| Example 27 | 8 | ≥64 | >64 | 8 | N.D. | N.D. |
| Example 28 | >64 | >64 | >64 | >64 | N.D. | N.D. |
| Example 29 | ≥32 | ≥32 | ≥32 | ≥32 | N.D. | N.D. |
| Example 30 | N.D. | N.D. | N.D. | N.D. | N.D. | N.D. |
| Example 31 | N.D. | N.D. | N.D. | N.D. | N.D. | N.D. |
| Example 32 | N.D. | N.D. | N.D. | N.D. | N.D. | N.D. |
| Example 33 | N.D. | N.D. | N.D. | N.D. | N.D. | N.D. |
| Example 34 | N.D. | N.D. | N.D. | N.D. | ≥64 | ≥64 |
| Example 35 | N.D. | N.D. | N.D. | N.D. | ≥64 | ≥64 |
| Example 36 | N.D. | N.D. | N.D. | N.D. | ≥64 | ≥64 |
| Example 37 | N.D. | N.D. | N.D. | N.D. | ≥64 | ≥64 |
| Example 38 | N.D. | N.D. | N.D. | N.D. | ≥64 | ≥64 |
| Example 39 | N.D. | N.D. | N.D. | N.D. | ≥64 | ≥64 |
| Example 40 | N.D. | N.D. | N.D. | N.D. | ≥64 | ≥64 |
| Example 41 | 4 | N.D. | 0.5 | N.D. | N.D. | N.D. |
| Example 42 | 0.25 | N.D. | 0.125 | N.D. | N.D. | N.D. |
| Example 43 | ≥64 | N.D. | 1 | N.D | N.D. | N.D. |
| Example 44 | N.D. | N.D. | N.D. | N.D. | N.D. | N.D. |
| Example 45 | N.D. | N.D. | N.D. | N.D. | N.D. | N.D. |

^{a}Not determined.

Activity Against Mycobacterium tuberculosis H37Rv

Activity against M. tuberculosis H37Rv was determined using a standard REMA assay (resazurin microtiter assay) to investigate bacterial viability after the treatment and incubation with compounds (Tables 3), as described (Palomino, J. C. et al. Antimicrob Agents Chemother 2002, 46, 2720). In the REMA assay, reduction of resazurin, e.i. the conversion of resazurin (blue, not fluorescent) to resorufin (pink, fluorescent) is measured, which is proportional to the number of metabolically active viable cells. Incubation time was 7 days. Rifampicin was used as control. Treatment with DMSO represents 100% viability. Minimum inhibitory concentration (MIC90) against M. tuberculosis H37Rv was determined for Example compound 18 (Table 4).

TABLE 3
Viability of Mycobacterium tuberculosis H37Rv (%) determined
in a resazurin-reduction microtiter assay (REMA).

| | Concentration (μM) | | | | |
|---|---|---|---|---|---|
| Example No. | 20 | 10 | 5 | 2.5 | 1.25 |
| Example 1 | −1.5 | −1.0 | 0.2 | 1.8 | 4.1 |
| Example 4 | N.D. | N.D. | 2.4 | 13.6 | 80.0 |
| Example 5 | N.D. | N.D. | 1.3 | 2.3 | 22.0 |
| Example 6 | N.D. | N.D. | 99.0 | 112 | 100 |
| Example 7 | N.D. | N.D. | 109 | 82.3 | 100 |
| Example 8 | 23.0 | 10.5 | 19.1 | 52.2 | 74.4 |
| Example 9 | −0.3 | 0.2 | 1.5 | 3.7 | 5.9 |
| Example 10 | N.D. | N.D. | 140 | 94.2 | 109 |
| Example 11 | N.D. | N.D. | 119 | 112 | 118 |
| Example 13 | 0.8 | 1.4 | 2.7 | 3.2 | 4.6 |
| Example 15 | N.D. | N.D. | 145 | 128 | 121 |
| Example 16 | N.D. | N.D. | 144 | 134 | 127 |
| Example 18 | −0.4 | 2.5 | 5.8 | 8.5 | 9.6 |
| Example 19 | 0.8 | 2.1 | 22.8 | 56.8 | 90.2 |
| Example 20 | −0.9 | 1.0 | 4.0 | 6.0 | 6.3 |
| Example 23 | 7.1 | 12.4 | 36.2 | 69.5 | 76.3 |
| Example 24 | 71.8 | 71.5 | 65.2 | 75.0 | 74.4 |
| Example 25 | N.D. | N.D. | 130 | 109 | 112 |
| Example 26 | 110 | 99.2 | 102 | 111 | 114 |
| Example 27 | 3.5 | 4.0 | 3.9 | 4.1 | 8.1 |
| Example 28 | 30.1 | 38.8 | 65.9 | 87.7 | 90.4 |
| Example 29 | 75.5 | 71.5 | 88.0 | 92.8 | 107 |
| Example 30 | 107 | 93.5 | 93.2 | 96.4 | 102 |
| Example 31 | 115 | 87.4 | 71.7 | 81.4 | 103 |
| Example 32 | 92.5 | 81.7 | 72.2 | 85.2 | 95.5 |
| Example 33 | 115 | 116 | 118 | 116 | 85.8 |
| Example 34 | 5.5 | 24.8 | 66.0 | 74.6 | 71.6 |
| Example 35 | N.D. | N.D. | 38.7 | 123 | 109 |
| Example 36 | N.D. | N.D. | 125 | 107 | 119 |
| Example 38 | N.D. | N.D. | 134 | 124 | 126 |
| Example 40 | 1.0 | 38.9 | 76.6 | 81.3 | 82.3 |
| Example 41 | N.D. | N.D. | 4.6 | 5.6 | 39.4 |
| Example 42 | N.D. | N.D. | 11.0 | 90.8 | 122 |
| Example 43 | N.D. | N.D. | 45.3 | 12 | 138 |
| Example 44 | N.D. | N.D. | 37.9 | 149 | 164 |

^{a} Not determined.

TABLE 4
MIC values of Example compound 22
against M. tuberculosis H37Rv

| Example No. | MIC90 (μM) |
|---|---|
| Example 18 | 2.5 |
| rifampicin | 0.0625 |
| isoniazid | 0.156 |

### Activity Against Non-Tuberculous Mycobacteria

Anti-bacterial activity of selected compounds (Example 1, 8, 9, 18) was determined against different non-tuberculous mycobacteria: Mycobacterium marinum (lab strain, model organism for Mtb), Mycobacterium smegmatis (lab strain, fast growing mycobacterium) and Mycobacterium abscessus (clinical isolate, fast growing Mycobacterium, clinical relevant organism). Resazurin-reduction assay was used to determine the bacterial viability after incubation with compounds. Incubation time for M. smegmatis was 24 h, for M. abscessus 48 h and M. marinum 4 days. Rifampicin was used as control. Treatment with DMSO represents 100% viability. Some compounds showed high activity (low μM IC₅₀) against the viability of fast growing mycobacteria: M. smegmatis and M. abscessus. The inhibition of M. marinum viability was also observed, however to a smaller extent compared to fast growing M. smegmatis and M. abscessus.

Activity Against Intracellular M. tuberculosis

Activity of selected compounds against intracellular M. tuberculosis was determined using ex vivo macrophage infection assay, where THP-1 macrophages are infected with M. tuberculosis H37Rv and treated with compounds (Table 5). In this assay, the viable bacteria express green fluorescent protein (GFP), which is measured as fluorescent signal. Additionally, the macrophage count in each treatment group is used as a readout to determine the ability of compounds to protect the infected macrophages. Treatment with active compounds results in low GFP signal (=low amount of viable bacteria) and a high number of macrophages. Such compounds are efficiently killing intracellular M. tuberculosis and protecting macrophage survival.

TABLE 5
Activity of example compounds against Mycobacterium tuberculosis
measured in THP-1 macrophages infected with
M. tuberculosis H37Rv

| | | % THP-1 |
|---|---|---|
| Example No. | % GFP^{a}-Mtb | macrophages |
| Example 1 | 7.6 | 25.8 |
| Example 8 | 7.6 | 30.4 |
| Example 9 | 7.8 | 46.4 |
| Example 13 | 9.4 | 31.9 |
| Example 18 | 4.2 | 35.0 |
| Example 19 | 14.0 | 19.3 |
| Example 20 | 5.6 | 17.0 |
| Example 23 | 33.5 | 19.6 |
| Example 24 | 71.9 | 21.0 |
| Example 27 | 9.0 | 14.4 |
| Example 34 | 46.5 | 15.3 |
| Example 40 | 38.9 | 17.5 |
| DMSO | 100.0 | 13.4 |
| rifampicin | 8.3 | 100.0 |
| isoniazid | 13.9 | 73.7 |

^{a}Green fluorescent protein.

1. The compound of formula I: 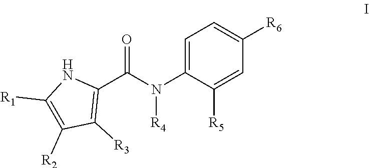
wherein:
R¹, R² and R³ are each independently selected from H, halogen, —CN, —CF₃, optionally substituted C₁₋₆ alkyl, optionally substituted C₁₋₆ alkoxy, and optionally substituted C₃₋₆ cycloalkyl;
R⁴ is (CH₂)₀₋₆-A
wherein A is H, carboxyl, NR^{6′}R^{6″} or is selected from optionally substituted monocyclic C₃₋₇ cycloalkyl, optionally substituted monocyclic C3-7 cycloalkenyl, optionally substituted saturated or unsaturated monocyclic 3-7 membered heterocycle, optionally substituted saturated or unsaturated fused bicyclic 8-10 membered-heterocycle, optionally substituted C₆-1o aryl and optionally substituted 5-10 membered heteroaryl;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1, 2 and 3;
R^{5′} is H and R^{5″} is optionally substituted 4-6-membered heterocyclyl;
R⁶ is an optionally substituted 5-membered heterocyclyl, —CONR^{6′}R^{6″} or —C(O)NHS(O)p-C₁₋₆ alkyl;
R^{6′} and R^{6″} are independently at each occurrence selected from H and optionally substituted C₁₋₆ alkyl or C₁₋₆ acyl;
or R^{6′} and R^{6″} may together with the nitrogen to which they are attached form a 5 or 6-membered heterocyclic ring, optionally substituted with 1 or 2 substituents independently selected from C₁₋₆ alkyl, C₂₋₆ alkenyl, C₂₋₆ alkynyl, hydroxyl, C₁₋₆ alkoxy, halogen, cyano, nitro, carboxyl, hydroxy-C₁₋₆ alkyl-, C₁₋₆ alkoxy-C₁₋₆ alkyl, halogeno-C₁₋₆ alkyl-, difluoromethyl, trifluoromethyl, formyl, —CO—C₁₋₆ alkyl, —COO—C₁₋₆ alkyl, —C(O)NH₂, —C(O)NH—C₁₋₆ alkyl, —C(O)N[C₁₋₆ alkyl]₂, —SO₂NH₂, —SO₂NH—C₁₋₆ alkyl, —SO₂N[C₁₋₆ alkyl]₂ and —S(O)p-C₁₋₆ alkyl;
p is (independently at each occurrence) 0, 1 or 2;
or a pharmaceutically acceptable salt, racemate, diastereomer, enantiomer, ester, carbamate, sulphate, phosphate or prodrug thereof.

2. The compound of claim 1, wherein two of R¹, R² and R³ are not H, and at least one of said two of R¹, R² and R³, which are not H, is halogen.

3. The compound of claim 1, wherein R¹ is methyl.

4. The compound of claim 1, wherein R² and R³ are chloro or fluoro.

5. The compound of claim 1, wherein R¹ and R² are bromo and R³ is H.

6. The compound of claim 1, wherein R⁴ is H.

7. The compound of claim 1, wherein R⁵ is optionally substituted (CH₂)ₘ-5-10-membered heterocycle with m being an integer selected from 0, 1, 2 and 3, or
wherein R⁵ is optionally substituted (CH₂)ₘNR^{5′}R^{5″}; wherein R^{5′} is H and R^{5″} is optionally substituted 4-6-membered heterocyclyl, or
wherein R⁵ is selected from the group consisting of O(CH₂)ₘ-pyrrolidine, O(CH₂)ₘ-piperidine, O(CH₂)ₘ-morpholine, O(CH₂)ₘ-pyridine, O(CH₂)ₘ-pyrimidine, O(CH₂)ₘ-thiophene, (CH₂)ₘ-pyrrolidine, (CH₂)ₘ-piperidine, (CH₂)ₘ-morpholine, (CH₂)ₘ-pyridine, (CH₂)ₘ-pyrimidine, and (CH₂)ₘ-thiophene, wherein each m is an integer independently selected from 0, 1 or 2, and wherein any alkyl, heterocyclyl or aryl may be optionally substituted, or wherein the optionally substituted heterocycle in R⁵ is selected from the group consisting of piperidine, piperidin-3-amine, piperidin-4-amine, piperidin-3-ylmethanamine, piperazine, pyrrolidine, pyrrolidin-3-amine, morpholine, isoindoline and 1-phenylpiperazine.

8. (canceled)

9. (canceled)

10. (canceled)

11. (canceled)

12. The compound of claim 1, wherein R⁶ is an optionally substituted 5-membered heterocyclyl selected from the group consisting of 1,2,4-oxadiazol-5-one, tetrazole, 1,3,4-oxadiazol-2-one and 1,4-dihydro-tetrazol-5-one, or wherein R⁶ is —CONR^{6′}R^{6″} or R⁶ is —C(O)NHS(O)p-C₁₋₆ alkyl.

13. (canceled)

14. (canceled)

15. The compound of claim 1, wherein:
R¹ is methyl;
R² and R³ are each independently selected from chloro, bromo or fluoro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2,
and
R⁶ is optionally substituted 5-membered heterocyclyl selected from the group consisting of 1,2,4-oxadiazol-5-one, tetrazole, 1,3,4-oxadiazol-2-one and 1,4-dihydro-tetrazol-5-one.

16. (canceled)

17. (canceled)

18. (canceled)

19. The compound of claim 1, wherein:
R¹ is methyl;
R² and R³ are each independently selected from chloro, bromo or fluoro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2; and
R⁶ is —CONR^{6′}R^{6″}.

20. (canceled)

21. (canceled)

22. (canceled)

23. The compound of claim 1, wherein:
R¹ is methyl;
R² and R³ are each independently selected from chloro, bromo or fluoro;
R⁴ is hydrogen;
R⁵ is optionally substituted (CH₂)ₘO(CH₂)ₘ-5-10-membered heterocycle, optionally substituted (CH₂)ₘ-5-10-membered heterocycle or optionally substituted (CH₂)ₘNR^{5′}R^{5″}, wherein each m is an integer independently selected from 0, 1 and 2; and
R⁶ is —C(O)NHS(O)p-C₁₋₆ alkyl.

24. (canceled)

25. (canceled)

26. (canceled)

27. The compound of claim 1, wherein:
R¹ is methyl;
R² and R³ are each independently selected from chloro, bromo or fluoro;
R⁴ is hydrogen;
R⁵ is selected from the group consisting of O(CH₂)ₘ-pyrrolidine, O(CH₂)ₘ-piperidine, O(CH₂)ₘ-morpholine, O(CH₂)ₘ-pyridine, O(CH₂)ₘ-pyrimidine, O(CH₂)ₘ-thiophene, (CH₂)ₘ-pyrrolidine, (CH₂)ₘ-piperidine, (CH₂)ₘ-morpholine, (CH₂)ₘ-pyridine, (CH₂)ₘ-pyrimidine, and (CH₂)ₘ-thiophene, wherein each m is an integer independently selected from 0, 1 or 2, and wherein any alkyl, heterocyclyl or aryl may be optionally substituted; and
R⁶ is optionally substituted 5-membered heterocyclyl selected from the group consisting of 1,2,4-oxadiazol-5-one, tetrazole, 1,3,4-oxadiazol-2-one and 1,4-dihydro-tetrazol-5-one.

28. (canceled)

29. (canceled)

30. The compound of claim 1, wherein:
R¹ is methyl;
R² and R³ are each independently selected from chloro, bromo or fluoro;
R⁴ is hydrogen;
R⁵ is optionally substituted heterocyclyl selected from the group consisting of piperidine, piperidin-3-amine, piperidin-4-amine, piperidin-3-ylmethanamine, piperazine, pyrrolidine, pyrrolidin-3-amine, morpholine, isoindoline and 1-phenylpiperazine;
R⁶ is optionally substituted 5-membered heterocyclyl selected from the group consisting of 1,2,4-oxadiazol-5-one, tetrazole, 1,3,4-oxadiazol-2-one and 1,4-dihydro-tetrazol-5-one.

31. The compound of claim 1, wherein:
R¹ is methyl;
R² and R³ are chloro;
R⁴ is hydrogen;
R⁵ is optionally substituted heterocyclyl selected from the group consisting of piperidine, piperidin-3-amine, piperidin-4-amine, piperidin-3-ylmethanamine, piperazine, pyrrolidine, pyrrolidin-3-amine, morpholine, isoindoline and 1-phenylpiperazine; and
R⁶ is optionally substituted 5-membered heterocyclyl selected from the group consisting of 1,2,4-oxadiazol-5-one, tetrazole, 1,3,4-oxadiazol-2-one or 1,4-dihydro-tetrazol-5-one.

32. The compound according to claim 1, which is selected from the group consisting of:
1-(2-(3,4-Dichloro-5-methyl-1H-pyrrole-2-carboxamido)-5-(5-oxo-4,5-dihydro-1,2,4-oxadiazol-3-yl)phenyl)piperidin-4-aminium chloride,
1-(2-(3,4-Dichloro-5-methyl-1H-pyrrole-2-carboxamido)-5-(5-oxo-4,5-dihydro-1,2,4-oxadiazol-3-yl)phenyl)piperidin-3-aminium chloride,
(1-(2-(3,4-Dichloro-5-methyl-1H-pyrrole-2-carboxamido)-5-(5-oxo-4,5-dihydro-1,2,4-oxadiazol-3-yl)phenyl)piperidin-3-yl)methanaminium chloride,
4-((2-(3,4-Dichloro-5-methyl-1H-pyrrole-2-carboxamido)-5-(5-oxo-4,5-dihydro-1,2,4-oxadiazol-3-yl)phenyl)amino)piperidin-1-ium chloride,
4-(2-(3,4-Dichloro-5-methyl-1H-pyrrole-2-carboxamido)-5-(5-oxo-4,5-dihydro-1,2,4-oxadiazol-3-yl)phenyl)piperazin-1-ium chloride,
3,4-Dichloro-5-methyl-N-(4-(5-oxo-4,5-dihydro-1,2,4-oxadiazol-3-yl)-2-(pyrrolidin-1-yl)phenyl)-1H-pyrrole-2-carboxamide,
3,4-Dichloro-5-methyl-N-(4-(5-oxo-4,5-dihydro-1,2,4-oxadiazol-3-yl)-2-(4-phenylpiperazin-1-yl)phenyl)-1H-pyrrole-2-carboxamide,
3,4-Dichloro-5-methyl-N-(2-morpholino-4-(5-oxo-4,5-dihydro-1,2,4-oxadiazol-3-yl)phenyl)-1H-pyrrole-2-carboxamide,
(1-(2-(3,4-Dichloro-5-methyl-1H-pyrrole-2-carboxamido)-5-(2H-tetrazol-5-yl)phenyl)piperidin-3-yl)methanaminium chloride,
3,4-Dichloro-5-methyl-N-(2-morpholino-4-(2H-tetrazol-5-yl)phenyl)-1H-pyrrole-2-carboxamide,
(S)-1-(2-(3,4-Dichloro-5-methyl-1H-pyrrole-2-carboxamido)-5-(5-oxo-4,5-dihydro-1,3,4-oxadiazol-2-yl)phenyl)piperidin-3-aminium chloride,
4-((2-(3,4-Dichloro-5-methyl-1H-pyrrole-2-carboxamido)-5-(5-oxo-4,5-dihydro-1,3,4-oxadiazol-2-yl)phenyl)amino)piperidin-1-ium chloride,
(S)-1-(2-(3,4-Dichloro-5-methyl-1H-pyrrole-2-carboxamido)-5-(5-oxo-4,5-dihydro-1,3,4-oxadiazol-2-yl)phenyl)pyrrolidin-3-aminium chloride,
(S)-1-(2-(3,4-Dichloro-5-methyl-1H-pyrrole-2-carboxamido)-5-(5-oxo-4,5-dihydro-1H-tetrazol-1-yl)phenyl)pyrrolidin-3-aminium chloride,
1-(2-(3,4-Dichloro-5-methyl-1H-pyrrole-2-carboxamido)-5-(5-oxo-4,5-dihydro-1H-tetrazol-1-yl)phenyl)piperidin-4-aminium chloride,
3,4-Dichloro-5-methyl-N-(4-(5-oxo-4,5-dihydro-1H-tetrazol-1-yl)-2-(4-phenylpiperazin-1-yl)phenyl)-1H-pyrrole-2-carboxamide,
3,4-Dichloro-N-(2-(isoindolin-2-yl)-4-(5-oxo-4,5-dihydro-1H-tetrazol-1-yl)phenyl)-5-methyl-1H-pyrrole-2-carboxamide,
3-(2-(3,4-Dichloro-5-methyl-1H-pyrrole-2-carboxamido)-5-(5-oxo-4,5-dihydro-1,3,4-oxadiazol-2-yl)phenoxy)pyrrolidin-1-ium chloride,
4-(2-(3,4-Dichloro-5-methyl-1H-pyrrole-2-carboxamido)-5-(5-oxo-4,5-dihydro-1,3,4-oxadiazol-2-yl)phenoxy)piperidin-1-ium chloride,
3-((2-(3,4-Dichloro-5-methyl-1H-pyrrole-2-carboxamido)-5-(5-oxo-4,5-dihydro-1,3,4-oxadiazol-2-yl)phenoxy)methyl)piperidin-1-ium chloride,
4-(2-(2-(3,4-Dichloro-5-methyl-1H-pyrrole-2-carboxamido)-5-(5-oxo-4,5-dihydro-1,3,4-oxadiazol-2-yl)phenoxy)ethyl)piperidin-1-ium chloride,
3,4-Dichloro-5-methyl-N-(4-(5-oxo-4,5-dihydro-1,3,4-oxadiazol-2-yl)-2-(pyridin-4-ylmethoxy)phenyl)-1H-pyrrole-2-carboxamide,
3,4-Dichloro-5-methyl-N-(4-(5-oxo-4,5-dihydro-1,3,4-oxadiazol-2-yl)-2-(pyrimidin-2-ylmethoxy)phenyl)-1H-pyrrole-2-carboxamide,
3,4-Dichloro-5-methyl-N-(4-(5-oxo-4,5-dihydro-1,3,4-oxadiazol-2-yl)-2-(thiophen-2-ylmethoxy)phenyl)-1H-pyrrole-2-carboxamide,
4-(2-(3,4-Dichloro-5-methyl-1H-pyrrole-2-carboxamido)-5-(5-oxo-4,5-dihydro-1,3,4-oxadiazol-2-yl)phenyl)piperazin-1-ium chloride,
3,4-Dichloro-5-methyl-N-(2-morpholino-4-(5-oxo-4,5-dihydro-1,3,4-oxadiazol-2-yl)phenyl)-1H-pyrrole-2-carboxamide,
3,4-dichloro-5-methyl-N-(2-(2-methylmorpholino)-4-(5-oxo-4,5-dihydro-1,3,4-oxadiazol-2-yl)phenyl)-1H-pyrrole-2-carboxamide,
3,4-Dichloro-N-(2-(2,6-dimethylmorpholino)-4-(hydrazinecarbonyl)phenyl)-5-methyl-1H-pyrrole-2-carboxamide, and
3,4-Dichloro-5-methyl-N-(4-((methylsulfonyl)carbamoyl)-2-morpholinophenyl)-1H-pyrrole-2-carboxamide.

33. (canceled)

34. (canceled)

35. (canceled)

36. A pharmaceutical composition comprising the compound of claim 1 and a pharmaceutically acceptable excipient or carrier.

37. A process for preparing the compound as defined in claim 1, which comprises:
Process step a) reacting a compound of formula III: 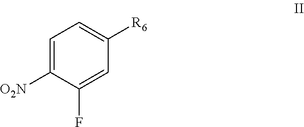
with compound of formula III: 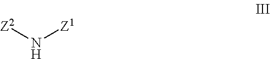
wherein Z¹ and Z² are independently selected from H, —R^{6′}R^{6″}, —CH(CO₂R^{6″})R^{6′}, heterocyclyl, aryl,
to a compound of formula IV: 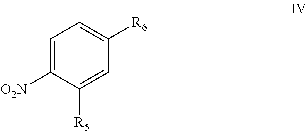
or
Process step b) coupling a compound of formula V: 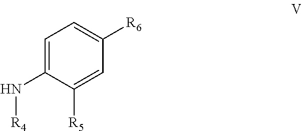
with compound of formula VI: 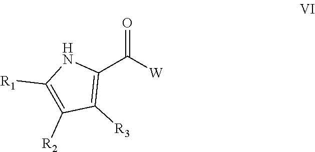
wherein W is selected from hydroxyl, halogen and 1,1,1-trichloromethyl,
to a compound of formula I: 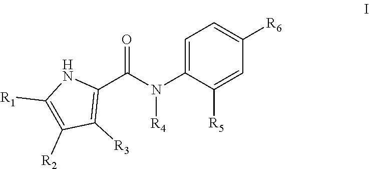
or
Process step c) reacting a compound of formula Ia: 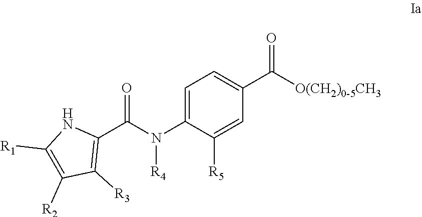
to a compound of formula Ib: 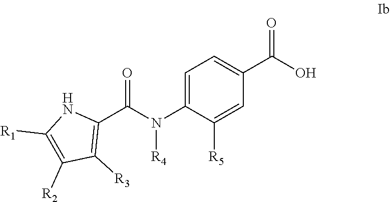
or
Process step d) reacting a compound of formula Ic: 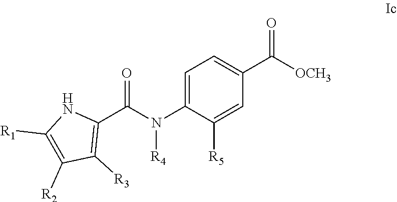
to a compound of formula Id: 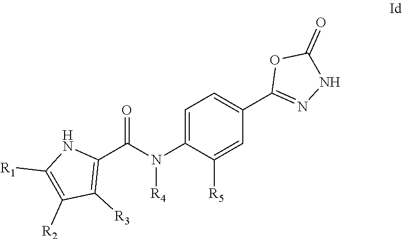
in a two-step procedure, using hydrazine monohydrate (step 1) and 1,1′-carbonyldiimidazole (step 2), as reagents;
Process step e) reacting a compound of formula Ie: 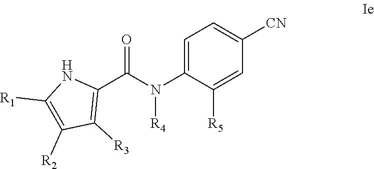
to a compound of formula If: 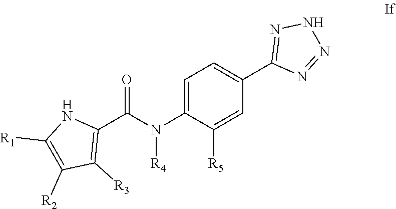
in a procedure using sodium azide as reagents.

38. A method for treating a bacterial infection in a warm-blooded animal comprising administering the compound of claim 1 to the warm-blooded animal.

39. The compound of claim 15, wherein R² and R³ are chloro.

40. The compound of claim 19, wherein R² and R³ are chloro.

41. The compound of claim 23, wherein R² and R³ are chloro.

42. The compound of claim 27, wherein R² and R³ are chloro.
